(19) United States
(12) Patent Application Publication
(10) Pub. No.: US 2026/0234130 A1
(43) Pub. Date: Aug. 13, 2026

(54) EP2 Antagonist Compounds

(71) Applicant: Reservoir Neuroscience, Inc., San Francisco, CA (US)

(72) Inventors: Matthew Alexander James Duncton, San Francisco, CA (US); Vladimir V. Senatorov, San Francisco, CA (US); Aaron R. Friedman, San Francisco, CA (US); Steven Howard Olson, San Francisco, CA (US)

(21) Appl. No.: 19/477,755

(22) PCT Filed: Dec. 22, 2023
(86) PCT No.: PCT/US2023/085848

§ 371 Date: Oct. 22, 2025

Related U.S. Application Data
(60) Provisional application No. 63/435,738, filed on Dec. 28, 2022.
(60) Provisional application No. 63/435,730, filed on Dec. 28, 2022.

(51) Int. Cl. C07D 403/12; A61K 31/4025; C07D 207/08; C07D 207/12; C07D 207/16

(52) U.S. Cl. CPC C07D 403/12; A61K 31/4025; C07D 207/08; C07D 207/12; C07D 207/16

(57) ABSTRACT

Disclosed herein are compounds that are EP2 antagonists, methods of making such compounds, pharmaceutical compositions and medicaments comprising such compounds, and methods of using such compounds in the treatment of diseases or conditions associated with EP2 activity.

### CROSS REFERENCE TO RELATED APPLICATION

This application claims the benefit of the earlier filing date of U.S. provisional patent application Nos. 63/435,730 and 63/435,738, both filed Dec. 28, 2022, both of which are incorporated herein by reference in their entireties.

### FIELD

Described herein are compounds that are inhibitors of prostaglandin E2 receptor 2, also known as EP2, methods of making such compounds, pharmaceutical compositions and medicaments comprising such compounds, and methods of using such compounds in the treatment of diseases or conditions associated with EP2 activity.

### BACKGROUND

EP2 is a prostaglandin receptor that functions, for example, as a mediator of inflammation. EP2 signaling is implicated in, for example, inflammatory conditions, allergic diseases, ocular diseases, nervous system diseases, bone diseases, fibrotic conditions, cardiovascular diseases, and certain forms of cancer.

### SUMMARY

Compounds described herein are antagonists of EP2. In some embodiments, the compounds described herein are used in the treatment or prevention of diseases or conditions in which EP2 activity contributes to the symptomology or progression of the disease or condition, such as, for example, inflammatory diseases or conditions. The disclosed EP2 antagonist compounds have useful pharmaceutical properties. In particular, one of skill in the art, upon consideration of the present disclosure, will recognize that EP2 antagonist compounds as disclosed herein are useful in ameliorating the pathological consequences of EP2 activation in inflammation, and in many other disorders. Such pathological consequences include but are not limited to, immune cell activation, cell growth and proliferation, cell metabolism, cell signaling, cellular oxidation state and cellular stress response, cellular aging, cellular transport, atherosclerosis, vascular health and blood-brain barrier integrity, neurological function, glial function, and neurodegeneration.

In another aspect, described herein is a pharmaceutical composition comprising a compound described herein, or a pharmaceutically acceptable salt, or solvate thereof, and at least one pharmaceutically acceptable excipient. In some embodiments, the pharmaceutical composition is formulated for administration to a mammal by intravenous administration, subcutaneous administration, oral administration, inhalation, or nasal administration. In some embodiments, the pharmaceutical composition is in the form of a tablet, a pill, a capsule, a liquid, a suspension, a dispersion, a solution, or an emulsion.

In another aspect, described herein is a method of modulating the activity of the prostaglandin E2 receptor 2 (EP2) in a mammal comprising administering to the mammal a compound described herein, or a pharmaceutically acceptable salt, or solvate thereof.

In yet another aspect, described herein is a method of treating a disease or condition that would benefit from the modulation of prostaglandin E2 receptor 2 (EP2) activity comprising administering to the mammal a compound described herein, or a pharmaceutically acceptable salt, or solvate thereof.

The foregoing and other objects, features, and advantages of the invention will become more apparent from the following detailed description.

### DETAILED DESCRIPTION

Prostaglandins act on prostaglandin receptors such as the prostaglandin DP1 receptor (DP1), prostaglandin DP2 receptor (DP2), prostaglandin EP1 receptor (EP1), prostaglandin EP2 receptor (EP2), prostaglandin EP3 receptor (EP3), prostaglandin EP4 receptor (EP4), prostaglandin F2α receptor (FP1), prostacyclin I2 receptor (IP), and thromboxane A2 receptor (TP), or a combination thereof.

Prostaglandin E2 (PGE2) is a metabolite of arachidonic acid, synthesized by the action of cyclooxygenase and prostaglandin E synthase. PGE2, which is produced in nearly all organs and tissues, has a variety of physiological effects, including mucosal protection, induction of gastric acid secretion in stomach, generation of fever, hyperalgesia, inflammation and immunity. The actions of PGE₂ are mediated by four receptors, EP1, EP2, EP3 and EP4. PGE2 has affinity not only for all four EP receptor subtypes but also for other prostanoid receptors, such as the PGD2 DP1 receptor.

PGE2 is a downstream product of the cyclooxygenase 2 (COX-2) pathway and a major modulator of inflammation.

At the cellular level, EP2 is activated and/or upregulated in response to many types of injuries, stressors, and other disease signals, and its activation causes damage or pathology that contributes to disease symptoms (Sluter, M. N. et al. EP2 Antagonists (2011-2021): A Decade's Journey from Discovery to Therapeutics. J. Med. Chem. 64, 11816-11836 (2021)).

EP2 is a G-protein coupled receptor that, when bound to PGE2, mobilizes Gₛ proteins and initiates signaling cascades involving adenylyl cyclase (and thereby elevates cAMP) and protein kinase A (PKA). Coupling of EP2 to Gₛ proteins stimulates adenylate cyclase and their activation increases intracellular cAMP levels. This signaling pathway has implications for inflammation, pain, immunoregulation, mitogenesis, plasticity, and cell injury. EP2 also interacts with β-arrestin/JNK pathways, which pathway can affect proliferation and metastasis.

Expression of EP2 receptors has been demonstrated in a broad range of cell types and tissues, including brain, lung, gastrointestinal tract, kidney, uterus, myeloid and thymus; and has been linked with PGE2-mediated vasodilation and smooth muscle relaxation in pulmonary, gastrointestinal and reproductive tracts.

One of the effects of EP2 activation is to induce inflammation that can damage or otherwise impair the function of affected cells, tissues, and organ systems. For example, EP2 signaling has been shown to activate immune cells in aging humans and mice (Minhas, P. S. et al. Restoring metabolism of myeloid cells reverses cognitive decline in ageing. Nature (2021) doi:10.1038/s41586-020-03160-0), which in turn contribute to impairments of various biological functions in the aging brain, including metabolic dysfunction and inflammation, that are associated with cognitive decline. Accordingly treating EP2-mediated inflammation using the compounds is effective in treating age-related dementias such as Alzheimer's disease. Similarly, antagonizing EP2 activity as described herein reduces inflammation and mediates symptom progression. For example, antagonizing EP2 has been shown to reduce poor outcomes in mice treated with lipopolysaccharide, which is a rodent model of the human condition of sepsis that is caused by pathogen infection (Jiang, C., Caskurlu, A., Ganesh, T. & Dingledine, R. Inhibition of the prostaglandin EP2 receptor prevents long-term cognitive impairment in a model of systemic inflammation. Brain Behav. Immun.—Health 8, 100132 (2020)). Accordingly, the presently disclosed compounds can antagonize EP2 to provide therapeutic benefit in a variety of infectious or inflammatory diseases by mediating or reducing the inflammatory immune response, or “cytokine storm,” that is activated in response to infection of pathogens such as bacteria, viruses, or parasitic organisms (Sheppe, A. E. F. & Edelmann, M. J. Roles of Eicosanoids in Regulating Inflammation and Neutrophil Migration as an Innate Host Response to Bacterial Infections. Infect. Immun. 89, e0009521 (2021)). Because inflammation is a fundamental process in many types of chronic and acute diseases and disorders, there are numerous additional examples in which modulation of EP2 activity as described herein, is therapeutic, for example inflammation in cancers (Wang, D. & Dubois, R. N. Eicosanoids and cancer. Nat. Rev. Cancer 10, 181-193 (2010)), following acute injuries and vascular injuries such as head injury, stroke, and aneurysm (Liu, Q. et al. PGE2 signaling via the neuronal EP2 receptor increases injury in a model of cerebral ischemia. Proc. Natl. Acad. Sci. U.S.A 116, 10019-10024 (2019); Aoki, T. et al. Prostaglandin E2-EP2-NF-κB signaling in macrophages as a potential therapeutic target for intracranial aneurysms. Sci. Signal. 10, eaah6037 (2017)); Aoki, T. et al. PGE(2)-EP(2) signalling in endothelium is activated by haemodynamic stress and induces cerebral aneurysm through an amplifying loop via NF-κB. Br. J. Pharmacol. 163, 1237-1249 (2011); Li, P. et al. AH6809 decreases production of inflammatory mediators by PGE2-EP2-cAMP signaling pathway in an experimentally induced pure cerebral concussion in rats. Brain Res. 1698, 11-28 (2018)), and in chronic inflammatory contexts such as endometriosis, multiple sclerosis, and amyotrophic lateral sclerosis (Noble, L. S. et al. Prostaglandin E2 stimulates aromatase expression in endometriosis-derived stromal cells. J. Clin. Endocrinol. Metab. 82, 600-606 (1997); Kawahara, K., Hohjoh, H., Inazumi, T., Tsuchiya, S. & Sugimoto, Y. Prostaglandin E2-induced inflammation: Relevance of prostaglandin E receptors. Biochim. Biophys. Acta 1851, 414-421 (2015); Liang, X. et al. The prostaglandin E2 EP2 receptor accelerates disease progression and inflammation in a model of amyotrophic lateral sclerosis. Ann. Neurol. 64, 304-314 (2008)).

The compounds disclosed herein have been found to have useful pharmaceutical properties. In particular, one of skill in the art, upon consideration of the present disclosure, would recognize that EP2 modulators as disclosed herein are useful in ameliorating the pathological consequences of EP2 activation not only in inflammation, but in many other disorders because EP2 regulates many other aspects of cellular biology that affect disease outcomes, for example, including but not limited to, immune cell activation, cell growth and proliferation, cell metabolism, cell signaling, cellular oxidation state and cellular stress response, cellular aging, cellular transport, atherosclerosis, vascular health and blood-brain barrier integrity, neurological function, glial function, and neurodegeneration (Sluter, M. N. et al. EP2 Antagonists (2011-2021): A Decade's Journey from Discovery to Therapeutics. J. Med. Chem. 64, 11816-11836 (2021)).

Thus, administration of the presently disclosed small molecule antagonists of EP2 has a therapeutic benefit in a broad range of diseases by affecting or improving underlying biological processes that cause disease pathology and symptoms.

In some embodiments, compounds described herein modulate the activity of EP2. In some embodiments, compounds described herein inhibit or reduce the magnitude of inflammatory PGE2 signaling through the EP2 receptor. In particular, the compounds disclosed herein exhibit activity as prostaglandin E2 (PGE2) receptor-2 (EP2) antagonists and are useful for treatment where EP2 receptor antagonism is indicated. In some embodiments, compounds described herein reduce or abolish one or more symptoms associated with an EP2 mediated disease or disorder (e.g., an EP2 mediated inflammatory disease or disorder.)

Aberrant EP2 expression is observed in several forms of cancers, including cancers of the colon, prostate, liver, and breast. EP2 activity (e.g., over-activity) has also been associated with risk factors for cancer including chronic inflammation, immunoregulation, angiogenesis, metastasis, and multidrug resistance. In some embodiments, disclosed herein are methods of treating cancer with a compound disclosed herein. The term “cancer” as used herein, refers to an abnormal growth of cells that tend to proliferate in an uncontrolled way and, in some cases, to metastasize (spread).

More generally, the present compounds are useful to treat proliferative disorders, including solid tumors, carcinoma of the brain, kidney, liver, adrenal gland, bladder, breast, stomach, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins lymphoma, a mammary carcinoma, follicular carcinoma, undifferentiated carcinoma, papillary carcinoma, seminoma, melanoma, IL-1 driven disorders, ABC diffuse large B-cell lymphoma (DLBCL), Waldenström's macroglobulinemia, Hodgkin's lymphoma, primary cutaneous T-cell lymphoma, smoldering or indolent multiple myeloma, or hematological malignancies (including leukemia, acute myeloid leukemia (AML), DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, myelodysplastic syndromes (MDS), myelofibrosis, polycythemia vera, Kaposi's sarcoma, splenic marginal zone lymphoma, multiple myeloma, plasmacytoma, intravascular large B-cell lymphoma).

In some embodiments, compounds described herein reduce one or more symptoms of an EP2 mediated cancer. In some embodiments, compounds described herein reduce or reverse the progression of an EP2 mediated cancer. In some embodiments, the cancer is a solid tumor. In some embodiments, the cancer is colon cancer.

In some embodiments, EP2 signaling (i.e., through activation by PGE₂), contributes to inflammation by enhancing edema and leukocyte infiltration from increased vascular permeability, thereby allowing more blood or fluid flow into an inflamed area of the body. In some embodiments, modulation of EP2 function has effects on B lymphocytes, T lymphocytes, cytotoxic T-cell function, or a combination thereof.

Activation of EP2 promotes dissemination of cancer cells following needle biopsy (Kameyama et al. Cell Reports Medicine, 4, 12, 101330). Accordingly, the present compounds are useful in antagonizing EP2 to inhibit cancer cell dissemination following needle biopsy, tumor resection or other tissue injury.

In certain embodiments, the present compounds are useful in the treatment of endometriosis, uterine fibroids (leiomyomata), menorrhagia, adenomyosis, primary and secondary dysmenorrhoea (including symptoms of dyspareunia, dyschexia and chronic pelvic pain), chronic pelvic pain syndrome, precocious puberty, cervical ripening, breast carcinoma, colon carcinoma, familial adenomatous polyposis, colorectal adenomas, endometrial carcinoma, prostate carcinoma, pulmonary carcinoma, testicular carcinoma, gastric carcinoma, macular degeneration, inflammatory and neuropathic pain conditions, cancer pain, polycystic kidney disease and polycystic ovarian syndrome.

Particular diseases or disorders that can be treated with the present compounds include, without limitation, endometriosis, uterine fibroids (leiomyomata), menorrhagia, adenomyosis, primary and secondary dysmenorrhoea (including symptoms of dyspareunia, dyschexia and chronic pelvic pain), chronic pelvic pain syndrome, polycystic kidney disease and polycystic ovarian syndrome.

In one embodiment the present compounds are useful in treating fibrotic conditions, including, without limitation, idiopathic pulmonary fibrosis, systemic sclerosis, low grade scarring, wound healing, uterine fibroids (leiomyomata), sclerodoma, autoimmune hepatitis, primary biliary cirrhosis, sclerosing cholangitis, partial liver resection, acute liver necrosis, necrosis caused by toxin, viral hepatitis, shock, or anoxia, B-virus hepatitis, non-A/non-B hepatitis, chronic hepatitis C virus (HCV) infection, cirrhosis, alcoholic liver disease, including alcoholic cirrhosis, non-alcoholic steatohepatitis (NASH), hepatic failure, fulminant hepatic failure, late-onset hepatic failure, “acute-on-chronic” liver failure, kidney disease, including chronic kidney disease.

In one embodiment, the present compounds are used to treat a kidney disease, including without limitation, chronic kidney injury, acute kidney injury, albuminaria, polycystic kidney disease, and the like.

More particularly, the compounds and derivatives of the present invention may be useful for treating endometriosis and/or uterine fibroids (leiomyomata).

In some embodiments, disclosed herein are methods of treating inflammation with a compound disclosed herein. In some embodiments, the compounds disclosed herein are used in the reduction or suppression of inflammation in a mammal. In some embodiments, the compounds disclosed herein are used in the treatment or prevention of inflammation-related conditions (e.g., allergies, pain, and the like).

In some embodiments, disclosed herein is a method of reducing inflammation in a tissue comprising contacting an inflamed cell or tissue with a compound disclosed herein, in an amount sufficient to decrease or inhibit the inflammation. In some embodiments, the inflammation includes an inflammatory or allergic condition.

In some embodiments, the compounds disclosed herein reduce one or more symptoms of a neuroinflammatory disease or disorder comprising reducing the activity of EP2 (e.g., by contacting the inflamed tissue with an EP2 antagonist disclosed herein). In some embodiments, disclosed herein is a method of reducing or halting the progression of a neuroinflammatory disease or disorder comprising administering a compound disclosed herein to an individual (e.g., a mammal, a human, etc.) in need thereof.

In one embodiment, the present compounds are used to treat brain aging, and its effects, such as cognitive decline (Minhas, P. S. et al. Restoring metabolism of myeloid cells reverses cognitive decline in ageing. Nature (2021) doi:10.1038/s41586-020-03160-0).

In some embodiments, disclosed herein are compounds and methods for their use to treat neurological disorders, including neurodegenerative disorders, such as neurological disease and neurodegenerative disease: amyotrophic lateral sclerosis (ALS), epilepsy, dementia, Alzheimer's disease, concussion, delirium, chemotherapy-associated cognitive decline, radiation-associated cognitive decline, post-operative cognitive dysfunction including post-operative neurocognitive disorder, post-operative delirium, delayed neurocognitive disorder, and delayed neurocognitive recovery, vascular dementia, frontotemporal dementia, Lewy body dementia, pre-senile dementia (mild cognitive impairment or MCI), Binswanger's dementia (subcortical arteriosclerotic encephalopathy), HIV-associated dementia (including asymptomatic neurocognitive impairment (ANI), minor neurocognitive disorder (MND), and HIV-associated dementia (HAD) (also called AIDS dementia complex [ADC] or HIV encephalopathy), multiple system atrophy (MSA), spinocerebellar ataxias, Steel-Richardson-Olszewski disease (progressive supranuclear palsy), head injury, concussion, chronic traumatic encephalopathy, intracerebral hemorrhage, hematoma, diabetic retinopathy, macular degeneration, macular edema, glaucoma, multiple sclerosis, vasogenic edema including edema caused by treatment with antibody therapies (e.g. ARIA-E), migraine, Huntington's disease, ALS, and Parkinson's disease.

In other embodiments, the present compounds are useful for treating disorders of the central nervous system (CNS). In one embodiment, the CNS disorder is a psychiatric, mental, mood or affective disorder, such as a disorder selected from addiction, a bipolar disorder, schizophrenia, general psychosis, drug-induced psychosis, a delusional disorder, a schizoaffective disorder, obsessive compulsive disorder (OCD), a depressive disorder, such as treatment resistant depression, suicidal ideation, major depressive disorder, an anxiety disorder, a panic disorder, post-traumatic stress disorder (PTSD), attention deficit disorder (ADD), attention deficit hyperactivity disorder (ADHD) and substance abuse disorder. In particular embodiments, the CNS disorder is selected from chemo brain, levo-dopa induced addictive behavior, alcoholism, narcotic dependence (including but not limited to amphetamine, opiates or other substances) and substance abuse.

In one embodiment the present compounds are useful in treating CNS disorders characterized by a relative reduction in synaptic plasticity and synaptic processes including, for example, Fragile X, Rhett's disorder, Williams syndrome, Renpenning's syndrome, autism spectrum disorders (ASD), autism, Asperger's syndrome, pervasive development disorder or childhood disintegrative disorder.

In one embodiment, the present compounds are useful for treating conditions wherein the neurovascular tissue is inflamed or impaired, or the blood-brain barrier is degraded, which in some cases is accompanied by altered cerebral blood flow, such as impaired cerebral blood flow, altered diffusion or perfusion, such as impaired diffusion, or damage to associated tissue, such as with white matter damage or white matter lesions. Examples of such conditions include Alzheimer's disease, vascular dementia, vascular cognitive impairment and dementia (VCID), cerebral small vessel disease, subcortical ischemic vascular disease, mixed dementia, ALS, multiple sclerosis, Parkinson's disease and Huntington's disease. The present compounds also are useful in treating vascular injuries such as head injury, stroke, aneurysm and ischemic vascular damage, for example.

The present compounds exert anti-inflammatory and analgesic effects by modulating aspects of the prostanoid signaling pathway. Accordingly, the present compounds are useful as non-steroidal anti-inflammatory drugs (NSAIDs), and can be used to treat conditions where NSAIDs are indicated. In certain embodiments, the present compounds are useful for treating pain, including nociceptive and neuropathic pain. More particularly the present compounds are useful as analgesics to treat acute pain, central pain syndrome, nerve pain, such as neuropathic pain, chemotherapy induced neuropathy and neuropathic pain, diabetic neuropathy, HIV-related neuropathy, fibromyalgia, neuralgia, such as post-herpetic neuralgia, sciatica, neuropathic pain associated with a CNS disease (such as multiple sclerosis), post-operative pain, tonic pain, menstrual pain, osteoarthritic pain, rheumatoid arthritis pain, visceral pain, orphan pain, migraine, chronic pain such as post-operative pain, lumbosacral pain, musculo-skeletal pain, headache, cluster headache, inflammation induced pain and cancer pain.

In some embodiments, reducing inflammation, or treatment of an inflammatory condition, includes reducing or inhibiting the activity of EP2. In some embodiments, reducing inflammation, or treatment of an inflammatory condition, includes administering an antagonist of EP2 (e.g., an EP2 antagonist disclosed herein).

In certain embodiments, the present compounds are used to treat a genetic disorder, such as Hirschsprung's disease, cerebral autosomal-dominant arteriopathy with subcortical infarcts and leukoencephalopathy (CADASIL or CADASIL syndrome), cerebral autosomal-recessive arteriopathy with subcortical infarcts and leukoencephalopathy (CARASIL or CARASIL syndrome), Wilson's disease, DiGeorge syndrome, retinitis pigmentosa, and the like.

In some embodiments, the inflammatory condition is an allergic condition. In some embodiments, the inflammatory condition is asthma. In some embodiments, the inflammatory condition is anaphylaxis. In some embodiments, the inflammatory condition is chronic inflammation. In some embodiments, disclosed herein is a method of treating chronic inflammation comprising administering an EP2 antagonist (e.g., a compound disclosed herein) to the individual in need thereof.

Particular examples of inflammatory and allergic conditions that can be treated using the presently disclosed compounds include, without limitation, systemic lupus erythematosus, rheumatoid arthritis, type I diabetes mellitus, inflammatory bowel disease, biliary cirrhosis, uveitis, multiple sclerosis, Crohn's disease, hyperimmunoglobulinemia D and periodic fever syndrome, cryopyrin-associated periodic syndromes, Schnitzler's syndrome, systemic juvenile idiopathic arthritis, adult's onset Still's disease, ulcerative colitis, necrotizing enterocolitis (NEC), peritonitis, gout, gout flares, gouty arthritis, bullous pemphigoid, sarcoidosis, psoriasis, autoimmune myositis, osteoarthritis, Wegener's granulomatosis, ichthyosis, Graves ophthalmopathy and asthma.

Examples of allergic disorders that may be treated using the presently disclosed compounds, include, but are not limited to, asthma (e.g. atopic asthma, allergic asthma, atopic bronchial IgE-mediated asthma, non-atopic asthma, bronchial asthma, non-allergic asthma, essential asthma, true asthma, intrinsic asthma caused by pathophysiologic disturbances, essential asthma of unknown or unapparent cause, emphysematous asthma, exercise-induced asthma, emotion-induced asthma, extrinsic asthma caused by environmental factors, cold air induced asthma, occupational asthma, infective asthma caused by or associated with bacterial, fungal, protozoal, or viral infection, incipient asthma, wheezy infant syndrome, bronchiolitis, cough variant asthma or drug-induced asthma), allergic bronchopulmonary aspergillosis (ABPA), allergic rhinitis, perennial allergic rhinitis, perennial rhinitis, vasomotor rhinitis, post-nasal drip, purulent or non-purulent sinusitis, acute or chronic sinusitis, and ethmoid, frontal, maxillary, and sphenoid sinusitis.

In one embodiment, the present compounds are used for ameliorating, treating or preventing immune regulatory disorders related to bone marrow or organ transplant rejection or graft-versus-host disease. Examples of inflammatory and immune regulatory disorders that can be treated with the present compounds include, but are not limited to, transplantation of organs or tissue, graft-versus-host diseases brought about by transplantation, autoimmune syndromes including rheumatoid arthritis, systemic lupus erythematosus, Hashimoto's thyroiditis, multiple sclerosis, systemic sclerosis, myasthenia gravis, type I diabetes, uveitis, posterior uveitis, allergic encephalomyelitis, glomerulonephritis, postinfectious autoimmune diseases including rheumatic fever and post-infectious glomerulonephritis, inflammatory and hyperproliferative skin diseases, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, celiac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, chronic lymphocytic leukemia, arteriosclerosis, atherosclerosis, aortitis syndrome, polyarteritis nodosa, myocardosis, scleroderma, Wegener's granuloma, Sjögren's syndrome, adiposis, eosinophilic fascitis, lesions of gingiva, periodontium, alveolar bone, substantia ossea dentis, glomerulonephritis, alopecia areata, male pattern alopecia or alopecia senilis by preventing epilation or providing hair germination and/or promoting hair generation and hair growth, vitiligo, muscular dystrophy, pyoderma and Sezary's syndrome, Addison's disease, ischemia-reperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA ballous dermatitis and cement dermatitis, gingivitis, periodontitis and sepsis.

In one embodiment, the present compounds are useful in treating cytokine release syndrome (CRS), which can result from a variety of factors, including severe viral infections such as influenza, administration of antibodies that are used for immunotherapy, such as cancer immunotherapy, and non-protein-based cancer drugs such as oxaliplatin and lenalidomide. Similarly, without being limited to any particular theory, the present compounds are useful in the treatment of infectious disease, such as by modulating the inflammatory cytokines associated with severe infections. Such infections include, without limitation, neurocysticercosis, trypanosomiasis, cerebral malaria, viral hemorrhagic fevers (such as ebola, Marburg, and the like), meningitis, dengue, Zika, nipah virus, Japanese encephalitis virus (JEV), West Nile Virus (WNV), Chikungunya virus, tick-borne encephalitis virus (TBEV), herpes simplex virus (HSV), human T-lymphotropic virus type 1 (HTLV-1), Naegleria fowleri, Toxoplasma gondii, listeriosis, and coronavirus infection, such as those causing SARS, MERS and COVID.

As described in the preceding paragraphs, the compounds disclosed herein are useful to treat diseases such as, without limitation, cancer, reproductive diseases, inflammatory diseases, pain, vascular diseases, neurological diseases, and neurodegenerative diseases. In some cases, these disease conditions involve multifactorial disease processes, and treatment with these compounds provides therapeutic benefit by treating multiple underlying disease pathways within a single disease condition or in comorbid conditions. Accordingly, use of the present compounds for the treatment of multiple diseases referenced in the foregoing paragraphs is intended.

### Compounds

Compounds described herein, including pharmaceutically acceptable salts, prodrugs, active metabolites and pharmaceutically acceptable solvates thereof, are EP2 antagonists. In some embodiments, a compound of any one of the formulae described herein, or a pharmaceutically acceptable salt thereof, is an EP2 antagonist.

In one aspect, provided herein is a compound of Formula (I) or Formula (II)

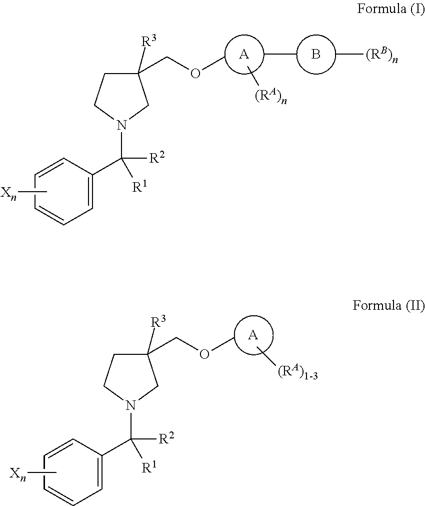

wherein
R¹ and R² are independently selected from R^{a}, —OR^{d}, —C₁₋₄ haloalkyl, or together R¹ and R² form an oxo, C₃₋₆ membered cycloalkyl or 3-6 membered cycloheteroalkyl;
R³ is —CH(R^{b})₂, —C(R^{b})₃, —S(O)₂R^{d}, —S(O)₂OR^{d}, —S(O)₂NR^{c}R^{c}, —S(O)(NR^{a})R^{d}, —S(O)₂N(R^{a})S(O)₂R^{a}, —S(O)₂N(R^{a})S(O)₂NR^{c}R^{c}, —C(O)R^{d}, —C(O)N(R^{a})C(O) R^{d}, —C(O)OR^{d}, —C(O)NR^{c}R^{c};
Ring A and Ring B independently are selected from phenyl and cycloalkyl;
each X is selected from R^{b};
each R^{A} is independently selected from halogen; —CN, —C₁₋₄ alkyl, and —C₁₋₄ haloalkyl;
each R^{B} is independently selected from the group consisting of halogen, —CN, —C₁₋₄ alkyl, —C₁₋₄ haloalkyl, —C₁₋₄ aminoalkyl, —C₁₋₄ hydroxyalkyl, —C₁₋₄ methoxyalkyl, —(C₁₋₄ alkyl)O(C₁₋₄ alkyl), —C(O)(C₁₋₄ alkyl), —C(O)OH, —C(O)O(C₁₋₄ alkyl), —C(O)NR^{c}R^{c}, —NR^{c}R^{c}, —NH(C₃₋₆ cycloalkyl), —NH(C₃₋₆ heterocycloalkyl) and substituted or unsubstituted C₃₋₆ cycloalkyl;
each R^{a} is independently selected from the group consisting of hydrogen, (C₁₋₆) alkyl and (C₃₋₈) cycloalkyl;
each R^{b} is independently selected from the group consisting of —OR^{d}, —OCF₂H, —OCF₃, —NR^{c}R^{c}, halogen, —CF₃, —CN, —S(O)₂R^{d}, —S(O)₂OR^{d}, —S(O)₂NR^{c}R^{c}, —C(O)R^{d}, —C(O)OR^{d}, —C(O)NR^{c}R^{c},
each R^{c} is independently R^{a}, or, alternatively, two R^{c} are taken together with the nitrogen atom to which they are bonded to form a 5 to 8-membered cycloheteroalkyl or 5-membered heteroaryl, which may optionally include one or more of the same or different additional heteroatoms and which may optionally be substituted with one or more of the same or different —C(O)R^{a} and R^{a} groups;
each R^{d} is independently selected from hydrogen and (C₁₋₆) alkyl optionally substituted with 1, 2 or 3 groups selected from halogen and —O(C₁₋₄ alkyl); and
n is, for each occurrence, independently 0 to 3;

provided that the compound is not a compound disclosed in PCT/US22/34903.

In one embodiment of compounds of Formula (I), Ring A or Ring B is C₃₋₆ cycloalkyl. In one embodiment, Ring A or Ring B is C₃₋₆ cycloalkyl that is bicyclo[1.1.1]pentanyl. Thus in one embodiment, compounds of Formula (I) have Formula (Ia):

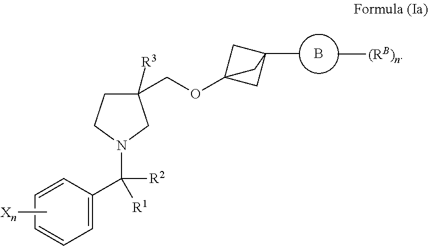

And in one embodiment compounds of Formula (I) can be represented by Formula (Ib):

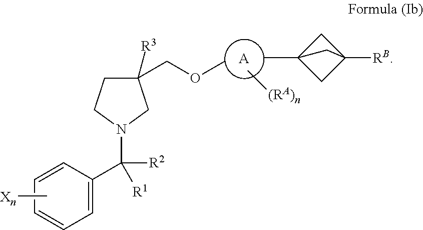

In one embodiment of compounds according to Formulas (I) and (Ib), Ring A is phenyl. In one embodiment of compounds according to Formulas (I) and (Ib), Ring A is unsubstituted phenyl.

In another embodiment of compounds according to Formulas (I) and (Ia), Ring B is phenyl. In particular embodiments of compounds according to Formulas (I), (Ia) and (Ib), Ring B is substituted with one, two or three R^{B}. In other embodiments of compounds according to Formulas (I), (Ia) and (Ib), Ring B is unsubstituted. In certain embodiments, compounds having Formulas (I), (Ia) and (Ib) are substituted with at least one R^{B}. In certain embodiments of compounds having Formulas (I), (Ia) and (Ib), Ring B is substituted with at least one R^{B} selected from —CN, —C(O)(C₁₋₄ alkyl), —C(O)OH, —C(O)O(C₁₋₄ alkyl) or —C(O)NR^{c}R^{c}.

In more particular embodiments, compounds of Formulas (I), (Ia) and (Ib) are substituted with at least one R^{B} that is —CN. In one embodiment compounds according to Formulas (I), (Ia) and (Ib) have Formula (Ic)

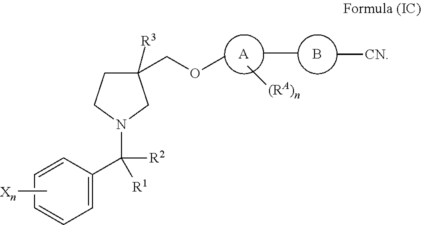

In one embodiment, compounds of Formula (I) have an unsubstituted Ring B. In one embodiment, compounds of Formula (I) have an unsubstituted cycloalkyl Ring B. In one embodiment, compounds that have an unsubstituted cycloalkyl Ring B have Formula (Id):

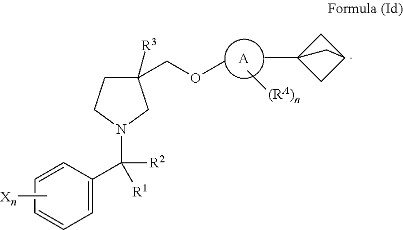

In one embodiment, disclosed herein are compounds of Formula (II), including wherein each R^{A} independently is halo. In one embodiment of such compounds of Formula (II), examples are represented by Formula (IIa):

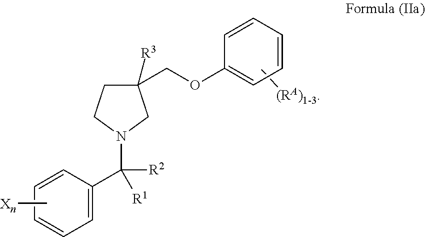

Further embodiments of compounds according to Formulas (II) and (IIa) have Formula (IIb):

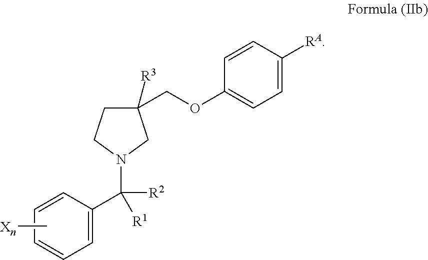

In certain embodiments of compounds according to Formulas (I), (Ia), (Ib), (Ic), (Id), (II), (IIa) and (IIb), R³ is selected from —CH(R^{b})₂ and —C(R^{b})₃ and in particular examples of such compounds, each R^{b} in R³ is independently selected from —OR^{d}, —OCF₂H, —OCF₃, and —CF₃. In certain embodiments of compounds having Formulas (I), (Ia), (Ib), (Ic), (Id) and (II), R³ is —CH(OH)CF₃. In other embodiments of compounds having Formulas (I), (Ia), (Ib), (Ic), (Id), (II), (IIa) and (IIb) R³ is —C(OH)₂CF₃.

In other embodiments of compounds having Formulas (I), (Ia), (Ib), (Ic), (Id), (II), (IIa) and (IIb), R³ is selected from S(O)₂R^{d}, —S(O)(NR^{a})R^{d}, —S(O)₂OR^{d}, —S(O)₂NR^{c}R^{c}, —S(O)₂N(R^{a})S(O)₂R^{a} and —S(O)₂N(R^{a})S(O)₂NR^{c}R^{c}. In particular embodiments, compounds of Formulas (I), (Ia), (Ib), (Ic), (Id), (II), (IIa) and (IIb) have R³ as S(O)₂NH₂. In other embodiments of compounds having Formulas (I), (Ia), (Ib), (Ic), (Id), (II), (IIa) and (IIb), R³ is S(O)₂CH₃. In certain embodiments of Formulas (I), (Ia), (Ib), (Ic), (Id), (II), (IIa) and (IIb), R³ is —S(O)(NR^{a})R^{d}. In still other embodiments, compounds of Formulas (I), (Ia), (Ib), (Ic), (Id), (II), (IIa) and (IIb) have R³ as S(O)(NH)CH₃.

In other embodiments of compounds according to Formulas (I), (Ia), (Ib), (Ic), (Id), (II), (IIa) and (IIb), R³ is selected from —C(O)R^{d}, —C(O)N(R^{a})C(O)R^{d}, —C(O)OR^{d} and —C(O)NR^{c}R^{c}. In one such embodiment, R³ is —C(O)OR^{d}, such as in compounds having Formulas (I), (Ia), (Ib), (Ic), (Id), (II), (IIa) and (IIb), wherein R³ is —COOH.

Particular examples of the presently disclosed compounds, including compounds according to Formulas (I), (Ia), (Ib), (Ic), (Id), (II), (IIa) and (IIb), have 1, 2 or 3 X substituents, wherein each X is independently selected from R^{b}. Certain examples of compounds disclosed herein according to Formulas (I), (Ia), (Ib) (Ic) and (Id), have a single X substituent at the ortho, meta or para position. When such compounds have a single X at the para position, the have the structure of Formula (Ie):

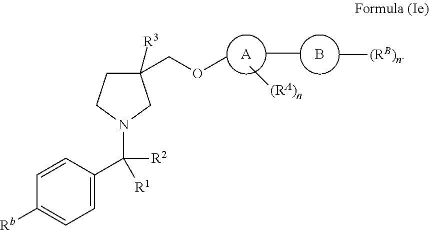

In one embodiment, compounds disclosed herein according to Formulas (I), (Ia), (Ib) (Ic), (Id) and (Ie), have the structure of Formula (If):

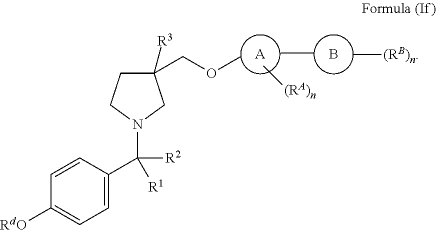

In another aspect, examples of compounds disclosed herein according to Formulas (I), (la), (Ib) (Ic), (Id) (Ie) and (If), have the structure of Formula (Ig):

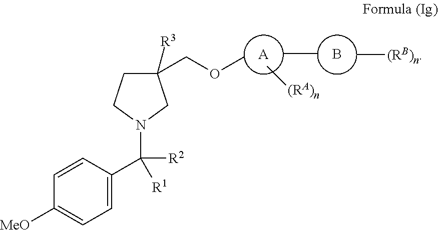

In still another embodiment of compounds disclosed herein compounds according to Formulas (I), (Ia), (Ib) (Ic), (Id) (Ie), (If) and (Ig) are substituted with one or more R^{B} that is cyano. In one aspect, certain embodiments of such compounds are represented by Formula (Ih):

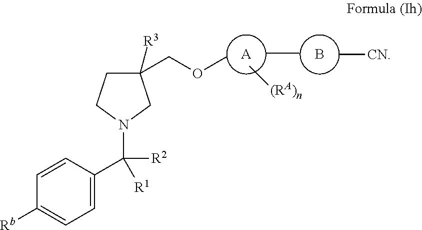

In particular examples of compounds having Formulas (I), (Ia), (Ib), (Ic), (Id), (Ie), (If), (Ig), (Ih), (II), (IIa) and (IIb), R¹ and R² together form an oxo. In other examples, compounds of Formulas (I), (Ia), (Ib), (Ic), (Id), (Ie), (If), (Ig), (Ih), (II), (IIa) and (IIb) have one of R¹ and R² being hydrogen and the other being —CF₃.

Any combination of the groups described above for the various variables is contemplated herein. Throughout the specification, groups and substituents thereof are chosen by one skilled in the field to provide stable moieties and compounds.

In one embodiment, disclosed compounds according to Formulas (I), (Ia), (Ib), (Ic), (Id), (Ie), (If), (Ig), (Ih), (II), (IIa) and/or (IIb) include those set forth in Table (I).

TABLE (I)

| ID No. | Structure |
|---|---|
| I-1 | 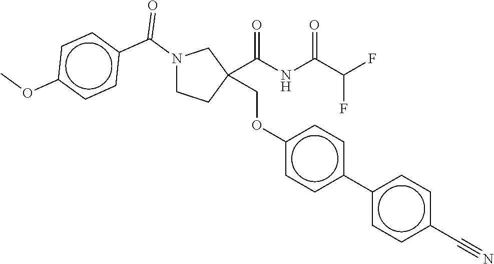 |
| I-2 | 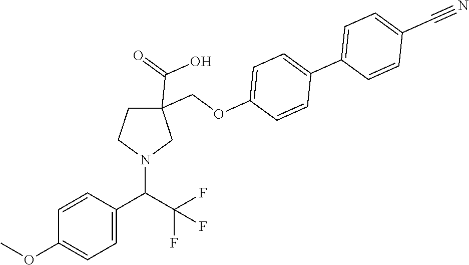 [SMILES: COc1ccc(C(N2CCC(COc3ccc(-c4ccc(C#N)cc4)cc3)(C(=O)O)C2)C(F)(F)F)cc1] |
| I-3 | 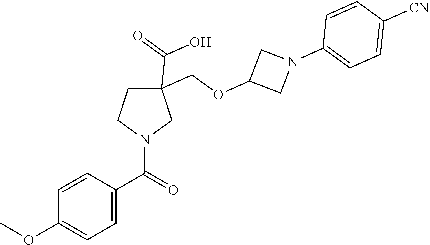 [SMILES: COc1ccc(C(=O)N2CCC(COC3CN(c4ccc(C#N)cc4)C3)(C(=O)O)C2)cc1] |
| I-4 | 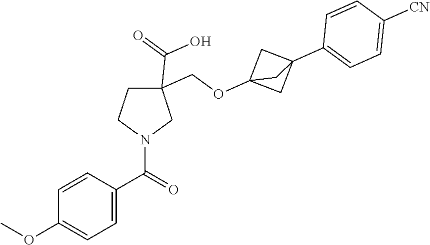 [SMILES: COc1ccc(C(=O)N2CCC(COC34CC(c5ccc(C#N)cc5)(C3)C4)(C(=O)O)C2)cc1] |
| I-5 | 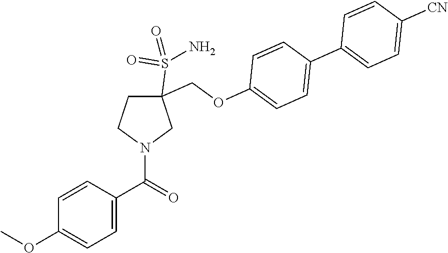 [SMILES: COc1ccc(C(=O)N2CCC(COc3ccc(-c4ccc(C#N)cc4)cc3)(S(N)(=O)=O)C2)cc1] |
| I-6 | 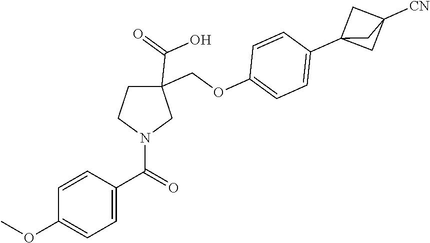 [SMILES: COc1ccc(C(=O)N2CCC(COc3ccc(C45CC(C#N)(C4)C5)cc3)(C(=O)O)C2)cc1] |
| I-7 | 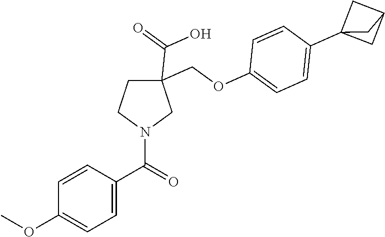 [SMILES: COc1ccc(C(=O)N2CCC(COc3ccc(C45CC(C4)C5)cc3)(C(=O)O)C2)cc1] |
| I-8 | 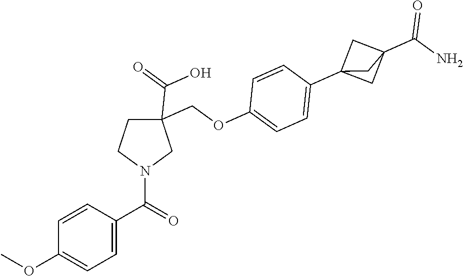 [SMILES: COc1ccc(C(=O)N2CCC(COc3ccc(C45CC(C(N)=O)(C4)C5)cc3)(C(=O)O)C2)cc1] |
| I-9 | 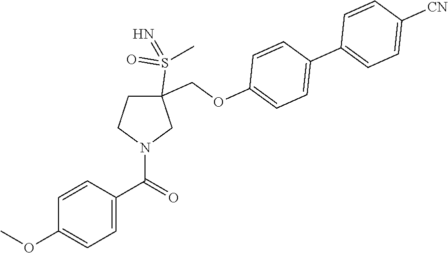 [SMILES: COc1ccc(C(=O)N2CCC(COc3ccc(-c4ccc(C#N)cc4)cc3)(S(C)(=N)=O)C2)cc1] |
| I-10 | 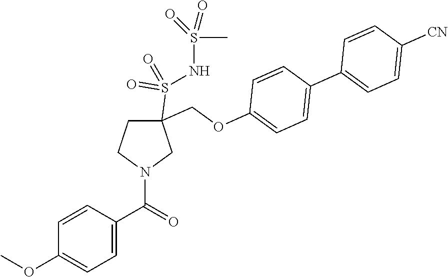 [SMILES: COc1ccc(C(=O)N2CCC(COc3ccc(-c4ccc(C#N)cc4)cc3)(S(=O)(=O)NS(C)(=O)=O)C2)cc1] |
| I-11 | 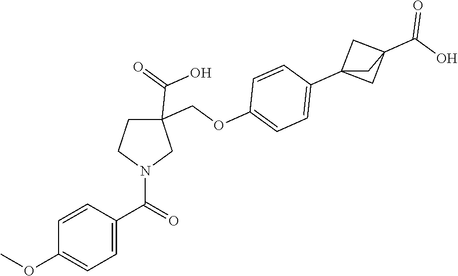 [SMILES: COc1ccc(C(=O)N2CCC(COc3ccc(C45CC(C(=O)O)(C4)C5)cc3)(C(=O)O)C2)cc1] |
| I-12 | 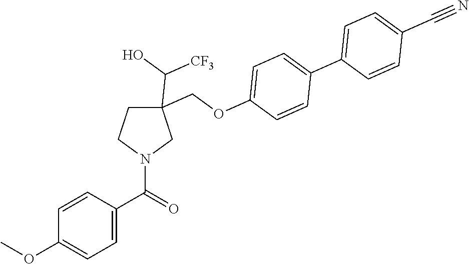 [SMILES: COc1ccc(C(=O)N2CCC(COc3ccc(-c4ccc(C#N)cc4)cc3)(C(O)C(F)(F)F)C2)cc1] |
| I-13 | 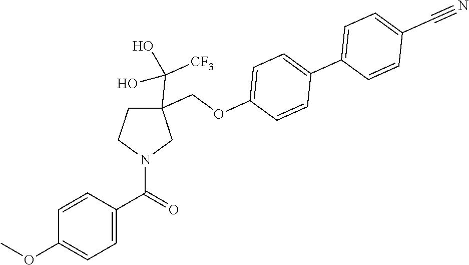 [SMILES: COc1ccc(C(=O)N2CCC(COc3ccc(-c4ccc(C#N)cc4)cc3)(C(O)(O)C(F)(F)F)C2)cc1] |
| I-14 | 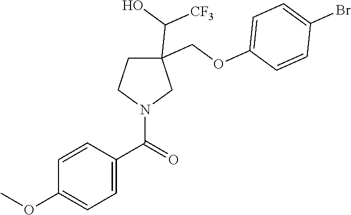 [SMILES: COc1ccc(C(=O)N2CCC(COc3ccc(Br)cc3)(C(O)C(F)(F)F)C2)cc1] |

### Compounds

Compounds described herein, including pharmaceutically acceptable salts, prodrugs, active metabolites and pharmaceutically acceptable solvates thereof, are EP2 antagonists. In some embodiments, a compound of any one of the formulae described herein, or a pharmaceutically acceptable salt thereof, is an EP2 antagonist.

In one aspect, provided herein is a compound of Formula (III)

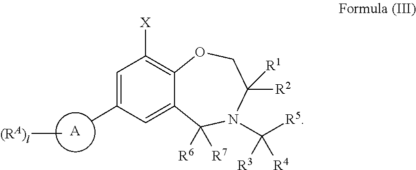

With reference to Formula (III), in one embodiment, Ring A is a bicyclic heterocycle having one or more nitrogen atoms; or Ring A is Ring A′; each R^{A} is selected from halogen; —CN, —C₁₋₄ alkyl, —C₁₋₄ haloalkyl, —(C₁₋₄ alkyl)O(C₁₋₄ alkyl), —C(O)OH, —C(O)O(C₁₋₄ alkyl), —C(O)NH₂, —C(O)NH(C₁₋₄ alkyl), —C(O)N(C₁₋₄ alkyl)₂, —NH₂, —NH(C₁₋₄ alkyl), —N(C₁₋₄ alkyl)₂, —OH, —O(C₁₋₄ alkyl), —O(C₁₋₄ haloalkyl), —S(C₁₋₄ alkyl), —SO₂C₁₋₄ alkyl, —SO₂NHC₁₋₄ alkyl, substituted or unsubstituted C₃₋₆ cycloalkyl, or substituted or unsubstituted 3- to 6-membered heterocyclyl; or two R^{A} taken together form a carbonyl; Ring A′ is selected from the group consisting of:

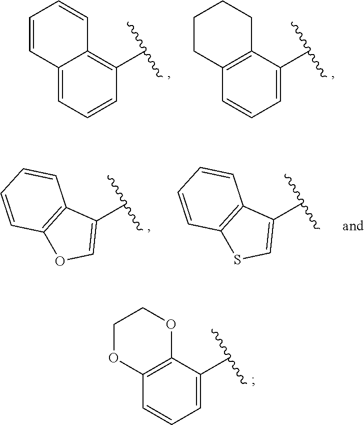 [SMILES: *c1cccc2ccccc12 ; *c1cccc2c1CCCC2 ; *c1coc2ccccc12 ; *c1csc2ccccc12 ; *c1cccc2c1OCCO2]

R¹ and R² are independently selected from hydrogen, —C₁₋₄ alkyl, —CN, —C(O)NR^{c}R^{c}, or together R¹ and R² form an oxo, C₃₋₆ membered cycloalkyl or 3-6 membered cycloheteroalkyl;
X is selected from hydrogen and halogen;
R³ and R⁴ are independently selected from hydrogen, —C₁₋₄ alkyl, together R³ and R⁴ form an oxo, or one of R³ and R⁴ form a double bond with R⁵ and the other of R³ and R⁴ is hydrogen or —C₁₋₄ alkyl;
R⁵ is selected
   from —(CH₂)ₘ—R^{b}, —(CHR^{a})ₘ—R^{b}, —O—(CH₂)ₘ—R^{b}, —O—(CHR^{a})ₘ—R^{b}, —C(O)NH—(CH₂)ₘ—R^{b}, —C(O)NH—(CHR^{a})ₘ—R^{b}, —NHC(O)OR^{d}, —NHC(O)NR^{c}R^{c};
R⁶ and R⁷ are independently selected from hydrogen, —C₁₋₄ alkyl, or together R⁶ and R⁷ form an oxo;
each R^{a} is independently selected from the group consisting of hydrogen, (C₁₋₆) alkyl and (C₃₋₈) cycloalkyl;
each R^{b} is independently selected from the group consisting of ═O, —OR^{d}, —OCF₂H, —OCF₃, —NR^{c}R^{c},
   halogen, —CF₃, —CN, —S(O)₂R^{d}, —S(O)₂OR^{d}, —S(O)₂NR^{c}R^{c}, —C(O)R^{d}, —C(O)OR^{d}, —C(O)NR^{c}R^{c}, and Ring B

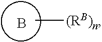

where Ring B is C₃₋₆ cycloalkyl, phenyl, 5-membered heteroaryl, 6-membered heteroaryl containing two or more nitrogen atoms, 9-10 membered heteroaryl; or Ring B is Ring B′;
each R^{B} is independently selected from the group consisting of halogen, —CN, —C₁₋₄ alkyl, —C₁₋₄ haloalkyl, —C₁₋₄ aminoalkyl, —C₁₋₄ hydroxyalkyl, —C₁₋₄ methoxyalkyl, —(C₁₋₄ alkyl)O(C₁₋₄ alkyl), —C(O)(C₁₋₄ alkyl), —C(O)OH, —C(O)O(C₁₋₄ alkyl), —C(O)NH₂, —C(O)NH(C₁₋₄ alkyl), —C(O)N(C₁₋₄ alkyl)₂, —NH₂, —NH(C₁₋₄ alkyl), —NH(C₃₋₆ cycloalkyl), —NH(C₃₋₆ heterocycloalkyl), —(CH₂)ₘ—N(R^{c})₂, —(CH₂)ₘ—NHC(O)C₁₋₄ alkyl, —(CH₂)ₘ—NHC(O)O(C₁₋₄ alkyl), —NHS(O)₂C₁₋₄ alkyl, —OH, —O(C₁₋₄ alkyl), —O(C₁₋₄ haloalkyl), —SH, —S(C₁₋₄ alkyl), —S(O)(C₁₋₄ alkyl), —S(O)(NH)(C₁₋₄ alkyl), —S(O)₂(C₁₋₄ alkyl), —S(O)₂NH₂, —S(O₂)NHCH₃, substituted or unsubstituted C₃₋₆ cycloalkyl, or substituted or unsubstituted 3- to 6-membered heterocyclyl; or two R^{B} taken together form a carbonyl;
each R^{c} is independently R^{a}, or, alternatively, two R^{c} are taken together with the nitrogen atom to which they are bonded to form a 5 to 8-membered cycloheteroalkyl or 5-membered heteroaryl, which may optionally include one or more of the same or different additional heteroatoms and which may optionally be substituted with one or more of the same or different —C(O)R^{a} and R^{a} groups;
each R^{d} is independently selected from hydrogen and (C₁₋₆) alkyl optionally substituted with 5-membered heteroaryl, 6-membered heteroaryl and 6-membered aryl, each optionally substituted with 1, 2 or 3 groups selected from halogen, C₁₋₄ alkyl, and —O(C₁₋₄ alkyl);
l is 0 to 3;
m is 0 to 3;
n is 0 to 3; and
Ring B′ is selected from the group consisting of:

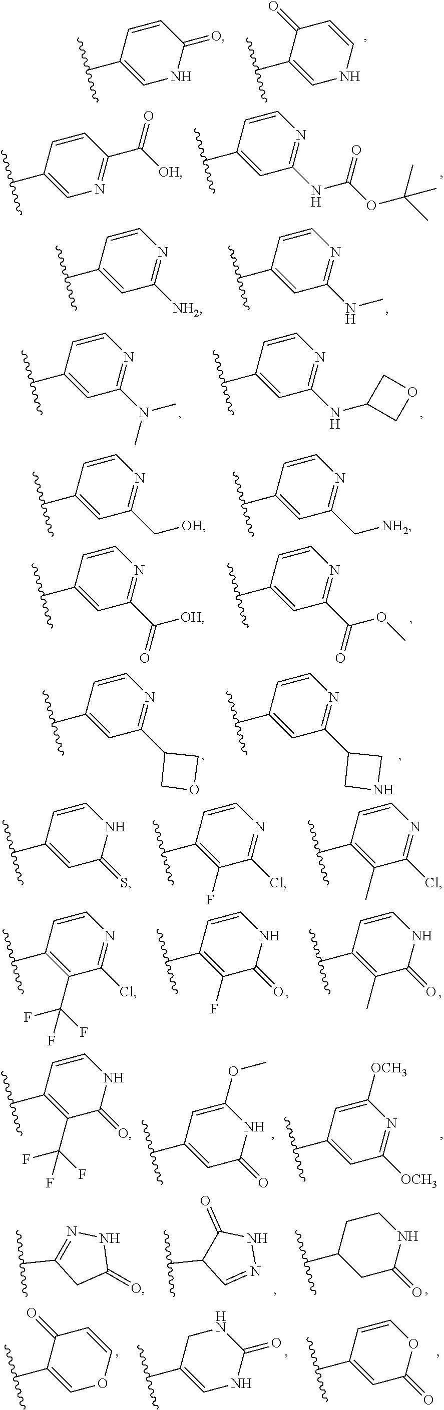

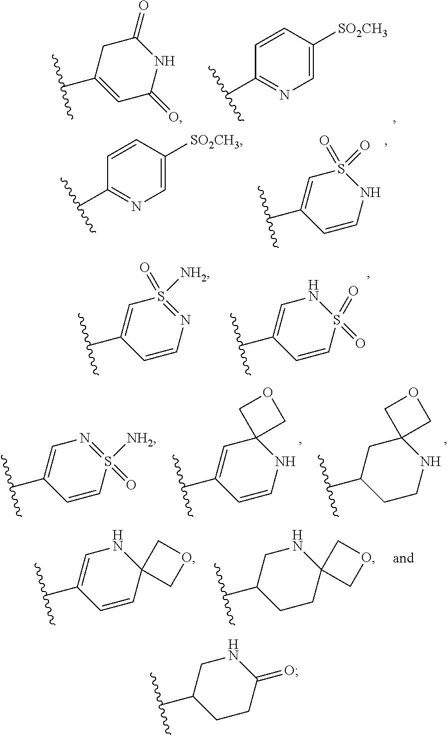

provided that the compound is not a compound disclosed in PCT/US2022/034901.

In one embodiment of a compound of Formula (III), EP2 antagonists disclosed herein have Formula (IV)

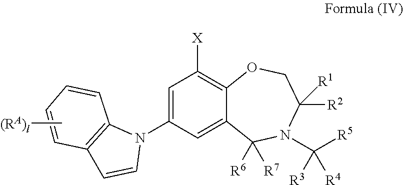

wherein, R^{A} is as defined herein above with respect to Formula (III). In a particular embodiment, at least one R^{A} of Formula (IV) is halogen. In one such embodiment, at least one R^{A} is fluoro, such as, by way of example, in compounds according to Formulas (II) and (IV) having Formula (IVa):

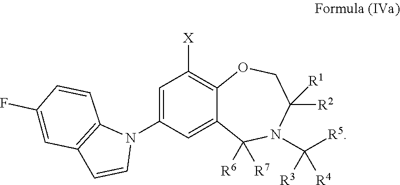

Thus, in one embodiment of compounds according to Formulas (III) and (IV), EP2 antagonists disclosed herein have Formula (IVa).

In one embodiment of Formula (III), EP2 antagonists disclosed herein have Formula (V)

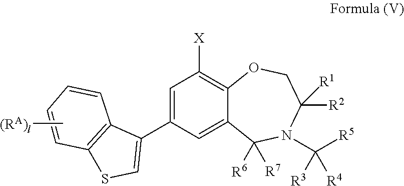

wherein, R^{A} is as defined herein above with respect to Formula (III). In a particular embodiment, at least one R^{A} of Formula (V) is halogen. In one such embodiment, at least one R^{A} is fluoro, such as, by way of example, in compounds according to Formulas (III) and (V) having Formula (Va):

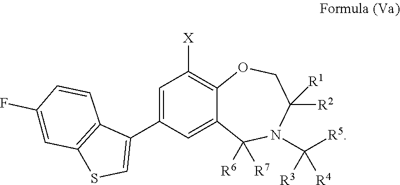

Thus, in one embodiment of compounds according to Formulas (III) and (V), EP2 antagonists disclosed herein have Formula (Va).

In one embodiment of Formula (III), EP2 antagonists disclosed herein have Formula (VI)

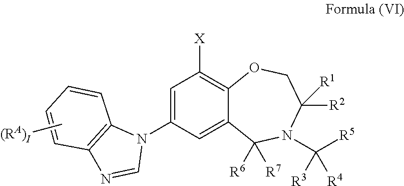

wherein, R^{A} is as defined herein above with respect to Formula (III). In a particular embodiment, at least one R^{A} of Formula (VI) is halogen. In one such embodiment, at least one R^{A} is fluoro, such as, by way of example, in compounds according to Formulas (III) and (VI) having Formula (VIa):

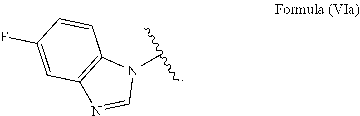 [SMILES: *n1cnc2cc(F)ccc21]

Thus, in one embodiment of compounds according to Formulas (III) and (VI), EP2 antagonists disclosed herein have Formula (VIa).

In one embodiment of Formula (III), EP2 antagonists disclosed herein have Formula (VII)

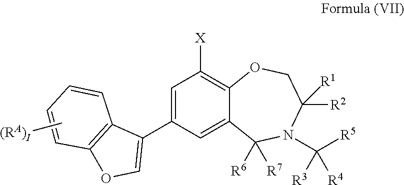

wherein, R^{A} is as defined herein above with respect to Formula (III). In a particular embodiment, at least one R^{A} of Formula (VII) is halogen. In one such embodiment, at least one R^{A} is fluoro, such as, by way of example, in compounds according to Formulas (III) and (VII) having Formula (VIIa):

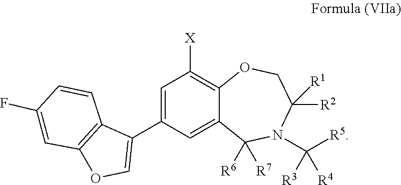

Thus, in one embodiment of compounds according to Formulas (III) and (VII), EP2 antagonists disclosed herein have Formula (VIIa). In further embodiments, compounds of Formulas (III) and (VII) may have two or more R^{A} as defined herein above with respect to Formula (III). In a particular embodiment, compounds of Formulas (III) and (VII) are substituted with two or three R^{A}, and in one such embodiment, R^{A} is halogen, such as fluoro. In one such embodiment, at least two R^{A} is fluoro, such as, by way of example, in compounds according to Formulas (III) and (VII) having Formula (VIIb):

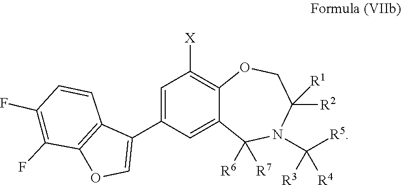

Thus, in one embodiment of compounds according to Formulas (III) and (VII), EP2 antagonists disclosed herein have Formula (VIIb).

In one embodiment, EP2 antagonists disclosed herein, including compounds of Formulas (III), (IV), (IVa), (V), (Va), (VI), (VIa), (VII), (VIIa) and (VIIb), have R⁵ selected from —(CH₂)ₘ—R^{b}, —(CHR^{a})ₘ—R^{b}, —O—(CH₂)ₘ—R^{b}, —O—(CHR^{a})ₘ—R^{b}, —C(O)NH—(CH₂)ₘ—R^{b}, —C(O)NH—(CHR^{a})ₘ—R^{b}, —NHC(O)OR^{d}, —NHC(O)NR^{c}R^{c}, and each R^{b} is independently selected from the group consisting of ═O, —OR, —OCF₂H, —OCF₃, —NR^{c}R^{c},

halogen, —CF₃, —CN, —S(O)₂R^{d}, —S(O)₂OR^{d}, —S(O)₂NR^{c}R^{c},
—C(O)R^{d}, —C(O)OR^{d}, —C(O)NR^{c}R^{c}, and Ring, B

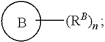

where Ring B is selected from C₃₋₆ cycloalkyl, phenyl, 5-membered heteroaryl, 6-membered heteroaryl containing two or more nitrogen atoms, 9-10 membered heteroaryl; or Ring B is Ring B′; Ring B′ is selected from the group consisting of:

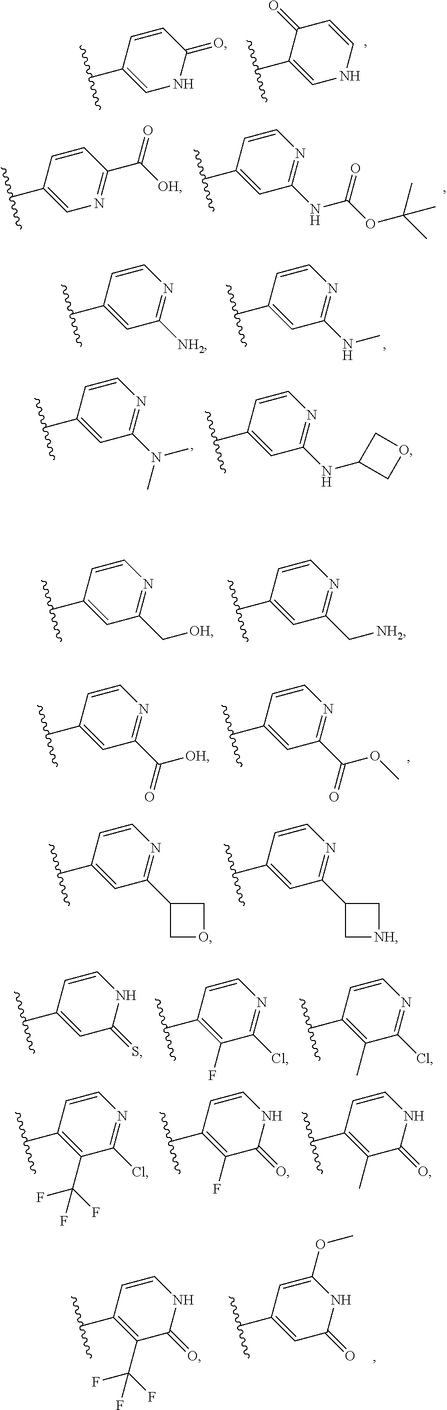

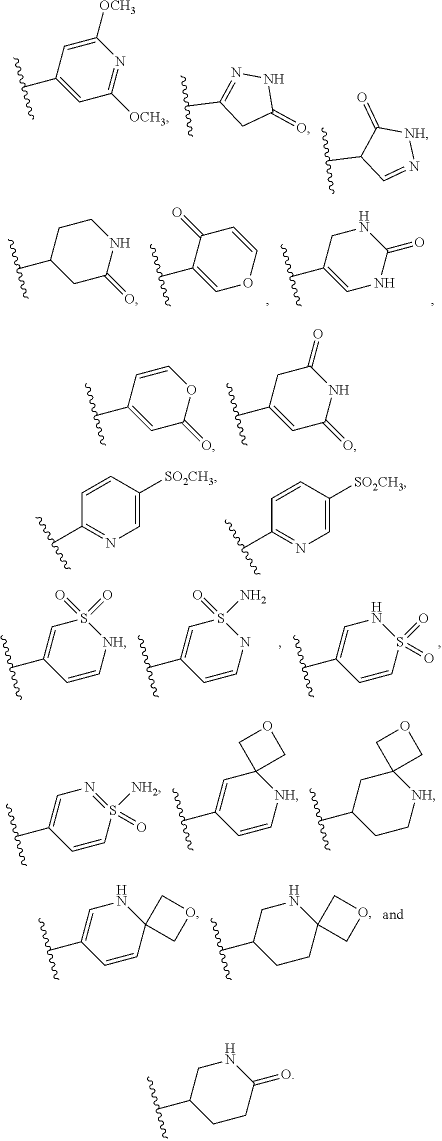

In one embodiment, EP2 antagonists disclosed herein, including compounds of Formulas (III), (IV), (IVa), (V), (Va), (VI), (VIa), (VII), (VIIa) and (VIIb) have R⁵ selected from —(CH₂)ₘ—R^{b}, —(CHR^{a})ₘ—R^{b}, —O—(CH₂)ₘ—R^{b}, —O—(CHR^{a})ₘ—R^{b}, —C(O)NH—(CH₂)ₘ—R^{b}, —C(O)NH—(CHR^{a})ₘ—R^{b}, —NHC(O)OR^{d}, —NHC(O)NR^{c}R^{c}, and each R^{b} is independently selected from the group consisting of ═O, —OR^{d}, —OCF₂H, —OCF₃, —NR^{c}R^{c},

halogen, —CF₃, —CN, —S(O)₂R^{d}, —S(O)₂0R^{d}, —S(O)₂NR^{c}R^{c},

—C(O)R^{d}, —C(O)OR^{d}, and —C(O)NR^{c}R^{c}. Certain embodiments of compounds according to Formulas Formulas (III), (IV), (IVa), (V), (Va), (VI), (VIa), (VII), (VIIa) and (VIIb)have R⁵ selected from —(CH₂)ₘ—R^{b}. In certain examples of compounds according to Formulas (III), (IV), (IVa), (V), (Va), (VI), (VIa), (VII), (VIIa) and (VIIb), R⁵ is —(CH₂)ₘ—R^{b} wherein R^{b} is selected from —NR^{c}R^{c}, halogen, —CF₃, —CN, —S(O)₂R^{d}, —S(O)₂OR^{d}, —S(O)₂NR^{c}R^{c},

—C(O)R^{d}, —C(O)OR^{d}, and —C(O)NR^{c}R^{c}.

In certain embodiments of Formulas (III), (IV), (IVa), (V), (Va), (VI), (VIa), (VII), (VIIa) and (VIIb), R⁵ is selected from

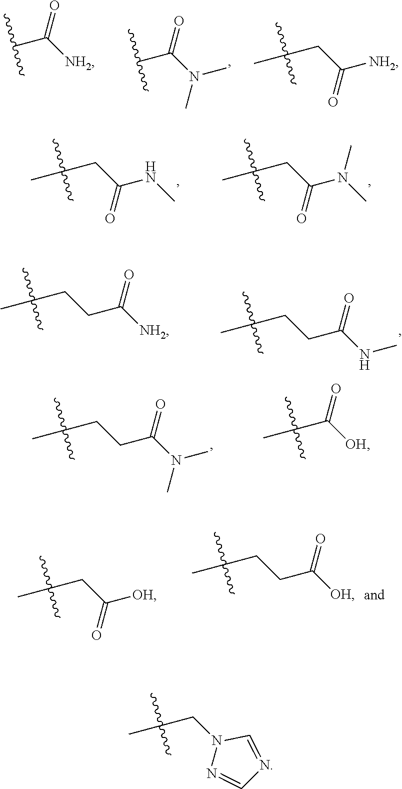

In one embodiment, compounds of Formulas (III), (IV), (IVa), (V), (Va), (VI), (VIa), (VII), (VIIa) and (VIIb), have Formula (VIII):

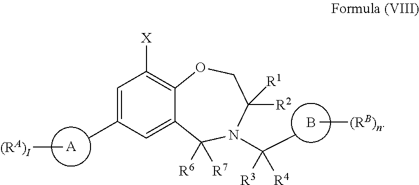

Thus, in one embodiment of compounds according to Formulas (III) (IV), (IVa), (V), (Va), (VI), (VIa), (VII), (VIIa) and (VIIb), EP2 antagonists disclosed herein have Formula (VIII).

In one embodiment, compounds of Formulas (III), (IV), (IVa), (V), (Va), (VI), (VIa), (VII), (VIIa), (VIIb) and (VIII) have Formula (VIIIa):

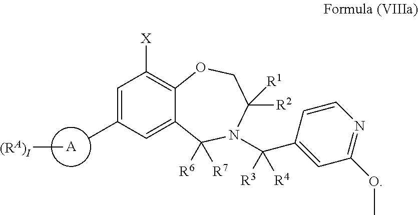

In one embodiment, compounds of (III), (IV), (IVa), (V), (Va), (VI), (VIa), (VII), (VIIa), (VIIb) and (VIII) have Formula (VIIIb):

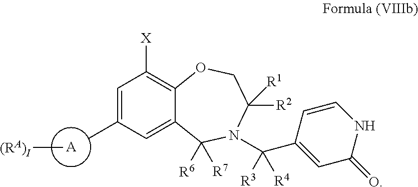

In one embodiment, compounds of Formulas (III), (IV), (IVa), (V), (Va), (VI), (VIa), (VII), (VIIa), (VIIb) and (VIII) have Formula (VIIIc):

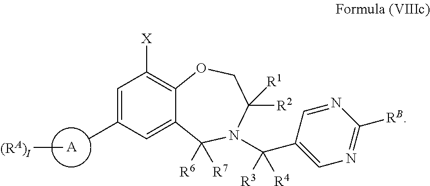

In one embodiment compounds of (III), (IV), (IVa), (V), (Va), (VI), (VIa), (VII), (VIIa), (VIIb) and (VIII) have Formula (VIIId):

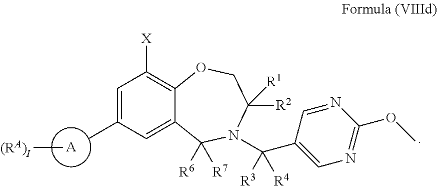

In one embodiment, compounds of Formulas (III), (IV), (IVa), (V), (Va), (VI), (VIa), (VII), (VIIa), (VIIb)and (VIII) have Formula (VIIIe):

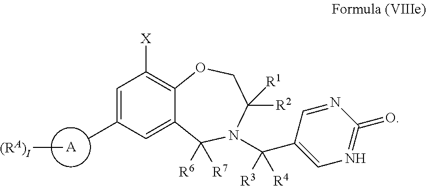

Thus, in certain embodiments of compounds according to (III), (IV), (IVa), (V), (Va), (VI), (VIa), (VII), (VIIa), (VIIb) and (VIII), EP2 antagonists disclosed herein have Formula (VIIIa), (VIIIb), (VIIIc), (VIIId) or (VIIIe).

In one embodiment of compounds of (III), (IV), (IVa), (V), (Va), (VI), (VIa), (VII), (VIIa), (VIIb) (VIII), (VIIIa), (VIIIb), (VIIIc), (VIIId) and (VIIIe), X is hydrogen. In one such embodiment, X is deuterium. In other embodiments of Formulas (III), (IV), (IVa), (V), (Va), (VI), (VIa), (VII), (VIIa), (VIIb), (VIII), (VIIIa), (VIIIb), (VIIIc) and (VIIId), X is halogen, such as fluoro, chloro, bromo or iodo. In particular embodiments of Formulas (III), (IV), (IVa), (V), (Va), (VI), (VIa), (VII), (VIIa), (VIIb), (VIII), (VIIIa), (VIIIb), (VIIIc), (VIII), (VIIIa), (VIIIb), (VIIIc), (VIIId) and (VIIIe), X is chloro. Accordingly, in one embodiment, compounds disclosed herein have Formula (IIIa):

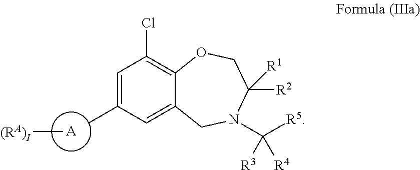

In one embodiment of the compounds disclosed herein, including compounds of Formulas (III), (IIIa), (VIII), (VIIIa), (VIIIb), (VIIIc), (VIIId) and (VIIIe), Ring A is a bicyclic heterocycle selected from the group consisting of:

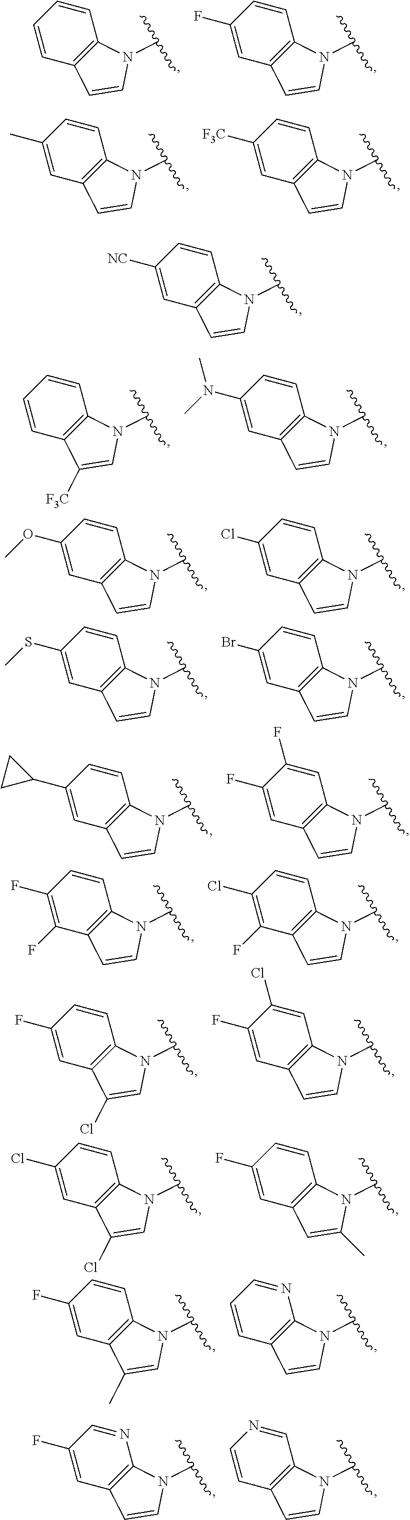

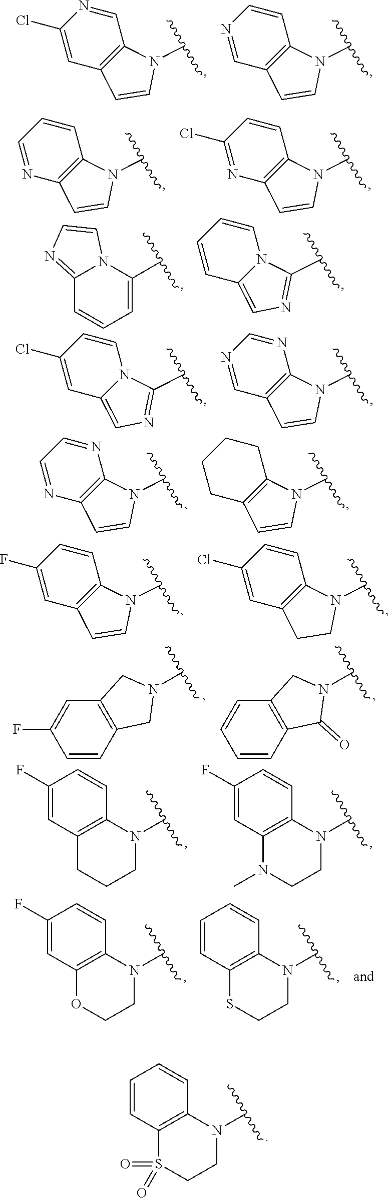

In one embodiment of compounds of Formulas (III), (IV), (IVa), (V), (Va), (VI), (VIa), (VII), (VIIa), (VIIb), (VIII), (VIIIa), (VIIIb), (VIIIc), (VIIId) and (VIIIe), R¹ and R² are independently selected from hydrogen, —C₁₋₄ alkyl, —CN, —C(O)NR^{c}R^{c}, or together R¹ and R² form an oxo, C₃₋₆ membered cycloalkyl or 3-6 membered cycloheteroalkyl. In one embodiment of compounds of Formulas (III), (IV), (IVa), (V), (Va), (VI), (VIa), (VII), (VIIa), (VIIb), (VIII), (VIIa), (VIIIb), (VIIIc), (VIIId) and (VIIIe), both R¹ and R² are hydrogen. In one such embodiment, at least one of R¹ and R² is enriched in deuterium. In one embodiment of compounds of Formulas (III), (IV), (IVa), (V), (Va), (VI), (VIa), (VII), (VIIa), (VIIb), (VIII), (VIIIa), (VIIIb), (VIIIc), (VIIId) and (VIIIe), one of R¹ and R² is hydrogen and the other is —C₁₋₄ alkyl. In other embodiments of compounds of Formulas (III), (IV), (IVa), (V), (Va), (VI), (VIa), (VII), (VIIa), (VIIb), (VIII), (VIIIa), (VIIIb), (VIIIc), (VIIId) and (VIIIe), both of R¹ and R² are —C₁₋₄ alkyl. In one embodiment of compounds of Formulas (III), (IV), (IVa), (V), (Va), (VI), (VIa), (VII), (VIIa), (VIIb), (VIII), (VIIIa), (VIIIb), (VIIIc), (VIIId) and (VIIIe), at least one of R¹ and R² is —C₁₋₄ alkyl, such as where at least one of R¹ and R² is methyl. In other embodiments of compounds of Formulas (III), (IV), (IVa), (V), (Va), (VI), (VIa), (VII), (VIIa), (VIIb), (VIII), (VIIIa), (VIIIb), (VIIIc), (VIIId) and (VIIIe), R¹ and R² together form an oxo, C₃₋₆ membered cycloalkyl or 3-6 membered cycloheteroalkyl. In one embodiment, R¹ and R² together form an oxo. In another embodiment of Formulas (III), (IV), (IVa), (V), (Va), (VI), (VIa), (VII), (VIIa), (VIIb), (VIII), (VIIIa), (VIIIb), (VIIIc), (VIIId) and (VIIIe), R¹ and R² together form a ring, such as a C₃₋₆ membered cycloalkyl or 3-6 membered cycloheteroalkyl. In one embodiment R¹ and R² together form a C₃₋₆ membered cycloalkyl, such as a cyclopropyl or cyclobutyl ring. In another embodiment of Formulas (III), (IV), (IVa), (V), (Va), (VI), (VIa), (VII), (VIIa), (VIIb), (VIII), (VIIIa), (VIIIb), (VIIIc), (VIIId) and (VIIIe), R¹ and R² together form a 3-6 membered cycloheteroalkyl, such as a cycloheteroalkyl containing oxygen, nitrogen, or both. In one embodiment of Formulas (III), (IV), (IVa), (V), (Va), (VI), (VIa), (VII), (VIIa), (VIIb), (VIII), (VIIIa), (VIIIb), (VIIIc), (VIIId) and (VIIIe), R¹ and R² together form an oxetane.

In one embodiment of compounds of Formulas (III), (IV), (IVa), (V), (Va), (VI), (VIa), (VII), (VIIa), (VIIb), (VIII), (VIIIa), (VIIIb), (VIIIc), (VIIId) and (VIIIe), R³ and R⁴ are independently selected from hydrogen, —C₁₋₄ alkyl, together R³ and R⁴ form an oxo, or one of R³ and R⁴ form a double bond with R⁵ and the other of R³ and R⁴ is hydrogen or —C₁₋₄ alkyl. In certain embodiments of Formulas (III), (IV), (IVa), (V), (Va), (VI), (VIa), (VII), (VIIa), (VIIb), (VIII), (VIIIa), (VIIIb), (VIIIc), (VIIId) and (VIIIe), R³ and R⁴ are each hydrogen. In other embodiments of Formulas (III), (IV), (IVa), (V), (Va), (VI), (VIa), (VII), (VIIa), (VIIb), (VIII), (VIIIa), (VIIIb), (VIIIc), (VIIId) and (VIIIe), at least one of R³ and R⁴ is —C₁₋₄ alkyl and the other is hydrogen or —C₁₋₄ alkyl. In certain embodiments of Formulas (III), (IV), (IVa), (V), (Va), (VI), (VIa), (VII), (VIIa), (VIIb), (VIII), (VIIIa), (VIIIb), (VIIIc), (VIIId) and (VIIIe), R³ and R⁴ are each —C₁₋₄ alkyl. In other embodiments of Formulas (III), (IV), (IVa), (V), (Va), (VI), (VIa), (VII), (VIIa), (VIIb), (VIII), (VIIIa), (VIIIb), (VIIIc), (VIIId) and (VIIIe), R³ and R⁴ together form an oxo, or one of R³ and R⁴ form a double bond with R⁵ and the other of R³ and R⁴ is hydrogen or —C₁₋₄ alkyl.

In one embodiment of compounds of Formulas (III), (IV), (IVa), (V), (Va), (VI), (VIa), (VII), (VIIa), (VIIb), (VIII), (VIIIa), (VIIIb), (VIIIc), (VIIId) and (VIIIe), R⁶ and R⁷ are independently selected from hydrogen, —C₁₋₄ alkyl, or together R⁶ and R⁷ form an oxo. In one embodiment of compounds of Formulas (III), (IV), (IVa), (V), (Va), (VI), (VIa), (VII), (VIIa), (VIIb), (VIII), (VIIIa), (VIIIb), (VIIIc), (VIIId) and (VIIIe), at least one of R⁶ and R⁷ is hydrogen, such as wherein both are hydrogen. In one embodiment of compounds of Formulas (III), (IV), (IVa), (V), (Va), (VI), (VIa), (VII), (VIIa), (VIIb), (VIII), (VIIIa), (VIIIb), (VIIIc), (VIIId) and (VIIIe), at least one of R⁶ and R⁷ is —C₁₋₄ alkyl, such as methyl. In one embodiment of compounds of Formulas (III), (IV), (IVa), (V), (Va), (VI), (VIa), (VII), (VIIa), (VIIb), (VIII), (VIIIa), (VIIIb), (VIIIc), (VIIId) and (VIIIe), both of R⁶ and R⁷ are —C₁₋₄ alkyl. In one embodiment of compounds of Formulas (III), (IV), (IVa), (V), (Va), (VI), (VIa), (VII), (VIIa), (VIIb), (VIII), (VIIIa), (VIIIb), (VIIIc), (VIIId) and (VIIIe), R⁶ and R⁷ together form an oxo.

Any combination of the groups described above for the various variables is contemplated herein. Throughout the specification, groups and substituents thereof are chosen by one skilled in the field to provide stable moieties and compounds.

In one embodiment, disclosed compounds according to Formulas (III), (IV), (IVa), (V), (Va), (VI), (VIa), (VII), (VIIa), (VIIb), (VIII), (VIIIa), (VIIIb), (VIIIc), (VIIId) and/or (VIIIe) include those set forth in Table (II).

TABLE (II)

| ID No. | Structure | Name |
|---|---|---|
| II-1 | 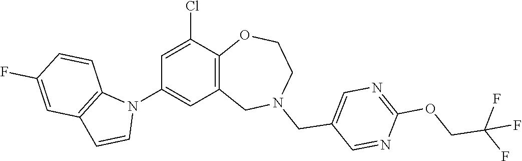 [SMILES: Fc1ccc2c(ccn2-c2cc(Cl)c3c(c2)CN(Cc2cnc(OCC(F)(F)F)nc2)CCO3)c1] | 9-chloro-7-(5-fluoro-1H-indol-1- yl)-4-((2-(2,2,2-trifluoroethoxy)pyrimidin- 5-yl)methyl)-2,3,4,5-tetrahydrobenzo [f][1,4]oxazepine |
| II-2 | 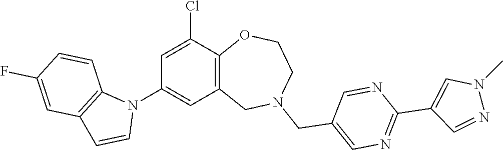 [SMILES: Cn1cc(-c2ncc(CN3CCOc4c(Cl)cc(-n5ccc6cc(F)ccc65)cc4C3)cn2)cn1] | 9-chloro-7-(5-fluoro-1H-indol-1- yl)-4-((2-(1-methyl-1H-pyrazol- 4-yl)pyrimidin-5-yl)methyl)- 2,3,4,5-tetrahydrobenzo[f][1,4] oxazepine |
| II-3 | 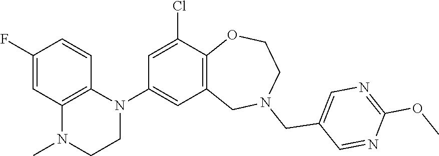 [SMILES: COc1ncc(CN2CCOc3c(Cl)cc(N4CCN(C)c5cc(F)ccc54)cc3C2)cn1] | 9-chloro-7-(6-fluoro-4-methyl- 3,4-dihydroquinoxalin-1(2H)-yl)- 4-((2-methoxypyrimidin-5- yl)methyl)-2,3,4,5-tetrahydrobenzo[f][1,4] oxazepine |
| II-4 | 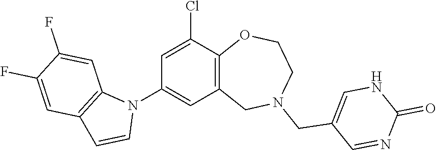 [SMILES: O=c1ncc(CN2CCOc3c(Cl)cc(-n4ccc5cc(F)c(F)cc54)cc3C2)c[nH]1] | 5-((9-chloro-7-(5,6-difluoro-1H- indol-1-yl)-2,3- dihydrobenzo[f][1,4]oxazepin- 4(5H)-yl)methyl)pyrimidin- 2(1H)-one |
| II-5 | 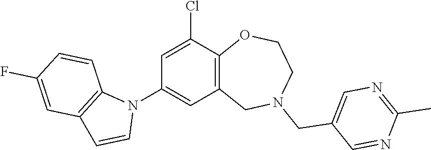 [SMILES: Cc1ncc(CN2CCOc3c(Cl)cc(-n4ccc5cc(F)ccc54)cc3C2)cn1] | 9-chloro-7-(5-fluoro-1H-indol-1- yl)-4-((2-methylpyrimidin-5- yl)methyl)-2,3,4,5-tetrahydrobenzo[f][1,4] oxazepine |
| II-6 | 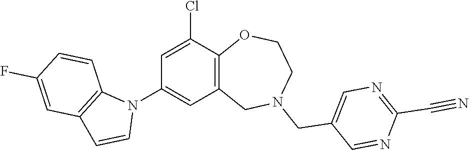 [SMILES: N#Cc1ncc(CN2CCOc3c(Cl)cc(-n4ccc5cc(F)ccc54)cc3C2)cn1] | 5-((9-chloro-7-(5-fluoro-1H- indol-1-yl)-2,3- dihydrobenzo[f][1,4]oxazepin- 4(5H)-yl)methyl)pyrimidine-2- carbonitrile |
| II-7 | 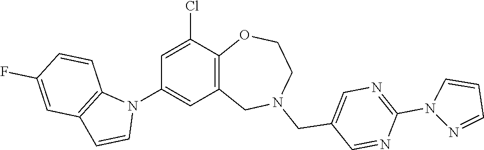 [SMILES: Fc1ccc2c(ccn2-c2cc(Cl)c3c(c2)CN(Cc2cnc(-n4cccn4)nc2)CCO3)c1] | 4-((2-(1H-pyrazol-1- yl)pyrimidin-5-yl)methyl)-9- chloro-7-(5-fluoro-1H-indol-1- yl)-2,3,4,5-tetrahydrobenzo[f][1,4] oxazepine |
| II-8 | 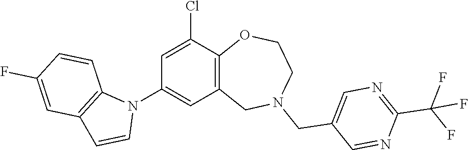 [SMILES: Fc1ccc2c(ccn2-c2cc(Cl)c3c(c2)CN(Cc2cnc(C(F)(F)F)nc2)CCO3)c1] | 9-chloro-7-(5-fluoro-1H-indol-1- yl)-4-((2- (trifluoromethyl)pyrimidin-5- yl)methyl)-2,3,4,5-tetrahydrobenzo[f][1,4] oxazepine |
| II-9 | 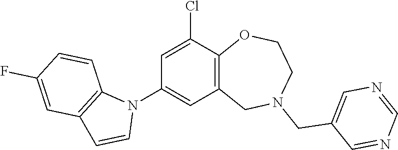 [SMILES: Fc1ccc2c(ccn2-c2cc(Cl)c3c(c2)CN(Cc2cncnc2)CCO3)c1] | 9-chloro-7-(5-fluoro-1H-indol-1- yl)-4-(pyrimidin-5-ylmethyl)- 2,3,4,5-tetrahydrobenzo[f][1,4] oxazepine |
| II-10 | 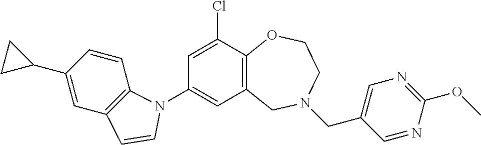 [SMILES: COc1ncc(CN2CCOc3c(Cl)cc(-n4ccc5cc(C6CC6)ccc54)cc3C2)cn1] | 9-chloro-7-(5-cyclopropyl-1H- indol-1-yl)-4-((2- methoxypyrimidin-5-yl)methyl)- 2,3,4,5-tetrahydrobenzo[f][1,4] oxazepine |
| II-11 | 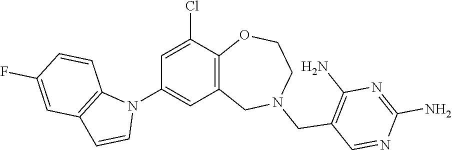 [SMILES: Nc1ncc(CN2CCOc3c(Cl)cc(-n4ccc5cc(F)ccc54)cc3C2)c(N)n1] | 5-((9-chloro-7-(5-fluoro-1H- indol-1-yl)-2,3- dihydrobenzo[f][1,4]oxazepin- 4(5H)-yl)methyl)pyrimidin-2,4- diamine |
| II-12 | 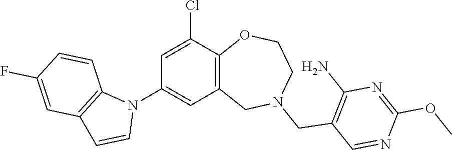 [SMILES: COc1ncc(CN2CCOc3c(Cl)cc(-n4ccc5cc(F)ccc54)cc3C2)c(N)n1] | 5-((9-chloro-7-(5-fluoro-1H- indol-1-yl)-2,3- dihydrobenzo[f][1,4]oxazepin- 4(5H)-yl)methyl)pyrimidin-2,4- diamine |
| II-13 | 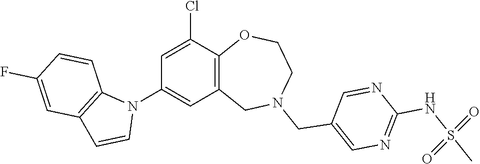 [SMILES: CS(=O)(=O)Nc1ncc(CN2CCOc3c(Cl)cc(-n4ccc5cc(F)ccc54)cc3C2)cn1] | N-(5-((9-chloro-7-(5-fluoro-1H- indol-1-yl)-2,3- dihydrobenzo[f][1,4]oxazepin- 4(5H)-yl)methyl)pyrimidin-2,4- diamine |
| II-14 | 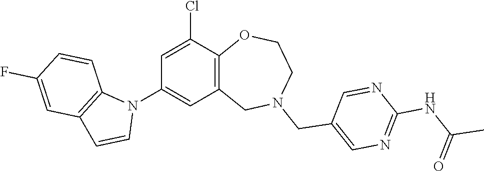 [SMILES: CC(=O)Nc1ncc(CN2CCOc3c(Cl)cc(-n4ccc5cc(F)ccc54)cc3C2)cn1] | N-(5-((9-chloro-7-(5-fluoro-1H- indol-1-yl)-2,3- dihydrobenzo[f][1,4]oxazepin- 4(5H)-yl)methyl)pyrimidin-2- yl)acetamide |
| II-15 | 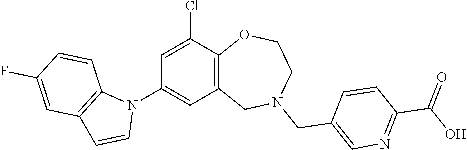 [SMILES: O=C(O)c1ccc(CN2CCOc3c(Cl)cc(-n4ccc5cc(F)ccc54)cc3C2)cn1] | 5-((9-chloro-7-(5-fluoro-1H- indol-1-yl)-2,3- dihydrobenzo[f][1,4]oxazepin- 4(5H)-yl)methyl)picolinic acid |
| II-16 | 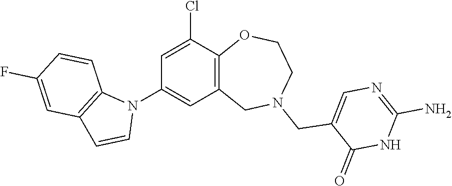 [SMILES: Nc1ncc(CN2CCOc3c(Cl)cc(-n4ccc5cc(F)ccc54)cc3C2)c(=O)[nH]1] | 2-amino-5-((9-chloro-7-(5- fluoro-1H-indol-1-yl)-2,3- dihydrobenzo[f][1,4]oxazepin- 4(5H)-yl)methyl)pyrimidin- 4(3H)-one |
| II-17 | 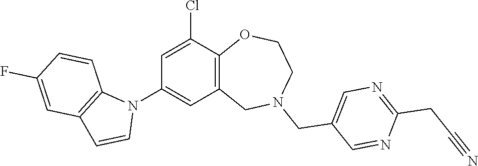 [SMILES: N#CCc1ncc(CN2CCOc3c(Cl)cc(-n4ccc5cc(F)ccc54)cc3C2)cn1] | 2-(5-((9-chloro-7-(5-fluoro-1H- indol-1-yl)-2,3- dihydrobenzo[f][1,4]oxazepin- 4(5H)-yl)methyl)pyrimidin-2- yl)acetonitrile |
| II-18 | 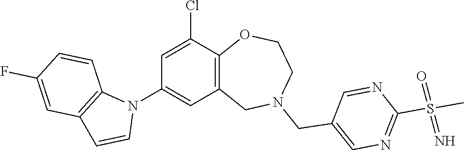 [SMILES: CS(=N)(=O)c1ncc(CN2CCOc3c(Cl)cc(-n4ccc5cc(F)ccc54)cc3C2)cn1] | 9-chloro-7-(5-fluoro-1H-indol-1- yl)-4-((2-(S- methylsulfonimidoyl)pyrimidin- 5-yl)methyl)-2,3,4,5- tetrahydrobenzo[f][1,4]oxazepine |
| II-19 | 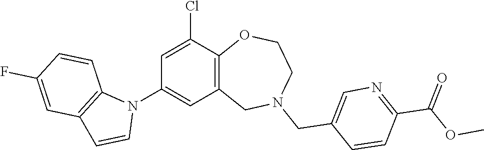 [SMILES: COC(=O)c1ccc(CN2CCOc3c(Cl)cc(-n4ccc5cc(F)ccc54)cc3C2)cn1] | methyl 5-((9-chloro-7-(5-fluoro- 1H-indol-1-yl)-2,3- dihydrobenzo[f][1,4]oxazepin- 4(5H)-yl)methyl)picolinate |
| II-20 | 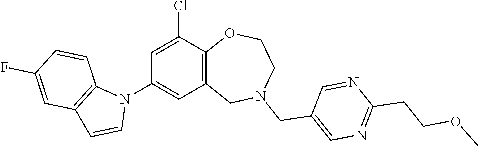 [SMILES: COCCc1ncc(CN2CCOc3c(Cl)cc(-n4ccc5cc(F)ccc54)cc3C2)cn1] | 9-chloro-7-(5-fluoro-1H-indol-1- yl)-4-((2-(2- methoxyethyl)pyrimidin-5- yl)methyl)-2,3,4,5- tetrahydrobenzo[f][1,4]oxazepine |
| II-21 | 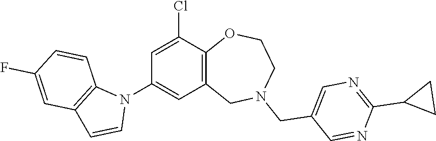 [SMILES: Fc1ccc2c(ccn2-c2cc(Cl)c3c(c2)CN(Cc2cnc(C4CC4)nc2)CCO3)c1] | 9-chloro-4-((2- cyclopropylpyrimidin-5- yl)methyl)-7-(5-fluoro-1H-indol- 1-yl)-2,3,4,5- tetrahydrobenzo[f][1,4]oxazepine |
| II-22 | 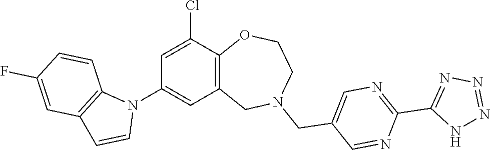 [SMILES: Fc1ccc2c(ccn2-c2cc(Cl)c3c(c2)CN(Cc2cnc(-c4nnn[nH]4)nc2)CCO3)c1] | 4-((2-(1H-tetrazol-5- yl)pyrimidin-5-yl)methyl)-9- chloro-7-(5-fluoro-1H-indol-1- yl)-2,3,4,5- tetrahydrobenzo[f][1,4]oxazepine |
| II-23 | 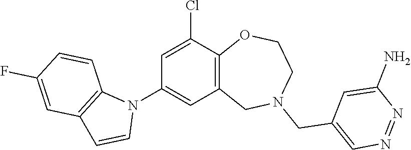 [SMILES: Nc1cc(CN2CCOc3c(Cl)cc(-n4ccc5cc(F)ccc54)cc3C2)cnn1] | 5-((9-chloro-7-(5-fluoro-1H- indol-1-yl)-2,3- dihydrobenzo[f][1,4]oxazepin- 4(5H)-yl)methyl)pyridazin-3- amine |
| II-24 | 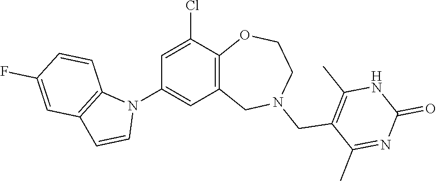 [SMILES: Cc1nc(=O)[nH]c(C)c1CN1CCOc2c(Cl)cc(-n3ccc4cc(F)ccc43)cc2C1] | 5-((9-chloro-7-(5-fluoro-1H- indol-1-yl)-2,3- dihydrobenzo[f][1,4]oxazepin- 4(5H)-yl)methyl)-4,6- dimethylpyrimidin-2(1H)-one |
| II-25 | 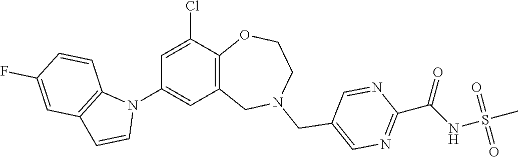 [SMILES: CS(=O)(=O)NC(=O)c1ncc(CN2CCOc3c(Cl)cc(-n4ccc5cc(F)ccc54)cc3C2)cn1] | 5-((9-chloro-7-(5-fluoro-1H- indol-1-yl)-2,3- dihydrobenzo[f][1,4]oxazepin- 4(5H)-yl)methyl)-N- (methylsulfonyl)pyrimidine-2- carboxamide |
| II-26 | 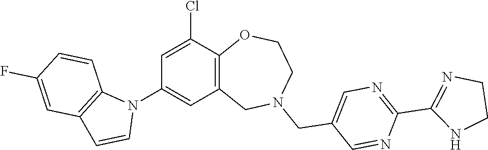 [SMILES: Fc1ccc2c(ccn2-c2cc(Cl)c3c(c2)CN(Cc2cnc(C4=NCCN4)nc2)CCO3)c1] | 9-chloro-4-((2-(4,5-dihydro-1H- imidazol-2-yl)pyrimidin-5- yl)methyl)-7-(5-fluoro-1H-indol- 1-yl)-2,3,4,5- tetrahydrobenzo[f][1,4]oxazepine |
| II-27 | 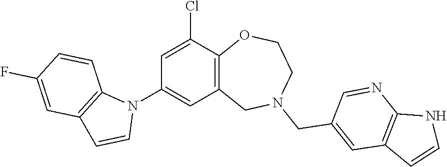 [SMILES: Fc1ccc2c(ccn2-c2cc(Cl)c3c(c2)CN(Cc2cnc4[nH]ccc4c2)CCO3)c1] | 4-((1H-pyrrolo[2,3-b]pyridin-5- yl)methyl)-9-chloro-7-(5-fluoro- 1H-indol-1-yl)-2,3,4,5- tetrahydrobenzo[f][1,4]oxazepine |
| II-28 | 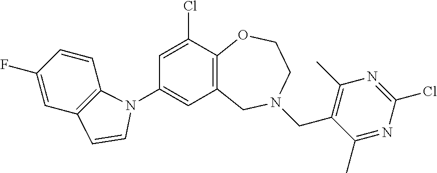 [SMILES: Cc1nc(Cl)nc(C)c1CN1CCOc2c(Cl)cc(-n3ccc4cc(F)ccc43)cc2C1] | 9-chloro-4-((2-chloro-4,6- dimethylpyrimidin-5-yl)methyl)- 7-(5-fluoro-1H-indol-1-yl)- 2,3,4,5- tetrahydrobenzo[f][1,4]oxazepine |
| II-29 | 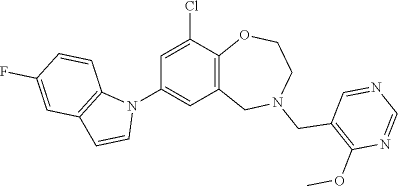 [SMILES: COc1ncncc1CN1CCOc2c(Cl)cc(-n3ccc4cc(F)ccc43)cc2C1] | 9-chloro-7-(5-fluoro-1H-indol-1- yl)-4-((4-methoxypyrimidin-5- yl)methyl)-2,3,4,5- tetrahydrobenzo[f][1,4]oxazepine |
| II-30 | 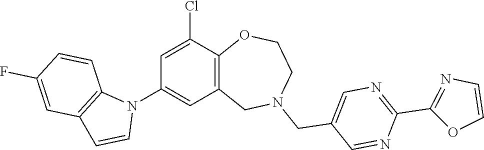 [SMILES: Fc1ccc2c(ccn2-c2cc(Cl)c3c(c2)CN(Cc2cnc(-c4ncco4)nc2)CCO3)c1] | 9-chloro-7-(5-fluoro-1H-indol-1- yl)-4-((2-(oxazol-2-yl)pyrimidin- 5-yl)methyl)-2,3,4,5- tetrahydrobenzo[f][1,4]oxazepine |
| II-31 | 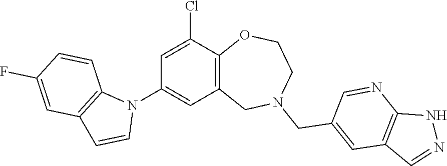 [SMILES: Fc1ccc2c(ccn2-c2cc(Cl)c3c(c2)CN(Cc2cnc4[nH]ncc4c2)CCO3)c1] | 4-((1H-pyrazolo[3,4-b]pyridin-5- yl)methyl)-9-chloro-7-(5-fluoro- 1H-indol-1-yl)-2,3,4,5- tetrahydrobenzo[f][1,4]oxazepine |
| II-32 | 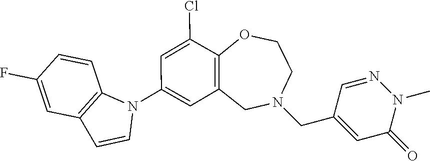 [SMILES: Cn1ncc(CN2CCOc3c(Cl)cc(-n4ccc5cc(F)ccc54)cc3C2)cc1=O] | 5-((9-chloro-7-(5-fluoro-1H- indol-1-yl)-2,3- dihydrobenzo[f][1,4]oxazepin- 4(5H)-yl)methyl)-2- methylpyridazin-3(2H)-one |
| II-33 | 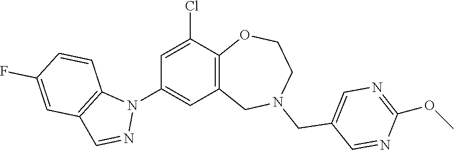 [SMILES: COc1ncc(CN2CCOc3c(Cl)cc(-n4ncc5cc(F)ccc54)cc3C2)cn1] | 9-chloro-7-(5-fluoro-1H-indazol- 1-yl)-4-((2-methoxypyrimidin-5- yl)methyl)-2,3,4,5- tetrahydrobenzo[f][1,4]oxazepine |
| II-34 | 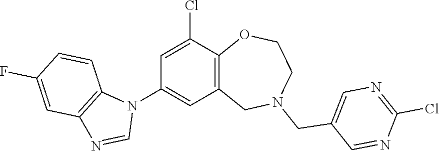 [SMILES: Fc1ccc2c(c1)ncn2-c1cc(Cl)c2c(c1)CN(Cc1cnc(Cl)nc1)CCO2] | 9-chloro-4-((2-chloropyrimidin- 5-yl)methyl)-7-(5-fluoro-1H- benzo[d]imidazol-1-yl)-2,3,4,5- tetrahydrobenzo[f][1,4]oxazepine |
| II-35 | 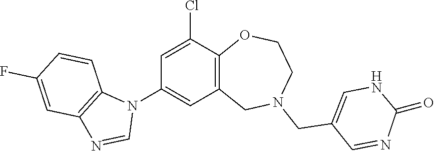 [SMILES: O=c1ncc(CN2CCOc3c(Cl)cc(-n4cnc5cc(F)ccc54)cc3C2)c[nH]1] | 5-((9-chloro-7-(5-fluoro-1H- benzo[d]imidazol-1-yl)-2,3- dihydrobenzo[f][1,4]oxazepin- 4(5H)-yl)methyl)pyrimidin- 2(1H)-one |
| II-36 | 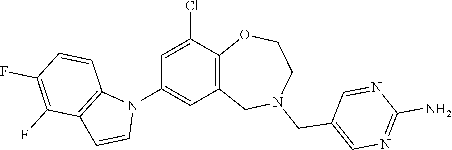 [SMILES: Nc1ncc(CN2CCOc3c(Cl)cc(-n4ccc5c(F)c(F)ccc54)cc3C2)cn1] | 5-((9-chloro-7-(4,5-difluoro-1H- indol-1-yl)-2,3- dihydrobenzo[f][1,4]oxazepin- 4(5H)-yl)methyl)pyrimidin-2- amine |
| II-37 | 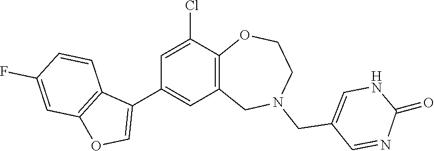 [SMILES: O=c1ncc(CN2CCOc3c(Cl)cc(-c4coc5cc(F)ccc45)cc3C2)c[nH]1] | 1-(5-((9-chloro-7-(5-fluoro-1H- indol-1-yl)-2,3- dihydrobenzo[f][1,4]oxazepin- 4(5H)-yl)methyl)pyrimidin-2- yl)ethanone |
| II-38 | 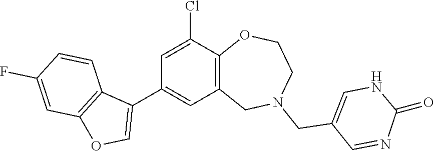 [SMILES: O=c1ncc(CN2CCOc3c(Cl)cc(-c4coc5cc(F)ccc45)cc3C2)c[nH]1] | 5-((9-chloro-7-(6- fluorobenzofuran-3-yl)-2,3- dihydrobenzo[f][1,4]oxazepin- 4(5H)-yl)methyl)pyrimidin- 2(1H)-one |
| II-39 | 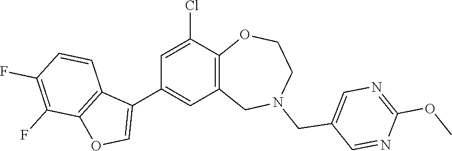 [SMILES: COc1ncc(CN2CCOc3c(Cl)cc(-c4coc5c(F)c(F)ccc45)cc3C2)cn1] | 9-chloro-7-(6,7- difluorobenzofuran-3-yl)-4-((2- methoxypyrimidin-5-yl)methyl)- 2,3,4,5- tetrahydrobenzo[f][1,4]oxazepine |
| II-40 | 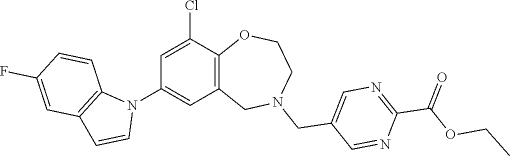 [SMILES: CCOC(=O)c1ncc(CN2CCOc3c(Cl)cc(-n4ccc5cc(F)ccc54)cc3C2)cn1] | ethyl 5-((9-chloro-7-(5-fluoro- 1H-indol-1-yl)-2,3- dihydrobenzo[f][1,4]oxazepin- 4(5H)-yl)methyl)pyrimidine-2- carboxylate |
| II-41 | 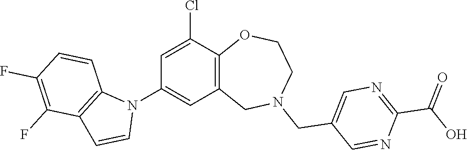 [SMILES: O=C(O)c1ncc(CN2CCOc3c(Cl)cc(-n4ccc5c(F)c(F)ccc54)cc3C2)cn1] | 5-((9-chloro-7-(4,5-difluoro-1H- indol-1-yl)-2,3- dihydrobenzo[f][1,4]oxazepin- 4(5H)-yl)methyl)pyrimidine-2- carboxylic acid |
| II-42 | 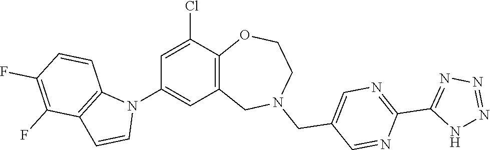 [SMILES: Fc1ccc2c(ccn2-c2cc(Cl)c3c(c2)CN(Cc2cnc(-c4nnn[nH]4)nc2)CCO3)c1F] | 4-((2-(1H-tetrazol-5- yl)pyrimidin-5-yl)methyl)-9- chloro-7-(4,5-difluoro-1H-indol- 1-yl)-2,3,4,5- tetrahydrobenzo[f][1,4]oxazepine |
| II-43 | 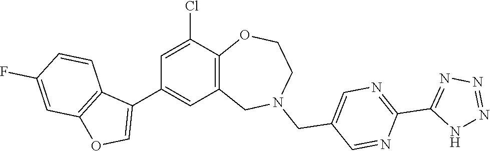 [SMILES: Fc1ccc2c(-c3cc(Cl)c4c(c3)CN(Cc3cnc(-c5nnn[nH]5)nc3)CCO4)coc2c1] | 4-((2-(1H-tetrazol-5- yl)pyrimidin-5-yl)methyl)-9- chloro-7-(6-fluorobenzofuran-3- yl)-2,3,4,5- tetrahydrobenzo[f][1,4]oxazepine |
| II-44 | 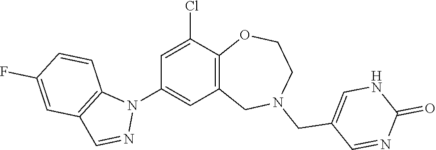 [SMILES: O=c1ncc(CN2CCOc3c(Cl)cc(-n4ncc5cc(F)ccc54)cc3C2)c[nH]1] | 5-((9-chloro-7-(5-fluoro-1H- indazol-1-yl)-2,3- dihydrobenzofuran[f][1,4]oxazepin- 4(5H)-yl)methyl)pyrimidin- 2(1H)-one |
| II-45 | 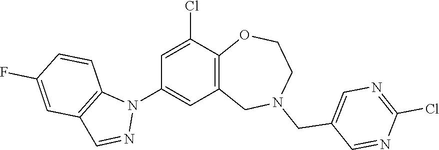 [SMILES: Fc1ccc2c(cnn2-c2cc(Cl)c3c(c2)CN(Cc2cnc(Cl)nc2)CCO3)c1] | 9-chloro-4-((2-chloropyrimidin- 5-yl)methyl)-7-(5-fluoro-1H- indazol-1-yl)-2,3,4,5- tetrahydrobenzo[f][1,4]oxazepine |
| II-46 | 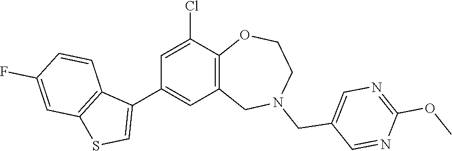 [SMILES: COc1ncc(CN2CCOc3c(Cl)cc(-c4csc5cc(F)ccc45)cc3C2)cn1] | 9-chloro-7-(6- fluorobenzo[b]thiophen-3-yl)-4- ((2-methoxypyrimidin-5- yl)methyl)-2,3,4,5- tetrahydrobenzo[f][1,4]oxazepine |
| II-47 | 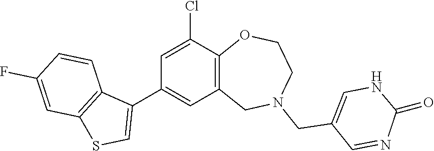 [SMILES: O=c1ncc(CN2CCOc3c(Cl)cc(-c4csc5cc(F)ccc45)cc3C2)c[nH]1] | 5-((9-chloro-7-(6- fluorobenzo[b]thiophen-3-yl)- 2,3- dihydrobenzo[f][1,4]oxazepin- 4(5H)-yl)methyl)pyrimidin- 2(1H)-one |
| II-48 | 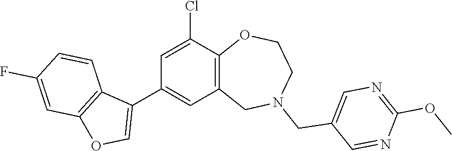 [SMILES: COc1ncc(CN2CCOc3c(Cl)cc(-c4coc5cc(F)ccc45)cc3C2)cn1] | 9-chloro-7-(6-fluorobenzofuran- 3-yl)-4-((2-methoxypyrimidin-5- yl)methyl)-2,3,4,5- tetrahydrobenzo[f][1,4]oxazepine |
| II-49 | 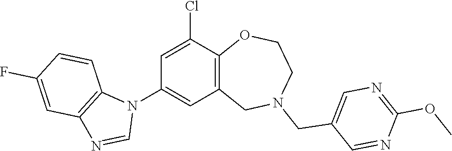 [SMILES: COc1ncc(CN2CCOc3c(Cl)cc(-n4cnc5cc(F)ccc54)cc3C2)cn1] | 9-chloro-7-(5-fluoro-1H- benzo[d]imidazol-1-yl)-4-((2- methoxypyrimidin-5-yl)methyl)- 2,3,4,5- tetrahydrobenzo[f][1,4]oxazepine |
| II-50 | 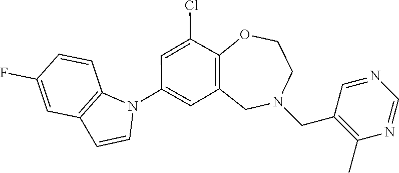 [SMILES: Cc1ncncc1CN1CCOc2c(Cl)cc(-n3ccc4cc(F)ccc43)cc2C1] | 9-chloro-7-(5-fluoro-1H-indol-1- yl)-4-((4-methylpyrimidin-5- yl)methyl)-2,3,4,5- tetrahydrobenzo[f][1,4]oxazepine |
| II-51 | 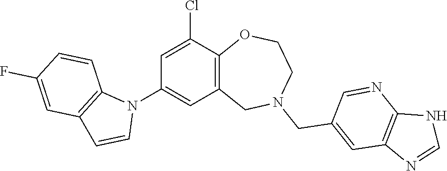 [SMILES: Fc1ccc2c(ccn2-c2cc(Cl)c3c(c2)CN(Cc2cnc4[nH]cnc4c2)CCO3)c1] | 4-((3H-imidazo[4,5-b]pyridin-6- yl)methyl)-9-chloro-7-(5-fluoro- 1H-indol-1-yl)-2,3,4,5- tetrahydrobenzo[f][1,4]oxazepine |
| II-52 | 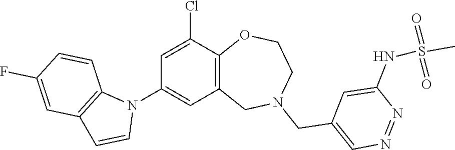 [SMILES: CS(=O)(=O)Nc1cc(CN2CCOc3c(Cl)cc(-n4ccc5cc(F)ccc54)cc3C2)cnn1] | N-(5-((9-chloro-7-(5-fluoro-1H- indol-1-yl)-2,3- dihydrobenzo[f][1,4]oxazepin- 4(5H)-yl)methyl)pyridazin-3- yl)methanesulfonamide |
| II-53 | 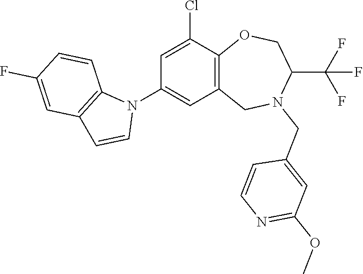 [SMILES: COc1cc(CN2Cc3cc(-n4ccc5cc(F)ccc54)cc(Cl)c3OCC2C(F)(F)F)ccn1] | 9-chloro-7-(5-fluoro-1H-indol-1- yl)-4-((2-methoxypyridin-4- yl)methyl)-3-(trifluoromethyl)-3,4- dihydrobenzo[f][1,4]oxazepin- 5(2H)-one |
| II-54 | 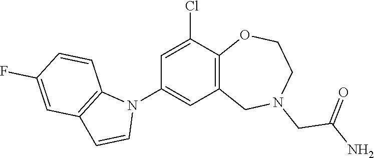 [SMILES: NC(=O)CN1CCOc2c(Cl)cc(-n3ccc4cc(F)ccc43)cc2C1] | 2-(9-chloro-7-(5-fluoro-1H- indol-1-yl)-2,3- dihydrobenzo[f][1,4]oxazepin- 4(5H)-yl)acetamide |
| II-55 | 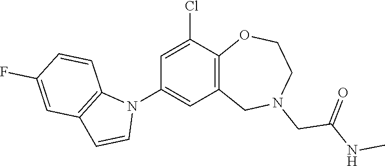 [SMILES: CNC(=O)CN1CCOc2c(Cl)cc(-n3ccc4cc(F)ccc43)cc2C1] | 2-(9-chloro-7-(5-fluoro-1H- indol-1-yl)-2,3- dihydrobenzo[f][1,4]oxazepin- 4(5H)-yl)-N-methylacetamide |
| II-56 | 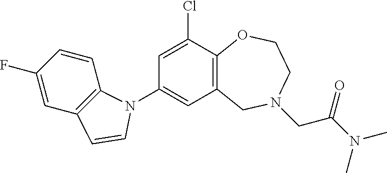 [SMILES: CN(C)C(=O)CN1CCOc2c(Cl)cc(-n3ccc4cc(F)ccc43)cc2C1] | 2-(9-chloro-7-(5-fluoro-1H- indol-1-yl)-2,3- dihydrobenzo[f][1,4]oxazepin- 4(5H)-yl)-N,N- dimethylacetamide |
| II-57 | 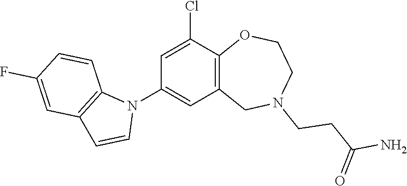 [SMILES: NC(=O)CCN1CCOc2c(Cl)cc(-n3ccc4cc(F)ccc43)cc2C1] | 3-(9-chloro-7-(5-fluoro-1H-indol-1- yl)-2,3-dihydrobenzo[f][1,4]oxazepin- 4(5H)-yl)propanamide |
| II-58 | 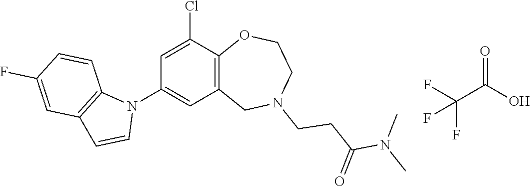 | 3-(9-chloro-7-(5-fluoro-1H- indol-1-yl)-2,3- dihydrobenzo[f][1,4]oxazepin- 4(5H)-yl)-N,N- dimethylpropanamide 2,2,2- trifluoroacetate |
| II-59 | 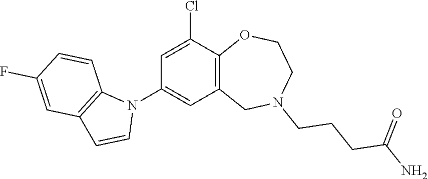 [SMILES: NC(=O)CCCN1CCOc2c(Cl)cc(-n3ccc4cc(F)ccc43)cc2C1] | 4-(9-chloro-7-(5-fluoro-1H- indol-1-yl)-2,3- dihydrobenzo[f][1,4]oxazepin- 4(5H)-yl)butanamide |
| II-60 | 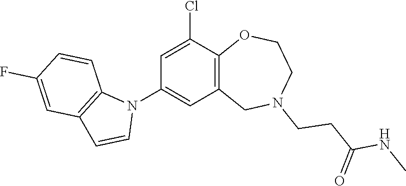 [SMILES: CNC(=O)CCN1CCOc2c(Cl)cc(-n3ccc4cc(F)ccc43)cc2C1] | 3-(9-chloro-7-(5-fluoro-1H- indol-1-yl)-2,3- dihydrobenzo[f][1,4]oxazepin- 4(5H)-yl)-N-methylpropanamide |
| II-61 | 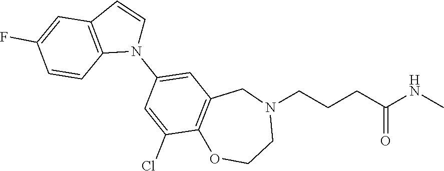 [SMILES: CNC(=O)CCCN1CCOc2c(Cl)cc(-n3ccc4cc(F)ccc43)cc2C1] | 4-(9-chloro-7-(5-fluoro-1H- indol-1-yl)-2,3- dihydrobenzo[f][1,4]oxazepin- 4(5H)-yl)-N-methylbutanamide |
| II-62 | 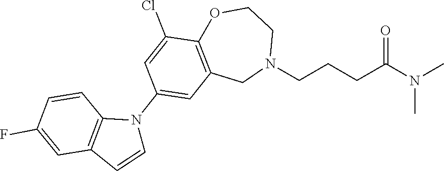 [SMILES: CN(C)C(=O)CCCN1CCOc2c(Cl)cc(-n3ccc4cc(F)ccc43)cc2C1] | 4-(9-chloro-7-(5-fluoro-1H- indol-1-yl)-2,3- dihydrobenzo[f][1,4]oxazepin- 4(5H)-yl)-N,N- dimethylbutanamide |
| II-63 | 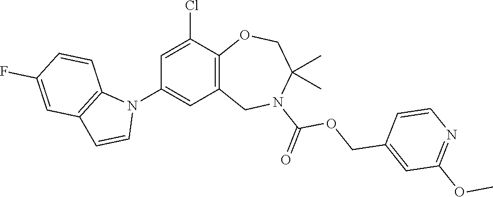 [SMILES: COc1cc(COC(=O)N2Cc3cc(-n4ccc5cc(F)ccc54)cc(Cl)c3OCC2(C)C)ccn1] | (2-methoxypyridin-4-yl)methyl 9-chloro-7-(5-fluoro-1H-indol-1- yl)-3,3-dimethyl-2,3- dihydrobenzo[f][1,4]oxazepine- 4(5H)-carboxylate |
| II-64 | 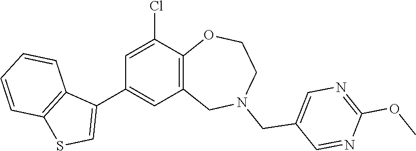 [SMILES: COc1ncc(CN2CCOc3c(Cl)cc(-c4csc5ccccc45)cc3C2)cn1] | 7-(benzo[b]thiophen-3-yl)-9- chloro-4-((2-methoxypyrimidin- 5-yl)methyl)-2,3,4,5- tetrahydrobenzo[f][1,4]oxazepine |
| II-65 | 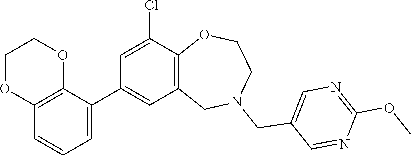 [SMILES: COc1ncc(CN2CCOc3c(Cl)cc(-c4cccc5c4OCCO5)cc3C2)cn1] | 9-chloro-7-(2,3- dihydrobenzo[b][1,4]dioxin-5- yl)-4-((2-methoxypyrimidin-5- yl)methyl)-2,3,4,5- tetrahydrobenzo[f][1,4]oxazepine |
| II-66 | 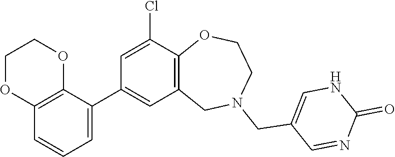 [SMILES: O=c1ncc(CN2CCOc3c(Cl)cc(-c4cccc5c4OCCO5)cc3C2)c[nH]1] | 5-((9-chloro-7-(2,3- dihydrobenzo[b][1,4]dioxin-5- yl)-2,3- dihydrobenzo[f][1,4]oxazepin- 4(5H)-yl)methyl)pyrimidin- 2(1H)-one |
| II-67 | 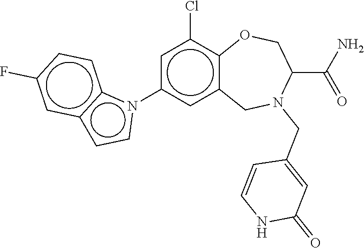 | 9-chloro-7-(5-fluoro-1H-indol-1- yl)-4-((2-oxo-1,2- dihydropyridin-4-yl)methyl)- 2,3,4,5- tetrahydrobenzo[f][1,4]oxazepine- 3-carboxamide |
| II-68 | 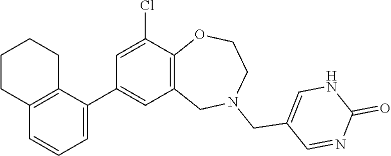 [SMILES: O=c1ncc(CN2CCOc3c(Cl)cc(-c4cccc5c4CCCC5)cc3C2)c[nH]1] | 5-((9-chloro-7-(5,6,7,8- tetrahydronaphthalen-1-yl)-2,3- dihydrobenzo[f][1,4]oxazepin- 4(5H)-yl)methyl)pyrimidin- 2(1H)-one |
| II-69 | 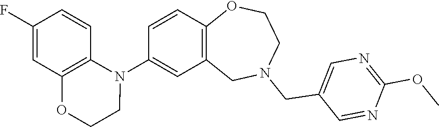 [SMILES: COc1ncc(CN2CCOc3ccc(N4CCOc5cc(F)ccc54)cc3C2)cn1] | 7-(7-fluoro-2H- benzo[f][1,4]oxazin-4(3H)-yl)-4- ((2-methoxypyrimidin-5- yl)methyl)-2,3,4,5- tetrahydrobenzo[f][1,4]oxazepine |
| II-70 | 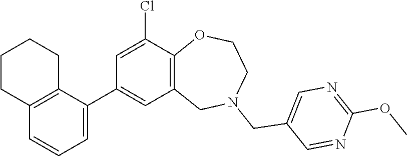 [SMILES: COc1ncc(CN2CCOc3c(Cl)cc(-c4cccc5c4CCCC5)cc3C2)cn1] | 9-chloro-4-((2- methoxypyrimidin-5-yl)methyl)- 7-(5,6,7,8-tetrahydronaphthalen- 1-yl)-2,3,4,5- tetrahydrobenzo[f][1,4]oxazepine |
| II-71 | 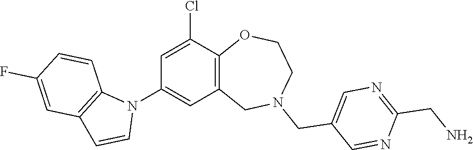 [SMILES: NCc1ncc(CN2CCOc3c(Cl)cc(-n4ccc5cc(F)ccc54)cc3C2)cn1] | (5-((9-chloro-7-(5-fluoro-1H- indol-1-yl)-2,3- dihydrobenzo[f][1,4]oxazepin- 4(5H)-yl)methyl)pyrimidin-2- yl)methanamine |
| II-72 | 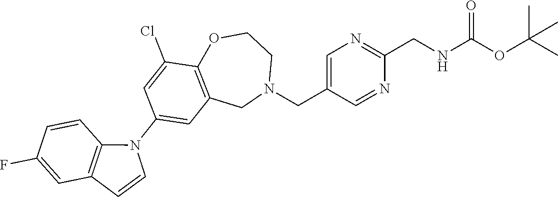 [SMILES: CC(C)(C)OC(=O)NCc1ncc(CN2CCOc3c(Cl)cc(-n4ccc5cc(F)ccc54)cc3C2)cn1] | tert-butyl ((5-((9-chloro-7-(5- fluoro-1H-indol-1-yl)-2,3- dihydrobenzo[f][1,4]oxazepin- 4(5H)-yl)methyl)pyrimidin-2- yl)methyl)carbamate |
| II-73 | 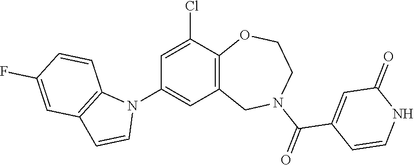 [SMILES: O=C(c1cc[nH]c(=O)c1)N1CCOc2c(Cl)cc(-n3ccc4cc(F)ccc43)cc2C1] | 4-(9-chloro-7-(5-fluoro-1H- indol-1-yl)-2,3,4,5- tetrahydrobenzo[f][1,4]oxazepine- 4-carbonyl)pyridin-2(1H)-one |
| II-74 | 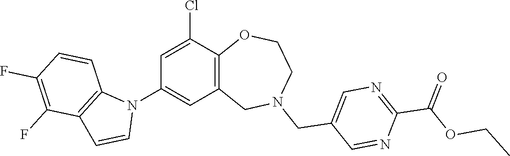 [SMILES: CCOC(=O)c1ncc(CN2CCOc3c(Cl)cc(-n4ccc5c(F)c(F)ccc54)cc3C2)cn1] | ethyl 5-((9-chloro-7-(4,5- difluoro-1H-indol-1-yl)-2,3- dihydrobenzo[f][1,4]oxazepin- 4(5H)-yl)methyl)pyrimidine-2- carboxylate |
| II-75 | 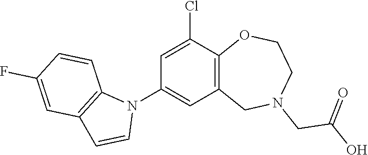 [SMILES: O=C(O)CN1CCOc2c(Cl)cc(-n3ccc4cc(F)ccc43)cc2C1] | 2-(9-chloro-7-(5-fluoro-1H- indol-1-yl)-2,3- dihydrobenzo[f][1,4]oxazepin- 4(5H)-yl)acetic acid |
| II-76 | 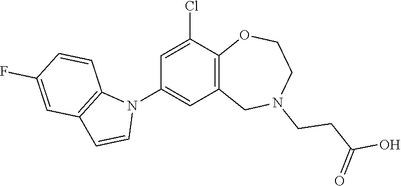 [SMILES: O=C(O)CCN1CCOc2c(Cl)cc(-n3ccc4cc(F)ccc43)cc2C1] | 3-(9-chloro-7-(5-fluoro-1H- indol-1-yl)-2,3- dihydrobenzo[f][1,4]oxazepin- 4(5H)-yl)propanoic acid |
| II-77 | 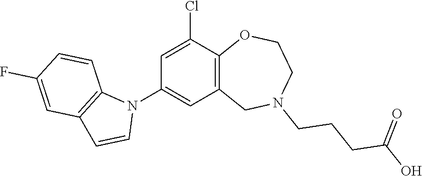 [SMILES: O=C(O)CCCN1CCOc2c(Cl)cc(-n3ccc4cc(F)ccc43)cc2C1] | 4-(9-chloro-7-(5-fluoro-1H- indol-1-yl)-2,3- dihydrobenzo[f][1,4]oxazepin- 4(5H)-yl)butanoic acid |
| II-78 | 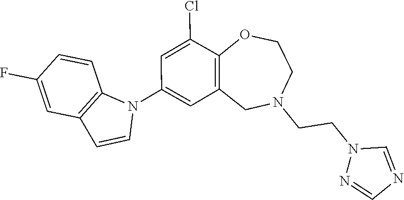 [SMILES: Fc1ccc2c(ccn2-c2cc(Cl)c3c(c2)CN(CCn2cncn2)CCO3)c1] | 4-(2-(1H-1,2,4-triazol-1- yl)ethyl)-9-chloro-7-(5-fluoro- 1H-indol-1-yl)-2,3,4,5- tetrahydrobenzo[f][1,4]oxazepine |

### Additional Forms of Compounds

In one aspect, compounds described herein are in the form of pharmaceutically acceptable salts. In addition active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zurich: Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible, and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound described herein with an acid. In some embodiments, the compound described herein (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; monomethyl fumarate, naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound described herein is prepared as a chloride salt, sulfate salt, bromide salt, mesylate salt, maleate salt, citrate salt or phosphate salt. In some embodiments, a compound described herein is prepared as a hydrochloride salt.

In some embodiments, pharmaceutically acceptable salts are obtained by reacting a compound described herein with a base. In some embodiments, the compound described herein is acidic and is reacted with a base. In such situations, an acidic proton of the compound described herein is replaced by a metal ion, for example, lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt. In some embodiments, the compounds provided herein are prepared as a sodium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), crystalline forms (also known as polymorphs), or pharmaceutically acceptable salts of compounds described herein, as well as active metabolites of these compounds having the same type of activity.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, 2H, 3H, 13C, 14C, 15N, 18O, 17O, 35S, 18F, 36Cl. In one aspect, isotopically labeled compounds described herein, for example those into which radioactive isotopes such as 3H and 14C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

The term “isotopic enrichment factor” as used herein means the ratio between the isotopic abundance and the natural abundance of a specified isotope. It will be recognized that some variation of natural isotopic abundance occurs in a synthesized compound depending upon the origin of chemical materials used in the synthesis. Thus, a preparation of any compound will inherently contain small amounts of isotopologues, including deuterated isotopologues. The concentration of naturally abundant stable hydrogen isotopes, notwithstanding this variation, is small and immaterial as compared to the degree of stable isotopic substitution of compounds of this disclosure. In a compound of this disclosure, when a particular position is designated as having a particular isotope, such as deuterium, it is understood that the abundance of deuterium at that position is substantially greater than the natural abundance of deuterium, which is about 0.015% (on a mol/mol basis). A position designated as a particular isotope will have a minimum isotopic enrichment factor of at least 3000 (45% incorporation of the indicated isotope). Thus, isotopically enriched compounds disclosed herein having deuterium will have a minimum isotopic enrichment factor of at least 3000 (45% deuterium incorporation) at each atom designated as deuterium in the compound. Such compounds may be referred to herein as “deuterated” compounds.

In other embodiments, disclosed compounds have an isotopic enrichment factor for each designated atom of at least 3500 (52.5%). For example, for such disclosed compounds that are deuterium isotopologues, the compounds have an isotopic enrichment factor for each designated hydrogen atom of at least 3500 (52.5% deuterium incorporation at each designated atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As above, such compounds also are referred to as “deuterated” compounds.

In the compounds of this disclosure any atom not specifically designated as a particular isotope is meant to represent any stable isotope of that atom. Unless otherwise stated, when a position is designated specifically as “H”, the position is understood to have hydrogen at about its natural abundance isotopic composition.

The term “isotopologue” refers to a species that has the same chemical structure and formula as another compound, with the exception of the isotopic composition at one or more positions, e.g., H vs. D. Thus, isotopologues differ in their isotopic composition.

In some embodiments, the compounds described herein possess one or more stereocenters and each stereocenter exists independently in either the R or S configuration. The compounds presented herein include all diastereomeric, enantiomeric, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as stereoselective synthesis and/or the separation of stereoisomers by chiral chromatographic columns. In certain embodiments, compounds described herein are prepared as their individual stereoisomers by reacting a racemic mixture of the compound with an optically active resolving agent to form a pair of diastereomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

### Terms

Unless otherwise stated, the following terms used in this application have the meaning described below. The use of the term “including” as well as other forms, such as “include”, “includes,” and “included,” is not limiting. The section headings used herein are for organizational purposes only and are not to be construed as limiting the subject matter described.

As used herein, C₁₋ₓ includes C₁₋₂, C₁₋₃ . . . C₁₋ₓ. By way of example only, a group designated as “C₁₋₄” indicates that there are one to four carbon atoms in the moiety, i.e. groups containing 1 carbon atom, 2 carbon atoms, 3 carbon atoms or 4 carbon atoms. Thus, by way of example only, “C₁₋₄alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁₋₆alkyl, such as a C₁₋₄alkyl In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

“Deuteroalkyl” refers to an alkyl group where 1 or more hydrogen atoms of an alkyl are replaced with deuterium.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. Non-limiting examples of an alkynyl group include —C≡CH, —C≡CCH3-C≡CCH2CH3, —CH2C≡CH.

An “alkoxy” group refers to a (alkyl)O— group, where alkyl is as defined herein.

The term “alkylamine” refers to the —N(alkyl)ₓH_{y} group, where x is 0 and y is 2, or where x is 1 and y is 1, or where x is 2 and y is 0.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a C₆-C₁₃aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e. skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicyclo[1.1.1]pentyl. In some embodiments, a cycloalkyl is a C₃-C₆cycloalkyl.

The term “halo” or, alternatively, “halogen” or “halide” means fluoro, chloro, bromo or iodo. In some embodiments, halo is fluoro, chloro, or bromo, such as fluoro or chloro.

The term “fluoroalkyl” refers to an alkyl in which one or more hydrogen atoms are replaced by a fluorine atom. In one aspect, a fluoroalkyl is a C1-C6fluoroalkyl.

The term “heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C1-C6heteroalkyl.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1 (2H)-onyl, 3,4-dihydroquinolin-2 (1H)-onyl, isoindoline-1,3-dithionyl, benzo[d]oxazol-2 (3H)-onyl, 1H-benzo[d]imidazol-2 (3H)-onyl, benzo[d]thiazol-2 (3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Monocyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C1-C9heteroaryl. In some embodiments, monocyclic heteroaryl is a C1-C5heteroaryl. In some embodiments, monocyclic heteroaryl is a 5-membered or 6-membered heteroaryl. In some embodiments, bicyclic heteroaryl is a C6-C9heteroaryl.

A “heterocycloalkyl” or “heteroalicyclic” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. The term heteroalicyclic also includes all ring forms of the carbohydrates, including but not limited to the monosaccharides, the disaccharides and the oligosaccharides. In one aspect, a heterocycloalkyl is a C₂₁₀ heterocycloalkyl. In another aspect, a heterocycloalkyl is a C₂₋₆heterocycloalkyl. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(O)₂NH₂, —S(O)₂NH(alkyl), —S(O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁₋₄ alkyl), —C(═O)NH₂, —C(═O)NH(C₁₋₄ alkyl), —C(O)N(C₁₋₄ alkyl)₂, —S(O)₂NH₂, —S(O)₂NH(C₁₋₄ alkyl), —S(O)₂N(C₁₋₄ alkyl)₂, C₁₋₄ alkyl, C₃₋₆cycloalkyl, C₁₋₄ fluoroalkyl, C₁₋₄heteroalkyl, C₁₋₄ alkoxy, C₁₋₄ fluoroalkoxy, —SC₁₋₄ alkyl, —S(O)C₁₋₄ alkyl, and —S(O)₂C₁₋₄ alkyl. In some embodiments, optional substituents are independently selected from halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an antagonist.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, with respect to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein, or a pharmaceutically acceptable salt thereof, and a co-agent, are both administered to a patient simultaneously in the form of a single entity or dosage. The term “non-fixed combination” means that the active ingredients, e.g. a compound described herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be performed by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

### Methods of Dosing and Treatment

In one embodiment, the compounds disclosed herein are used in the preparation of medicaments for the treatment or prevention of diseases or conditions that would benefit from or by the reduction or inhibition of EP2 activity. In addition, a method for treating any of the diseases or conditions described herein in a mammal in need of such treatment, involves administration of pharmaceutical compositions that include at least one compound of Formula (I), (Ia), (Ib), (Ic), (Id), (Ic), (If), (Ig), (Ih), (II), (IIIa) and (IIb), (III), (IV), (IVa), (V), (Va), (VI), (VIa), (VII), (VIIa), (VIIb), (VIII), (VIIIa), (VIIIb), (VIIIc), (VIIId) and/or (VIIIe) or a pharmaceutically acceptable salt, active metabolite, prodrug, or solvate thereof, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In prophylactic applications, compositions containing the compounds described herein are administered to a patient susceptible to or otherwise at risk of a particular disease, disorder or condition.

In certain embodiments, the dose of drug being administered may be temporarily reduced or temporarily suspended for a certain length of time (i.e., a “drug holiday”).

Doses employed for adult human treatment are typically in the range of 0.01 mg to 5000 mg per day or from about 1 mg to about 1000 mg per day. In one embodiment, the desired dose is conveniently presented in a single dose or in divided doses.

In certain instances, it is appropriate to administer at least one compound of Formulas (I), (Ia), (Ib), (Ic), (Id), (Ie), (If), (Ig), (Ih), (II), (IIa) and (IIb), (III), (IV), (IVa), (V), (Va), (VI), (VIa), (VII), (VIIa), (VIIb), (VIII), (VIIIa), (VIIIb), (VIIIc), (VIIId) and/or (VIIIe) in combination with another therapeutic agent. In one specific embodiment, a compound of Formula (I), (Ia), (Ib), (Ic), (Id), (Ie), (If), (Ig), (Ih), (II), (IIa) and (IIb), (III), (IV), (IVa), (V), (Va), (VI), (VIa), (VII), (VIIa), (VIIb), (VIII), (VIIIa), (VIIIb), (VIIIc), (VIIId) and/or (VIIIe), is co-administered with a second therapeutic agent, wherein the compound of Formula (I), (Ia), (Ib), (Ic), (Id), (Ie), (If), (Ig), (Ih), (Ii), (IIa) and (IIb), (III), (IV), (IVa), (V), (Va), (VI), (VIa), (VII), (VIIa), (VIIb), (VIII), (VIIIa), (VIIIb), (VIIIc), (VIIId) and/or (VIIIe) and the second therapeutic agent modulate different aspects of the disease or condition being treated, thereby providing a greater overall benefit than administration of either therapeutic agent alone.

For combination therapies described herein, dosages of the co-administered compounds vary depending on the type of co-administered compound(s) employed, on the specific drug(s) employed, on the disease or condition being treated and so forth. In additional embodiments, when co-administered with one or more other therapeutic agents, the compound provided herein is administered either simultaneously with the one or more other therapeutic agents, or sequentially.

If administration is simultaneous, the multiple therapeutic agents are, by way of example only, provided in a single, unified form, or in multiple forms.

### EXAMPLES

The following examples are provided for illustrative purposes only and not to limit the scope of the claims provided herein.

Compounds described herein are synthesized using standard synthetic techniques or using methods known in the art in combination with methods described herein. One of skill in the art, on consideration of the present disclosure, will envision variations of reagents and conditions to produce the molecules described herein.

Compounds are prepared using standard organic chemistry techniques such as those described in, for example, March's Advanced Organic Chemistry, 6th Edition, John Wiley and Sons, Inc. Alternative reaction conditions for the synthetic transformations may be employed such as variation of solvent, reaction temperature, reaction time, as well as different chemical reagents and other reaction conditions as is understood by those of skill in the art of organic synthesis. The starting materials are available from commercial sources or are readily prepared.

### Preparation of 3-((4-(3-cyanobicyclo[1.1.1]pentan-1-yl)phenoxy)methyl)-1-(4-methoxybenzoyl)pyrrolidine-3-carboxylic acid (Cpd 6)

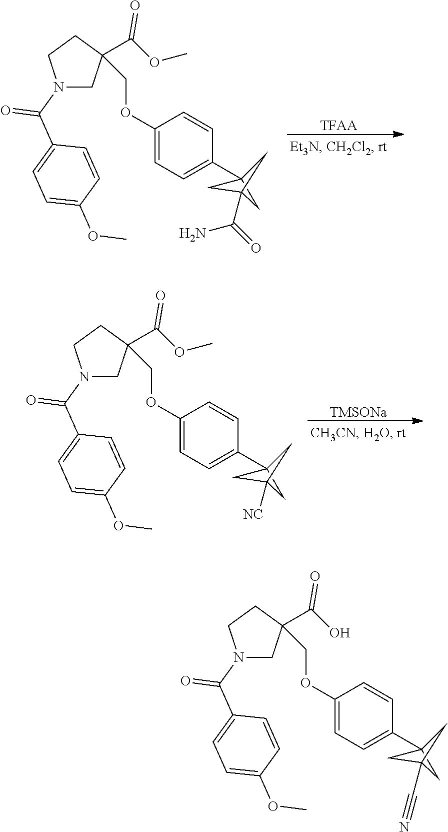 [SMILES: COC(=O)C1(COc2ccc(C34CC(C(N)=O)(C3)C4)cc2)CCN(C(=O)c2ccc(OC)cc2)C1 ; "TMSONa" ; "TFAA" ; COC(=O)C1(COc2ccc(C34CC(C#N)(C3)C4)cc2)CCN(C(=O)c2ccc(OC)cc2)C1 ; COc1ccc(C(=O)N2CCC(COc3ccc(C45CC(C#N)(C4)C5)cc3)(C(=O)O)C2)cc1]

A mixture of methyl 3-(4-{3-carbamoylbicyclo[1.1.1]pentan-1-yl}phenoxymethyl)-1-(4-methoxybenzoyl)pyrrolidine-3-carboxylate (80 mg, 0.167 mmol, 1 equiv) in CH₂Cl₂ (2 mL) was added TFAA (70 mg, 0.334 mmol, 2 equiv) and Et₃N (50.75 mg, 0.501 mmol, 3 equiv) at 0° C. The mixture was stirred at rt for 12 h. The resulting mixture was extracted with CH₂Cl₂ (5 mL). The combined organic layers were washed with water (3×5 mL), dried over anhydrous MgSO₄. After filtration, the filtrate was concentrated under reduced pressure. The residue was purified by Prep-TLC (PE/EA 1:1) to afford methyl 3-(4-{3-cyanobicyclo[1.1.1]pentan-1-yl}phenoxymethyl)-1-(4-methoxybenzoyl)pyrrolidine-3-carboxylate (60 mg, 65.56%) as a colorless solid. LC/MS: mass calcd. For C₂₇H₂₈N₂O₅: 460.20, found: 461.15 [M+H]+

A mixture of methyl 3-(4-{3-cyanobicyclo[1.1.1]pentan-1-yl}phenoxymethyl)-1-(4-methoxybenzoyl)pyrrolidine-3-carboxylate (55 mg, 0.119 mmol, 1 equiv) in CH₃CN (1.5 mL) and H₂O (0.020 mL) was added trimethyl(sodiooxy)silane (0.18 mL, 0.002 mmol, 1.5 equiv). The mixture was stirred at rt for 6 h. Desired product could be detected by LCMS. The resulting mixture was concentrated under reduced pressure. The mixture was purified by Prep-HPLC with followed conditions (Column: XBridge Prep OBD C18 Column, 30*150 mm, 5 μm; Mobile Phase A: Water (10 mmol/L NH4HCO3+0.1% NH3·H2O), Mobile Phase B: ACN; Flow rate: 60 mL/min; Gradient: 16% B to 46% B in 7 min, 46% B; Wave Length: 254 nm; RT1 (min): 5.62; Number Of Runs: 0) to afford 3-(4-{3-cyanobicyclo[1.1.1]pentan-1-yl}phenoxymethyl)-1-(4-methoxybenzoyl)pyrrolidine-3-carboxylic acid (24.6 mg, 44.83%) as a white solid.

LC/MS: mass calcd. For C26H26N2O5: 446.18, found: 447.15 [M+H]⁺. ¹H NMR (300 MHz, Methanol-d4) δ 7.47-7.57 (m, 2H), 7.08-7.20 (m, 2H), 6.79-7.04 (m, 4H), 3.98-4.33 (m, 3H), 3.85 (s, 3H), 3.63-3.79 (m, 3H), 2.34-2.53 (m, 7H), 2.07-2.25 (m, 1H).

### Preparation of 3-((4-(bicyclo[1.1.1]pentan-1-yl)phenoxy)methyl)-1-(4-methoxybenzoyl)pyrrolidine-3-carboxylic acid (Cpd. 7)

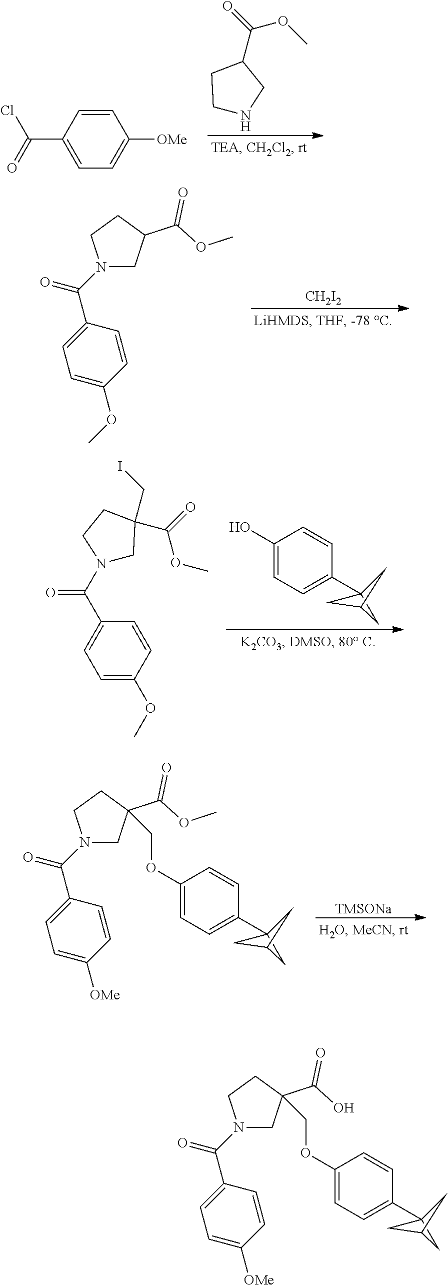

To a stirred mixture of benzoyl chloride, 4-methoxy- (5 g, 29.310 mmol, 1 equiv) and methyl pyrrolidine-3-carboxylate (4.55 g, 35.228 mmol, 1.20 equiv) in DCM (50 mL, 786.530 mmol, 26.83 equiv) were added triethylamine (8.9 g, 87.951 mmol, 3.00 equiv). The resulting mixture was extracted with CH₂Cl₂ (3×100 mL). The combined organic layers were washed with water (3×30 mL), dried over anhydrous Na₂SO₄. After filtration, the filtrate was concentrated under reduced pressure. The residue was purified by silica gel column chromatography, eluted with PE/EA (1:1) to afford methyl 1-(4-methoxybenzoyl)pyrrolidine-3-carboxylate (3.6 g, 46.41%) as a colorless oil. LC/MS: mass calcd. For C₁₄H₁₇NO₄:263.12, found: 264.05 [M+H]⁺.

To a stirred mixture of methyl 3-(iodomethyl)-1-(4-methoxybenzoyl)pyrrolidine-3-carboxylate (150 mg, 0.372 mmol, 1 equiv) and 4-{bicyclo[1.1.1]pentan-1-yl}phenol (71.52 mg, 0.446 mmol, 1.2 equiv) in DMSO was added K2CO3 (77.12 mg, 0.558 mmol, 1.5 equiv). The resulting mixture was stirred overnight at 80° C. Desired product could be detected by LCMS. The residue was purified by silica gel column chromatography, eluted with PE/EA (10:1) to afford methyl 3-(4-{bicyclo[1.1.1]pentan-1-yl}phenoxymethyl)-1-(4-methoxybenzoyl)pyrrolidine-3-carboxylate (74 mg, 45.67%) as a colorless oil. LC/MS: mass calcd. For C14H28N2O3: 435.20, found: 436.15 [M+H]⁺.

To a stirred solution of methyl 3-(4-{bicyclo[1.1.1]pentan-1-yl}phenoxymethyl)-1-(4-methoxybenzoyl)pyrrolidine-3-carboxylate (74 mg, 0.170 mmol, 1 equiv) in H2O (2 mL) and CH3CN (2 mL) was added trimethyl(sodiooxy)silane (152.48 mg, 1.360 mmol, 8 equiv) in portions at room temperature for 16 h. The resulting mixture was concentrated under reduced pressure. The crude product (50 mg) was purified by Prep-HPLC with the following conditions (Column: XBridge Shield RP18 OBD Column, 19*250 mm, 10 μm; Mobile Phase A: Water (0.05% HCl), Mobile Phase B: ACN; Flow rate: 25 mL/min; Gradient: 55% B to 74% B in 7 min, 74% B; Wave Length: 254 nm; RT1 (min): 5.25; Number Of Runs: 0) to afford 3-(4-{bicyclo[1.1.1]pentan-1-yl}phenoxymethyl)-1-(4-methoxybenzoyl)pyrrolidine-3-carboxylic acid (24.6 mg, 34.31%) as a white solid. LC/MS: mass calcd. For C25H27NO5: 421.19, found: 422.20 [M+H]⁺. ¹H NMR (300 MHz, DMSO-d6) δ 7.49 (m, 2H), 7.08 (m, 2H), 6.97 (d, J=8.3 Hz, 2H), 6.93-6.69 (m, 2H), 4.18 (m, 1H), 4.02 (m, 1H), 3.93 (d, J=11.5 Hz, 1H), 3.80 (s, 3H), 3.57 (m, 3H), 2.50 (s, 1H), 2.27-2.08 (m, 2H), 2.00 (s, 6H).

### Preparation of 4′-((1-(4-methoxybenzoyl)-3-(2,2,2-trifluoro-1-hydroxyethyl)pyrrolidin-3-yl)methoxy)-[1,1′-biphenyl]-4-carbonitrile (Cpd. 12)

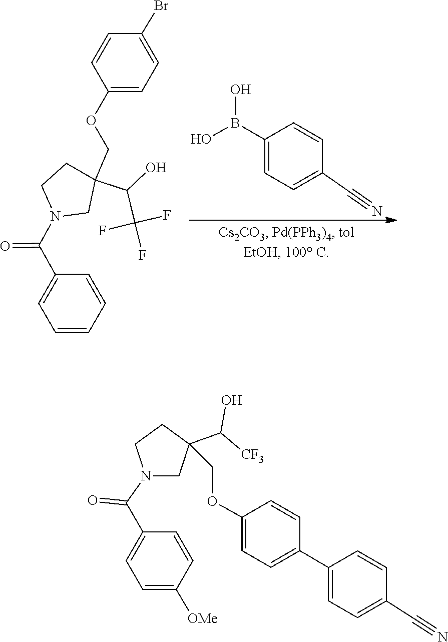 [SMILES: O=C(c1ccccc1)N1CCC(COc2ccc(Br)cc2)(C(O)C(F)(F)F)C1 ; N#Cc1ccc(B(O)O)cc1 ; COc1ccc(C(=O)N2CCC(COc3ccc(-c4ccc(C#N)cc4)cc3)(C(O)C(F)(F)F)C2)cc1]

To a stirred mixture of 1-[3-(4-bromophenoxymethyl)-1-(4-methoxybenzoyl)pyrrolidin-3-yl]-2,2,2-trifluoroethanol (80 mg, 0.164 mmol, 1 equiv) and 4-cyanophenylboronic acid (28.7 mg, 0.195 mmol, 1.19 equiv) in 2M Na2CO3.aq (0.2 ml) and DEM (0.8 ml) was added Pd(PPh3)4 (18.9 mg, 0.016 mmol, 0.10 equiv). The reaction was stirred at 85° C. for 16 h. Desired product could be detected by LCMS. The resulting mixture was extracted with EtOAc (3×10 mL). The combined organic layers were washed with water (3×8 mL), dried over anhydrous Na2SO4. After filtration, the filtrate was concentrated under reduced pressure. The residue was dissolved in DMF (1 mL). The crude product was purified by Prep-HPLC with the following conditions (Column: XBridge Shield RP18 OBD Column, 19*250 mm, 10 μm; Mobile Phase A: Water (10 mmol/L NH4HCO3+0.1% NH3·H2O), Mobile Phase B: MeOH—HPLC; Flow rate: 25 mL/min; Gradient: 65% B to 85% B in 7 min, 85% B; Wave Length: 220 nm; RT1 (min): 6.35; Number Of Runs: 0) to afford 4′-{[1-(4-methoxybenzoyl)-3-(2,2,2-trifluoro-1-hydroxyethyl)pyrrolidin-3-yl]methoxy}-[1,1′-biphenyl]-4-carbonitrile (5.7 mg, 6.81%) as a white solid. LC/MS: mass calcd. For C28H25F3N2O4:510.18, found: 511.15 [M+H]⁺. ¹H NMR (300 MHz, DMSO-d6) δ 7.82-7.90 (m, 4H), 7.72 (s, 2H), 7.54 (s, 2H), 6.98-7.12 (m, 4H), 6.79 (s, 1H), 3.96-4.23 (m, 3H), 3.79 (s, 3H), 3.55-3.72 (m, 5.1H), 1.87-2.21 (m, 10.2H). ¹⁹F NMR (282 MHz, DMSO-d6) δ −72.034.

### Preparation of (3-((4-bromophenoxy)methyl)-3-(2,2,2-trifluoro-1-hydroxyethyl)pyrrolidin-1-yl)(4-methoxyphenyl)methanone; [1-[3-(4-bromophenoxymethyl)-1-(4-methoxybenzoyl)pyrrolidin-3-yl]-2,2,2-trifluoroethanol](Cpd. 14)

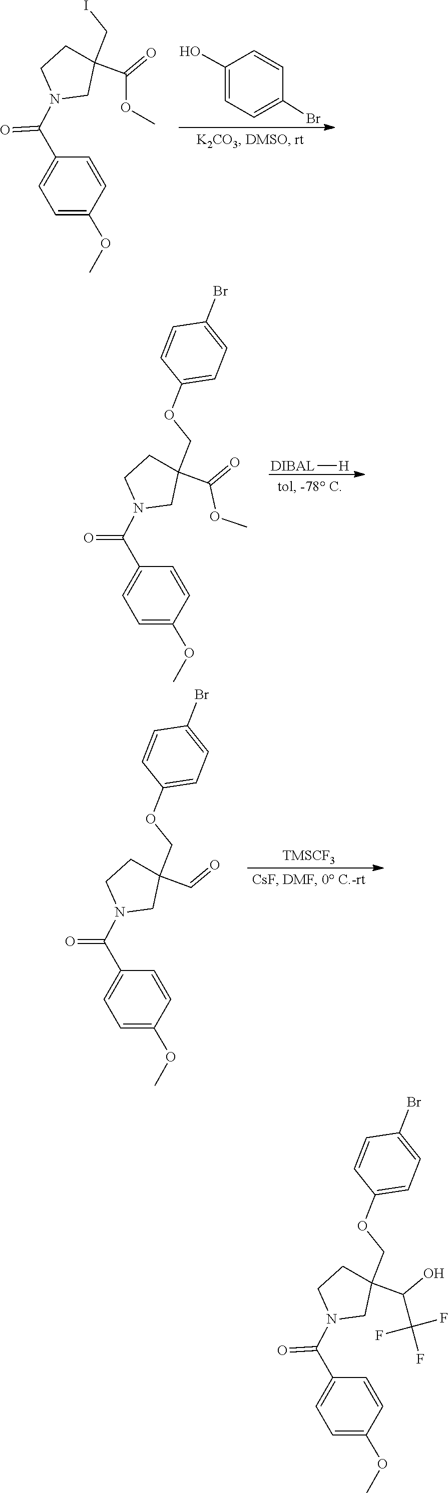

To a stirred mixture of methyl 3-(iodomethyl)-1-(4-methoxybenzoyl)pyrrolidine-3-carboxylate (1 g, 2.480 mmol, 1 equiv) and 4-bromophenol (514 mg, 2.971 mmol, 1.20 equiv) in DMSO (8 mL, 112.633 mmol, 45.42 equiv) was added K2CO3 (512 mg, 3.705 mmol, 1.49 equiv). The reaction was stirred at 80° C. for 16 h. Desired product could be detected by LCMS. The resulting mixture was filtered, the filter cake was washed with EtOAc (3×10 mL). The filtrate was concentrated under reduced pressure. The resulting mixture was extracted with EtOAc (3×50 mL). The combined organic layers were washed with water (3×50 mL), dried over anhydrous Na2SO4. After filtration, the filtrate was concentrated under reduced pressure. The residue was dissolved in DMF (2 mL). The residue was purified by silica gel column chromatography, eluted with PE/EA (1:1) to afford methyl 3-(4-bromophenoxymethyl)-1-(4-methoxybenzoyl)pyrrolidine-3-carboxylate (900 mg, 80.95%) as a light yellow oil. LC/MS: mass calcd. For C21H22BrNO5: 447.07, found: 447.95 [M+H]⁺

To a stirred mixture of methyl 3-(4-bromophenoxymethyl)-1-(4-methoxybenzoyl)pyrrolidine-3-carboxylate (720 mg, 1.606 mmol, 1 equiv) in toluene (7 mL) was added DIBAL-H (3.3 mL, 16.265 mmol, 10.13 equiv) in portions at −78° C. under nitrogen atmosphere. The reaction was stirred at −78° C. for 2 h. Desired product could be detected by LCMS. The resulting mixture was extracted with EtOAc (3×50 mL). The combined organic layers were washed with water (3×10 mL), dried over anhydrous Na2SO4. After filtration, the filtrate was concentrated under reduced pressure. The residue was purified by silica gel column chromatography, eluted with PE/EA (1:1) to afford 3-(4-bromophenoxymethyl)-1-(4-methoxybenzoyl)pyrrolidine-3-carbaldehyde (167.2 mg, 21.34%) as a light yellow oil. LC/MS: mass calcd. For C20H20BrNO4: 417.06, found: 417.95[M+H]⁺

To a stirred mixture of 3-(4-bromophenoxymethyl)-1-(4-methoxybenzoyl)pyrrolidine-3-carbaldehyde (140 mg, 0.335 mmol, 1 equiv) and CsF (66.09 mg, 0.436 mmol, 1.30 equiv) in DMF (2.00 mL, 25.865 mmol, 77.21 equiv) was added TMSCF3 (61.87 mg, 0.436 mmol, 1.30 equiv) in portions at 0° C. The reaction was stirred at room temperature for 12 h. Desired product could be detected by LCMS. The resulting mixture was extracted with EtOAc (3×40 mL). The combined organic layers were washed with water (3×30 mL), dried over anhydrous Na2SO4. After filtration, the filtrate was concentrated under reduced pressure. The residue was purified by Prep-TLC (PE/EA 1:1) to afford 1-[3-(4-bromophenoxymethyl)-1-(4-methoxybenzoyl)pyrrolidin-3-yl]-2,2,2-trifluoroethanol (3.5 mg, 2.11%) as a light yellow oil. The crude product (15 mg) was purified by Prep-HPLC with the following conditions (Column: Kinetex EVO C18, 21.2*250 mm, 5 μm; Mobile Phase A: Water (0.05% TFA), Mobile Phase B: ACN; Flow rate: 25 mL/min; Gradient: 40% B to 54% B in 15 min, 54% B; Wave Length: 254 nm; RT1 (min): 13.5; Number Of Runs: 0) to afford 1-[3-(4-bromophenoxymethyl)-1-(4-methoxybenzoyl)pyrrolidin-3-yl]-2,2,2-trifluoroethanol (3.5 mg, 2.11%) as a white solid. 1-[3-(4-bromophenoxymethyl)-1-(4-methoxybenzoyl)pyrrolidin-3-yl]-2,2,2-trifluoroethanol (3.5 mg, 2.11%) was delivered. LC/MS: mass calcd. For C21H21BrF3NO4: 487.06, found: 488.00[M+H]⁺. ¹H NMR (300 MHz, DMSO-d6) δ7.45 (d, J=22.5 Hz, 4H), 6.76-7.01 (m, 5H), 4.25 (s, 1H), 4.06 (d, J=10.8 Hz, 1H), 3.91 (s, 1H), 3.79 (s, 3H), 3.69 (d, J=12.3 Hz, 3H), 2.12 (d, J=8.7 Hz, 2H). ¹⁹F NMR (282 MHz, DMSO-d6) δ −71.944, −72.159, −73.446.

### Preparation of 4′-((1-(4-methoxybenzoyl)-3-(2,2,2-trifluoro-1,1-dihydroxyethyl)pyrrolidin-3-yl)methoxy)-[1,1′-biphenyl]-4-carbonitrile (Cpd. 13)

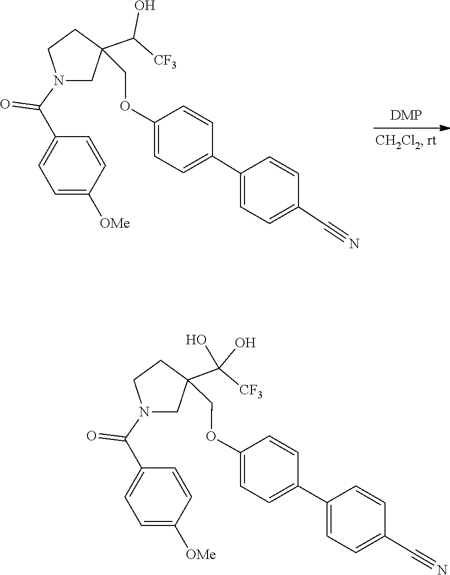 [SMILES: COc1ccc(C(=O)N2CCC(COc3ccc(-c4ccc(C#N)cc4)cc3)(C(O)C(F)(F)F)C2)cc1 ; COc1ccc(C(=O)N2CCC(COc3ccc(-c4ccc(C#N)cc4)cc3)(C(O)(O)C(F)(F)F)C2)cc1]

To a stirred mixture of 4′-{[1-(4-methoxybenzoyl)-3-(2,2,2-trifluoro-1-hydroxyethyl)pyrrolidin-3-yl]methoxy}-[1,1′-biphenyl]-4-carbonitrile (60 mg, 0.118 mmol, 1 equiv) in CH2Cl2 (1 mL) were added 1,1-bis(acetyloxy)-3-oxo-3H-11\hat{ }[5],2-benziodaoxol-1-yl acetate (99.5 mg). The reaction was stirred at room temperature for 2 h. Desired product could be detected by LCMS. The resulting mixture was concentrated under vacuum. The residue was dissolved in DMF (1 mL). The crude product was purified by Prep-HPLC with the following conditions (Column: Kinetex EVO C18, 21.2*250 mm, 5 μm; Mobile Phase A: Water (0.05% TFA), Mobile Phase B: ACN; Flow rate: 25 mL/min; Gradient: 47% B to 62% B in 10 min, 62% B; Wave Length: 254 nm; RT1 (min): 6.87; Number Of Runs: 0) to afford 4′-{[1-(4-methoxybenzoyl)-3-(2,2,2-trifluoro-1,1-dihydroxyethyl)pyrrolidin-3-yl]methoxy}-[1,1′-biphenyl]-4-carbonitrile (3.8 mg, 5.91%) as a white solid. LC/MS: mass calcd. For C28H25F3N2O5: 526.17, found: 527.10 [M+H]⁺. ¹H NMR (300 MHz, DMSO-d6) δ 7.85-7.90 (m, 3H), 7.74 (t, J=7.8 Hz, 2H), 7.51 (dd, J=8.4, 18.6 Hz, 2H), 6.95-7.19 (m, 5H), 4.19 (s, 1H), 4.09 (d, J=12.3 Hz, 1H), 3.92 (d, J=11.4 Hz, 1H), 3.79 (s, 3H), 3.70 (s, 3H), 2.36-2.51 (m, 1H), 1.97-2.01 (m, 1H). ¹⁹F NMR (282 MHz, DMSO-d6) δ −72.491, −74.626, −78.644.

### Preparation of 3-(((4′-cyano-[1,1′-biphenyl]-4-yl)oxy)methyl)-1-(4-methoxybenzoyl)-N-(methylsulfonyl)pyrrolidine-3-sulfonamide; [4′-{[3-(methanesulfonylsulfamoyl)-1-(4-methoxybenzoyl)pyrrolidin-3-yl]methoxy}-[1,1′-biphenyl]-4-carbonitrile](Cpd. 10)

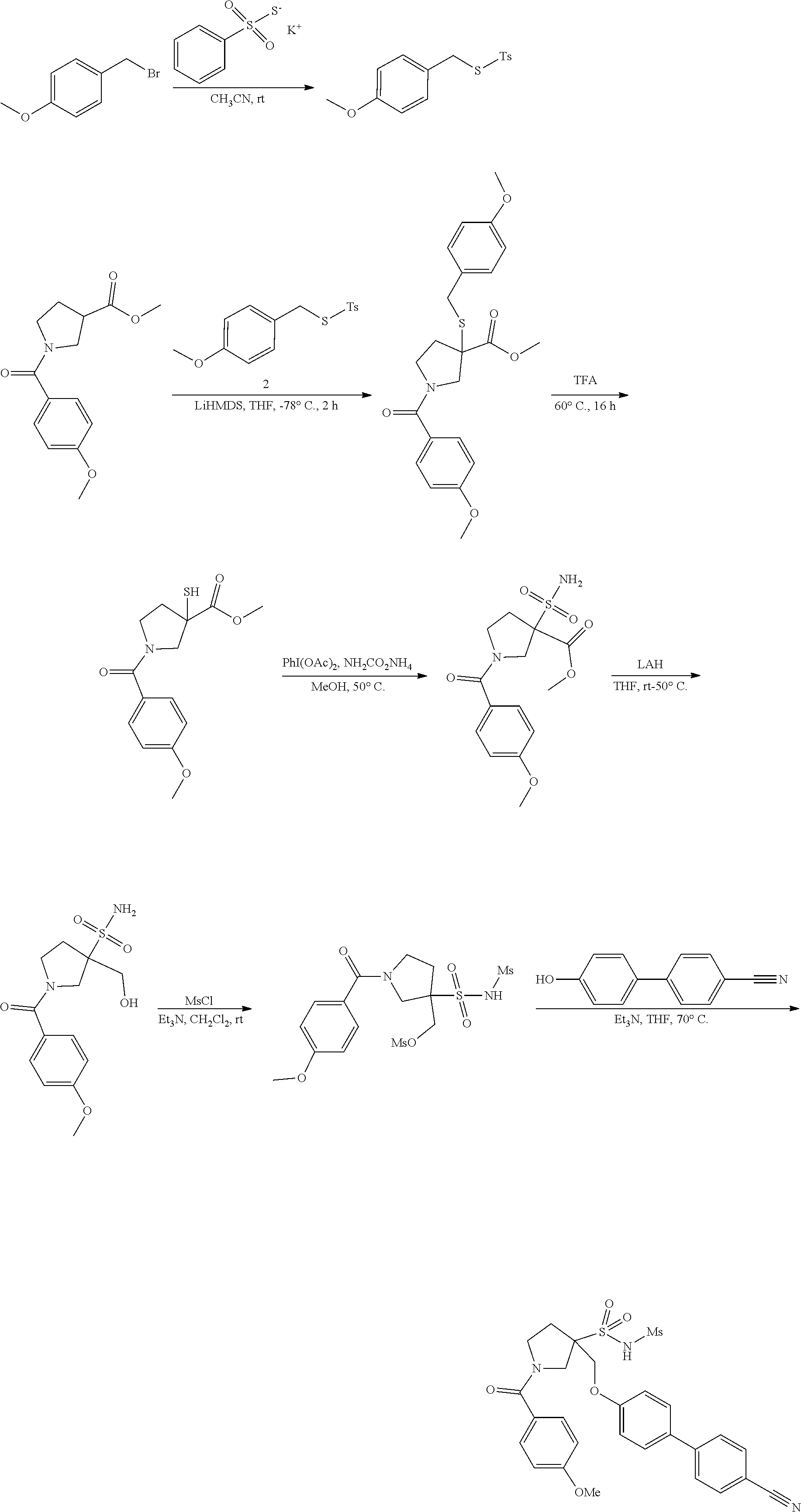

A mixture of potassium (benzenesulfonyl)sulfanide (5 g, 23.549 mmol, 1 equiv) in CH3CN (50 mL, 951.209 mmol, 40.39 equiv) was added 1-(bromomethyl)-4-methoxybenzene (3.14 g, 15.617 mmol, 0.66 equiv). The mixture was stirred at rt for 1 h. Desired product could be detected by TLC. The resulting mixture was extracted with EtOAc (50 mL) and H2O (50 ml). The combined organic layers were washed with water (3×50 mL), dried over anhydrous Na2SO4. After filtration, the filtrate was concentrated under reduced pressure to afford 1-methoxy-4-{[(4-methylbenzenesulfonyl)sulfanyl]methyl}benzene (3.8 g, 52.32%) as a colorless oil.

The mixture of methyl 1-(4-methoxybenzoyl)pyrrolidine-3-carboxylate (1.7 g, 6.457 mmol, 1 equiv) in THF (20 mL, 246.855 mmol, 38.23 equiv) was added LiHMDS (1.62 g, 9.685 mmol, 1.5 equiv) at −78° C. The mixture was stirred at −78° C. for 1 h. The mixture was added 1-methoxy-4-{[(4-methylbenzenesulfonyl)sulfanyl]methyl}benzene (2.99 g, 9.685 mmol, 1.5 equiv) at −78° C. The mixture was stirred at −78° C. for 0.5 h. The whole process was under nitrogen atmosphere. Desired product could be detected by LCMS. The reaction was quenched with sat. NH4Cl (aq.) at room temperature. The resulting mixture was extracted with EtOAc (50 mL) and H2O (50 ml). The combined organic layers were washed with brine (3×50 mL), dried over anhydrous Na2SO4. After filtration, the filtrate was concentrated under reduced pressure. The residue was purified by silica gel column chromatography, eluted with PE/EA (1:1) to afford methyl 1-(4-hydroxybenzoyl)-3-{[(4-methoxyphenyl)methyl]sulfanyl}pyrrolidine-3-carboxylate (1.8 g, 49.74%) as a yellow oil. LC/MS: mass calcd. For C₂₂H₂₅NO₅S: 415.15, found: 416.05 [M+H]⁺

The mixture of methyl 1-(4-methoxybenzoyl)-3-{[(4-methoxyphenyl)methyl]sulfanyl}pyrrolidine-3-carboxylate (1.5 g, 3.610 mmol, 1 equiv) was added trifluoroacetaldehyde (20 mL, 204.032 mmol, 56.52 equiv). The mixture was stirred at 60° C. for 30 h. Desired product could be detected by LCMS. The resulting mixture was concentrated under reduced pressure to afford methyl 1-(4-methoxybenzoyl)-3-sulfanylpyrrolidine-3-carboxylate (1.5 g, 140.68%) as ad black oil. LC/MS: mass calcd. For C14H17NO4S: 295.09, found: 296.05[M+H]+

The mixture of methyl 1-(4-hydroxybenzoyl)-3-sulfanylpyrrolidine-3-carboxylate (1.5 g, 5.332 mmol, 1 equiv) in MeOH (15 mL, 370.482 mmol, 72.95 equiv) was added ammonium carbamate (0.79 g, 10.158 mmol, 2 equiv) and (acetyloxy)(phenyl)-1\hat{ }[3]-iodanyl acetate (5.73 g, 17.776 mmol, 3.5 equiv). The mixture was stirred at 50° C. for 3 h. The resulting mixture was concentrated under reduced pressure. The residue was purified by silica gel column chromatography, eluted with PE/EA (0:1) to afford methyl 1-(4-methoxybenzoyl)-3-sulfamoylpyrrolidine-3-carboxylate (650 mg, 35.38%) as a light yellow oil. LC/MS: mass calcd. For C₁₄H₁₈N₂O₆S: 342.09, found: 343.10 [M+H]⁺

The mixture of methyl 1-(4-methoxybenzoyl)-3-sulfamoylpyrrolidine-3-carboxylate (640 mg, 1.869 mmol, 1 equiv) in THF (8 mL, 98.742 mmol, 52.82 equiv) was added LiBH4 (162.86 mg, 7.476 mmol, 4 equiv). The mixture was stirred at room temperature for 2 h. Desired product could be detected by LCMS. The resulting mixture was concentrated under reduced pressure. The residue was purified by silica gel column chromatography, eluted with PE/EA (1:1) to afford 3-(hydroxymethyl)-1-(4-methoxybenzoyl)pyrrolidine-3-sulfonamide (310 mg, 50.12%) as a light yellow oil. LC/MS: mass calcd. For C₁₃H₁₈N₂O₅S: 314.09, found: 315.00[M+H]+

The mixture of 3-(hydroxymethyl)-1-(4-methoxybenzoyl)pyrrolidine-3-sulfonamide (20 mg, 0.064 mmol, 1 equiv) in CH2Cl2 (2.00 mL, 31.649 mmol, 494.51 equiv) was added methanesulfonyl chloride (10.93 mg, 0.096 mmol, 1.5 equiv) and Et3N (12.88 mg, 0.128 mmol, 2 equiv). The mixture was stirred at room temperature for 1 h under nitrogen atmosphere. The reaction was monitored by TLC. The mixture was concentrated under reduced pressure to afford (1-(4-methoxybenzoyl)-3-(N-(methylsulfonyl)sulfamoyl)pyrrolidin-3-yl)methyl methanesulfonate (20 mg) as a brown oil.

The mixture of (1-(4-methoxybenzoyl)-3-(N-(methylsulfonyl)sulfamoyl)pyrrolidin-3-yl)methyl methanesulfonate (20 mg, 0.051 mmol, 1 equiv) in THF (1 mL, 12.343 mmol, 242.19 equiv) was added 4′-hydroxy-[1,1′-biphenyl]-4-carbonitrile (9.95 mg, 0.051 mmol, 1 equiv) and TEA (10.31 mg, 0.102 mmol, 2 equiv). The mixture was stirred at 60° C. for 2 h. Desired product could be detected by LCMS. The mixture was purified by Prep-HPLC with followed conditions (Column: XBridge Prep Phenyl OBD Column, 19*250 mm, 5 μm; Mobile Phase A: Water (10 mmol/L NH4HCO3+0.1% NH3·H2O), Mobile Phase B: ACN; Flow rate: 25 mL/min; Gradient: 40% B to 70% B in 7 min, 70% B; Wave Length: 254 nm; RT1 (min): 6; Number Of Runs: 0) to afford 4′-{[3-(methanesulfonylsulfamoyl)-1-(4-methoxybenzoyl)pyrrolidin-3-yl]methoxy}-[1,1′-biphenyl]-4-carbonitrile (3.5 mg, 11.64%) as a off-white solid.

LC/MS: mass calcd. For C₂₇H₂₇N₃O₇S₂: 569.13, found: 570.00[M+H]⁺. ¹H NMR (300 MHz, DMSO-d6) δ 7.79-7.98 (m, 6H), 7.46-7.67 (m, 4H), 6.87-7.00 (m, 2H), 4.32-4.43 (m, 1H), 3.91-3.91 (m, 1H), 3.76-3.81 (m, 4H), 3.66-3.69 (m, 3H), 3.03 (s, 3H), 2.44-2.64 (m, 3H), 1.25 (s, 2H).

### Preparation of 4′-((1-(4-methoxybenzoyl)-3-(S-methylsulfonimidoyl)pyrrolidin-3-yl)methoxy)-[1,1′-biphenyl]-4-carbonitrile; [4′-({3-[imino(methyl)oxo-lambda6-sulfanyl]-1-(4-methoxybenzoyl)pyrrolidin-3-yl}methoxy)-[1,1′-biphenyl]-4-carbonitrile](Cpd. 9)

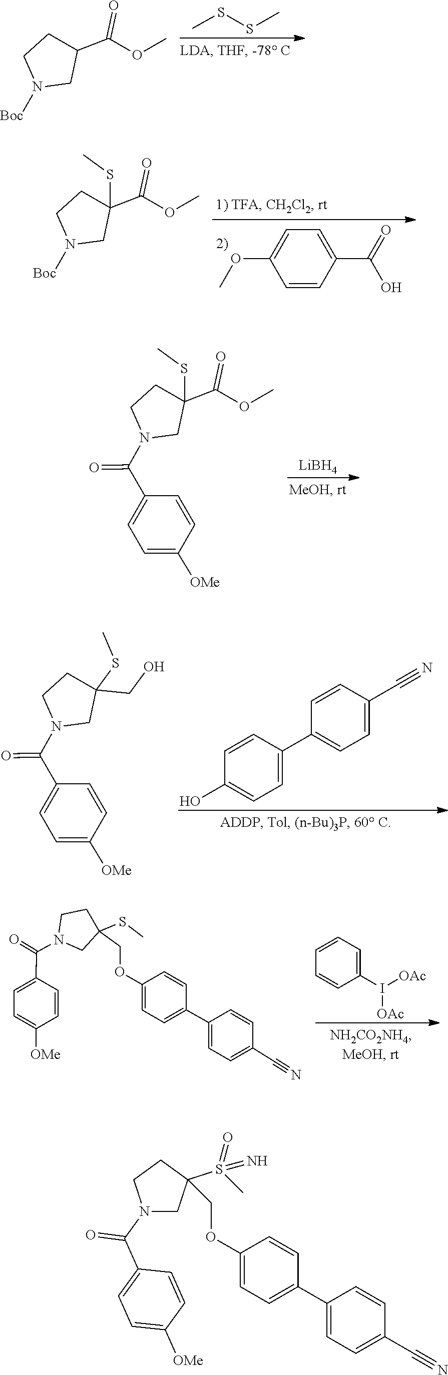

To the mixture of 1-tert-butyl 3-methyl pyrrolidine-1,3-dicarboxylate (5 g, 21.808 mmol, 1 equiv) in THF (100 mL, 1234.277 mmol, 56.60 equiv) was added LDA (9.35 g, 87.232 mmol, 4 equiv) and the mixture was stirred at −78° C. The mixture was added dimethyl disulfide (8.22 g, 87.232 mmol, 4 equiv) at −78° C. The mixture was stirred at room temperature for 12 h. Desired product could be detected by LCMS. The reaction was quenched with sat. NH4Cl (aq.) at room temperature. The resulting mixture was extracted with EtOAc (100×mL). The combined organic layers were dried over anhydrous Na2SO4. After filtration, the filtrate was concentrated under reduced pressure. The residue was purified by silica gel column chromatography, eluted with PE/EA (5:1) to afford 1-tert-butyl 3-methyl 3-(methylsulfanyl)pyrrolidine-1,3-dicarboxylate (2.9 g, 45.41%) as a brown oil. LC/MS: mass calcd. For C₁₂H₂₁NO₄S: 275.12, found: 176.05 [M+H−100]⁺

Step 1: To the mixture of 1-tert-butyl 3-methyl 3-(methylsulfanyl)pyrrolidine-1,3-dicarboxylate (1 g, 3.632 mmol, 1 equiv) in CH2Cl2 (6 mL, 94.384 mmol, 25.99 equiv) was added TFA (6 mL, 80.778 mmol, 22.24 equiv) which was stirred at room temperature for 2 h. Step 2: To the residue of P-anisic acid (0.50 g, 3.269 mmol, 0.9 equiv) was added SOCl2 (10 mL, 137.861 mmol, 37.96 equiv). The mixture was stirred at 80° C. for 2 h. Step 3: The mixture of 1 and 2 was T was concentrated under reduced pressure. The residue of 1 in DCM (15 mL, 235.959 mmol, 64.97 equiv) was added residue 2 and TEA (14.50 mL, 104.311 mmol, 28.72 equiv). The mixture was stirred at room temperature for 1 h. Desired product could be detected by LCMS. The resulting mixture was extracted with CH2Cl2 (10 mL). The combined organic layers were washed with water (3×20 mL), dried over anhydrous MgSO4. After filtration, the filtrate was concentrated under reduced pressure. The residue was purified by silica gel column chromatography, eluted with PE/EA (1:1) to afford methyl 1-(4-methoxybenzoyl)-3-(methylsulfanyl)pyrrolidine-3-carboxylate (500 mg, 42.14%) as a brown oil. LC/MS: mass calcd. For C₁₅H₁₉NO₄S: 309.10, found: 310.05[M+H]⁺

To the mixture of methyl 1-(4-methoxybenzoyl)-3-(methylsulfanyl)pyrrolidine-3-carboxylate (450 mg, 1.455 mmol, 1 equiv) in CH3OH (10 mL, 246.988 mmol, 169.81 equiv) was added LiBH4 (126.72 mg, 5.820 mmol, 4 equiv). The mixture was stirred at room temperature for 1 h. Desired product could be detected by LCMS. The reaction was quenched with Water at room temperature. The resulting mixture was extracted with EtOAc (10 mL). The combined organic layers were washed with water (3×10 mL), dried over anhydrous MgSO4. After filtration, the filtrate was concentrated under reduced pressure to afford [1-(4-methoxybenzoyl)-3-(methylsulfanyl)pyrrolidin-3-yl]methanol (440 mg, 101.30%) as a light yellow oil. LC/MS: mass calcd. For C₁₄H₁₉NO₃S: 281.11, found: 282.05[M+H]⁺

To the mixture of [1-(4-methoxybenzoyl)-3-(methylsulfanyl)pyrrolidin-3-yl]methanol (360 mg, 1.279 mmol, 1 equiv) in toluene (10 mL, 0.109 mmol, 0.08 equiv) was added 4′-hydroxy-[1,1′-biphenyl]-4-carbonitrile (499.55 mg, 2.558 mmol, 2 equiv), (NE)-N-[[(E)-(piperidin-1-yl)carbonyl]imino]piperidine-1-carboxamide (807.06 mg, 3.197 mmol, 2.5 equiv) and tributylphosphine (647.15 mg, 3.197 mmol, 2.5 equiv). The mixture was stirred at 60° C. for 2 h. Desired product could be detected by LCMS. The resulting mixture was concentrated under reduced pressure. The residue was purified by silica gel column chromatography, eluted with PE/EA (1:1) to afford 4′-{[1-(4-methoxybenzoyl)-3-(methylsulfanyl)pyrrolidin-3-yl]methoxy}-[1,1′-biphenyl]-4-carbonitrile (600 mg, 97.09%) as a light yellow oil. LC/MS: mass calcd. For C₂₇H₂₆N₂O₃S: 458.17, found: 459.10[M+H]⁺

To the mixture of 4′-{[1-(4-methoxybenzoyl)-3-(methylsulfanyl)pyrrolidin-3-yl]methoxy}-[1,1′-biphenyl]-4-carbonitrile (100 mg, 0.218 mmol, 1 equiv) in CH₃OH (2 mL, 49.398 mmol, 226.53 equiv) was added (acetyloxy)(phenyl)-lambda3-iodanyl acetate (175.60 mg, 0.545 mmol, 2.5 equiv) and ammonium carbamate (34.05 mg, 0.436 mmol, 2 equiv). The mixture was stirred at room temperature for 3 h. Desired product could be detected by LCMS. The mixture was purified by Prep-HPLC with followed conditions (Column: XBridge Prep Phenyl OBD Column, 19*250 mm, 5 μm; Mobile Phase A: Water (10 mmol/L NH4HCO3+0.1% NH3·H2O), Mobile Phase B: ACN; Flow rate: 25 mL/min; Gradient: 40% B to 70% B in 7 min, 70% B; Wave Length: 254 nm; RT1 (min): 6.3; Number Of Runs: 0) to afford 4′-({3-[imino(methyl)oxo-lambda6-sulfanyl]-1-(4-methoxybenzoyl)pyrrolidin-3-yl}methoxy)-[1,1′-biphenyl]-4-carbonitrile (6.1 mg, 5.65%) as a white solid.

LC/MS: mass calcd. For C₂₇H₂₇N₃O₄S: 489.17, found: 490.10[M+H]⁺. ¹H NMR (300 MHz, Methanol-d4) δ 7.80 (s, 4H), 7.70-7.72 (d, 2H), 7.54-7.57 (m, 2H), 7.14-7.21 (m, 2H), 7.01 (d, J=7.8 Hz, 2H), 4.12-4.57 (m, 3H), 3.66-4.12 (m, 6H), 3.19-3.23 (m, 3H), 2.75 (br, 1H), 2.31-2.46 (m, 1H).

### Preparation of 3-((4-(3-carboxybicyclo[1.1.1]pentan-1-yl)phenoxy)methyl)-1-(4-methoxybenzoyl)pyrrolidine-3-carboxylic acid (Cpd. 11)

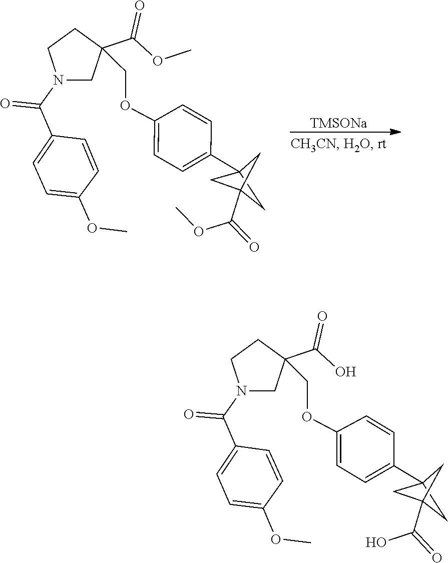 [SMILES: "TMSONa" ; COC(=O)C1(COc2ccc(C34CC(C(=O)OC)(C3)C4)cc2)CCN(C(=O)c2ccc(OC)cc2)C1 ; COc1ccc(C(=O)N2CCC(COc3ccc(C45CC(C(=O)O)(C4)C5)cc3)(C(=O)O)C2)cc1]

The mixture of methyl 1-(4-methoxybenzoyl)-3-{4-[3-(methoxycarbonyl)bicyclo[1.1.1]pentan-1-yl]phenoxymethyl}pyrrolidine-3-carboxylate (70 mg, 0.142 mmol, 1 equiv) in CH3CN (2 mL, 38.048 mmol, 268.27 equiv) and H2O (0.01 mL, 0.555 mmol, 3.91 equiv) was added trimethyl(sodiooxy)silane (0.245 mL, 0.002 mmol, 1.5 equiv). The mixture was stirred at rt for 12 h. The resulting mixture was concentrated under reduced pressure. The mixture was purified by Prep-HPLC with followed conditions (Column: XBridge Shield RP18 OBD Column, 19*250 mm, 10 μm; Mobile Phase A: Water (10 mmol/L NH4HCO3), Mobile Phase B: ACN; Flow rate: 25 mL/min; Gradient: 5% B to 25% B in 7 min, 25% B; Wave Length: 254 nm; RT1 (min): 5.3/6.9; Number Of Runs: 0) to afford 3-(4-{3-carboxybicyclo[1.1.1]pentan-1-yl}phenoxymethyl)-1-(4-methoxybenzoyl)pyrrolidine-3-carboxylic acid (20.1 mg, 30.03%) as a white solid.

LC/MS: mass calcd. For C26H27NO7: 465.18, found: 466.15 [M+H]⁺, 1H NMR (300 MHz, Methanol-d4) δ 7.51-7.56 (m, 2H), 7.12-7.19 (m, 2H), 6.99-7.01 (m, 2H), 6.82-6.93 (m, 2H), 4.19-4.33 (m, 1H), 4.11-3.99 (m, 2H), 3.86 (s, 3H), 3.67-3.78 (m, 3H), 2.42 (s, 1H), 2.25 (d, J=3.9 Hz, 7H).

### Preparation of 3-(4-{3-carbamoylbicyclo[1.1.1]pentan-1-yl}phenoxymethyl)-1-(4-methoxybenzoyl)pyrrolidine-3-carboxylic acid (Cpd. 8)

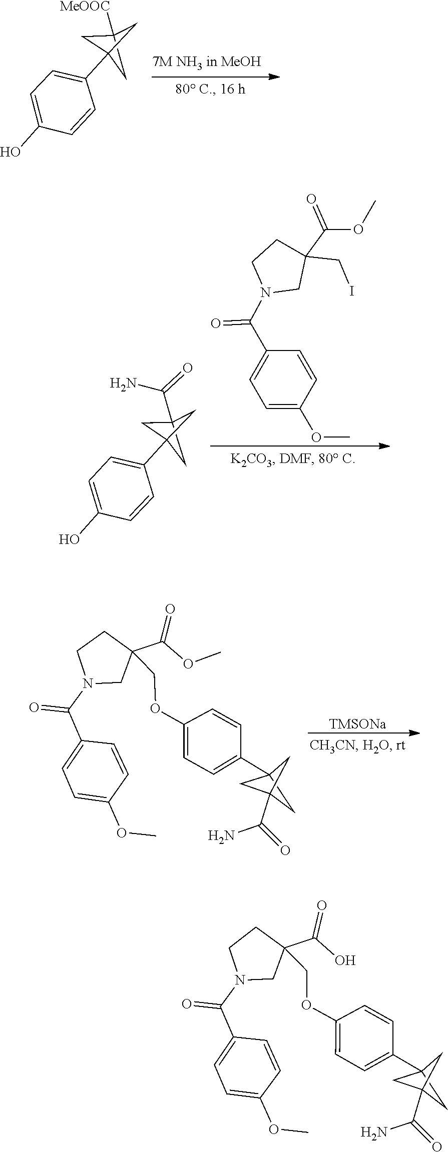 [SMILES: COC(=O)C12CC(c3ccc(O)cc3)(C1)C2 ; "TMSONa" ; NC(=O)C12CC(c3ccc(O)cc3)(C1)C2 ; COC(=O)C1(CI)CCN(C(=O)c2ccc(OC)cc2)C1 ; COC(=O)C1(COc2ccc(C34CC(C(N)=O)(C3)C4)cc2)CCN(C(=O)c2ccc(OC)cc2)C1 ; COc1ccc(C(=O)N2CCC(COc3ccc(C45CC(C(N)=O)(C4)C5)cc3)(C(=O)O)C2)cc1]

A mixture of methyl 3-(4-hydroxyphenyl)bicyclo[1.1.1]pentane-1-carboxylate (100 mg, 0.458 mmol, 1 equiv) in NH3 (g) in MeOH (3 mL, 105.690 mmol, 230.67 equiv) was stirred at 80° C. for 12 h. Desired product could be detected by LCMS. The resulting mixture was concentrated under reduced pressure to afford 3-(4-hydroxyphenyl)bicyclo[1.1.1]pentane-1-carboxamide (95 mg, 91.81%) as a brown solid. LC/MS: mass calcd. For C12H13NO2: 203.09, found: 204.10 [M+H]+

The mixture of methyl 3-(iodomethyl)-1-(4-methoxybenzoyl)pyrrolidine-3-carboxylate (237 mg, 0.588 mmol, 1 equiv) in DMF (4 mL, 51.686 mmol, 87.94 equiv) was added 3-(4-hydroxyphenyl)bicyclo[1.1.1]pentane-1-carboxamide (149.32 mg, 0.735 mmol, 1.25 equiv) and Cs2CO3 (383.02 mg, 1.176 mmol, 2 equiv). The mixture was stirred at 80° C. for 2 h. Desired product could be detected by LCMS. The resulting mixture was extracted with EtOAc (5 mL). The combined organic layers were washed with water (3×5 mL), dried over anhydrous Na2SO4. After filtration, the filtrate was concentrated under reduced pressure. The residue was purified by Prep-TLC (PE/EA 0:1) to afford methyl 3-(4-{3-carbamoylbicyclo[1.1.1]pentan-1-yl}phenoxymethyl)-1-(4-methoxybenzoyl)pyrrolidine-3-carboxylate (85 mg, 25.08%) as a light brown solid. LC/MS: mass calcd. For C27H30N2O6: 478.21, found: 479.15 [M+H]+

The mixture of methyl 3-(4-{3-carbamoylbicyclo[1.1.1]pentan-1-yl}phenoxymethyl)-1-(4-methoxybenzoyl)pyrrolidine-3-carboxylate (25 mg, 0.052 mmol, 1 equiv) in CH3CN (1 mL, 19.024 mmol, 364.16 equiv) and H2O (0.01 mL, 0.555 mmol, 10.63 equiv) was added trimethyl(sodiooxy)silane (0.078 mL, 0.001 mmol, 1.5 equiv). The mixture was stirred at rt for 12 h. Desired product was detected by LCMS. The resulting mixture was concentrated under reduced pressure. The mixture was purified by Prep-HPLC with followed conditions (Column: XBridge Prep OBD C18 Column, 30*150 mm, 5 μm; Mobile Phase A: Water (10 mmol/L NH4HCO3+0.1% NH3·H2O), Mobile Phase B: ACN; Flow rate: 60 mL/min; Gradient: 15% B to 42% B in 9 min, 42% B; Wave Length: 254 nm; RT1 (min): 7; Number Of Runs: 0) to afford 3-(4-{3-carbamoylbicyclo[1.1.1]pentan-1-yl}phenoxymethyl)-1-(4-methoxybenzoyl)pyrrolidine-3-carboxylic acid (3.3 mg, 13.58%) as a white solid.

LC/MS: mass calcd. For C26H28N2O6: 464.19, found: 465.10 [M+H]+, 1H NMR (300 MHz, Methanol-d4) δ 7.50-7.55 (m, 2H), 7.12-7.19 (m, 2H), 6.97-7.02 (m, 2H), 6.83-6.93 (m, 2H), 3.93-4.32 (m, 3H), 3.86 (s, 3H), 3.61-3.78 (m, 3H), 2.38-2.47 (m, 1H), 2.24 (d, J=3.6 Hz, 6H), 2.14 (d, J=6 Hz, 1H).

### Preparation of 3-(((4′-cyano-[1,1′-biphenyl]-4-yl)oxy)methyl)-1-(2,2,2-trifluoro-1-(4-methoxyphenyl)ethyl)pyrrolidine-3-carboxylic acid (Cpd. 2)

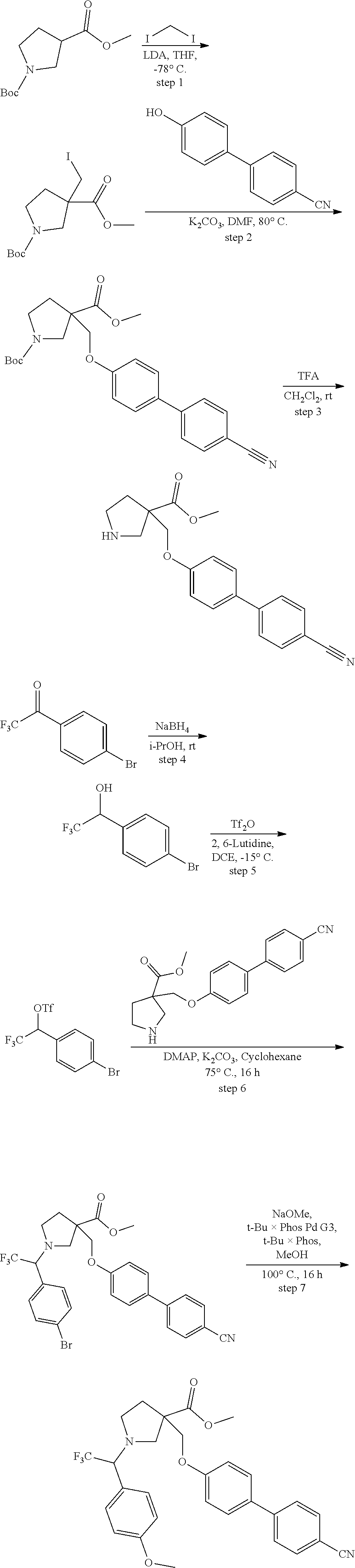

To a mixture of 1-tert-butyl 3-methyl pyrrolidine-1,3-dicarboxylate (2 g, 8.723 mmol, 1 equiv) in THF (100 mL, 1234.277 mmol, 141.50 equiv) was added LDA (1.40 g, 13.085 mmol, 1.5 equiv) at −78° C. The mixture was stirred at the same temperature for 1 h under N2 atmosphere. To the above mixture was added diiodomethane (3.04 g, 11.340 mmol, 1.3 equiv) at −78° C. The mixture was stirred at rt for 1 h. The reaction was monitored by TLC. The mixture was added with H2O (100 mL) and extracted by EtOAc (80 mL×3). The organic layer was combined, washed by brine, dried over Na2SO4, filtered and concentrated to afford 1-tert-butyl 3-methyl 3-(iodomethyl)pyrrolidine-1,3-dicarboxylate (2 g, 62.10%) as yellow oil.

Step 2: To the mixture of 1-tert-butyl 3-methyl 3-(iodomethyl)pyrrolidine-1,3-dicarboxylate (2 g, 5.417 mmol, 1 equiv) and 4′-hydroxy-[1,1′-biphenyl]-4-carbonitrile (1.59 g, 8.125 mmol, 1.5 equiv) in DMF (50 mL, 646.077 mmol, 119.27 equiv) was added K2CO3 (74.87 mg, 0.542 mmol, 2 equiv). The mixture was stirred at 80° C. for 16 h. Desired product could be detected by LCMS. The resulting mixture was filtered, the filter cake was washed with EtOAc. The filtrate was added with H2O (100 mL) and extracted by EtOAc (80 mL×3). The organic was combined, washed by H2O and brine, dried over Na2SO4, filtered and concentrated to afford 1-tert-butyl 3-methyl 3-[({4′-cyano-[1,1′-biphenyl]-4-yl}oxy)methyl]pyrrolidine-1,3-dicarboxylate (2 g, 84.58%) as brown oil. LC/MS: mass calcd. For C25H28N2O5: 436.51, found: 381.15[M-tBu]⁺

Step 3: To the mixture of 1-tert-butyl 3-methyl 3-[({4′-cyano-[1,1′-biphenyl]-4-yl}oxy)methyl]pyrrolidine-1,3-dicarboxylate (3 g, 6.873 mmol, 1 equiv) in DCM (50 mL, 786.530 mmol, 114.44 equiv) was added TFA (0.2 mL, 2.693 mmol, 117.53 equiv). The mixture was stirred at rt for 1 h. Desired product could be detected by LCMS. The mixture was concentrated under reduced pressure. The residue was purified by reverse flash chromatography with the following conditions: column, C18 silica gel; mobile phase, MeCN in Water (0.05% TFA), 30% to 80% gradient in 30 min; detector, UV 254 nm to afford methyl 3-[({4′-cyano-[1,1′-biphenyl]-4-yl}oxy)methyl]pyrrolidine-3-carboxylate (1.2 g, 51.90%) as a brown, yellow oil. LC/MS: mass calcd. For C20H20N2O3: 336.39, found: 337.10[M+H]⁺

Step 4: To the mixture of 1-(4-bromophenyl)-2,2,2-trifluoroethanone (500 mg, 1.976 mmol, 1 equiv) in i-PrOH (10 mL, 130.841 mmol, 66.21 equiv) was added NaBH4 (149.52 mg, 3.952 mmol, 2 equiv). The mixture was added with HCl (1M, 15 mL) and extracted by MTBE (10 mL×3). The organic layer was combined, washed by brine, dried over aNa2SO4, filtered and concentrated to afford 1-(4-bromophenyl)-2,2,2-trifluoroethanol (200 mg, 39.68%) as colorless oil. The reaction worked according to TLC. LC/MS: mass calcd. For C8H6BrF3O: 225.03.

Step 5: To the mixture of 1-(4-bromophenyl)-2,2,2-trifluoroethanol (30 mg, 0.118 mmol, 1 equiv) and 2,6-Lutidine (18.91 mg, 0.177 mmol, 1.5 equiv) in DCE (1 mL, 12.633 mmol, 107.39 equiv) was added Tf2O (49.78 mg, 0.177 mmol, 1.5 equiv) at −15° C. The mixture was stirred at rt for 1 h. The mixture was added with DCM (5 mL) and washed by H2O, HCl (1 M) and brine. The organic layer was dried over Na2SO4, filtered and concentrated to afford 1-(4-bromophenyl)-2,2,2-trifluoroethyl trifluoromethanesulfonate (30 mg, 65.88%) as colorless oil. The reaction was monitored by TLC. LC/MS: mass calcd. For C9H5BrF6O3S: 387.09.

Step 6: To the mixture of methyl 3-[({4′-cyano-[1,1′-biphenyl]-4-yl}oxy)methyl]pyrrolidine-3-carboxylate (200 mg, 0.595 mmol, 1 equiv) and 1-(4-bromophenyl)-2,2,2-trifluoroethyl trifluoromethanesulfonate (690.43 mg, 1.785 mmol, 3 equiv) in Cyclohexane (5 mL, 0.012 mmol, 0.20 equiv) was added DMAP (72.64 mg, 0.595 mmol, 1 equiv) and K2CO3 (164.34 mg, 1.190 mmol, 2 equiv). The mixture was stirred at 75° C. for 16 h. Desired product could be detected by LCMS. The mixture was added DCM (10 mL) and washed by H₂O and brine, dried over Na2SO₄, concentrated. The residue was purified by reverse flash chromatography with the following conditions: column, C18 silica gel; mobile phase, MeCN in Water (0.05% TFA), 50% to 100% gradient in 20 min; detector, UV 254 nm to afford methyl 1-[1-(4-bromophenyl)-2,2,2-trifluoroethyl]-3-[({4′-cyano-[1,1′-biphenyl]-4-yl}oxy)methyl]pyrrolidine-3-carboxylate (40 mg, 11.73%) as colorless oil. LC/MS: mass calcd. For C28H24BrF3N203: 572.09, found: 573.20[M+H]⁺.

Step 7: To the mixture of methyl 1-[1-(4-bromophenyl)-2,2,2-trifluoroethyl]-3-[({4′-cyano-[1,1′-biphenyl]-4-yl}oxy)methyl]pyrrolidine-3-carboxylate (25 mg, 0.044 mmol, 1 equiv) in MeOH (1 mL, 24.699 mmol, 566.50 equiv) was added NaOMe (4.71 mg, 0.088 mmol, 2 equiv), di-tert-butyl[2′,4′,6′-tris(propan-2-yl)-[1,1′-biphenyl]-2-yl]phosphane; {2′-amino-[1,1′-biphenyl]-2-yl}palladio methanesulfonate (3.46 mg, 0.004 mmol, 0.1 equiv) and di-tert-butyl[2′,4′,6′-tris(propan-2-yl)-[1,1′-biphenyl]-2-yl]phosphane (1.85 mg, 0.004 mmol, 0.1 equiv). The mixture was stirred at 100° C. for 16 h. Desired product could be detected by LCMS. The mixture was purified by Prep-HPLC with the following conditions (Column: XBridge Prep OBD C18 Column, 19*250 mm, 5 μm; Mobile Phase A: Water (0.05% TFA), Mobile Phase B: ACN; Flow rate: 25 mL/min; Gradient: 50% B to 80% B in 7 min, 80% B; Wave Length: 254 nm; RT1 (min): 6; Number Of Runs: 0) to afford 3-[({4′-cyano-[1,1′-biphenyl]-4-yl}oxy)methyl]-1-[2,2,2-trifluoro-1-(4-methoxyphenyl)ethyl]pyrrolidine-3-carboxylic acid; trifluoroacetic acid (2.6 mg, 9.46%) as a white solid.

LC/MS: mass calcd. For C₃₀H₂₆F6N₂O₆: 510.18, found: 511.20[M+H]⁺. ¹H NMR (300 MHz, DMSO-d6) δ 7.80-7.93 (m, 4H), 7.70 (d, J=9.0 Hz, 2H), 7.40-7.25 (m, 2H), 7.13-7.01 (m, 2H), 7.00-6.90 (m, 2H), 4.37-4.22 (m, 1H), 4.12 (s, 2H), 3.77 (d, J=3.0 Hz, 3H), 3.05-2.56 (m, 4H), 2.25-2.12 (m, 1H), 1.93-1.77 (m, 1H). ¹⁹F NMR (282 MHz, Methanol-d4) δ −68.112, −74.071.

### Method A: Preparation of 9-chloro-4-((1,5-dimethyl-1H-pyrazol-4-yl)methyl)-7-(5-fluoro-1H-indol-1-yl)-2,3,4,5-tetrahydrobenzo[f][1,4]oxazepine

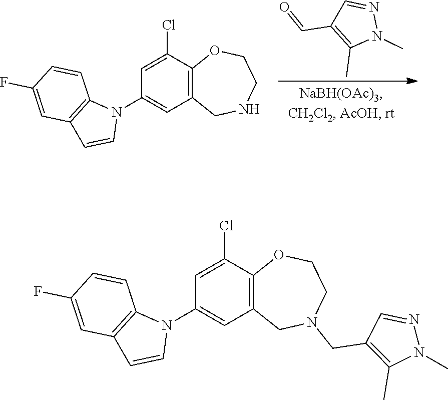

To a stirred mixture of 9-chloro-7-(5-fluoroindol-1-yl)-2,3,4,5-tetrahydro-1,4-benzoxazepine (50 mg, 0.158 mmol, 1 equiv) and 1,5-dimethylpyrazole-4-carbaldehyde (40 mg, 0.316 mmol, 2 equiv) in CH₂Cl₂ (1 ml) was added NaBH(OAc)₃ (70 mg, 0.316 mmol, 2 equiv) and AcOH (40 uL) at room temperature. The resulting mixture was stirred overnight at room temperature. The resulting mixture was concentrated under reduced pressure. The residue was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 51% B to 71% B in 7 min, 71% B; Wave Length: 254 nm; RT1 (min): 5.82; Number Of Runs: 0) to afford 9-chloro-4-[(1,5-dimethylpyrazol-4-yl)methyl]-7-(5-fluoroindol-1-yl)-3,5-dihydro-2H-1,4-benzoxazepine (14.4 mg, 21.42%) as a white solid. LC/MS: mass calcd. For C₂₃H₂₃ClFN₄O: 424.2, found: 425.1 [M+H]⁺.

¹H NMR (300 MHz, Methanol-d₄) δ 7.47 (s, 1H), 7.35-7.42 (m, 3H), 7.25-7.29 (m, 1H), 7.19 (s, 1H), 6.95 (t, J=9.3, 1H), 6.61 (d, J=3.3 Hz, 1H), 4.15-4.17 (m, 2H), 3.85 (s, 2H), 3.73 (s, 3H), 3.57 (s, 2H), 3.05 (m, 2H), 2.22 (s, 3H). ¹⁹F NMR (282 MHz, Methanol-d4) δ −126.0

### Method B: Preparation of 4-((1H-pyrazol-4-yl)methyl)-9-chloro-7-(5-fluoro-1H-indol-1-yl)-2,3,4,5-tetrahydrobenzo[f][1,4]oxazepine

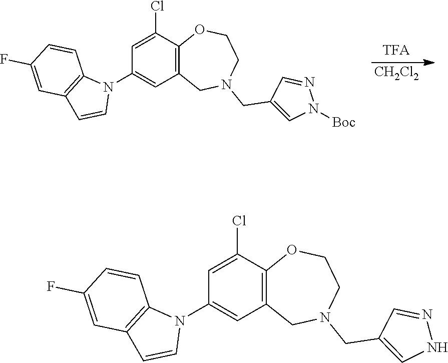 [SMILES: CC(C)(C)OC(=O)n1cc(CN2CCOc3c(Cl)cc(-n4ccc5cc(F)ccc54)cc3C2)cn1 ; "CH2Cl2" ; Fc1ccc2c(ccn2-c2cc(Cl)c3c(c2)CN(Cc2cn[nH]c2)CCO3)c1]

To a stirred solution of tert-butyl 4-{[9-chloro-7-(5-fluoroindol-1-yl)-3,5-dihydro-2H-1,4-benzoxazepin-4-yl]methyl}pyrazole-1-carboxylate (40 mg, 0.080 mmol, 1 equiv) in CH₂Cl₂ (2 mL) was added TFA (0.4 mL) at room temperature. The resulting mixture was stirred for 1 h at room temperature. Desired product could be detected by LCMS. The resulting mixture was concentrated under reduced pressure. The residue was purified by Prep-HPLC with the following conditions (Column: XBridge Prep Phenyl OBD Column, 19*250 mm, 5 μm; Mobile Phase A: Water (10 mmol/L NH₄HCO₃+0.1% NH₃·H₂O), Mobile Phase B: ACN; Flow rate: 25 mL/min; Gradient: 45% B to 75% B in 7 min, 75% B; Wave Length: 254 nm; RT1 (min): 5; Number Of Runs: 0) to afford 9-chloro-7-(5-fluoroindol-1-yl)-4-(1H-pyrazol-4-ylmethyl)-3,5-dihydro-2H-1,4-benzoxazepine (7.2 mg, 22.22%) as a white solid.

LC/MS: mass calcd. For C₂₁H₁₈ClFN₄O: 396.1, found: 397.0 [M+H]⁺.

¹H NMR (300 MHz, Methanol-d₄) δ 7.59-7.62 (br, 2H), 7.51 (d, J=2.7 Hz, 1H), 7.40-7.45 (m, 2H), 7.24-7.31 (m, 2H), 6.97 (t, J=9.3 Hz, 1H), 6.63 (d, J=3.3 Hz, 1H), 4.18-4.24 (m, 2H), 3.90 (s, 2H), 3.73 (s, 2H), 3.09-3.12 (m, 2H). ¹⁹F NMR (282 MHz, Methanol-d₄) δ −126.2

### Method C: Preparation of 9-chloro-7-(5-fluoro-1H-pyrrolo[2,3-b]pyridin-1-yl)-4-((2-methoxypyrimidin-5-yl)methyl)-2,3,4,5-tetrahydrobenzo[f][1,4]oxazepine

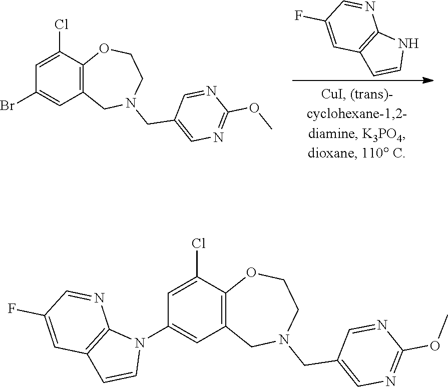 [SMILES: COc1ncc(CN2CCOc3c(Cl)cc(Br)cc3C2)cn1 ; Fc1cnc2[nH]ccc2c1 ; COc1ncc(CN2CCOc3c(Cl)cc(-n4ccc5cc(F)cnc54)cc3C2)cn1]

To a stirred mixture of 7-bromo-9-chloro-4-[(2-methoxypyrimidin-5-yl)methyl]-3,5-dihydro-2H-1,4-benzoxazepine (50 mg, 0.130 mmol, 1 equiv) and 5-fluoro-1H-pyrrolo[2,3-b]pyridine (27 mg, 0.195 mmol, 1.5 equiv) in dioxane (2 mL) was added trans-cyclohexane-1,2-diamine (7 mg, 0.065 mmol, 0.5 equiv), CuI (7 mg, 0.039 mmol, 0.3 equiv) and K₃PO₄ (83 mg, 0.390 mmol, 3 equiv) under nitrogen atmosphere. The resulting mixture was stirred overnight at 110° C. under nitrogen atmosphere. The mixture was filtered and the filtrate was concentrated under reduced pressure. The residue was purified by Prep-HPLC with the following conditions (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 30% B to 60% B in 7 min, 60% B; Wave Length: 254 nm; RT1 (min): 6.12; Number Of Runs: 0) to afford 9-chloro-7-{5-fluoropyrrolo[2,3-b]pyridin-1-yl}-4-[(2-methoxypyrimidin-5-yl)methyl]-3,5-dihydro-2H-1,4-benzoxazepine (5.1 mg, 8.76%) as a white solid.

LC/MS: mass calcd. for C₂₂H₁₉ClFN₅O₂, 439.1, found: 440.1. [M+H]⁺.

¹H NMR (300 MHz, DMSO-d6) δ 8.55 (s, 2H), 8.38 (s, 1H), 7.91-8.04 (m, 3H), 7.65 (d, J=2.7 Hz, 1H), 6.74 (d, J=3.7 Hz, 1H), 4.17-4.18 (m, 2H), 3.90-3.92 (m, 5H), 3.64 (s, 2H), 3.09-3.10 (m, 2H). ¹⁹F NMR (282 MHz, DMSO-d6) δ −173.1

### Method D: Preparation of 5-((9-chloro-7-(5-fluoro-1H-pyrrolo[2,3-b]pyridin-1-yl)-2,3-dihydrobenzo[f][1,4]oxazepin-4 (5H)-yl)methyl)pyrimidin-2 (1H)-one

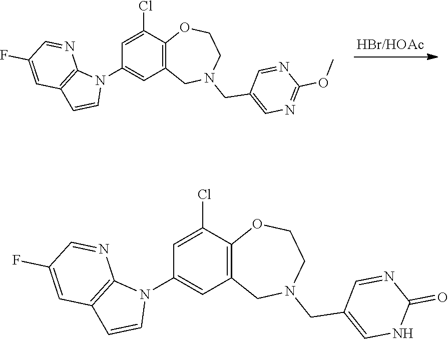 [SMILES: COc1ncc(CN2CCOc3c(Cl)cc(-n4ccc5cc(F)cnc54)cc3C2)cn1 ; O=c1ncc(CN2CCOc3c(Cl)cc(-n4ccc5cc(F)cnc54)cc3C2)c[nH]1]

To a solution of 33% HBr in AcOH (1 mL) was added with 9-chloro-7-{5-fluoropyrrolo[2,3-b]pyridin-1-yl}-4-[(2-methoxypyrimidin-5-yl)methyl]-3,5-dihydro-2H-1,4-benzoxazepine (50 mg, 0.114 mmol, 1 equiv). The resulting mixture was stirred for 2 h at room temperature. Desired product could be detected by LCMS. The resulting mixture was concentrated under reduced pressure. The residue was purified by Prep-HPLC with the following conditions (Column: XBridge Prep OBD C18 Column, 30*150 mm, 5 μm; Mobile Phase A: Water (10 mmol/L NH₄HCO₃+0.1% NH₃·H2O), Mobile Phase B: ACN; Flow rate: 60 mL/min; Gradient: 25% B to 55% B in 7 min, 55% B; Wave Length: 254 nm; RT1 (min): 5.87; Number Of Runs: 0) to afford 5-[(9-chloro-7-{5-fluoropyrrolo[2,3-b]pyridin-1-yl}-3,5-dihydro-2H-1,4-benzoxazepin-4-yl)methyl]-1H-pyrimidin-2-one (5.0 mg, 10.26%) as a white solid.

LC/MS: mass calcd. for C₂₁H₁₇ClFN₅O₂, 425.1, found: 426.0. [M+H]⁺.

¹H NMR (400 MHz, Methanol-d4) δ 8.25-8.31 (m, 3H), 7.77-7.85 (m, 3H), 7.57 (d, J=2.8 Hz, 1H), 6.70 (d, J=3.6 Hz, 1H), 4.19-4.21 (m, 2H), 3.98 (s, 2H), 3.59 (s, 2H), 3.22-3.24 (m, 2H). ¹⁹F NMR (376 MHz, Methanol-d4) δ −140.0.

### Method E: Preparation of 7-(benzo[b]thiophen-3-yl)-9-chloro-4-((2-methoxypyrimidin-5-yl)methyl)-2,3,4,5-tetrahydrobenzo[f][1,4]oxazepine

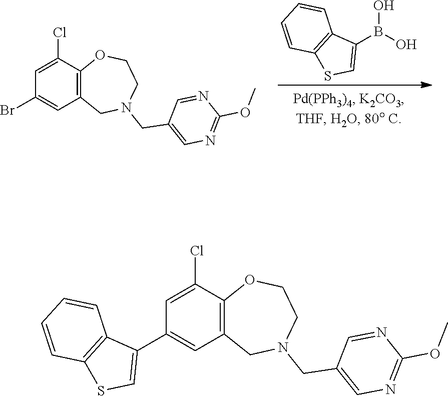 [SMILES: COc1ncc(CN2CCOc3c(Cl)cc(Br)cc3C2)cn1 ; OB(O)c1csc2ccccc12 ; COc1ncc(CN2CCOc3c(Cl)cc(-c4csc5ccccc45)cc3C2)cn1]

To the mixture of 7-bromo-9-chloro-4-[(2-methoxypyrimidin-5-yl)methyl]-3,5-dihydro-2H-1,4-benzoxazepine (50 mg, 0.130 mmol, 1 equiv) and 1-benzothiophen-3-ylboronic acid (35 mg, 0.195 mmol, 1.5 equiv) in THF (0.8 mL) and H₂O (0.2 mL) was added K₂CO₃ (36 mg, 0.260 mmol, 2 equiv) and Pd(PPh₃)₄ (15 mg, 0.013 mmol, 0.1 equiv). The mixture was stirred at 80° C. for 16 h under nitrogen atmosphere. Desired product could be detected by LCMS. The mixture was filtered and the filtrate was concentrated under reduced pressure. The residue was purified by Prep-HPLC with the following conditions (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 50% B to 80% B in 7 min, 80% B; Wave Length: 254 nm; RT1 (min): 6; Number Of Runs: 0) to afford 7-(1-benzothiophen-3-yl)-9-chloro-4-[(2-methoxypyrimidin-5-yl)methyl]-3,5-dihydro-2H-1,4-benzoxazepine (6.0 mg, 10.34%) as an off-white solid.

LC/MS: mass calcd. For C₂₃H₂₀ClN₃O₂S: 437.1, found: 438.1 [M+H]⁺.

¹H NMR (300 MHz, DMSO-d6) δ 8.54 (s, 2H), 8.08 (d, J=6.0 Hz, 1H), 7.56-7.94 (m, 3H), 7.34-7.53 (m, 3H), 4.18-4.19 (m, 2H), 3.95 (s, 2H), 3.90 (s, 3H), 3.67 (s, 2H), 3.05-3.06 (m, 2H).

### Method F: Preparation of 9-chloro-7-(6-fluoro-3,4-dihydroquinolin-1 (2H)-yl)-4-((2-methoxypyrimidin-5-yl)methyl)-2,3,4,5-tetrahydrobenzo[f][1,4]oxazepine

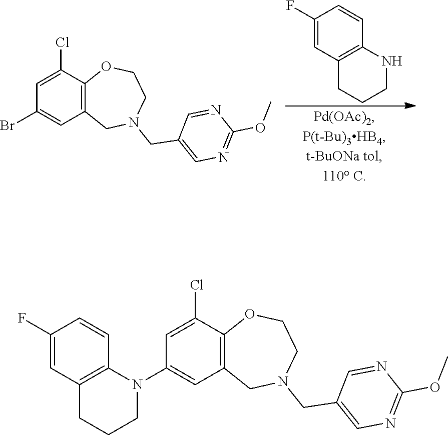 [SMILES: COc1ncc(CN2CCOc3c(Cl)cc(Br)cc3C2)cn1 ; Fc1ccc2c(c1)CCCN2 ; COc1ncc(CN2CCOc3c(Cl)cc(N4CCCc5cc(F)ccc54)cc3C2)cn1]

A solution of 7-bromo-9-chloro-4-[(2-methoxypyrimidin-5-yl)methyl]-3,5-dihydro-2H-1,4-benzoxazepine (50 mg, 0.130 mmol, 1 equiv) in toluene (1.5 mL) was treated with 6-fluoro-1,2,3,4-tetrahydroquinoline (24 mg, 0.156 mmol, 1.2 equiv), Pd(OAc)₂ (1 mg, 0.004 mmol, 0.03 equiv), tri-tert-butylphosphonium tetrafluoroboranuide (2 mg, 0.008 mmol, 0.06 equiv), t-BuONa (25 mg, 0.260 mmol, 2 equiv) for 16 h at 110° C. under nitrogen atmosphere. The resulting mixture was concentrated under reduced pressure. The residue was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 46% B to 76% B in 10 min, 76% B; Wave Length: 254 nm; RT1 (min): 8.95; Number Of Runs: 0) to afford 9-chloro-7-(6-fluoro-3,4-dihydro-2H-quinolin-1-yl)-4-[(2-methoxypyrimidin-5-yl)methyl]-3,5-dihydro-2H-1,4-benzoxazepine (7.3 mg, 12.23%) as a white solid.

LC/MS: mass calcd. For C₂₄H₂₄ClFN₄O₂: 454.2, found: 455.1 [M+H]⁺.

¹H NMR (300 MHz, DMSO-d₆) δ 8.49 (s, 2H), 7.15 (d, J=2.4 Hz, 1H), 6.88-6.95 (m, 2H), 6.76-6.83 (m, 1H), 6.62-6.66 (m, 1H), 4.07-4.08 (m, 2H), 3.90 (s, 3H), 3.78 (s, 2H), 3.60 (s, 2H), 3.49 (t, J=5.7 Hz, 2H), 3.02-3.03 (m, 2H), 2.75 (t, J=5.7 Hz, 2H), 1.85-1.93 (m, 2H). ¹⁹F (282 MHz, DMSO-de) δ −125.5.

### Method G: Preparation of 9-chloro-7-(5-fluoro-1H-indol-1-yl)-4-(oxazol-5-ylmethyl)-2,3,4,5-tetrahydrobenzo[f][1,4]oxazepine

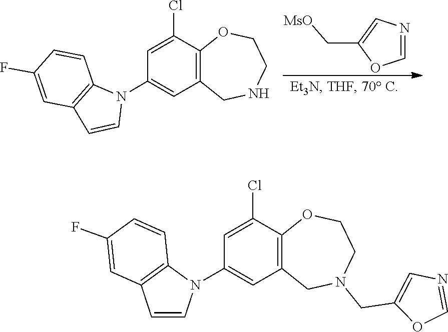 [SMILES: Fc1ccc2c(ccn2-c2cc(Cl)c3c(c2)CNCCO3)c1 ; CS(=O)(=O)OCc1cnco1 ; Fc1ccc2c(ccn2-c2cc(Cl)c3c(c2)CN(Cc2cnco2)CCO3)c1]

To a stirred solution of 1,3-oxazol-5-ylmethyl methanesulfonate (56 mg, 0.316 mmol, 2 equiv) and 9-chloro-7-(5-fluoroindol-1-yl)-2,3,4,5-tetrahydro-1,4-benzoxazepine (50 mg, 0.158 mmol, 1.00 equiv) in THF (1 mL) were added Et₃N (35 mg, 0.316 mmol, 2 equiv). The resulting mixture was stirred for 16 h at 70° C. The resulting mixture was concentrated under reduced pressure. The residue was purified by Prep-HPLC with the following conditions (Column: XBridge Prep C18 OBD Column, 19*150 mm, 5 μm; Mobile Phase A: Water (10 mmol/L NH₄HCO₃+0.1% NH₃·H₂O), Mobile Phase B: ACN; Flow rate: 25 mL/min; Gradient: 47% B to 77% B in 7 min, 77% B; Wave Length: 254 nm; RT1 (min): 6; Number of Runs: 0) to afford 9-chloro-7-(5-fluoroindol-1-yl)-4-(1,3-oxazol-5-ylmethyl)-3,5-dihydro-2H-1,4-benzoxazepine (4.2 mg, 6.59%) as a white solid.

LC/MS: mass calcd. for C₂₁H₁₇ClFN₃O₂, 397.1, found: 398.0 [M+H]⁺.

¹H NMR (300 MHz, Methanol-d₄) δ 8.20 (s, 1H), 7.50 (d, J=2.7 Hz, 1H), 7.41-7.45 (m, 2H), 7.26-7.30 (m, 2H), 7.09 (s, 1H), 6.93-6.99 (m, 1H), 6.62-6.63 (m, 1H), 4.17-4.20 (m, 2H), 3.95 (s, 2H), 3.89 (s, 2H), 3.13-3.16 (m, 2H). ¹⁹F NMR (282 MHz, Methanol-d₄) δ −126.1.

### Method I: 9-chloro-7-(5-fluoroindol-1-yl)-4-[(2-oxo-1H-pyridin-4-yl)methyl]-3-(trifluoromethyl)-2,3-dihydro-1,4-benzoxazepin-5-one

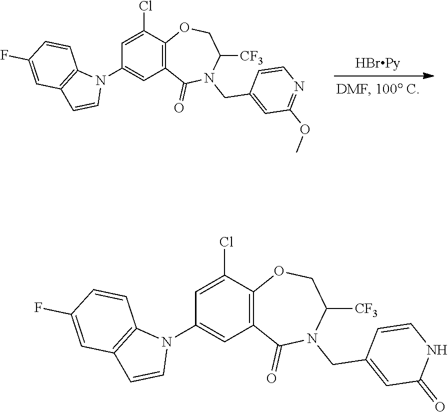 [SMILES: COc1cc(CN2C(=O)c3cc(-n4ccc5cc(F)ccc54)cc(Cl)c3OCC2C(F)(F)F)ccn1 ; O=C1c2cc(-n3ccc4cc(F)ccc43)cc(Cl)c2OCC(C(F)(F)F)N1Cc1cc[nH]c(=O)c1]

To the mixture of 9-chloro-7-(5-fluoroindol-1-yl)-4-[(2-methoxypyridin-4-yl)methyl]-3-(trifluoromethyl)-2,3-dihydro-1,4-benzoxazepin-5-one (25 mg, 0.048 mmol, 1 equiv) in DMF (2 mL) was added pyridine hydrobromide (50 mg, 0.288 mmol, 6 equiv). The mixture was stirred at 60° C. for 24 h. Desired product could be detected by LCMS. The mixture was purified by Prep-HPLC with the following conditions (Column: XBridge Prep Phenyl OBD Column, 19*250 mm, 5 μm; Mobile Phase A: Water (10 mmol/L NH₄HCO₃+0.1% NH₃·H₂O), Mobile Phase B: ACN; Flow rate: 25 mL/min; Gradient: 48% B to 58% B in 10 min, 58% B; Wave Length: 254 nm; RT1 (min): 8.8; Number Of Runs: 0) to afford 9-chloro-7-(5-fluoroindol-1-yl)-4-[(2-oxo-1H-pyridin-4-yl)methyl]-3-(trifluoromethyl)-2,3-dihydro-1,4-benzoxazepin-5-one (1.8 mg, 7.26%) as an off-white solid.

LC/MS: mass calcd. For C₂₄H₁₆ClF₄N₃O₃: 505.1, found: 506.1 [M+H]⁺.

¹H NMR (400 MHz, DMSO-d6) δ 11.55 (br, 1H), 8.10 (d, J=4.0 Hz, 1H), 7.82-7.88 (m, 2H), 7.40-7.55 (m, 3H), 7.11 (m, 1H), 6.74 (d, J=4.0 Hz, 1H), 6.44 (s, 1H), 6.32 (d, J=8.0 Hz, 1H), 5.24 (m, 1H), 4.93 (s, 2H), 4.70 (m, 1H), 4.57 (m, 1H). ¹⁹F NMR (376 MHz, DMSO-d₆) δ −74.5, −123.1

### Method J: Preparation of cis-4-{[9-chloro-7-(5-fluoroindol-1-yl)-3,5-dihydro-2H-1,4-benzoxazepin-4-yl]methyl}-N-methylcyclohexane-1-carboxamide[f][1,4]oxazepine

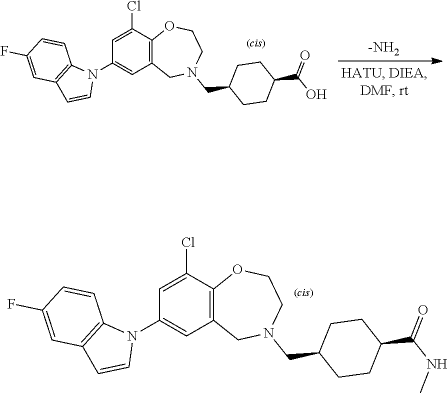 [SMILES: O=C(O)[C@H]1CC[C@@H](CN2CCOc3c(Cl)cc(-n4ccc5cc(F)ccc54)cc3C2)CC1 ; CNC(=O)[C@H]1CC[C@@H](CN2CCOc3c(Cl)cc(-n4ccc5cc(F)ccc54)cc3C2)CC1]

To a stirred solution of cis-4-{[9-chloro-7-(5-fluoroindol-1-yl)-3,5-dihydro-2H-1,4-benzoxazepin-4-yl]methyl}cyclohexane-1-carboxylic acid (35 mg, 0.077 mmol, 1 equiv) and CH₃NH₂·HCl (11 mg, 0.154 mmol, 2 equiv) in DMF (1 mL) was added HATU (45 mg, 0.115 mmol, 1.5 equiv) and DIEA (25 mg, 0.193 mmol, 2.5 equiv). The resulting mixture was stirred for additional 2 h at room temperature. Desired product could be detected by LCMS. The mixture was concentrated under reduced pressure. The residue was purified by Prep-HPLC with the following conditions (Column: XBridge Prep OBD C₁₈ Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 55% B to 85% B in 7 min, 85% B; Wave Length: 254 nm; RT1 (min): 6; Number Of Runs: 0) to afford cis-4-{[9-chloro-7-(5-fluoroindol-1-yl)-3,5-dihydro-2H-1,4-benzoxazepin-4-yl]methyl}-N-methylcyclohexane-1-carboxamide (3.3 mg, 9.16%) as a white solid.

LC/MS: mass calcd. For C₂₆H₂₉ClFN₃O₂: 469.1, found: 469.9 [M+H]⁺

¹H NMR (300 MHz, DMSO-d₆) δ 7.32-7.36 (m, 3H), 7.16-7.29 (m, 2H), 6.81-6.88 (m, 1H), 6.52-6.53 (m, 1H), 4.03-4.06 (m, 2H), 3.83 (s, 2H), 3.05-3.06 (m, 2H), 2.58 (s, 3H), 2.40 (d, J=7.8 Hz, 2H) 2.13-2.18 (m, 1H), 1.75-1.76 (m, 1H), 1.40-1.67 (m, 8H). ¹⁹F NMR (400 MHz, Methanol-d₄) δ −126.1.

### Method K: Preparation of trans-4-{[9-chloro-7-(5-fluoroindol-1-yl)-3,5-dihydro-2H-1,4-benzoxazepin-4-yl]methyl}-N-methylcyclohexane-1-carboxamide [f][1,4]oxazepine

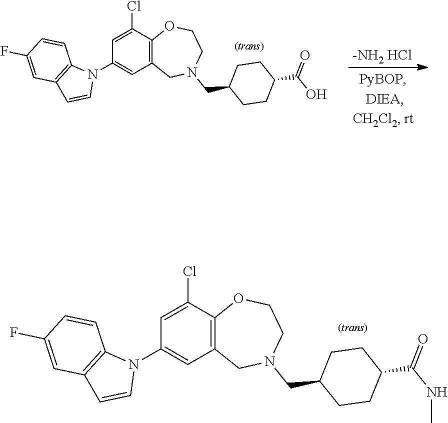 [SMILES: O=C(O)[C@H]1CC[C@H](CN2CCOc3c(Cl)cc(-n4ccc5cc(F)ccc54)cc3C2)CC1 ; "-NH2 HCl" ; CNC(=O)[C@H]1CC[C@H](CN2CCOc3c(Cl)cc(-n4ccc5cc(F)ccc54)cc3C2)CC1]

The mixture of trans-4-{[9-chloro-7-(5-fluoroindol-1-yl)-3,5-dihydro-2H-1,4-benzoxazepin-4-yl]methyl}cyclohexane-1-carboxylic acid (50 mg, 0.109 mmol, 1 equiv) in CH₂Cl₂ (1 mL) was added CH₃NH₂° HCl (15 mg, 0.218 mmol, 2 equiv), PyBOP (86 mg, 0.164 mmol, 1.5 equiv) and DIEA (30 mg, 0.218 mmol, 2 equiv). The mixture was stirred at room temperature for 16 h. Desired product could be detected by LCMS. The resulting mixture was concentrated under reduced pressure. The residue was purified by Prep-HPLC with the following conditions (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 55% B to 85% B in 7 min, 85% B; Wave Length: 254 nm; RT1 (min): 6; Number Of Runs: 0) to afford trans-4-{[9-chloro-7-(5-fluoroindol-1-yl)-3,5-dihydro-2H-1,4-benzoxazepin-4-yl]methyl}-N-methylcyclohexane-1-carboxamide (15.5 mg, 29.05%) as a white solid.

LC/MS: mass calcd. For C₂₆H29ClFN₃O₂: 469.1, found: 469.9 [M+H]⁺

¹H NMR (300 MHz, DMSO-d₆) δ 7.72 (d, J=3.2 Hz, 1H), 7.54-7.59 (m, 3H), 7.45-7.54 (m, 2H), 7.03-7.04 (m, 1H), 6.68-6.69 (m, 1H), 4.10-4.12 (m, 2H), 3.89 (s, 2H), 3.31-3.34 (m, 2H), 2.50-2.53 (m, 3H), 2.25 (d, J=6.9 Hz, 2H), 2.01-2.08 (m, 1H), 1.68-1.78 (m, 4H), 1.51-1.56 (m, 1H), 1.31-1.42 (m, 2H), 0.78-0.92 (m, 2H). ¹⁹F NMR (400 MHz, Methanol-d₄) δ −123.5.

### Method L: Preparation of 4-{[9-chloro-7-(5-fluoroindol-1-yl)-3,5-dihydro-2H-1,4-benzoxazepin-4-yl]methyl}pyran-2-one

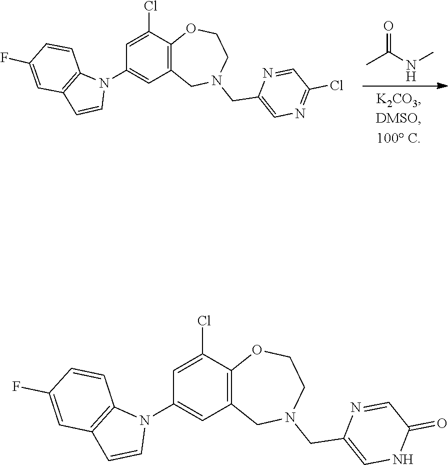

To a stirred solution of 9-chloro-4-[(5-chloropyrazin-2-yl)methyl]-7-(5-fluoroindol-1-yl)-3,5-dihydro-2H-1,4-benzoxazepine (50 mg, 0.113 mmol, 1 equiv) and acetohydroxamic acid (25 mg, 0.339 mmol, 3 equiv) in DMSO (1 mL) was added K₂CO₃ (80 mg, 0.565 mmol, 5 equiv). The resulting mixture was stirred overnight at 80° C. Desired product could be detected by LCMS. The solid was filtered out. The filtrate was purified by Prep-HPLC with the following conditions (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 37% B to 67% B in 7 min, 67% B; Wave Length: 254 nm; RT1 (min): 6; Number Of Runs: 0) to afford 5-{[9-chloro-7-(5-fluoroindol-1-yl)-3,5-dihydro-2H-1,4-benzoxazepin-4-yl]methyl}-1H-pyrazin-2-one (7.9 mg, 16.30%) as a white solid.

LC/MS: mass calcd. For C₂₂H₁₈ClFN₄O₂: 424.1, found: 425.0 [M+H]⁺.

¹H NMR (300 MHz, DMSO-d₆) δ 12.07 (s, 1H), 7.98 (d, J=2.4 Hz, 1H), 7.71 (d, J=3.0 Hz, 1H), 7.60 (d, J=2.7 Hz, 1H), 7.54 (d, J=9.0 Hz, 1H), 7.39-7.45 (m, 3H), 7.07 (t, J=9.1 Hz, 1H), 6.69 (d, J=3.3 Hz, 1H), 4.17-4.18 (m, 2H), 3.94 (s, 2H), 3.57 (s, 2H), 3.09-3.10 (m, 2H). ¹⁹F NMR (282 MHz, DMSO-d₆) δ −123.6.

### Method M: 5-[(9-chloro-7-{pyrrolo[2,3-b]pyrazin-5-yl}-3,5-dihydro-2H-1,4-benzoxazepin-4-yl)methyl]-1H-pyrimidin-2-one trifluoroacetic acid

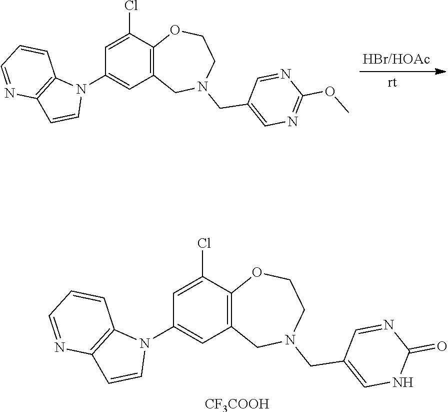 [SMILES: COc1ncc(CN2CCOc3c(Cl)cc(-n4ccc5ncccc54)cc3C2)cn1 ; O=c1ncc(CN2CCOc3c(Cl)cc(-n4ccc5ncccc54)cc3C2)c[nH]1 ; "CF3COOH"]

This title compound was prepared following Method D. But the crude product was purified by Prep-HPLC with the following conditions (Column: Xselect CSH C18 OBD Column 30*150 mm 5 m, n; Mobile Phase A: Water (0.05% TFA), Mobile Phase B: ACN; Flow rate: 60 mL/min; Gradient: 10% B to 40% B in 7 min, 40% B; Wave Length: 254 nm; RT1 (min): 6.12; Number Of Runs: 0) to afford 5-[(9-chloro-7-{pyrrolo[2,3-b]pyrazin-5-yl}-3,5-dihydro-2H-1,4-benzoxazepin-4-yl)methyl]-1H-pyrimidin-2-one trifluoroacetic acid (2.8 mg, 7.35%) as a light yellow solid.

LC/MS: mass calcd. For C₂₀H₁₇ClN₆O2: 408.1, found: 409.1 [M+H]⁺.

¹H NMR (400 MHz, DMSO-d₆) δ 8.58 (d, J=2.6 Hz, 1H), 8.24-8.41 (m, 4H), 8.14 (s, 1H), 7.93 (s, 1H), 6.98 (d, J=4.0 Hz, 1H), 4.20-4.50 (m, 4H), 4.00 (br, 2H), 3.00-3.10 (m, 2H). ¹⁹F NMR (376 MHz, DMSO-d6) δ −73.8, −77.7

### Method N: 4′-chloro-5-fluoro-1′-[(2-methoxypyridin-4-yl)methyl]-1,6′-biindole

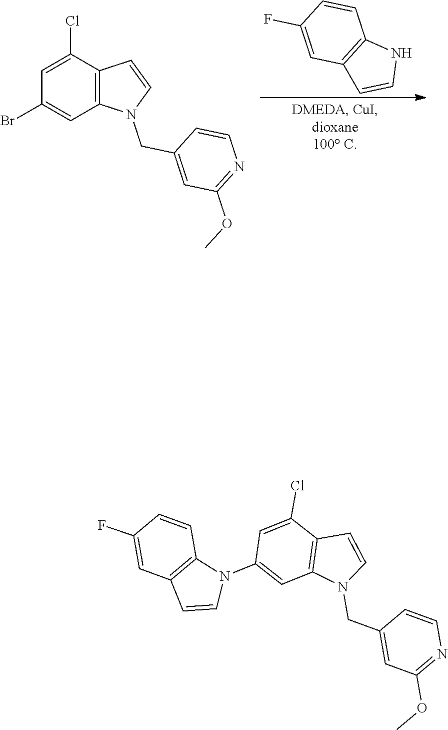 [SMILES: COc1cc(Cn2ccc3c(Cl)cc(Br)cc32)ccn1 ; Fc1ccc2[nH]ccc2c1 ; COc1cc(Cn2ccc3c(Cl)cc(-n4ccc5cc(F)ccc54)cc32)ccn1]

To a stirred mixture of 6-bromo-4-chloro-1-[(2-methoxypyridin-4-yl)methyl]indole (210 mg, 0.597 mmol, 1 equiv) and 5-fluoro-1H-indole (160 mg, 1.194 mmol, 2 equiv) in dioxane (6 mL) were added K₂CO₃ (165 mg, 1.194 mmol, 2 equiv) and CuI (6 mg, 0.030 mmol, 0.05 equiv), DMEDA (11 mg, 0.119 mmol, 0.2 equiv) at room temperature under nitrogen atmosphere. The resulting mixture was stirred for 16 h at 100° C. under nitrogen atmosphere. The resulting mixture was filtered, and the filter cake was washed with EtOAc (3×10 mL). The filtrate was concentrated under reduced pressure. The residue was purified by silica gel column chromatography, eluted with petroleum ether/ethyl acetate (10:1) to afford 4′-chloro-5-fluoro-1′-[(2-methoxypyridin-4-yl)methyl]-1,6′-biindole (170 mg, 67.76%) as a white solid.

LC/MS: mass calcd. For C₂₃H₁₇ClFN₃O: 405.1, found: 406.0 [M+H]⁺.

¹H NMR (400 MHz, Methanol-d4) δ 8.06 (d, J=5.4 Hz, 1H), 7.52 (d, J=3.2 Hz, 1H), 7.46 (d, J=3.2 Hz, 1H), 7.34-7.43 (m, 1H), 7.25-7.27 (m, 2H), 7.19 (s, 1H), 6.86 (t, J=9.1 Hz, 1H), 6.70-6.71 (m, 2H), 6.61 (s, 1H), 6.52 (s, 1H), 5.48 (s, 2H), 3.87 (s, 3H). ¹⁹F NMR (400 MHz, Methanol-d4) δ −126.53.

### Method R: Preparation of 9-chloro-7-(5-fluoroindol-1-yl)-4-[(2-methoxypyrimidin-5-yl)methyl]-3,5-dihydro-2H-1,4-benzoxazepine

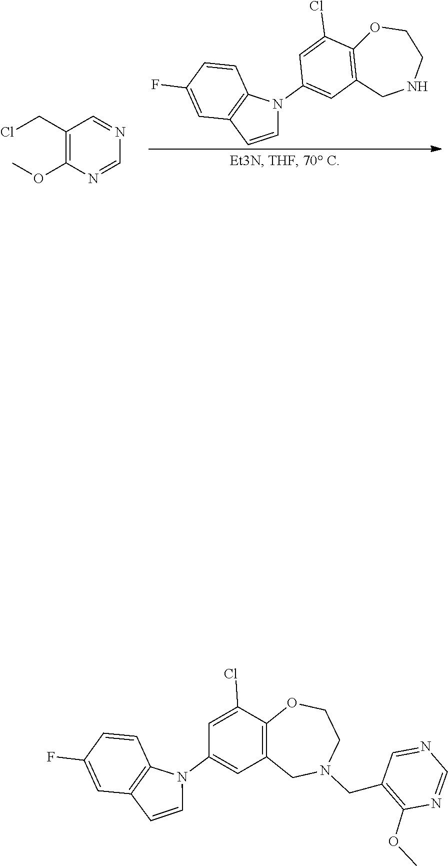

To a stirred mixture of 9-chloro-7-(5-fluoroindol-1-yl)-2,3,4,5-tetrahydro-1,4-benzoxazepine (50 mg, 0.158 mmol, 1 equiv) in THF (3 mL) were added 5-(chloromethyl)-4-methoxypyrimidine (273 mg, 0.474 mmol, 3.00 equiv) and Et₃N (32 mg, 0.316 mmol, 2 equiv) in portions at room temperature. The resulting mixture was stirred for 16 h at 70° C. The reaction was monitored by LCMS. The resulting mixture was concentrated under reduced pressure. The residue was dissolved in DMF (2 mL). The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 30% B to 60% B in 7 min, 60% B; Wave Length: 254 nm; RT1 (min): 5.87; Number Of Runs: 0) to afford 9-chloro-7-(5-fluoroindol-1-yl)-4-[(2-methoxypyrimidin-5-yl)methyl]-3,5-dihydro-2H-1,4-benzoxazepine (2.2 mg, 3.14%) as an off-white solid.

LC/MS: mass calcd. For C₂₃H₂₀ClFN₄O₂:438.13, found: 439.05[M+Na]⁺. ¹H NMR (300 MHz, DMSO-d6) δ 8.71 (s, 1H), 8.48 (s, 1H), 7.72 (d, J=3.3 Hz, 1H), 7.63 (d, J=2.7 Hz, 1H), 7.49-7.53 (m, 1H), 7.41-7.46 (m, 2H), 7.01-7.08 (m, 1H), 6.69 (d, J=3.3 Hz, 1H), 4.18-4.21 (m, 2H), 3.98 (s, 2H), 3.90 (s, 3H), 3.65 (s, 2H), 3.08-3.11 (m, 2H). ¹⁹F NMR (282 MHz, Methanol-d4) δ −126.14.

### Method P: Preparation of 9-chloro-7-(5-fluoroindazol-1-yl)-4-[(2-methoxypyrimidin-5-yl)methyl]-3,5-dihydro-2H-1,4-benzoxazepine

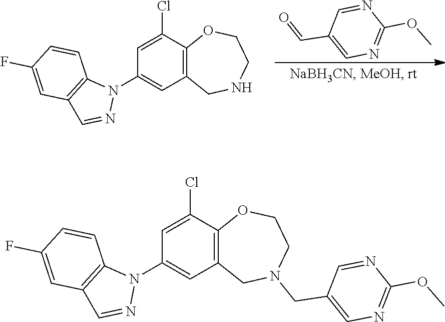

To the mixture of 9-chloro-7-(5-fluoroindazol-1-yl)-2,3,4,5-tetrahydro-1,4-benzoxazepine (50 mg, 0.157 mmol, 1 equiv) and 2-methoxypyrimidine-5-carbaldehyde (43 mg, 0.314 mmol, 2 equiv) in MeOH (3 mL) was added NaBH₃CN (3 mg, 0.048 mmol, 3 equiv). The mixture was stirred at room temperature for 2 h. Desired product could be detected by LCMS. The mixture was filtered. The filtrate was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 40% B to 70% B in 10 min, 70% B; Wave Length: 254 nm; RT1 (min): 8.89; Number Of Runs: 0) to afford 9-chloro-7-(5-fluoroindazol-1-yl)-4-[(2-methoxypyrimidin-5-yl)methyl]-3,5-dihydro-2H-1,4-benzoxazepine (4.2 mg, 6.05%) as a light yellow solid.

LC/MS: mass calcd. For C₂₂H₁₉ClFN₅O₂: 439.1, found: 440.1 [M+H]⁺.

¹H NMR (300 MHz, DMSO-d₆) δ 8.53 (s, 2H), 8.37 (d, J=3.0 Hz, 1H), 7.80-7.90 (m, 1H), 7.76 (d, J=3.0 Hz, 1H), 7.62-7.70 (m, 1H), 7.58 (d, J=3.0 Hz, 1H), 7.31-7.41 (m, 1H), 4.14-4.22 (m, 2H), 4.00 (s, 2H), 3.90 (s, 3H), 3.67 (s, 2H), 3.05 (s, 2H). ¹⁹F NMR (282 MHz, DMSO-d₆) δ −119.1.

### Method Q: Preparation of 9-chloro-7-(5-fluoroindol-1-yl)-4-[(4-methylpyrimidin-5-yl)methyl]-3,5-dihydro-2H-1,4-benzoxazepine

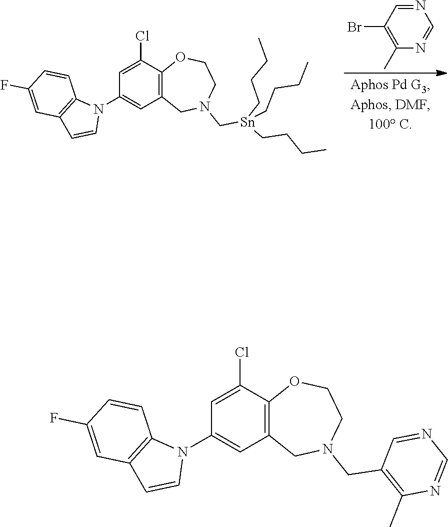

A mixture of 9-chloro-7-(5-fluoroindol-1-yl)-4-[(tributylstannyl)methyl]-3,5-dihydro-2H-1,4-benzoxazepine (100 mg, 0.161 mmol, 1 equiv), Aphos (10 mg, 0.016 mmol, 0.10 equiv) and Aphos Pd G₃ (4 mg, 0.015 mmol, 0.09 equiv) in DMF (3 mL) was added 5-bromo-4-methylpyrimidine (42 mg, 0.241 mmol, 1.5 equiv). The mixture was stirred for 16 h at 100° C. under nitrogen atmosphere. Desired product could be detected by LCMS. The residue was purified by reversed-phase flash chromatography with the following conditions: column, C18 silica gel; mobile phase, MeCN in Water (0.05% NH4HCO3), 10% to 50% gradient in 15 min; detector, UV 254 nm. The crude product was purified by Prep-HPLC with the following conditions (Column: Xselect CSH C18 OBD Column 30*150 mm 5 m, n; Mobile Phase A: Water (0.1% FA), Mobile Phase B: ACN; Flow rate: 60 mL/min; Gradient: 50% B to 80% B in 7 min; Wave Length: 254 nm; RT1 (min): 5.87) to afford 9-chloro-7-(5-fluoroindol-1-yl)-4-[(4-methylpyrimidin-5-yl)methyl]-3,5-dihydro-2H-1,4-benzoxazepine (3.9 mg, 5.60%) as a white solid.

LC/MS: mass calcd. For C₂₃H₂₀ClFN₄O: 422.1, found: 423.1 [M+H]⁺.

¹H NMR (300 MHz, DMSO-d6) δ 8.92 (s, 1H), 8.51 (s, 1H), 7.71 (d, J=3.3 Hz, 1H), 7.64 (d, J=2.6 Hz, 1H), 7.53 (d, J=9.0, 4.4 Hz, 1H), 7.44-7.40 (m, 2H), 7.05 (t, J=9.2 Hz, 1H), 6.69 (d, J=3.2 Hz, 1H), 4.21 (d, J=5.7, 2.9 Hz, 2H), 3.98 (s, 2H), 3.70 (s, 2H), 3.36 (s, 1H), 3.05-3.03 (m, 2H). ¹⁹F NMR (282 MHz, DMSO-d6) δ −123.62.

Characterization data for exemplary compounds produced according to the methods set forth above are provided in Table (III).

TABLE III

| Name | ¹H-NMR | MS(M + H)⁺ | Method |
|---|---|---|---|
| 9-chloro-7-(5- | ¹H NMR (400 MHz, DMSO- | 423.0 | A |
| fluoro-1H-indol-1- | d₆) δ 8.62 (s, 2H), 7.71 (d, J = | | |
| yl)-4-((2- | 3.3 Hz, 1H), 7.63 (d, J = 2.6 | | |
| methylpyrimidin-5- | Hz, 1H), 7.49-7.51 (m, 1H), | | |
| yl)methyl)-2,3,4,5-tetra- | 7.38-7.44 (m, 2H), 7.05 (t, J = | | |
| hydrobenzo[f][1,4]oxazepine | 9.2 Hz, 1H), 6.69 (d, J = | | |
| | 3.3 Hz, 1H), 4.15-4.22 (m, | | |
| | 2H), 3.97 (s, 2H), 3.70 (s, | | |
| | 2H), 3.03-3.10 (m, 2H), 2.60 | | |
| | (s, 3H). ¹⁹F NMR (376 MHz, | | |
| | DMSO-d₆) δ −123.4. | | |
| 5-((9-chloro-7-(5- | ¹H NMR (400 MHz, DMSO- | 434.0 | A |
| fluoro-1H-indol-1- | d₆) δ 8.96 (s, 2H), 7.70 (d, J = | | |
| yl)-2,3-dihydro- | 3.3 Hz, 1H), 7.64 (d, J = 2.6 | | |
| benzo[f][1,4]oxazepin-4(5H)- | Hz, 1H), 7.48-7.49 (m, 1H), | | |
| yl)methyl)pyrimidine- | 7.41-7.44 (m, 2H), 7.05 (t, J = | | |
| 2-carbonitrile | 9.2 Hz, 1H), 6.69 (d, J = | | |
| | 3.2 Hz, 1H), 4.19-4.21 (m, | | |
| | 2H), 3.98 (s, 2H), 3.85 (s, | | |
| | 2H), 3.12-3.14 (m, 2H). ¹⁹F | | |
| | NMR (376 MHz, DMSO-d₆) | | |
| | δ −123.4. | | |
| 9-chloro-7-(5- | ¹H NMR (400 MHz, DMSO- | 507.0 | A |
| fluoro-1H-indol-1- | d₆) δ 8.61 (s, 2H), 7.71 (d, J = | | |
| yl)-4-((2-(2,2,2- | 3.2 Hz, 1H), 7.63 (d, J = 2.6 | | |
| trifluoroethoxy)pyrimidin- | Hz, 1H), 7.49-7.51 (m, 1H), | | |
| 5-yl)methyl)-2,3,4,5-tetra- | 7.41-7.44 (m, 2H), 7.02- | | |
| hydrobenzo[f][1,4]oxazepine | 7.05 (m, 1H), 6.69 (d, J = 3.3 | | |
| | Hz, 1H), 4.98-5.05 (m, 2H), | | |
| | 4.17-4.19 (m, 2H), 3.98 (s, | | |
| | 2H), 3.71 (s, 2H), 3.06-3.08 | | |
| | (m, 2H). ¹⁹F NMR (376 MHz, | | |
| | DMSO-d₆) δ −72.4, −123.6. | | |
| 5-((9-chloro-7-(5- | ¹H NMR (300 MHz, DMSO- | 454.0 | A |
| fluoro-1H-indol-1- | d₆) δ 7.77 (s, 1H), 7.72 (d, J = | | |
| yl)-2,3-dihydro- | 3.3 Hz, 1H), 7.63 (d, J = 2.6 | | |
| benzo[f][1,4]oxazepin-4(5H)- | Hz, 1H), 7.52-7.55 (m, 1H), | | |
| yl)methyl)-2- | 7.37-7.43 (m, 2H), 7.02- | | |
| methoxypyrimidin- | 7.06 (m, 1H), 6.84 (br, 2H), | | |
| 4-amine | 6.69 (d, J = 3.3 Hz, 1H), 7.15- | | |
| | 4.17 (m, 2H), 3.92 (s, 2H), | | |
| | 3.75 (s, 3H), 3.51 (s, 2H), | | |
| | 3.01-3.04 (m, 2H). ¹⁹F NMR | | |
| | (376 MHz, DMSO-d₆) δ −123.6. | | |
| 5-((9-chloro-7-(5- | ¹H NMR (300 MHz, | 439.1 | A |
| fluoro-1H-indol-1- | Methanol-d₄) δ 7.54 (d, J = | | |
| yl)-2,3-dihydro- | 2.7 Hz, 2H), 7.42-7.46 (m, | | |
| benzo[f][1,4]oxazepin-4(5H)- | 2H), 7.26-7.31 (m, 1H), 7.24 | | |
| yl)methyl)pyrimidine- | (d, J = 2.6 Hz, 1H), 6.95- | | |
| 2,4-diamine | 7.07 (m, 1H), 6.65 (d, J = 3.3 | | |
| | Hz, 1H), 4.20-4.23 (m, 2H), | | |
| | 3.94 (s, 2H), 3.58 (s, 2H), | | |
| | 3.13-3.19 (m, 2H). ¹⁹F NMR | | |
| | (376 MHz, DMSO-d₆) δ −123.6. | | |
| methyl 5-((9- | ¹H NMR (300 MHz, DMSO- | 466.0 | A |
| chloro-7-(5-fluoro- | d₆) δ 8.66 (s, 1H), 8.04-8.06 | | |
| 1H-indol-1-yl)-2,3-dihydro- | (m, 1H), 7.91-7.95 (m, 1H), | | |
| benzo[f][1,4]oxazepin-4(5H)- | 7.71 (d, J = 3.3 Hz, 1H), 7.63 | | |
| yl)methyl)picolinate | (d, J = 2.6 Hz, 1H), 7.40- | | |
| | 7.48 (m, 2H), 7.34 (d, J = 2.7 | | |
| | Hz, 1H), 7.01-7.06 (m, 1H), | | |
| | 6.68 (d, J = 3.3 Hz, 1H), 4.17- | | |
| | 4.19 (m, 2H), 3.96 (s, 2H), | | |
| | 3.87 (s, 3H), 3.85 (s, 2H), | | |
| | 3.09-3.11 (m, 2H). ¹⁹F NMR | | |
| | (282 MHz, DMSO-d₆) δ −123.2. | | |
| 9-chloro-7-(5- | ¹H NMR (300 MHz, DMSO- | 461.2 | C |
| cyclopropyl-1H- | d₆) δ 8.53 (s, 2H), 7.56-7.63 | | |
| indol-1-yl)-4-((2- | (m, 2H), 7.32-7.46 (m, 3H), | | |
| methoxypyrimidin- | 6.90-6.98 (m, 1H), 6.59 (d, | | |
| 5-yl)methyl)- | J = 3.0 Hz, 1H), 4.13-4.22 | | |
| 2,3,4,5-tetrahydro- | (m, 2H), 3.96 (s, 2H), 3.91 (s, | | |
| benzo[f][1,4]oxazepine | 3H), 3.66 (s, 2H), 3.03-3.10 | | |
| | (m, 2H), 1.94-2.11 (m, 1H), | | |
| | 0.88-1.00 (m, 2H), 0.60- | | |
| | 0.68 (m, 2H). | | |
| 9-chloro-7-(5- | ¹H NMR (300 MHz, DMSO- | 467.1 | A |
| fluoro-1H-indol-1- | d₆) δ 8.66 (s, 2H), 7.71 (d, J = | | |
| yl)-4-((2-(2- | 3.3 Hz, 1H), 7.63 (d, J = 2.6 | | |
| methoxyethyl)pyrimidin- | Hz, 1H), 7.49-7.51 (m, 1H), | | |
| 5-yl)methyl)- | 7.38-7.47 (m, 2H), 7.01- | | |
| 2,3,4,5-tetrahydro- | 7.05 (m, 1H), 6.69 (d, J = 3.3 | | |
| benzo[f][1,4]oxazepine | Hz, 1H), 4.17-4.19 (m, 2H), | | |
| | 3.98 (s, 2H), 3.79 (t, J = 6.6 | | |
| | Hz, 2H), 3.71 (s, 2H), 3.21 (s, | | |
| | 3H), 3.03-3.14 (m, 4H). ¹⁹F | | |
| | NMR (282 MHz, DMSO-d₆) | | |
| | δ −123.6. | | |
| 9-chloro-4-((2- | ¹H NMR (300 MHz, DMSO- | 449.0 | A |
| cyclopropylpyrimidin- | d₆) δ 8.56 (s, 2H), 7.72 (d, J = | | |
| 5-yl)methyl)-7- | 3.3 Hz, 1H), 7.63 (d, J = 2.6 | | |
| (5-fluoro-1H-indol- | Hz, 1H), 7.39-7.47 (m, 3H), | | |
| 1-yl)-2,3,4,5-tetrahydro- | 7.01-7.05 (m, 1H), 6.69 (d, J = | | |
| benzo[f][1,4]oxazepine | 3.3 Hz, 1H), 4.16-4.19 (m, | | |
| | 2H), 3.97 (s, 2H), 3.67 (s, | | |
| | 2H), 3.01-3.05 (m, 2H), 2.11- | | |
| | 2.15 (m, 1H), 0.94-1.06 | | |
| | (m, 4H). ¹⁹F NMR (282 MHz, | | |
| | DMSO-d₆) δ −123.6. | | |
| 5-((9-chloro-7-(5,6- | ¹H NMR (300 MHz, | 443.1 | D |
| difluoro-1H-indol- | Methanol-d₄) δ 8.28 (s, 1.7H), | | |
| 1-yl)-2,3-dihydro- | 7.97 (s, 0.3H), 7.57-7.41 | | |
| benzo[f][1,4]oxazepin-4(5H)- | (m, 2.3H), 7.41-7.26 (m, | | |
| yl)methyl)pyrimidin- | 1.7H), 7.26-7.10 (m, 0.7H), | | |
| 2(1H)-one | 6.80-6.63 (m, 1H), 6.45- | | |
| | 6.31 (m, 0.3H), 4.18-4.21 | | |
| | (m, 2H), 3.97 (s, 1.4H), 3.87 | | |
| | (s, 0.6H), 3.58 (s, 1.4H), 3.54 | | |
| | (s, 0.6H), 3.13-3.19 (m, | | |
| | 2H). ¹⁹F NMR (282 MHz, | | |
| | Methanol-d₄) δ −145.2, −149.3 | | |
| | (A mixture of tautomer) | | |
| 9-chloro-7-(5- | ¹H NMR (300 MHz, DMSO- | 477.1 | A |
| fluoro-1H-indol-1- | d₆) δ 9.01 (s, 2H), 7.71 (d, J = | | |
| yl)-4-((2-(trifluoro- | 3.0 Hz, 1H), 7.64 (d, J = 2.7 | | |
| methyl)pyrimidin-5- | Hz, 1H), 7.40-7.64 (m, 3H), | | |
| yl)methyl)-2,3,4,5- | 7.03 (t, J = 9.3 Hz, 1H), 6.69 | | |
| tetrahydro- | (d, J = 3.0 Hz, 1H), 4.19- | | |
| benzo[f][1,4]oxazepine | 4.21 (m, 2H), 4.02 (s, 2H), | | |
| | 3.86 (s, 2H), 3.12-3.14 (m, | | |
| | 2H). ¹⁹F NMR (282 MHz, | | |
| | DMSO-d₆) δ −68.8, −123.6. | | |
| 9-chloro-7-(5- | ¹H NMR (300 MHz, DMSO- | 409.1 | A |
| fluoro-1H-indol-1- | d₆) δ 9.01 (s, 2H), 7.71 (d, J = | | |
| yl)-4-(pyrimidin-5- | 3.0 Hz, 1H), 7.64 (d, J = 2.7 | | |
| ylmethyl)-2,3,4,5- | Hz, 1H), 7.40-7.64 (m, 3H), | | |
| tetrahydro- | 7.03 (t, J = 9.3 Hz, 1H), 6.69 | | |
| benzo[f][1,4]oxazepine | (d, J = 3.0 Hz, 1H), 4.19- | | |
| | 4.21 (m, 2H), 4.02 (s, 2H), | | |
| | 3.86 (s, 2H), 3.12-3.14 (m, | | |
| | 2H). ¹⁹F NMR (282 MHz, | | |
| | DMSO-d₆) δ −123.6. | | |
| 5-((9-chloro-7-(5- | ¹H NMR (400 MHz, DMSO- | 425.1 | B |
| fluoro-1H-indol-1- | d₆) δ 8.36 (d, J = 1.8 Hz, 1H), | | |
| yl)-2,3-dihydro- | 7.71 (d, J = 3.3 Hz, 1H), 7.63 | | |
| benzo[f][1,4]oxazepin-4(5H)- | (d, J = 2.6 Hz, 1H), 7.48- | | |
| yl)methyl)pyridazin- | 7.55 (m, 1H), 7.39-7.46 (m, | | |
| 3-amine | 1H), 7.37 (d, J = 2.7 Hz, 1H), | | |
| | 7.02-7.11 (m, 1H), 6.74 (d, | | |
| | J = 1.8 Hz, 1H), 6.66-6.71 | | |
| | (m, 1H), 6.31 (s, 2H), 4.15- | | |
| | 4.21 (m, 2H), 3.93 (s, 2H), | | |
| | 3.61 (s, 2H), 3.35 (s, 2H), | | |
| | 3.05-3.12 (m, 2H). ¹⁹F | | |
| | NMR (376 MHz, DMSO-d₆) | | |
| | δ −123.6. | | |
| 9-chloro-4-((2- | ¹H NMR (400 MHz, DMSO- | 471.1 | G |
| chloro-4,6- | d₆) δ 7.70 (d, J = 3.6 Hz, 1H), | | |
| dimethylpyrimidin- | 7.62 (d, J = 2.4 Hz, 1H), 7.42- | | |
| 5-yl)methyl)-7-(5- | 7.53 (m, 3H), 7.03- | | |
| fluoro-1H-indol-1- | 7.09(m, 1H), 6.68-6.69 (m, | | |
| yl)-2,3,4,5-tetrahydro- | 1H), 4.14-4.17 (m, 2H), 3.94 | | |
| benzo[f][1,4]oxazepine | (s, 2H), 3.73 (s, 2H), 2.92- | | |
| | 2.96 (m, 2H), 2.48-2.51 (m, | | |
| | 6H). ¹⁹F NMR (376 MHz, | | |
| | DMSO-d₆) δ −123.63. | | |
| 5-((9-chloro-7-(5- | ¹H NMR (400 MHz, DMSO- | 453.1 | L |
| fluoro-1H-indol-1- | d₆) δ 7.52 (s, 1H), 7.41-7.47 | | |
| yl)-2,3-dihydro- | (m, 2H), 7.29-7.32 (m, 2H), | | |
| benzo[f][1,4]oxazepin-4(5H)- | 6.97-7.02 (m, 1H), 6.65 (d, J = | | |
| yl)methyl)-4,6- | 5.6 Hz, 1H), 4.19-4.21 (m, | | |
| dimethylpyrimidin- | 2H), 3.92 (s, 2H), 3.63 (s, | | |
| 2(1H)-one | 2H), 3.07-3.09 (m, 2H), | | |
| | 2.41 (s, 6H). ¹⁹F NMR (376 | | |
| | MHz, DMSO-d₆) δ −126.18. | | |
| 5-((9-chloro-7-(6- | ¹H NMR (300 MHz, DMSO- | 442.0 | E |
| fluorobenzo[b]thiophen- | d6) δ 8.30 (s, 2H), 7.81 (d, J = | | |
| 3-yl)-2,3-dihydro- | 2.1 Hz, 1H), 7.69-7.71 | | |
| benzo[f][1,4]oxazepin-4(5H)- | (m, 1H), 7.55 (s, 2H), 7.29- | | |
| yl)methyl)pyrimidin- | 7.30 (m, 1H), 7.20-7.25 (m, | | |
| 2(1H)-one | 1H), 4.18-4.20 (m, 2H), | | |
| | 3.90 (s, 2H), 3.65 (s, 2H), | | |
| | 3.04 (s, 2H). ¹⁹F NMR (282 | | |
| | MHz, DMSO-d6) δ −117.55. | | |
| 4-((1H-pyrrolo[2,3- | ¹H NMR (300 MHz, DMSO- | 447.0 | A |
| b]pyridin-5- | d6) δ 11.60 (s, 1H), 8.16 (s, | | |
| yl)methyl)-9- | 1H), 7.90 (s, 1H), 7.71 (d, J = | | |
| chloro-7-(5-fluoro- | 3.3 Hz, 1H), 7.63 (d, J = 2.6 | | |
| 1H-indol-1-yl)- | Hz, 1H), 7.56-7.39 (m, 3H), | | |
| 2,3,4,5-tetrahydro- | 7.31 (d, J = 2.6 Hz, 1H), 7.11- | | |
| benzo[f][1,4]oxazepine | 7.00 (m, 1H), 6.68 (d, J = | | |
| | 3.3 Hz, 1H), 6.41 (s, 1H), | | |
| | 4.19-4.20 (m, 2H), 3.96 (s, | | |
| | 2H), 3.79 (s, 2H), 3.08-3.09 | | |
| | (m, 2H). ¹⁹F NMR (282 MHz, | | |
| | DMSO-d6) δ −123.62. | | |
| ethyl 5-((9-chloro- | ¹H NMR (300 MHz, DMSO- | 480.1 | R |
| 7-(5-fluoro-1H- | d6) δ 8.92 (s, 2H), 7.71 (d, J = | | |
| indol-1-yl)-2,3-dihydro- | 3.3 Hz, 1H), 7.64 (d, J = | | |
| benzo[f][1,4]oxazepin-4(5H)- | 2.6 Hz, 1H), 7.47-7.52 (m, | | |
| yl)methyl)pyrimidine- | 1H), 7.48-7.38 (m, 2H), | | |
| 2-carboxylate | 7.06-7.07 (m, 1H), 6.68 (d, J = | | |
| | 3.3 Hz, 1H), 4.37 (t, J = 7.1 | | |
| | Hz, 2H), 4.20-4.21 (m, 2H), | | |
| | 3.98 (s, 2H), 3.84 (s, 2H), | | |
| | 3.17-3.08 (m, 2H), 1.33 (t, J = | | |
| | 7.1 Hz, 3H). ¹⁹F NMR (282 | | |
| | MHz, DMSO-d6) δ 123.64. | | |
| 5-((9-chloro-7-(5- | ¹H NMR (300 MHz, | 530.0 | H |
| fluoro-1H-indol- | DMSO-d₆) δ 8.78 (s, 2H), | | |
| 1-yl)-2,3-dihydro- | 7.72 (d, J = 3.3 Hz, 1H), | | |
| benzo[f][1,4]oxazepin- | 7.64 (d, J = 2.7 Hz, 1H), | | |
| 4(5H)-yl)methyl)-N- | 7.48-7.57 (m, 1H), 7.38- | | |
| (methylsulfonyl)pyrimidine- | 7.47 (m, 2H), 7.02-7.14 | | |
| 2-carboxamide | (m, 1H), 6.69 (d, J = 3.2 | | |
| | Hz, 1H), 4.15-4.23 (m, | | |
| | 2H), 4.00 (s, 2H), 3.79 (s, | | |
| | 2H), 3.06-3.12 (m, 2H), | | |
| | 3.03 (s, 3H). ¹⁹F NMR | | |
| | (282 MHz, DMSO-d₆) δ −123.5. | | |
| 5-((9-chloro-7- | ¹H NMR (300 MHz, | 422.1 | A |
| (4,5-difluoro-1H- | DMSO-d₆) δ 8.15 (s, 2H), | | |
| indol-1-yl)-2,3-dihydro- | 7.77 (d, J = 3.4 Hz, 1H), | | |
| benzo[f][1,4]oxazepin-4(5H)- | 7.65 (d, J = 2.6 Hz, 1H), | | |
| yl)methyl)pyrimidin- | 7.39 (d, J = 2.7 Hz, 1H), | | |
| 2-amine) | 7.18-7.36 (m, 2H), 6.83 | | |
| | (d, J = 3.3 Hz, 1H), 6.57 (s, | | |
| | 2H), 4.13-4.20 (m, 2H), | | |
| | 3.92 (s, 2H), 3.50 (s, 2H), | | |
| | 3.01-3.07 (s, 2H). ¹⁹F | | |
| | NMR (282 MHz, DMSO- | | |
| | d₆) δ −148.5, 151.3. | | |
| 9-chloro-7-(6- | ¹H NMR (300 MHz, | 440.0 | A |
| fluorobenzofuran- | DMSO-d₆) δ 8.53 (s, 2H), | | |
| 3-yl)-4-((2- | 8.42 (s, 1H), 7.87-7.97 | | |
| methoxypyrimidin- | (m, 1H), 7.72 (d, J = 2.2 | | |
| 5-yl)methyl)- | Hz, 1H), 7.61-7.69 (m, | | |
| 2,3,4,5-tetrahydro- | 1H), 7.51 (d, J = 2.2 Hz, | | |
| benzo[f][1,4]oxazepine | 1H), 7.17-7.30 (m, 1H), | | |
| | 4.13-4.20 (m, 2H), 3.97 | | |
| | (s, 2H), 3.91 (s, 3H), 3.65 | | |
| | (s, 2H), 3.00-3.06 (m, | | |
| | 2H). ¹⁹F NMR (282 MHz, | | |
| | DMSO-d₆) δ −116.8. | | |
| 5-((9-chloro-7-(6- | ¹H NMR (300 MHz, | 322.1 | L |
| fluorobenzofuran- | DMSO-d₆) δ 11.87 (s, 1H), | | |
| 3-yl)-2,3-dihydro- | 8.42 (s, 1H), 8.26 (s, 2H), | | |
| benzo[f][1,4]oxazepin-4(5H)- | 7.87-7.97 (m, 1H), 7.72 | | |
| yl)methyl)pyrimidin- | (d, J = 2.1 Hz, 1H), 7.61- | | |
| 2(1H)-one | 7.69 (m, 1H), 7.54 (d, J = | | |
| | 2.2 Hz, 1H), 7.20-7.32 | | |
| | (m, 1H), 4.10-4.17 (m, | | |
| | 2H), 3.96 (s, 2H), 3.45 (s, | | |
| | 2H), 3.01-3.08 (m, 2H). | | |
| | ¹⁹F NMR (282 MHz, | | |
| | DMSO-d₆) δ −116.9. | | |
| 5-((9-chloro-7-(5- | ¹H NMR (300 MHz, DMSO- | 426.0 | L |
| fluoro-1H-indazol- | d₆) δ 8.37 (d, J = 0.9 Hz, 1H), | | |
| 1-yl)-2,3-dihydro- | 8.16 (s, 2H), 7.80-7.90 (m, | | |
| benzo[f][1,4]oxazepin-4(5H)- | 1H), 7.75 (d, J = 2.6 Hz, 1H), | | |
| yl)methyl)pyrimidin- | 7.72-7.80 (m, 1H), 7.61 (d, J = | | |
| 2(1H)-one | 2.7 Hz, 1H), 7.35-7.56 | | |
| | (m, 1H), 4.12-4.20 (m, 2H), | | |
| | 3.99 (s, 2H), 3.46 (s, 2H), | | |
| | 3.00-3.08 (m, 2H). ¹⁹F NMR | | |
| | (282 MHz, DMSO-d₆) δ −119.1, | | |
| | 121.8. | | |
| 9-chloro-4-((2- | ¹H NMR (300 MHz, DMSO- | 444.1 | A |
| chloropyrimidin-5- | d₆) δ 8.72 (s, 2H), 8.37 (d, J = | | |
| yl)methyl)-7-(5- | 3.0 Hz, 1H), 7.81-7.88 (m, | | |
| fluoro-1H-indazol- | 1H), 7.77 (d, J = 2.6 Hz, 1H), | | |
| 1-yl)-2,3,4,5-tetra- | 7.65-7.71 (m, 1H), 7.61 (d, J = | | |
| hydrobenzo[f][1,4]oxazepine | 2.6 Hz, 1H), 7.31-7.41 (m, | | |
| | 1H), 4.15-4.24 (m, 2H), | | |
| | 4.02 (s, 2H), 3.76 (s, 2H), | | |
| | 3.03-3.11 (m, 2H). ¹⁹F NMR | | |
| | (282 MHz, DMSO-d₆) δ −121.2. | | |
| 9-chloro-7-(5- | ¹H NMR (300 MHz, DMSO- | 440.1 | P |
| fluoro-1H- | d₆) δ 8.50-8.62 (m, 3H), | | |
| benzo[d]imidazol- | 7.79-7.83(m, 1H), 7.39- | | |
| 1-yl)-4-((2- | 7.66 (m, 3H), 7.12-7.27 (m, | | |
| methoxypyrimidin- | 1H), 4.18-4.22 (m, 2H), 3.98 | | |
| 5-yl)methyl)- | (s, 2H), 3.90 (d, J = 3.0 Hz, | | |
| 2,3,4,5-tetrahydro- | 3H), 3.66 (s, 2H), 3.00-3.10 | | |
| benzo[f][1,4]oxazepine | (m, 2H). ¹⁹F NMR (282 MHz, | | |
| | DMSO-d₆) δ −117.9, 120.9. | | |
| 9-chloro-4-((2- | ¹H NMR (300 MHz, | 444.0 | A |
| chloropyrimidin-5- | DMSO-d₆) δ 8.72 (d, J = 1.0 | | |
| yl)methyl)-7-(5- | Hz, 2H), 8.50-8.59 (m, 1H), | | |
| fluoro-1H- | 7.72-7.81 (m, 1H), 7.60- | | |
| benzo[d]imidazol- | 7.69 (m, 1H), 7.54 (d, J = 2.7 | | |
| 1-yl)-2,3,4,5-tetra- | Hz, 1H), 7.40-7.48 (m, 1H), | | |
| hydrobenzo[f][1,4]oxazepine | 7.12-7.27 (m, 1H), 4.18- | | |
| | 4.25 (m, 2H), 3.98 (ds, 2H), | | |
| | 3.75 (s, 2H), 3.06-3.14 (m, | | |
| | 2H). ¹⁹F NMR (376 MHz, | | |
| | DMSO-d₆) δ −117.9, 120.5. | | |
| 5-((9-chloro-7-(5- | ¹H NMR (400 MHz, DMSO- | 426.1 | L |
| fluoro-1H- | d₆) δ 8.50-8.60 (m, 1H), | | |
| benzo[d]imidazol- | 8.13-8.18 (m, 2H), 7.76- | | |
| 1-yl)-2,3-dihydro- | 7.82 (m, 1H), 7.60-7.66 (m, | | |
| benzo[f][1,4]oxazepin-4(5H)- | 1H), 7.54-7.59 (m, 1H), 7.41- | | |
| yl)methyl)pyrimidin- | 7.48 (m, 1H), 7.10-7.22 | | |
| 2(1H)-one | (m, 1H), 4.14-4.21 (m, 2H), | | |
| | 3.96 (d, J = 5.6 Hz, 2H), 3.45 | | |
| | (s, 2H), 3.00-3.10 (m, | | |
| | 2H). ¹⁹F NMR (376 MHz, | | |
| | DMSO-d₆) δ −117.9, 120.5. | | |
| 4-((3H- | ¹H NMR (300 MHz, DMSO- | 448.0 | Q |
| imidazo[4,5- | d₆) δ 8.32-8.41 (m, 2H), | | |
| b]pyridin-6- | 7.95 (s, 1H), 7.63-7.71 (m, | | |
| yl)methyl)-9- | 2H), 7.43-7.51 (m, 2H), 7.33 | | |
| chloro-7-(5-fluoro- | (s, 1H), 7.07 (s, 1H), 6.68 (s, | | |
| 1H-indol-1-yl)- | 1H), 4.15-4.25 (m, 2H), 3.97 | | |
| 2,3,4,5-tetrahydro- | (s, 2H), 3.85 (s, 2H), 3.08- | | |
| benzo[f][1,4]oxazepine | 3.20 (m, 2H). ¹⁹F NMR (282 | | |
| | MHz, DMSO-d₆) δ −124.1. | | |
| 4-((1H- | ¹H NMR (300 MHz, DMSO- | 448.0 | Q |
| pyrazolo[3,4- | d₆) δ 13.61 (br, 1H), 8.49 (d, | | |
| b]pyridin-5- | J = 3.0 Hz, 1H), 8.08-8.19 | | |
| yl)methyl)-9- | (m, 2H), 7.71 (d, J = 3.0 Hz, | | |
| chloro-7-(5-fluoro- | 1H), 7.63 (d, J = 3.0 Hz, 1H), | | |
| 1H-indol-1-yl)- | 7.40-7.50 (m, 2H), 7.34 (d, J = | | |
| 2,3,4,5-tetrahydro- | 2.6 Hz, 1H), 6.98-7.08 (m, | | |
| benzo[f][1,4]oxazepine | 1H), 6.68 (d, J = 3.0 Hz, 1H), | | |
| | 4.15-4.23 (m, 2H), 3.98 (s, | | |
| | 2H), 3.84 (s, 2H), 3.00-3.10 | | |
| | (m, 2H). ¹⁹F NMR (282 MHz, | | |
| | DMSO-d₆) δ −123.6. | | |
| 9-chloro-7-(6,7- | ¹H NMR (300 MHz, DMSO- | 458.0 | P |
| difluorobenzofuran- | d₆) δ 8.49-8.58 (m, 3H), | | |
| 3-yl)-4-((2- | 7.70-7.80 (m, 2H), 7.52 (d, J = | | |
| methoxypyrimidin- | 3.0 Hz, 1H), 7.38-7.47 (m, | | |
| 5-yl)methyl)- | 1H), 4.12-4.18 (m, 2H), 3.98 | | |
| 2,3,4,5-tetrahydro- | (s, 2H), 3.91 (s, 3H), 3.65 (s, | | |
| benzo[f][1,4]oxazepine | 2H), 2.99-3.08 (m, 2H). ¹⁹F | | |
| | NMR (282 MHz, DMSO-d₆) | | |
| | δ −144.1, −161.8. | | |
| 9-chloro-7-(6- | ¹H NMR (300 MHz, DMSO- | 456.0208 | E |
| fluorobenzo[b]thiophen- | d6) δ 8.53 (s, 2H), 8.02 (d, J = | | |
| 3-yl)-4-((2- | 2.1 Hz, 1H), 7.83-7.99 | | |
| methoxypyrimidin- | (m, 2H), 7.49 (d, J = 2.1 Hz, | | |
| 5-yl)methyl)- | 1H), 7.32-7.37 (m, 2H), | | |
| 2,3,4,5-tetrahydro- | 4.18 (s, 2H), 3.92-3.95 (m, | | |
| benzo[f][1,4]oxazepine | 2H), 3.90 (s, 3H), 3.65 (s, | | |
| | 2H), 3.04 (s, 2H). ¹⁹F NMR | | |
| | (300 MHz, DMSO-d6) δ −117.53. | | |

### Preparation of N-(5-((9-chloro-7-(5-fluoro-1H-indol-1-yl)-2,3-dihydrobenzo[f][1,4]oxazepin-4 (5H)-yl)methyl)pyrimidin-2-yl)methanesulfonamide

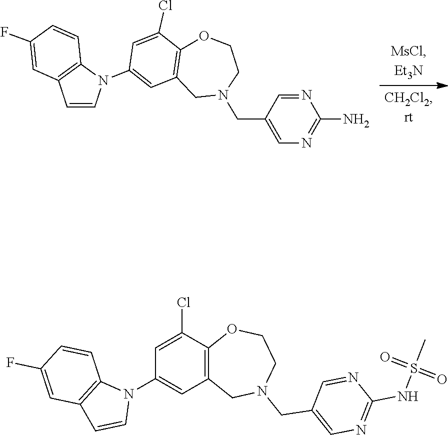 [SMILES: Nc1ncc(CN2CCOc3c(Cl)cc(-n4ccc5cc(F)ccc54)cc3C2)cn1 ; CS(=O)(=O)Nc1ncc(CN2CCOc3c(Cl)cc(-n4ccc5cc(F)ccc54)cc3C2)cn1]

To a stirred solution of 5-{[9-chloro-7-(5-fluoroindol-1-yl)-3,5-dihydro-2H-1,4-benzoxazepin-4-yl]methyl}pyrimidin-2-amine (50 mg, 0.118 mmol, 1 equiv) in CH₂Cl₂ (1 mL) was added methanesulfonyl chloride (20 uL, 0.236 mmol, 2.00 equiv) and Et3N (33 uL, 0.236 mmol, 2 equiv) at 0° C. The resulting mixture was stirred for 2 h at room temperature. Desired product could be detected by LCMS. After concentration, the residue was purified by Prep-HPLC with the following conditions (Column: XBridge Prep Phenyl OBD Column, 19*250 mm, 5 μm; Mobile Phase A: Water (10 mmol/L NH₄HCO₃+0.1% NH₃·H₂O), Mobile Phase B: ACN; Flow rate: 25 mL/min; Gradient: 30% B to 60% B in 9 min, 60% B; Wave Length: 254 nm; RT1 (min): 8.23; Number Of Runs: 0) to afford N-(5-{[9-chloro-7-(5-fluoroindol-1-yl)-3,5-dihydro-2H-1,4-benzoxazepin-4-yl]methyl}pyrimidin-2-yl)methanesulfonamide (4.4 mg, 7.43%) as a white solid.

LC/MS: mass calcd. For C₂₃H₂₁ClFN₅O₃S: 501.1, found: 502.1 [M+H]⁺.

¹H NMR (300 MHz, DMSO-d₆) δ 11.31 (s, 1H), 8.50 (s, 2H), 7.72 (d, J=3.3 Hz, 1H), 7.64 (d, J=2.6 Hz, 1H), 7.49-7.53 (m 1H), 7.42-7.45 (m, 2H), 7.02-7.14 (m, 1H), 6.69 (d, J=3.2 Hz, 1H), 4.17-4.18 (m, 2H), 3.98 (s, 2H), 3.64 (s, 2H), 3.27 (s, 3H), 3.05-3.06 (m, 2H). ¹⁹F NMR (282 MHz, DMSO-d6) δ −123.6.

### Preparation of 9-chloro-7-(6-fluoro-4-methyl-3,4-dihydroquinoxalin-1 (2H)-yl)-4-((2-methoxypyrimidin-5-yl)methyl)-2,3,4,5-tetrahydrobenzo[f][1,4]oxazepine

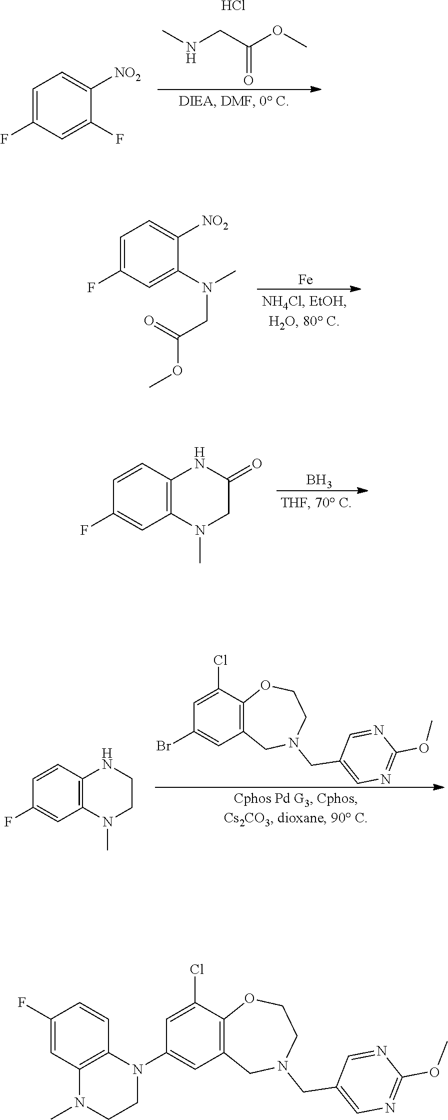

### Methyl 2-[(5-fluoro-2-nitrophenyl)(methyl)amino]acetate

To a stirred mixture of 2,4-difluoro-1-nitro- (2 g, 12.571 mmol, 1 equiv) and methyl 2-(methylamino)acetate hydrochloride (1.75 g, 12.571 mmol, 1 equiv) in DMF (25 mL) was added DIEA (2.44 g, 18.857 mmol, 1.5 equiv) at 0° C. The resulting mixture was stirred for additional 6 h at 0° C. and warmed naturally to room temperature. The mixture was stirred at room temperature overnight. To the mixture was added H₂O (30 mL) and the resulting mixture was extracted with EtOAc (3×20 mL). The combined organic layers were washed with water (3×20 mL), dried over anhydrous Na₂SO₄. After filtration, the filtrate was concentrated under reduced pressure. The residue was purified by silica gel column chromatography, eluted with petroleum ether/ethyl acetate (10:1) to afford methyl 2-[(5-fluoro-2-nitrophenyl)(methyl)amino]acetate (1.6 g, 52.55%) as a white solid.

### 6-fluoro-4-methyl-1,3-dihydroquinoxalin-2-one

To a stirred mixture of methyl 2-[(5-fluoro-2-nitrophenyl)(methyl)amino]acetate (1 g, 4.129 mmol, 1 equiv) and NH₄Cl (2.21 g, 41.290 mmol, 10 equiv) in EtOH (20 mL) was added H₂O (15 mL). To the above mixture was added iron powder (2.31 g, 41.290 mmol, 10 equiv) portion wise at 80° C. with stirring. The resulting mixture was stirred for 1 h at 80° C. Desired product could be detected by LCMS. The resulting mixture was filtered, and the filter cake was washed with EtOAc (4×50 mL). The filtrate was concentrated under reduced pressure to dryness. The residue was triturated with EtOAc (50 mL) and filtered. The filter cake was washed with EtOAc (50 mL). The combined filtrate was concentrated under reduced pressure. To afford crude 6-fluoro-4-methyl-1,3-dihydroquinoxalin-2-one (0.75 g, 101%) as a white solid. The crude product was used for next step without further purification.

### 7-fluoro-1-methyl-3,4-dihydro-2H-quinoxaline

To a stirred mixture of 6-fluoro-4-methyl-1,3-dihydroquinoxalin-2-one (500 mg, 2.775 mmol, 1 equiv) and THF (15 mL) was added 1M BH₃ in THF (14 mL, 13.875 mmol, 5.0 equiv) dropwise at room temperature under nitrogen atmosphere. The resulting mixture was stirred for 3 h at 70° C. under nitrogen atmosphere. To the above mixture was added 2 M aqueous HCl (12 mL) dropwise over 2 min at 70° C. The resulting mixture was stirred for 20 min at 70° C. The resulting mixture was cooled to room temperature and was neutralized with aq. Na₂CO₃. The resulting mixture was extracted with EtOAc (3×20 mL). The combined organic layers were washed with brine (50 mL), dried over anhydrous Na₂SO₄. After filtration, the filtrate was concentrated under reduced pressure to afford 7-fluoro-1-methyl-3,4-dihydro-2H-quinoxaline (180 mg, 39.03%) as a brown solid. The crude product was not stable and it was used for next step without further purification.

### 9-chloro-7-(6-fluoro-4-methyl-3,4-dihydroquinoxalin-1 (2H)-yl)-4-((2-methoxypyrimidin-5-yl)methyl)-2,3,4,5-tetrahydrobenzo[f][1,4]oxazepane

To a stirred mixture of 7-fluoro-1-methyl-3,4-dihydro-2H-quinoxaline (36 mg, 0.217 mmol, 1 equiv) and 7-bromo-9-chloro-4-[(2-methoxypyrimidin-5-yl)methyl]-3,5-dihydro-2H-1,4-benzoxazepine (100 mg, 0.260 mmol, 1.2 equiv) in dioxane (2 mL) was added Cphos pd G₃ (18 mg, 0.022 mmol, 0.1 equiv), Cphos (10 mg, 0.022 mmol, 0.1 equiv) and Cs₂CO₃ (141 mg, 0.434 mmol, 2 equiv). The resulting mixture was stirred for 16 h at 90° C. under nitrogen atmosphere. The mixture was filtered, the filtrated was concentrated under reduced pressure. The residue was purified by Prep-HPLC with the following conditions (Column: XBridge Prep OBD C18 Column, 30*150 mm, 5 μm; Mobile Phase A: Water (10 mmol/L NH4HCO3+0.1% NH3·H2O), Mobile Phase B: ACN; Flow rate: 60 mL/min; Gradient: 50% B to 80% B in 7 min, 80% B; Wave Length: 254 nm; RT1 (min): 5.87; Number Of Runs: 0) to afford 9-chloro-7-(6-fluoro-4-methyl-2,3-dihydroquinoxalin-1-yl)-4-[(2-methoxypyrimidin-5-yl)methyl]-3,5-dihydro-2H-1,4-benzoxazepine (3.5 mg, 3.32%) as a white solid.

LC/MS: mass calcd. For C₂₄H₂₅ClFN₅O₂: 469.2, found: 470.1 [M+H]⁺.

¹H NMR (300 MHz, DMSO-d₆) δ 8.49 (s, 2H), 7.03 (d, J=3.0 Hz, 1H), 6.80 (d, J=3.0 Hz, 1H), 6.60-6.65 (m, 1H), 6.46-6.51 (m, 1H), 6.25-6.32 (m, 1H), 4.03-4.06 (m, 2H), 3.91 (d, J=6.0 Hz, 3H), 3.76 (s, 2H), 3.56-3.58 (m, 4H), 3.23-3.31 (m, 2H), 3.02 (s, 2H), 2.89 (s, 3H). ¹⁹F (282 MHz, DMSO-d₆) δ −120.7

### Preparation of bis(5-cyclopropyl-1H-indole)

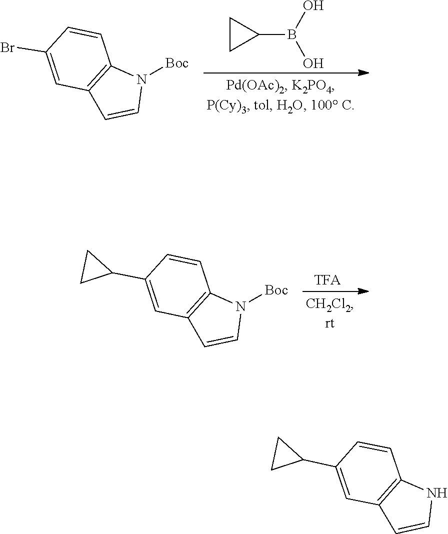 [SMILES: CC(C)(C)OC(=O)n1ccc2cc(Br)ccc21 ; OB(O)C1CC1 ; CC(C)(C)OC(=O)n1ccc2cc(C3CC3)ccc21 ; c1cc2cc(C3CC3)ccc2[nH]1]

### tert-butyl 5-cyclopropylindole-1-carboxylate

To the mixture of tert-butyl 5-bromoindole-1-carboxylate (500 mg, 1.688 mmol, 1 equiv) and cyclopropylboronic acid (188 mg, 2.194 mmol, 1.3 equiv) in toluene (10 mL) was added Pd(OAc)₂ (19 mg, 0.084 mmol, 0.05 equiv), tricyclohexylphosphane (47 mg, 0.169 mmol, 0.1 equiv), K₃PO₄ (895 mg, 4.220 mmol, 2.5 equiv) and H₂O (0.5 mL). The mixture was stirred at 100° C. for 3 h under nitrogen atmosphere. Desired product could be detected by LCMS. The mixture was added with H₂O (20 mL) and extracted by EtOAc (3×15 mL). The organic layer was combined, washed by brine (30 mL), dried over Na₂SO₄, filtered and concentrated under reduced pressure. The residue was purified by silica gel column chromatography, eluted with petroleum ether/ethyl acetate (10:1) to afford tert-butyl 5-cyclopropylindole-1-carboxylate (300 mg, 69.05%) as red oil.

### bis(5-cyclopropyl-1H-indole)

To the mixture of bis(tert-butyl 5-cyclopropylindole-1-carboxylate) (500 mg, 0.972 mmol, 1 equiv) in CH₂Cl₂ (10 mL) was added TFA (2 mL). The mixture was stirred at room temperature for 1 h. Desired product could be detected by LCMS. After concentration, the mixture was diluted by CH₂Cl₂ (20 mL) and washed by sat. aq. NaHCO₃ (20 mL), brine (20 mL). The organic layer was dried over MgSO₄, filtered and concentrated to afford bis(5-cyclopropyl-1H-indole) (300 mg, 98.21%) as yellow oil. The crude product was used for next step without further purification.

### Preparation of 4-((2-(1H-pyrazol-1-yl)pyrimidin-5-yl)methyl)-9-chloro-7-(5-fluoro-1H-indol-1-yl)-2,3,4,5-tetrahydrobenzo[f][1,4]oxazepine

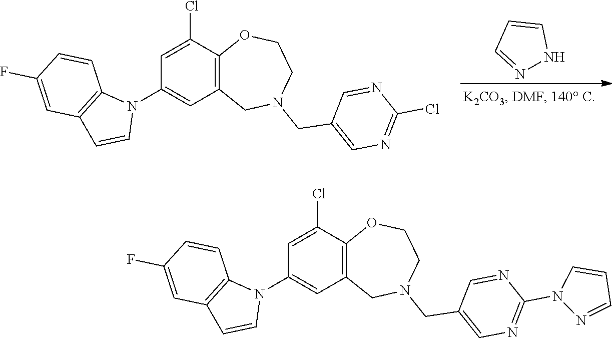 [SMILES: Fc1ccc2c(ccn2-c2cc(Cl)c3c(c2)CN(Cc2cnc(Cl)nc2)CCO3)c1 ; c1cn[nH]c1 ; Fc1ccc2c(ccn2-c2cc(Cl)c3c(c2)CN(Cc2cnc(-n4cccn4)nc2)CCO3)c1]

To a stirred mixture of 9-chloro-4-[(2-chloropyrimidin-5-yl)methyl]-7-(5-fluoroindol-1-yl)-3,5-dihydro-2H-1,4-benzoxazepine (50 mg, 0.113 mmol, 1 equiv) and pyrazole (15 mg, 0.226 mmol, 2 equiv) in DMF was added K₂CO₃ (31 mg, 0.226 mmol, 2 equiv). The resulting mixture was stirred overnight at 140° C. Desired product could be detected by LCMS. The resulting mixture was filtered, the filtrate was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 48% B to 78% B in 7 min, 78% B; Wave Length: 254 nm; RT1 (min): 6.23; Number Of Runs: 0) to afford 9-chloro-7-(5-fluoroindol-1-yl)-4-{[2-(pyrazol-1-yl)pyrimidin-5-yl]methyl}-3,5-dihydro-2H-1,4-benzoxazepine (11.5 mg, 21.45%) as a white solid.

LC/MS: mass calcd. For C₂₅H₂₀ClFN₆O: 474.1, found: 475.1 [M+H]⁺

¹H NMR (300 MHz, DMSO-d₆) δ 8.79 (s, 2H), 8.66 (d, J=2.1 Hz, 1H), 7.85 (s, 1H), 7.70 (d, J=3.3 Hz, 1H), 7.63 (d, J=2.4 Hz, 1H), 7.51 (d, J=9.0 Hz, 1H), 7.42-7.44 (m, 2H), 7.05 (t, J=9.3 Hz, 1H), 6.66 (d, J=3.3 Hz, 1H), 6.59 (d, J=2.7 Hz, 1H), 4.20-4.21 (m, 2H), 4.00 (s, 2H), 3.78 (s, 2H), 3.14-3.15 (m, 2H). ¹⁹F NMR (282 MHz, DMSO-d₆) δ −123.7.

### Preparation of 9-chloro-7-(5-fluoro-1H-indol-1-yl)-4-((2-(1-methyl-1H-pyrazol-4-yl)pyrimidin-5-yl)methyl)-2,3,4,5-tetrahydrobenzo[f][1,4]oxazepane

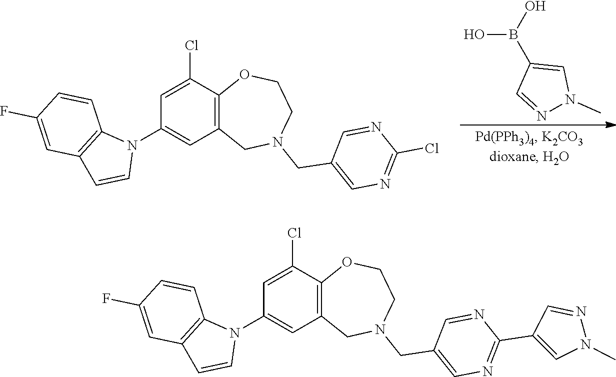

To a mixture of 9-chloro-4-[(2-chloropyrimidin-5-yl)methyl]-7-(5-fluoroindol-1-yl)-3,5-dihydro-2H-1,4-benzoxazepine (50 mg, 0.113 mmol, 1 equiv) and 1-methylpyrazol-4-ylboronic acid (28 mg, 0.226 mmol, 2 equiv) in 1,4-dioxane (1 mL) and water (0.2 mL) was added K₂CO₃ (31 mg, 0.226 mmol, 2 equiv) and Pd(PPh₃)₄ (13 mg, 0.011 mmol, 0.1 equiv). The resulting mixture was stirred overnight at 80° C. under nitrogen atmosphere. Desired product could be detected by LCMS. The resulting mixture was concentrated under reduced pressure. The residue was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 58% B to 88% B in 7 min, 88% B; Wave Length: 254 nm; RT1 (min): 5.98; Number Of Runs: 0) to afford 9-chloro-7-(5-fluoroindol-1-yl)-4-{[2-(1-methylpyrazol-4-yl)pyrimidin-5-yl]methyl}-3,5-dihydro-2H-1,4-benzoxazepine (14.1 mg, 25.50%) as a white solid.

LC/MS: mass calcd. For C₂₆H₂₂ClFN₆O: 488.1, found: 489.1 [M+H]⁺

¹H NMR (300 MHz, DMSO-d₆) δ 8.66 (s, 2H), 8.35 (s, 1H), 8.00 (d, J=0.6 Hz, 1H), 7.71 (d, J=3.3 Hz, 1H), 7.62 (d, J=2.4 Hz, 1H), 7.50 (d, J=9.0 Hz, 1H), 7.42 (d, J=7.5 Hz, 2H), 7.02 (t, J=6.9 Hz, 1H), 6.68 (d, J=2.7 Hz, 1H), 4.19-4.20 (m, 2H), 43.99 (s, 2H), 3.89 (s, 3H), 3.70 (s, 2H), 3.08-3.09 (m, 2H). ¹⁹F NMR (282 MHz, DMSO-d₆) δ −123.6.

### Preparation of N-(5-((9-chloro-7-(5-fluoro-1H-indol-1-yl)-2,3-dihydrobenzo[f][1,4]oxazepin-4 (5H)-yl)methyl)pyrimidin-2-yl)acetamide

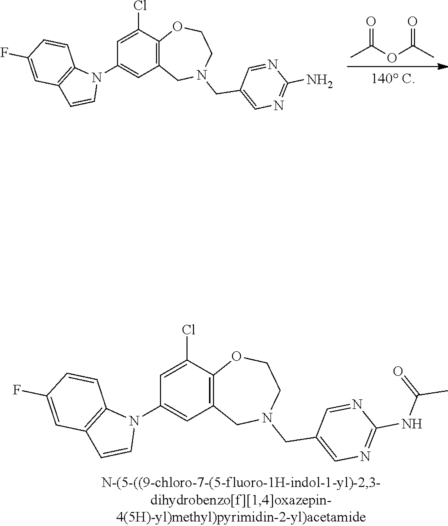

A solution of 5-{[9-chloro-7-(5-fluoroindol-1-yl)-3,5-dihydro-2H-1,4-benzoxazepin-4-yl]methyl}pyrimidin-2-amine (50 mg, 0.120 mmol, 1 equiv) in acetic anhydride (1 mL) was stirred for overnight at 140° C. After concentration, to the residue was added aq. NH₃·H₂O (2 mL). The resulting mixture was stirred for additional 1 h at room temperature. Desired product could be detected by LCMS. The aqueous layer was extracted with CH₂Cl₂ (3×2 mL). The combined organic layers were washed with brine (2 mL), dried over anhydrous Na₂SO₄. After filtration, the filtrate was concentrated under reduced pressure. The residue was purified by Prep-HPLC with the following conditions (Column: XBridge Prep Phenyl OBD Column, 19*250 mm, 5 μm; Mobile Phase A: Water (10 mmol/L NH₄HCO₃+0.1% NH₃·H₂O), Mobile Phase B: ACN; Flow rate: 25 mL/min; Gradient: 30% B to 60% B in 9 min, 60% B; Wave Length: 254 nm; RT1 (min): 8.23; Number Of Runs: 0) to afford N-(5-((9-chloro-7-(5-fluoro-1H-indol-1-yl)-2,3-dihydrobenzo[f][1,4]oxazepin-4 (5H)-yl)methyl)pyrimidin-2-yl)acetamide (9.0 mg, 13.56%) as a white solid.

LC/MS: mass calcd. For C₂₄H₂₁ClFN₅O₂: 465.1, found: 466.0 [M+H]⁺

¹H NMR (300 MHz, DMSO-d₆) δ 10.54 (s, 1H), 8.57 (s, 2H), 7.71 (d, J=3.3 Hz, 1H), 7.63 (d, J=2.6 Hz, 1H), 7.51 (d, J=9.0 Hz, 1H), 7.47-7.37 (m, 2H), 7.07 (t, J=9.2 Hz, 1H), 6.69 (d, J=3.2 Hz, 1H), 4.18-4.19 (m, 2H), 3.97 (s, 2H), 3.67 (s, 2H), 3.07-3.08 (m, 2H), 2.17 (s, 3H).

¹⁹F NMR (282 MHz, DMSO-d₆) δ −123.6.

### Preparation of methyl 5-((9-chloro-7-(5-fluoro-1H-indol-1-yl)-2,3-dihydrobenzo[f][1,4]oxazepin-4 (5H)-yl)methyl)picolinate

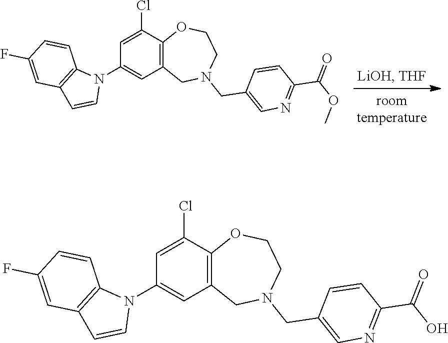 [SMILES: COC(=O)c1ccc(CN2CCOc3c(Cl)cc(-n4ccc5cc(F)ccc54)cc3C2)cn1 ; O=C(O)c1ccc(CN2CCOc3c(Cl)cc(-n4ccc5cc(F)ccc54)cc3C2)cn1]

To a stirred solution of methyl 5-{[9-chloro-7-(5-fluoroindol-1-yl)-3,5-dihydro-2H-1,4-benzoxazepin-4-yl]methyl}pyridine-2-carboxylate (50 mg, 0.105 mmol, 1 equiv) in THE (1 mL) and was added 1 M aq LiOH (0.5 mL, 0.5 mmol, 5 equiv) at room temperature. The resulting mixture was stirred for 2 h at room temperature. Desired product could be detected by LCMS. The mixture was acidified to pH 6 with 1 M aq. HCl. The aqueous layer was extracted with CH₂Cl₂ (3×2 mL). The combined organic layers were washed with brine (2 mL), dried over anhydrous Na₂SO₄. After filtration, the filtrate was concentrated under reduced pressure. The residue was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 27% B to 57% B in 7 min, 57% B; Wave Length: 254 nm; RT1 (min): 5.87; Number Of Runs: 0) to afford 5-{[9-chloro-7-(5-fluoroindol-1-yl)-3,5-dihydro-2H-1,4-benzoxazepin-4-yl]methyl}pyridine-2-carboxylic acid (7.3 mg, 14.91%) as a white solid.

LC/MS: mass calcd. For C₂₄H₁₉ClFN₃O₃: 451.1, found: 452.1 [M+H]⁺

¹H NMR (300 MHz, DMSO-d₆) δ 8.57 (s, 1H), 7.97 (d, J=8.0 Hz, 1H), 7.85 (d, J=8.1 Hz, 1H), 7.72 (d, J=3.3 Hz, 1H), 7.64 (d, J=2.6 Hz, 1H), 7.39-7.51 (m, 2H), 7.35 (d, J=2.7 Hz, 1H), 7.14-7.02 (m, 1H), 6.68 (d, J=3.3 Hz, 1H), 4.18-4.19 (m, 2H), 3.96 (s, 2H), 3.80 (s, 2H), 3.08-3.09 (m, 2H). ¹⁹F NMR (282 MHz, DMSO-d₆) δ −123.5.

### Preparation of 4-((2-(1H-tetrazol-5-yl)pyrimidin-5-yl)methyl)-9-chloro-7-(5-fluoro-1H-indol-1-yl)-2,3,4,5-tetrahydrobenzo[f][1,4]oxazepine

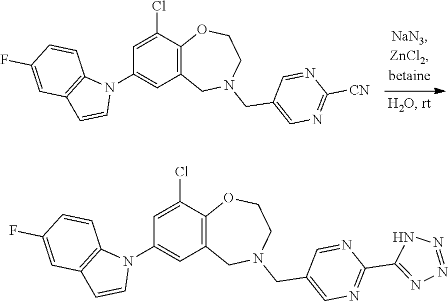 [SMILES: N#Cc1ncc(CN2CCOc3c(Cl)cc(-n4ccc5cc(F)ccc54)cc3C2)cn1 ; Fc1ccc2c(ccn2-c2cc(Cl)c3c(c2)CN(Cc2cnc(-c4nnn[nH]4)nc2)CCO3)c1]

To a stirred mixture of 5-{[9-chloro-7-(5-fluoroindol-1-yl)-3,5-dihydro-2H-1,4-benzoxazepin-4-yl]methyl}pyrimidine-2-carbonitrile (50 mg, 0.115 mmol, 1 equiv) and NaN₃ (22 mg, 0.345 mmol, 3 equiv) in water (2 mL) was added ZnCl₂ (47 mg, 0.345 mmol, 3 equiv) and 2-(trimethylazaniumyl)acetate (2 mg, 0.012 mmol, 0.1 equiv). The resulting mixture was stirred for 3 days at room temperature. Desired product could be detected by LCMS. The precipitated solids were collected by filtration and washed with water (3×2 mL). The crude product (30 mg) was purified by Prep-HPLC with the following conditions (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 25% B to 55% B in 7 min, 55% B; Wave Length: 254 nm; RT1 (min): 5.87; Number Of Runs: 0) to afford 9-chloro-7-(5-fluoroindol-1-yl)-4-{[2-(1H-1,2,3,4-tetrazol-5-yl)pyrimidin-5-yl]methyl}-3,5-dihydro-2H-1,4-benzoxazepine (3.6 mg, 6.44%) as a light yellow solid.

LC/MS: mass calcd. For C₂₃H₁₈ClFN₈O: 476.1, found: 477.1 [M+H]⁺

¹H NMR (300 MHz, DMSO-d₆) δ 8.76 (s, 2H), 7.69 (d, J=3.2 Hz, 1H), 7.61 (d, J=2.6 Hz, 1H), 7.51 (d, J=9.0 Hz, 1H), 7.41 (d, J=9.4 Hz, 2H), 7.08 (t, J=9.2 Hz, 1H), 6.68 (d, J=3.4 Hz, 1H), 4.19-4.20 (m, 2H), 4.00 (s, 2H), 3.76 (s, 2H), 3.10-3.11 (m, 2H). ¹⁹F NMR (282 MHz, DMSO-d₆) δ −123.5

### Preparation of 2-(5-((9-chloro-7-(5-fluoro-1H-indol-1-yl)-2,3-dihydrobenzo[1][1,4]oxazepin-4 (5H)-yl)methyl)pyrimidin-2-yl)acetonitrile

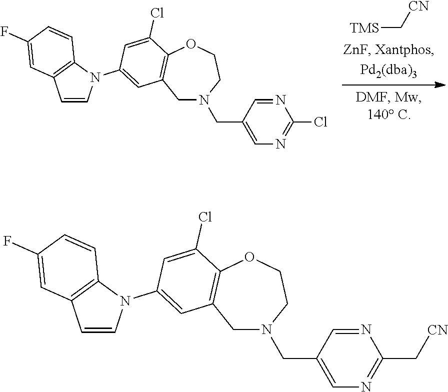 [SMILES: Fc1ccc2c(ccn2-c2cc(Cl)c3c(c2)CN(Cc2cnc(Cl)nc2)CCO3)c1 ; C[Si](C)(C)CC#N ; N#CCc1ncc(CN2CCOc3c(Cl)cc(-n4ccc5cc(F)ccc54)cc3C2)cn1]

To the mixture of 9-chloro-4-[(2-chloropyrimidin-5-yl)methyl]-7-(5-fluoroindol-1-yl)-3,5-dihydro-2H-1,4-benzoxazepine (100 mg, 0.226 mmol, 1 equiv) in DMF (2 mL) was added 2-(trimethylsilyl)acetonitrile (38 mg, 0.339 mmol, 1.5 equiv), Xantphos (5 mg, 0.009 mmol, 0.04 equiv), Pd₂(dba)₃ (4 mg, 0.005 mmol, 0.02 equiv) and zinc fluoride (14 mg, 0.136 mmol, 0.6 equiv). The final reaction mixture was irradiated with microwave radiation for 0.5 h at 140° C. under nitrogen atmosphere. Desired product could be detected by LCMS. The resulting mixture was filtered. The filtrate was concentrated under reduced pressure. The mixture was purified by Prep-HPLC with followed conditions (Column: XBridge Prep OBD C18 Column, 30*150 mm, 5 μm; Mobile Phase A: Water (Column: XBridge Prep Phenyl OBD Column, 19*250 mm, 5 μm; Mobile Phase A: Water (10 mmol/L NH₄HCO₃+0.1% NH₃·H₂O), Mobile Phase B: ACN; Flow rate: 25 mL/min; Gradient: 60% B to 80% B in 8 min, 80% B; Wave Length: 254 nm; RT1 (min): 7.23; Number Of Runs: 0) to afford 2-(5-{[9-chloro-7-(5-fluoroindol-1-yl)-3,5-dihydro-2H-1,4-benzoxazepin-4-yl]methyl}pyrimidin-2-yl)acetonitrile (3.3 mg, 3.21%) as a white solid.

LC/MS: mass calcd. For C₂₄H₁₉ClFN₅O: 447.1, found: 448.0 [M+H]⁺

¹H NMR (300 MHz, DMSO-d₆) δ 8.77 (s, 2H), 7.71 (d, J=3.3 Hz, 1H), 7.64 (d, J=2.7 Hz, 1H), 7.54-7.59 (m, 1H), 7.41-7.45 (m, 2H), 7.03-7.10 (m, 1H), 6.69 (d, J=3.3 Hz, 1H), 4.40 (s, 2H), 4.18-4.20 (m, 2H), 3.99 (s, 2H), 3.75 (s, 2H), 3.06-3.09 (m, 2H) ¹⁹F NMR (282 MHz, DMSO-d₆) δ −123.6

### Preparation of 9-chloro-7-(5-fluoro-1H-indol-1-yl)-4-((2-(S-methylsulfonimidoyl)pyrimidin-5-yl)methyl)-2,3,4,5-tetrahydrobenzo[f][1,4]oxazepine

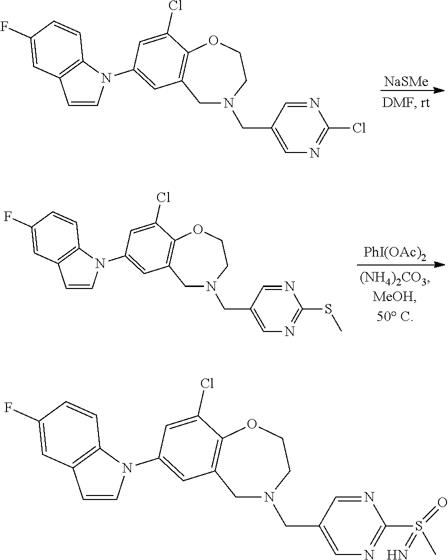 [SMILES: "PhI(OAc)2" ; Fc1ccc2c(ccn2-c2cc(Cl)c3c(c2)CN(Cc2cnc(Cl)nc2)CCO3)c1 ; CSc1ncc(CN2CCOc3c(Cl)cc(-n4ccc5cc(F)ccc54)cc3C2)cn1 ; CS(=N)(=O)c1ncc(CN2CCOc3c(Cl)cc(-n4ccc5cc(F)ccc54)cc3C2)cn1 ; "NaSMe"]

### 9-chloro-7-(5-fluoroindol-1-yl)-4-{[2-(methylsulfanyl)pyrimidin-5-yl]methyl}-3,5-dihydro-2H-1,4-benzoxazepine

To the mixture of 9-chloro-4-[(2-chloropyrimidin-5-yl)methyl]-7-(5-fluoroindol-1-yl)-3,5-dihydro-2H-1,4-benzoxazepine (100 mg, 0.226 mmol, 1 equiv) in DMF (3 mL) was added (methylsulfanyl)sodium (32 mg, 0.452 mmol, 2 equiv). The mixture was stirred at 80° C. for 12 h. Desired product could be detected by LCMS. The mixture was purified directly by reversed-phase flash chromatography with the following conditions: column, C18 silica gel; mobile phase, MeCN in Water (0.1% TFA), 10% to 50% gradient in 10 min; detector, UV 254 nm to afford 9-chloro-7-(5-fluoroindol-1-yl)-4-{[2-(methylsulfanyl)pyrimidin-5-yl]methyl}-3,5-dihydro-2H-1,4-benzoxazepine (60 mg, 57.53%) as a light yellow oil.

### 9-chloro-7-(5-fluoro-1H-indol-1-yl)-4-((2-(S-methylsulfonimidoyl)pyrimidin-5-yl)methyl)-2,3,4,5-tetrahydrobenzo[f][1,4]oxazepane

The mixture of 9-chloro-7-(5-fluoroindol-1-yl)-4-{[2-(methylsulfanyl)pyrimidin-5-yl]methyl}-3,5-dihydro-2H-1,4-benzoxazepine (60 mg, 0.132 mmol, 1 equiv) in MeOH (1.5 mL) was added (NH₄)₂CO₃ (25 mg, 0.264 mmol, 2 equiv) and (acetyloxy)(phenyl)-lambda3-iodanyl acetate (148.68 mg, 0.462 mmol, 3.5 equiv). The mixture was stirred at 50° C. for 3 h. Desired product could be detected by LCMS. The mixture was purified directly by Prep-HPLC with followed conditions (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 35% B to 65% B in 10 min, 65% B; Wave Length: 254 nm; RT1 (min): 8.25; Number Of Runs: 0) to afford (5-{[9-chloro-7-(5-fluoroindol-1-yl)-3,5-dihydro-2H-1,4-benzoxazepin-4-yl]methyl}pyrimidin-2-yl)(imino)methyl-lambda6-sulfanone (9.1 mg, 13.55%) as an off-white solid.

LC/MS: mass calcd. For C₂₃H₂₁ClFN₅O₂S: 485.1, found: 486.0 [M+H]⁺

¹H NMR (300 MHz, DMSO-d₆) δ 8.95 (s, 2H), 7.72 (d, J=3.3 Hz, 1H), 7.65 (d, J=2.7 Hz, 1H), 7.42-7.55 (m, 3H), 7.01-7.13 (m, 1H), 6.69 (d, J=3.3 Hz, 1H), 4.59 (s, 1H), 4.19-4.22 (m, 2H), 4.04 (s, 2H), 3.84 (s, 2H), 3.29 (d, J=2.7 Hz, 3H), 3.09-3.12 (m, 2H). ¹⁹F NMR (282 MHz, DMSO-d₆) δ −123.5

### Preparation of 2-amino-5-((9-chloro-7-(5-fluoro-1H-indol-1-yl)-2,3-dihydrobenzo[f][1,4]oxazepin-4 (5H)-yl)methyl)pyrimidin-4 (3H)-one

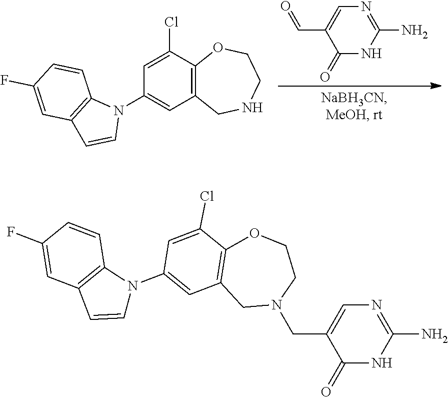 [SMILES: Fc1ccc2c(ccn2-c2cc(Cl)c3c(c2)CNCCO3)c1 ; Nc1ncc(C=O)c(=O)[nH]1 ; Nc1ncc(CN2CCOc3c(Cl)cc(-n4ccc5cc(F)ccc54)cc3C2)c(=O)[nH]1]

To a stirred mixture of 9-chloro-7-(5-fluoroindol-1-yl)-2,3,4,5-tetrahydro-1,4-benzoxazepine (30 mg, 0.095 mmol, 1 equiv) and 2-amino-4-oxo-3H-pyrimidine-5-carbaldehyde (20 mg, 0.143 mmol, 1.5 equiv) in MeOH (1 mL) was added NaBH₃CN (12 mg, 0.190 mmol, 2 equiv). The reaction was stirred at room temperature for 12 h. Desired product could be detected by LCMS. The resulting mixture was concentrated under vacuum. The crude product was purified directly by Prep-HPLC with the following conditions (Column: XBridge Shield RP18 OBD Column, 19*250 mm, 10 μm; Mobile Phase A: Water (10 mmol/L NH₄HCO₃+0.1% NH₃·H₂O), Mobile Phase B: ACN; Flow rate: 25 mL/min; Gradient: 45% B to 75% B in 9 min, 75% B; Wave Length: 254 nm; RT1 (min): 8.23; Number Of Runs: 0) to afford 2-amino-5-{[9-chloro-7-(5-fluoroindol-1-yl)-3,5-dihydro-2H-1,4-benzoxazepin-4-yl]methyl}-3H-pyrimidin-4-one (1.1 mg, 2.56%) as a white solid.

LC/MS: mass calcd. For C₂₂H₁₉ClFN₅O₂: 439.1, found: 424.1 [M+H]⁺

¹H NMR (300 MHz, DMSO-d₆) δ 10.89 (s, 1H), 7.73 (d, J=3.3 Hz, 1H), 7.48-7.67 (m, 3H), 7.38-7.48 (m, 2H), 7.00-7.13 (m, 1H), 6.68 (d, J=3.3 Hz, 1H), 6.47 (s, 2H), 4.12-4.18 (m, 2H), 3.91 (s, 2H), 3.34 (s, 2H), 3.01-3.06 (m, 2H). ¹⁹F NMR (282 MHz, DMSO-d6) δ −123.6.

### Preparation of tert-butyl N-(tert-butoxycarbonyl)-N-(5-{[9-chloro-7-(5-fluoroindol-1-yl)-3,5-dihydro-2H-1,4-benzoxazepin-4-yl]methyl}pyridazin-3-yl)carbamate

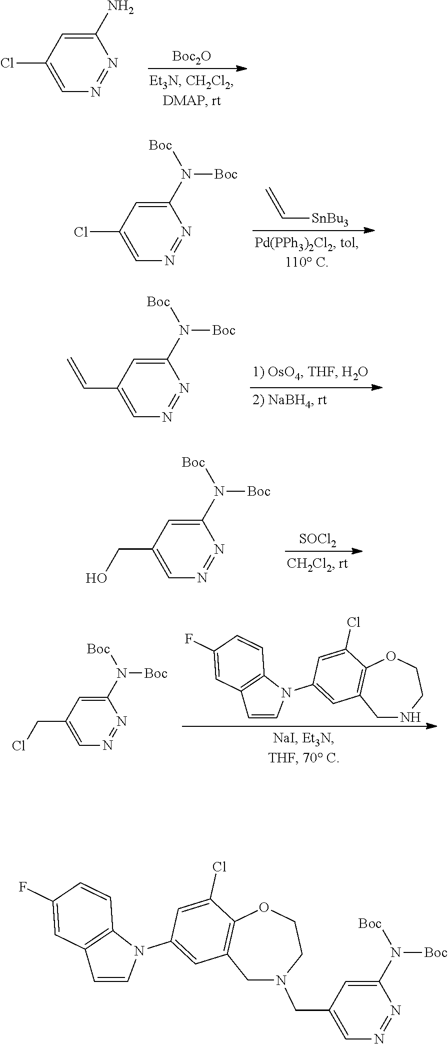

### tert-butyl N-(tert-butoxycarbonyl)-N-(5-chloropyridazin-3-yl)carbamate

To a stirred mixture of 5-chloropyridazin-3-amine (500 mg, 3.860 mmol, 1 equiv) and Boc₂O (1.26 g, 5.790 mmol, 1.5 equiv) in CH₂Cl₂ (10 mL) was added Et₃N (590 mg, 5.790 mmol, 1.5 equiv) and DMAP (47 mg, 0.386 mmol, 0.1 equiv). The reaction was stirred for 2 h at 40° C. Desired product could be detected by LCMS. To the above mixture was added water (50 mL) and extracted with CH₂Cl₂ (3×50 mL). The combined organic layers were washed with water (10 mL), dried over anhydrous MgSO₄. After filtration, the filtrate was concentrated under reduced pressure. The residue was purified by silica gel column chromatography, eluted with petroleum ether/ethyl acetate (20:1). The fractions were combined and concentrated to afford tert-butyl N-(tert-butoxycarbonyl)-N-(5-chloropyridazin-3-yl)carbamate (350 mg, 39.49%) as a white solid.

### tert-butyl N-(tert-butoxycarbonyl)-N-(5-ethenylpyridazin-3-yl)carbamate

To a stirred mixture of tert-butyl N-(tert-butoxycarbonyl)-N-(5-chloropyridazin-3-yl)carbamate (300 mg, 0.910 mmol, 1 equiv) in toluene (3 mL) was added tributyl(vinyl)stannane (763 mg, 1.820 mmol, 2 equiv) and Pd(pph₃)₂Cl₂ (64 mg, 0.091 mmol, 0.1 equiv). The reaction was stirred at 110° C. for 2 h under nitrogen atmosphere. Desired product could be detected by LCMS. The resulting mixture was filtered, and the filter cake was washed with EtOAc (3×10 mL). The filtrate was concentrated under vacuum. The residue was purified by silica gel column chromatography, eluted with petroleum ether/ethyl acetate (5:1). The fractions were combined and concentrated to afford tert-butyl N-(tert-butoxycarbonyl)-N-(5-ethenylpyridazin-3-yl)carbamate (280 mg, 90.99%) as a light yellow oil.

### tert-butyl N-(tert-butoxycarbonyl)-N-[5-(hydroxymethyl)pyridazin-3-yl]carbamate

To a stirred mixture of tert-butyl N-(tert-butoxycarbonyl)-N-(5-ethenylpyridazin-3-yl)carbamate (270 mg, 0.840 mmol, 1 equiv) in THF (2.5 mL) and H2O (2.5 mL) was added OSO₄ (22 mg, 0.084 mmol, 0.10 equiv) at room temperature. The reaction was stirred at room temperature for 2 min. To the above mixture was added NaIO₄ (361 mg, 1.688 mmol, 2.01 equiv) over 2 min at room temperature. The resulting mixture was stirred for additional 5 h at room temperature. To the above mixture was added NaBH₄ (81 mg, 2.142 mmol, 2.55 equiv) at 0° C. The resulting mixture was stirred for additional 15 min at room temperature. Desired product could be detected by LCMS. To the above residue was added water (30 mL) and extracted with EtOAc (3×30 mL). The combined organic layers were washed with water (10 mL), dried over anhydrous Na₂SO₄. After filtration, the filtrate was concentrated under reduced pressure. The residue was purified by silica gel column chromatography, eluted with petroleum ether/ethyl acetate (5:1). The fractions were combined and concentrated to afford tert-butyl N-(tert-butoxycarbonyl)-N-[5-(hydroxymethyl)pyridazin-3-yl]carbamate (160 mg, 52.68%) as yellow oil.

### tert-butyl N-(tert-butoxycarbonyl)-N-[5-(chloromethyl)pyridazin-3-yl]carbamate

To a stirred mixture of tert-butyl N-(tert-butoxycarbonyl)-N-[5-(hydroxymethyl)pyridazin-3-yl]carbamate (100 mg, 0.307 mmol, 1 equiv) in CH₂Cl₂ (2 mL) was added SOCl₂ (44 mg, 0.370 mmol, 1.20 equiv) at 0° C. The reaction was stirred at room temperature for 1 h. Desired product could be detected by LCMS. The resulting mixture was concentrated under vacuum to afford tert-butyl N-(tert-butoxycarbonyl)-N-[5-(chloromethyl)pyridazin-3-yl]carbamate (100 mg, 85.17%) as a light yellow oil. The crude product was used for next step without further purification.

### tert-butyl N-(tert-butoxycarbonyl)-N-(5-{[9-chloro-7-(5-fluoroindol-1-yl)-3,5-dihydro-2H-1,4-benzoxazepin-4-yl]methyl}pyridazin-3-yl)carbamate

To a stirred mixture of 9-chloro-7-(5-fluoroindol-1-yl)-2,3,4,5-tetrahydro-1,4-benzoxazepine (70 mg, 0.221 mmol, 1 equiv) in THF (1 mL) was added tert-butyl N-(tert-butoxycarbonyl)-N-[5-(chloromethyl)pyridazin-3-yl]carbamate (76 mg, 0.221 mmol, 1 equiv) and Et₃N (45 mg, 0.442 mmol, 2 equiv) and NaI (66 mg, 0.442 mmol, 2 equiv). The reaction was stirred at 70° C. for 2 h. Desired product could be detected by LCMS. The resulting mixture was concentrated under vacuum. The residue was purified by reversed-phase flash chromatography with the following conditions: column, C18 silica gel; mobile phase, MeCN in Water (0.05% NH₄HCO₃), 20% to 100% gradient in 10 min; detector, UV 254 nm. The fractions were combined and concentrated under vacuum to afford tert-butyl N-(tert-butoxycarbonyl)-N-(5-{[9-chloro-7-(5-fluoroindol-1-yl)-3,5-dihydro-2H-1,4-benzoxazepin-4-yl]methyl}pyridazin-3-yl)carbamate (75 mg, 51.66%) as a light yellow solid.

### Preparation of 2-(6-fluoro-1-benzothiophen-3-yl)-4,4,5,5-tetramethyl-1,3,2-dioxaborolane

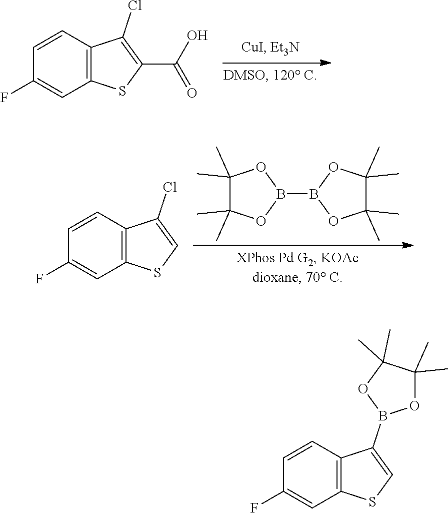

### 3-chloro-6-fluoro-1-benzothiophene

To a stirred mixture of 3-chloro-6-fluoro-1-benzothiophene-2-carboxylic acid (400 mg, 1.734 mmol, 1 equiv) in DMSO (5 mL) was added CuI (99 mg, 0.520 mmol, 0.30 equiv). The resulting mixture was stirred for 16 h at 120° C. under nitrogen atmosphere. The resulting mixture was diluted with H₂O (10 mL). The resulting mixture was extracted with EtOAc (3×10 mL). The combined organic layers were washed with water (3×10 mL), dried over anhydrous Na₂SO₄. After filtration, the filtrate was concentrated under reduced pressure. The residue was purified by silica gel column chromatography, eluted with PE/EA (10:1) to afford 3-chloro-6-fluoro-1-benzothiophene (240 mg, 74.15%) as a white solid.

### 2-(6-fluoro-1-benzothiophen-3-yl)-4,4,5,5-tetramethyl-1,3,2-dioxaborolane

To the mixture of 3-chloro-6-fluoro-1-benzothiophene (300 mg, 1.607 mmol, 1 equiv) and bis(pinacolato)diboron (816 mg, 3.214 mmol, 2 equiv) in dioxane (5.22 mL) was added KOAc (473 mg, 4.821 mmol, 3 equiv) and [2-(2-aminoethyl)phenyl](chloro)palladium dicyclohexyl[2′,4′,6′-tris(propan-2-yl)-[1,1′-biphenyl]-2-yl]phosphane (237 mg, 0.321 mmol, 0.2 equiv). The resulting mixture was stirred for 16 h at 70° C. under nitrogen atmosphere. Desired product could be detected by TLC (PE). The residue was purified by Prep-TLC (PE) to afford 2-(6-fluoro-1-benzothiophen-3-yl)-4,4,5,5-tetramethyl-1,3,2-dioxaborolane (200 mg, 44.73%) as a yellow solid.

### Preparation of 9-chloro-4-((2-(4,5-dihydro-1H-imidazol-2-yl)pyrimidin-5-yl)methyl)-7-(5-fluoro-1H-indol-1-yl)-2,3,4,5-tetrahydrobenzo[f][1,4]oxazepine

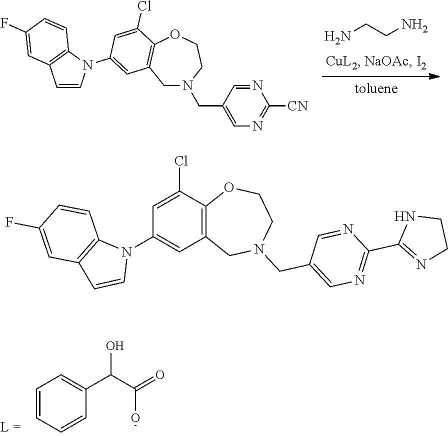

To a stirred mixture of 5-{[9-chloro-7-(5-fluoroindol-1-yl)-3,5-dihydro-2H-1,4-benzoxazepin-4-yl]methyl}pyrimidine-2-carbonitrile (50 mg, 0.115 mmol, 1 equiv) and ethylenediamine (10.39 mg, 0.173 mmol, 1.5 equiv) in toluene (3 mL) were added [(2-hydroxy-2-phenylacetyl)oxy]cuprio 2-hydroxy-2-phenylacetate (5 mg, 0.012 mmol, 0.1 equiv) and NaOAc (3 mg, 0.035 mmol, 0.3 equiv). The resulting mixture was stirred overnight at 90° C. Desired product could be detected by LCMS. The resulting mixture was filtered; the filter cake was washed with MeOH. The filtrate was concentrated under reduced pressure. The crude product (50 mg) was purified by Prep-HPLC with the following conditions (Column: Xselect CSH F-Phenyl OBD column, 19*250 mm, 5 μm; Mobile Phase A: Water (0.1% FA), Mobile Phase B: ACN; Flow rate: 25 mL/min; Gradient: 22% B to 52% B in 7 min; Wave Length: 254 nm; RT1 (min): 5.76.8) to afford 9-chloro-4-{[2-(4,5-dihydro-1H-imidazol-2-yl)pyrimidin-5-yl]methyl}-7-(5-fluoroindol-1-yl)-3,5-dihydro-2H-1,4-benzoxazepine (19.9 mg, 34.02%) as a white solid.

LC/MS: mass calcd. For C₂₅H₂₂ClFN₆O: 476.15, found: 477.15 [M+H]⁺.

¹H NMR (300 MHz, DMSO-d6) δ 8.90 (s, 2H), 7.68 (m, 2H), 7.32-7.56 (m, 3H), 7.08 (t, J=8.8 Hz, 1H), 6.69 (d, J=3.3 Hz, 1H), 4.22-4.23 (m, 2H), 4.00 (s, 2H), 3.89 (s, 2H), 3.75-3.83 (m, 4H), 3.12-3.13 (m, 2H). ¹⁹F NMR (282 MHz, DMSO-d6) δ −123.58.

### Preparation of 9-chloro-7-(5-fluoro-1H-indol-1-yl)-4-((2-(oxazol-2-yl)pyrimidin-5-yl)methyl)-2,3,4,5-tetrahydrobenzo[f][1,4]oxazepine

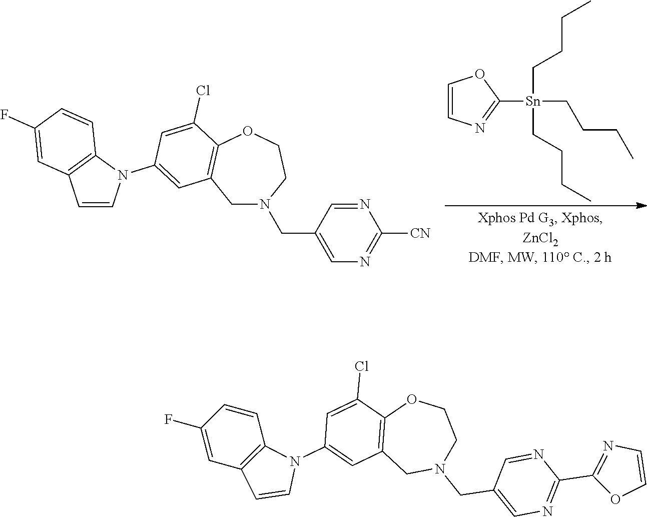 [SMILES: N#Cc1ncc(CN2CCOc3c(Cl)cc(-n4ccc5cc(F)ccc54)cc3C2)cn1 ; CCC[CH2][Sn]([CH2]CCC)([CH2]CCC)[c]1ncco1 ; Fc1ccc2c(ccn2-c2cc(Cl)c3c(c2)CN(Cc2cnc(-c4ncco4)nc2)CCO3)c1]

To a stirred mixture of 9-chloro-4-[(2-chloropyrimidin-5-yl)methyl]-7-(5-fluoroindol-1-yl)-3,5-dihydro-2H-1,4-benzoxazepine (10 mg, 0.023 mmol, 1 equiv) and 2-(tributylstannyl)-1,3-oxazole (10 mg, 0.028 mmol, 1.2 equiv) in DMF (3 mL) were added XPhos Pd G3 (4 mg, 0.005 mmol, 0.2 equiv) and ZnCl₂ (3 mg, 0.023 mmol, 1 equiv) in portions. The final reaction mixture was irradiated with microwave radiation for 2 h at 110° C. Desired products could be detected by LCMS. The mixture was allowed to cool down to room temperature. The resulting mixture was diluted with water (10 mL). The aqueous layer was extracted with EtOAc (3×10 mL). The combined organic layers were washed with brine (10 mL), dried over anhydrous Na₂SO₄. After filtration, the filtrate was concentrated under reduced pressure. The crude product (30 mg) was purified by Prep-HPLC with the following conditions (Column: XBridge Shield RP18 OBD Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 43% B to 73% B in 7 min, 73% B; Wave Length: 254 nm; RT1 (min): 6; Number Of Runs: 0) to afford 9-chloro-7-(5-fluoroindol-1-yl)-4-{[2-(1,3-oxazol-2-yl)pyrimidin-5-yl]methyl}-3,5-dihydro-2H-1,4-benzoxazepine (2.1 mg, 19.11%) as a white solid.

LC/MS: mass calcd. For C₂₅H₁₉ClFN₅O₂: 474.9, found: 476.0 [M+H]⁺.

¹H NMR (300 MHz, Methanol-d4) δ 8.94 (s, 2H), 8.18 (d, J=0.8 Hz, 1H), 7.55 (d, J=2.6 Hz, 1H), 7.51-7.39 (m, 3H), 7.35-7.22 (m, 2H), 6.99 (t, J=9.1 Hz, 1H), 6.64 (d, J=3.3 Hz, 1H), 4.30-4.21 (m, 2H), 4.01 (s, 2H), 3.91 (s, 2H), 3.25 (d, J=5.1 Hz, 2H). ¹⁹F NMR (282 MHz, DMSO-d6) δ −123.62.

### Preparation of 4-((2-(1H-tetrazol-5-yl)pyrimidin-5-yl)methyl)-9-chloro-7-(4,5-difluoro-1H-indol-1-yl)-2,3,4,5-tetrahydrobenzo[f][1,4]oxazepine

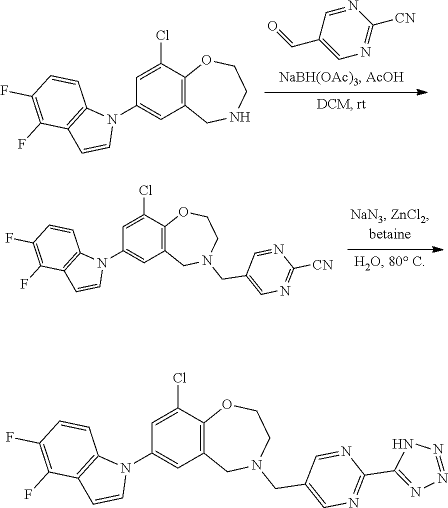 [SMILES: Fc1ccc2c(ccn2-c2cc(Cl)c3c(c2)CNCCO3)c1F ; N#Cc1ncc(C=O)cn1 ; N#Cc1ncc(CN2CCOc3c(Cl)cc(-n4ccc5c(F)c(F)ccc54)cc3C2)cn1 ; Fc1ccc2c(ccn2-c2cc(Cl)c3c(c2)CN(Cc2cnc(-c4nnn[nH]4)nc2)CCO3)c1F]

### 5-{[9-chloro-7-(4,5-difluoroindol-1-yl)-3,5-dihydro-2H-1,4-benzoxazepin-4-yl]methyl}pyrimidine-2-carbonitrile

To a stirred mixture of 9-chloro-7-(4,5-difluoroindol-1-yl)-2,3,4,5-tetrahydro-1,4-benzoxazepine (100 mg, 0.299 mmol, 1 equiv) and 5-formylpyrimidine-2-carbonitrile (60 mg, 0.449 mmol, 1.5 equiv) in DCM (2 mL) were added NaBH(OAc)₃ (127 mg, 0.598 mmol, 2 equiv) and AcOH (0.2 mL). The reaction was stirred at room temperature for 2 h. Desired product could be detected by LCMS. The resulting mixture was filtered; the filter cake was washed with CH₂Cl₂ (3×3 mL). The filtrate was concentrated under reduced pressure. The residue was dissolved in DMF (2 mL). The residue was purified by reversed-phase flash chromatography with the following conditions: column, C18 silica gel; mobile phase, MeCN in Water (0.1% TFA), 10% to 100% gradient in 20 min; detector, UV 254 nm. The fractions were combined and concentrated to afford 5-{[9-chloro-7-(4,5-difluoroindol-1-yl)-3,5-dihydro-2H-1,4-benzoxazepin-4-yl]methyl}pyrimidine-2-carbonitrile (50 mg, 35.19%) as a light yellow oil.

LC/MS: mass calcd. For C₂₃H₁₆ClF₂N₅O: 451.10, found: 452.05 [M+H]⁺.

### 4-((2-(1H-tetrazol-5-yl)pyrimidin-5-yl)methyl)-9-chloro-7-(4,5-difluoro-1H-indol-1-yl)-2,3,4,5-tetrahydrobenzo[f][1,4]oxazepine

To a stirred mixture of 5-{[9-chloro-7-(5,6-difluoroindol-1-yl)-3,5-dihydro-2H-1,4-benzoxazepin-4-yl]methyl}pyrimidine-2-carbonitrile (50 mg, 0.111 mmol, 1 equiv) and NaN₃ (22 mg, 0.333 mmol, 3 equiv) in water were added ZnCl₂ (46 mg, 0.333 mmol, 3 equiv) and 2-(trimethylazaniumyl)acetate (13 mg, 0.111 mmol, 1 equiv). The resulting mixture was stirred for 16 h at 80° C. Desired products could be detected by LCMS. The precipitated solids were collected by filtration. The crude product (50 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 28% B to 58% B in 7 min, 58% B; Wave Length: 254 nm; RT1 (min): 6.23; Number Of Runs: 0) to afford 4-((2-(1H-tetrazol-5-yl)pyrimidin-5-yl)methyl)-9-chloro-7-(4,5-difluoro-1H-indol-1-yl)-2,3,4,5-tetrahydrobenzo[f][1,4]oxazepine (2.6 mg, 4.70%) as an off-white solid.

LC/MS: mass calcd. For C₂₃H17ClF₂N80: 494.10, found: 495.10 [M+H]⁺.

¹H NMR (300 MHz, DMSO-d6) δ 8.80 (s, 2H), 7.78 (d, J=3.4 Hz, 1H), 7.67 (d, J=2.5 Hz, 1H), 7.44 (d, J=2.7 Hz, 1H), 7.21-7.34 (m, 2H), 7.15 (br, 1H), 6.82 (d, J=3.3 Hz, 1H), 4.21-4.22 (m, 2H), 4.01 (s, 2H), 3.79 (s, 2H), 3.12-3.13 (m, 2H). ¹⁹F NMR (282 MHz, DMSO-d6) δ 148.52, 148.26.

### Method R: Preparation of 9-chloro-7-(5-fluoro-1H-indol-1-yl)-4-((4-methoxypyrimidin-5-yl)methyl)-2,3,4,5-tetrahydrobenzo[f][1,4]oxazepine

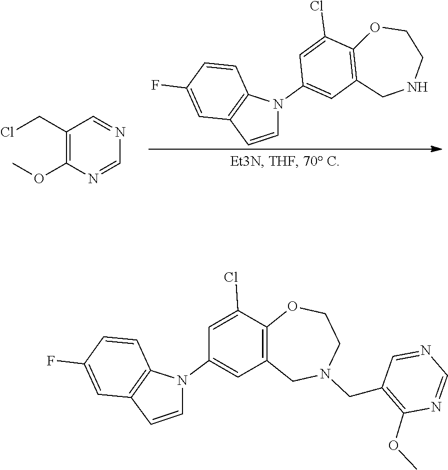

To a stirred mixture of 9-chloro-7-(5-fluoroindol-1-yl)-2,3,4,5-tetrahydro-1,4-benzoxazepine (50 mg, 0.158 mmol, 1 equiv) in THF (3 mL) were added 5-(chloromethyl)-4-methoxypyrimidine (273 mg, 0.474 mmol, 3.00 equiv) and Et₃N (32 mg, 0.316 mmol, 2 equiv) in portions at room temperature. The resulting mixture was stirred for 16 h at 70 C. The reaction was monitored by LCMS. The resulting mixture was concentrated under reduced pressure. The residue was dissolved in DMF (2 mL). The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 30% B to 60% B in 7 min, 60% B; Wave Length: 254 nm; RT1 (min): 5.87; Number Of Runs: 0) to afford 9-chloro-7-(5-fluoroindol-1-yl)-4-[(2-methoxypyrimidin-5-yl)methyl]-3,5-dihydro-2H-1,4-benzoxazepine (2.2 mg, 3.14%) as an off-white solid.

LC/MS: mass calcd. For C₂₃H₂₀ClFN₄O₂:438.13, found: 439.05[M+Na]⁺. ¹H NMR (300 MHz, DMSO-d6) δ 8.71 (s, 1H), 8.48 (s, 1H), 7.72 (d, J=3.3 Hz, 1H), 7.63 (d, J=2.7 Hz, 1H), 7.49-7.53 (m, 1H), 7.41-7.46 (m, 2H), 7.01-7.08 (m, 1H), 6.69 (d, J=3.3 Hz, 1H), 4.18-4.21 (m, 2H), 3.98 (s, 2H), 3.90 (s, 3H), 3.65 (s, 2H), 3.08-3.11 (m, 2H). ¹⁹F NMR (282 MHz, Methanol-d4) δ −126.14.

### Preparation of 9-chloro-7-(5-fluoroindol-1-yl)-4,5-dihydro-2H-spiro[1,4-benzoxazepine-3,1′-cyclopropane]

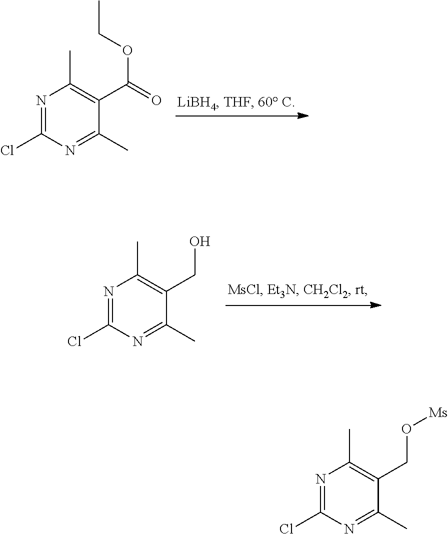

### (2-chloro-4,6-dimethylpyrimidin-5-yl)methanol

To a stirred mixture of ethyl 2-chloro-4,6-dimethylpyrimidine-5-carboxylate (300 mg, 1.398 mmol, 1 equiv) in THF (12 mL)was added LiBH₄ (60.88 mg, 2.796 mmol, 2 equiv) in portions at 0° C. under air atmosphere. The resulting mixture was stirred for additional overnight at 60° C. The reaction was monitored by LCMS. Desired product could be detected by LCMS. The mixture was allowed to cool down to room temperature. The reaction was quenched by the addition of sodium sulfate decahydrate (3 g) at 0° C. The resulting mixture was filtered, the filter cake was washed with EtOAc (3×10 mL), dried over anhydrous Na₂SO₄. After filtration, the filtrate was concentrated under reduced pressure. This resulted in (2-chloro-4,6-dimethylpyrimidin-5-yl)methanol (300 mg, 124.36%) as a yellow oil. The crude product was used in the next step directly without further purification.

### (2-chloro-4,6-dimethylpyrimidin-5-yl)methyl methanesulfonate

To a stirred mixture of (2-chloro-4,6-dimethylpyrimidin-5-yl)methanol (100 mg, 0.579 mmol, 1 equiv) in CH₂Cl₂ (5 mL)were added Et₃N (176 mg, 1.737 mmol, 3 equiv) and MsCl (133 mg, 1.158 mmol, 2 equiv) dropwise at 0° C. under air atmosphere. The resulting mixture was stirred for additional 1 h at room temperature. The reaction was monitored by LCMS. Desired product could be detected by LCMS. The resulting mixture was concentrated under reduced pressure. This resulted in (2-chloro-4,6-dimethylpyrimidin-5-yl)methyl methanesulfonate (145 mg, 99.83%) as a brown oil. The crude product was used in the next step directly without further purification.

### Preparation of 5-(chloromethyl)-4-methoxypyrimidine

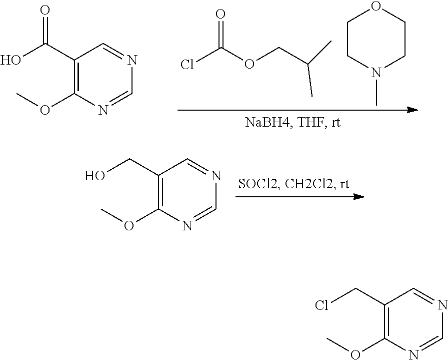

### (4-methoxypyrimidin-5-yl)methanol

To a stirred mixture of 4-methoxypyrimidine-5-carboxylic acid (500 mg, 3.244 mmol, 1 equiv) in THF (15 mL) were added 2-methylpropyl carbonochloridate (665 mg, 4.866 mmol, 1.5 equiv) and N-methylmorpholine (493 mg, 4.866 mmol, 1.5 equiv) dropwise at −10° C. under air atmosphere. The resulting mixture was stirred for additional 30 min at −10° C. The reaction was monitored by TLC. To the above mixture was added NaBH₄ (615 mg, 16.220 mmol, 5 equiv) in portions over 3 min at −10° C. The resulting mixture was stirred for additional overnight at room temperature. The reaction was monitored by LCMS. Desired product could be detected by LCMS. Add water (20 mL) to the resulting mixture. The aqueous layer was extracted with EtOAc (3×30 mL). The combined organic layers were washed with brine (100 mL), dried over anhydrous Na₂SO₄. After filtration, the filtrate was concentrated under reduced pressure. This resulted in (4-methoxypyrimidin-5-yl)methanol (825 mg, 181.46%) as a yellow oil. The crude product was used in the next step directly without further purification.

### 5-(chloromethyl)-4-methoxypyrimidine

To a stirred mixture of (4-methoxypyrimidin-5-yl) methanol (200 mg, 1.427 mmol, 1 equiv) in CH₂Cl₂ (5 mL) was added SOCl₂ (849 mg, 7.135 mmol, 5 equiv) dropwise at room temperature under air atmosphere. The resulting mixture was stirred for additional 1 h at room temperature. The reaction was monitored by LCMS. Desired product could be detected by LCMS. The resulting mixture was concentrated under reduced pressure. The residue was dissolved in CH₂Cl₂ (5 mL). The resulting mixture was concentrated under reduced pressure. This resulted in 5-(chloromethyl)-4-methoxypyrimidine (269 mg, 118.63%) as a yellow oil. The crude product was used in the next step directly without further purification.

### Preparation of 5-{[9-chloro-7-(5-fluoroindol-1-yl)-3,5-dihydro-2H-1,4-benzoxazepin-4-yl]methyl}pyrimidine-2-carboxylic acid

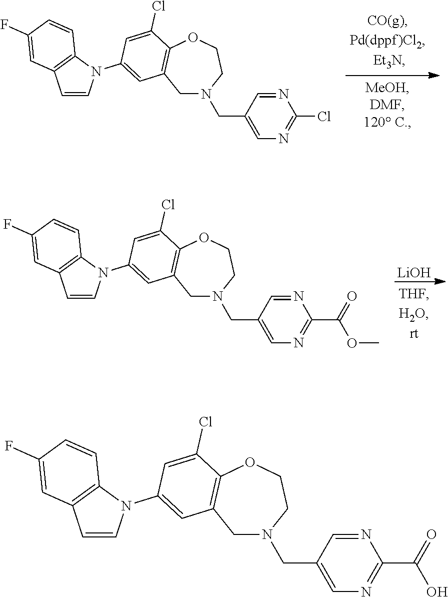 [SMILES: Fc1ccc2c(ccn2-c2cc(Cl)c3c(c2)CN(Cc2cnc(Cl)nc2)CCO3)c1 ; COC(=O)c1ncc(CN2CCOc3c(Cl)cc(-n4ccc5cc(F)ccc54)cc3C2)cn1 ; "LiOH" ; O=C(O)c1ncc(CN2CCOc3c(Cl)cc(-n4ccc5cc(F)ccc54)cc3C2)cn1]

### methyl 5-{[9-chloro-7-(5-fluoroindol-1-yl)-3,5-dihydro-2H-1,4-benzoxazepin-4-yl]methyl}pyrimidine-2-carboxylate

To a stirred mixture of 9-chloro-4-[(2-chloropyrimidin-5-yl)methyl]-7-(5-fluoroindol-1-yl)-3,5-dihydro-2H-1,4-benzoxazepine (150 mg, 0.338 mmol, 1 equiv) in MeOH (2 mL) and DMF (0.4 mL) were added Pd(dppf)Cl₂ (37 mg, 0.051 mmol, 0.15 equiv) and Et₃N (103 mg, 1.018 mmol, 3.01 equiv) at 120° C. under carbon monoxide (3 MPa) atmosphere. Desired product could be detected by LCMS. The resulting mixture was filtered; the filter cake was washed with MeOH (3×5 mL). The filtrate was concentrated under reduced pressure. The residue was dissolved in DMF (3 mL). The residue was purified by reversed-phase flash chromatography with the following conditions: column, C18 silica gel; mobile phase, MeCN in Water (0.1% TFA), 20% to 100% gradient in 10 min; detector, UV 254 nm. The fractions was concentrated under vacuum to afford methyl 5-{[9-chloro-7-(5-fluoroindol-1-yl)-3,5-dihydro-2H-1,4-benzoxazepin-4-yl]methyl}pyrimidine-2-carboxylate (100 mg, 60.13%) as a light yellow oil.

### 5-{[9-chloro-7-(5-fluoroindol-1-yl)-3,5-dihydro-2H-1,4-benzoxazepin-4-yl]methyl}pyrimidine-2-carboxylic acid

To a stirred mixture of methyl 5-{[9-chloro-7-(5-fluoroindol-1-yl)-3,5-dihydro-2H-1,4-benzoxazepin-4-yl]methyl}pyrimidine-2-carboxylate (100 mg, 0.214 mmol, 1 equiv) in THE (2 mL) was added 2 M LiOH (0.54 mL). The reaction was stirred at room temperature 2 h. Desired product could be detected by LCMS. To the above mixture was added water (10 mL) and extracted with EtOAc (3×10 mL). The combined organic layers were washed with water (5 mL), dried over anhydrous Na₂SO₄. After filtration, the filtrate was concentrated under reduced pressure to afford 5-{[9-chloro-7-(5-fluoroindol-1-yl)-3,5-dihydro-2H-1,4-benzoxazepin-4-yl]methyl}pyrimidine-2-carboxylic acid (80 mg, 78.35%) as a light yellow oil.

### Preparation of 4-((1H-pyrazolo[3,4-b]pyridin-5-yl)methyl)-9-chloro-7-(5-fluoro-1H-indol-1-yl)-2,3,4,5-tetrahydrobenzo[f][1,4]oxazepine

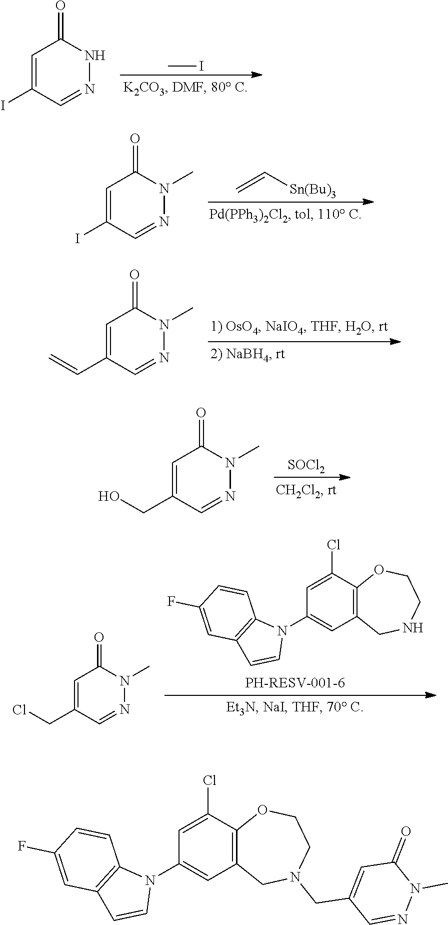

### 5-iodo-2-methylpyridazin-3-one

To a stirred mixture of 5-iodo-2H-pyridazin-3-one (500 mg, 2.252 mmol, 1 equiv) and K₂CO₃ (623 mg, 4.504 mmol, 2 equiv) in DMF (10.00 mL) were added methyl iodide (639 mg, 4.504 mmol, 2 equiv) dropwise for 16 h at 80° C. under air atmosphere. Desired product could be detected by LCMS. To the above residue was added water (50 mL) and extracted with EtOAc (3×50 mL). The combined organic layers were washed with water (3×30 mL), dried over anhydrous Na₂SO₄. After filtration, the filtrate was concentrated under reduced pressure to afford 5-iodo-2-methylpyridazin-3-one (0.6 g, 112.87%) as a white solid.

### 5-ethenyl-2-methylpyridazin-3-one

To a stirred mixture of 5-iodo-2-methylpyridazin-3-one (570 mg, 2.415 mmol, 1 equiv) and tributyl(ethenyl)stannane (1532 mg, 4.830 mmol, 2 equiv) in toluene (10 mL) were added Pd(pph₃)₂Cl₂ (169 mg, 0.242 mmol, 0.1 equiv) dropwise for 2 h at 110° C. under nitrogen atmosphere. Desired product could be detected by LCMS. The resulting mixture was concentrated under reduced pressure. The residue was purified by silica gel column chromatography, eluted with PE/EA (2:1) to afford 5-ethenyl-2-methylpyridazin-3-one (280 mg, 85.15%) as an off-white solid.

### tert-butyl N-(tert-butoxycarbonyl)-N-[5-(hydroxymethyl)pyridazin-3-yl]carbamate

To a stirred mixture of 5-ethenyl-2-methylpyridazin-3-one (250 mg, 1.836 mmol, 1 equiv) in THF (1.5 mL) and H₂O (1.5 mL) were added OsO₄ (52 mg, 0.202 mmol, 0.11 equiv). The reaction was stirred at room temperature for 2 min. NaIO₄ (789 mg, 3.690 mmol, 2.01 equiv) was added. The reaction was stirred at room temperature for 5 h. To the above mixture was added NaBH₄ (140 mg, 3.709 mmol, 2.02 equiv) at 0° C. The resulting mixture was stirred for additional 15 min at room temperature. Desired product could be detected by LCMS. To the above mixture was added water (30 mL) and extracted with EtOAc (3×30 mL). The combined organic layers were washed with water (10 mL), dried over anhydrous Na₂SO₄. After filtration, the filtrate was concentrated under reduced pressure. The residue was purified by silica gel column chromatography, eluted with PE/EA (1:1) to afford 5-(hydroxymethyl)-2-methylpyridazin-3-one (220 mg, 81.22%) as a brown oil.

### 5-(chloromethyl)-2-methylpyridazin-3-one

To a stirred mixture of 5-(hydroxymethyl)-2-methylpyridazin-3-one (100 mg, 0.714 mmol, 1 equiv) in DCM (1.5 mL) were added SOCl2 (127 mg, 1.071 mmol, 1.5 equiv) in DCM (0.5 mL) at 0° C. The reaction was stirred at room temperature for 1 h. Desired product could be detected by TLC (PE/EA 2:1, Rf=0.4). The resulting mixture was concentrated under vacuum to afford 5-(chloromethyl)-2-methylpyridazin-3-one (100 mg, 79.53%) as a light brown oil.

### 4-((1H-pyrazolo[3,4-b]pyridin-5-yl)methyl)-9-chloro-7-(5-fluoro-1H-indol-1-yl)-2,3,4,5-tetrahydrobenzo[f][1,4]oxazepane

To a stirred mixture of 9-chloro-7-(5-fluoroindol-1-yl)-2,3,4,5-tetrahydro-1,4-benzoxazepine (70 mg, 0.221 mmol, 1 equiv) and 5-(chloromethyl)-2-methylpyridazin-3-one (70 mg, 0.442 mmol, 2 equiv) in THF (1 mL) were added Et₃N (45 mg, 0.442 mmol, 2 equiv) and NaI (66 mg, 0.442 mmol, 2 equiv). The reaction was stirred at 70° C. for 12 h. Desired product could be detected by LCMS. The resulting mixture was concentrated under vacuum. The residue was dissolved in MeOH (1 mL). The crude product was purified by Prep-HPLC with the following conditions (Column: YMC-Actus Triart C18 ExRS, 30*150 mm, 5 μm; Mobile Phase A: Water (10 mmol/L NH₄HCO₃), Mobile Phase B: MeOH—HPLC; Flow rate: 60 mL/min; Gradient: 55% B to 85% B in 7 min, 85% B; Wave Length: 254 nm; RT1 (min): 5.25; Number of Runs: 0). The fractions ware combined and lyophilized to afford 4-((1H-pyrazolo[3,4-b]pyridin-5-yl)methyl)-9-chloro-7-(5-fluoro-1H-indol-1-yl)-2,3,4,5-tetrahydrobenzo[f][1,4]oxazepine (19.3 mg, 19.84%) as an off-white solid.

LC/MS: mass calcd. For C₂₃H₂₀ClFN₄O₂: 438.1, found: 439.1 [M+H]⁺. ¹H NMR (300 MHz, DMSO-d6) δ 7.87 (d, J=2.0 Hz, 1H), 7.71 (d, J=3.3 Hz, 1H), 7.63 (d, J=2.6 Hz, 1H), 7.48-7.57 (m, 1H), 7.38-7.47 (m, 2H), 7.13-7.00 (m, 1H), 6.81 (s, 1H), 6.69 (d, J=3.2 Hz, 1H), 4.15-4.21 (m, 2H), 3.97 (s, 2H), 3.58-3.65 (m, 5H), 3.10-3.16 (m, 2H). ¹⁹F NMR (282 MHz, DMSO-d6) δ −123.6.

### Preparation of 5-((9-chloro-7-(4,5-difluoro-1H-indol-1-yl)-2,3-dihydrobenzo[f][1,4]oxazepin-4 (5H)-yl)methyl)pyrimidine-2-carboxylic acid

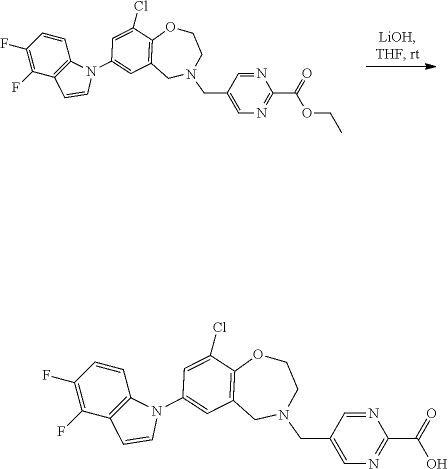 [SMILES: CCOC(=O)c1ncc(CN2CCOc3c(Cl)cc(-n4ccc5c(F)c(F)ccc54)cc3C2)cn1 ; O=C(O)c1ncc(CN2CCOc3c(Cl)cc(-n4ccc5c(F)c(F)ccc54)cc3C2)cn1]

To a stirred mixture of ethyl 5-{[9-chloro-7-(4,5-difluoroindol-1-yl)-3,5-dihydro-2H-1,4-benzoxazepin-4-yl]methyl}pyrimidine-2-carboxylate (100 mg, 0.200 mmol, 1 equiv) in THF (2 mL) were added 2M LiOH·H₂O (2 mL, 0.200 mmol). The reaction was stirred at room temperature for 2 h. Desired product could be detected by LCMS. The mixture was neutralized to pH 7 with 2M HCl (aq.). The resulting mixture was concentrated under vacuum. The residue was dissolved in DMSO (2 mL). The crude product was purified by Prep-HPLC with the following conditions (Column: XBridge Prep OBD C18 Column, 19*250 mm, 5 μm; Mobile Phase A: Water (10 mmol/L NH₄HCO3+0.1% NH₃·H₂O), Mobile Phase B: ACN; Flow rate: 25 mL/min; Gradient: 30% B to 60% B in 7 min, 60% B; Wave Length: 254 nm; RT1 (min): 6.12; Number of Runs: 0). The fractions were combined and lyophilized to afford 5-{[9-chloro-7-(4,5-difluoroindol-1-yl)-3,5-dihydro-2H-1,4-benzoxazepin-4-yl]methyl}pyrimidine-2-carboxylic acid (31.7 mg, 33.15%) as an off-white solid.

LC/MS: mass calcd. For C₂₃H₁₇ClF₂N₄O₃: 470.1, found: 471.0 [M+H]⁺. ¹H NMR (300 MHz, DMSO-d₆) δ 8.75 (s, 2H), 7.78 (d, J=3.4 Hz, 1H), 7.67 (d, J=2.6 Hz, 1H), 7.43 (d, J=2.7 Hz, 1H), 7.19-7.34 (m, 2H), 6.83 (d, J=3.3 Hz, 1H), 4.17-4.23 (m, 2H), 3.98 (s, 2H), 3.77 (s, 2H), 3.07-3.13 (m, 2H). ¹⁹F NMR (282 MHz, DMSO-d₆) δ −148.596, 151.2.

### Preparation of 4-((2-(1H-tetrazol-5-yl)pyrimidin-5-yl)methyl)-9-chloro-7-(6-fluorobenzofuran-3-yl)-2,3,4,5-tetrahydrobenzo[f][1,4]oxazepine

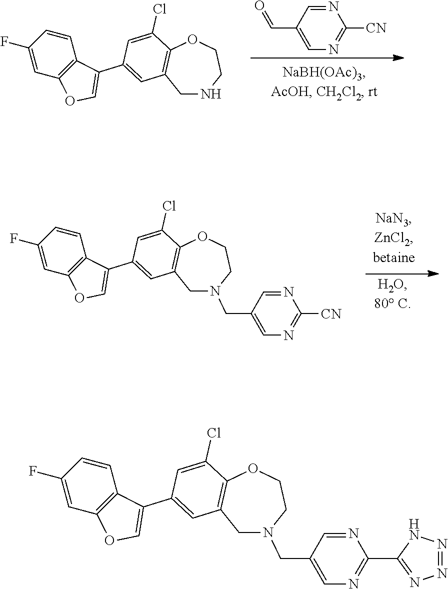 [SMILES: Fc1ccc2c(-c3cc(Cl)c4c(c3)CNCCO4)coc2c1 ; N#Cc1ncc(C=O)cn1 ; N#Cc1ncc(CN2CCOc3c(Cl)cc(-c4coc5cc(F)ccc45)cc3C2)cn1 ; Fc1ccc2c(-c3cc(Cl)c4c(c3)CN(Cc3cnc(-c5nnn[nH]5)nc3)CCO4)coc2c1]

### 5-{[9-chloro-7-(6-fluoro-1-benzofuran-3-yl)-3,5-dihydro-2H-1,4-benzoxazepin-4-yl]methyl}pyrimidine-2-carbonitrile

To a stirred mixture of 9-chloro-7-(6-fluoro-1-benzofuran-3-yl)-2,3,4,5-tetrahydro-1,4-benzoxazepine (60 mg, 0.189 mmol, 1 equiv) and 5-formylpyrimidine-2-carbonitrile (38 mg, 0.283 mmol, 1.5 equiv) in CH₂Cl₂ (2 mL) were added NaBH(OAc)₃ (80 mg, 0.378 mmol, 2 equiv) and AcOH (0.08 mg, 0.001 mmol, 0.01 equiv). The reaction was stirred at room temperature for 3 h. Desired product could be detected by LCMS. The resulting mixture was filtered; the filter cake was washed with CH₂Cl₂ (3×3 mL). The filtrate was concentrated under reduced pressure. The resulting mixture was concentrated under vacuum. The residue was dissolved in DMF (1 mL). The residue was purified by reversed-phase flash chromatography with the following conditions: column, C18 silica gel; mobile phase, MeCN in Water (0.1% TFA), 10% to 100% gradient in 20 min; detector, UV 254 nm. The fractions were combined and concentrated to afford 5-{[9-chloro-7-(6-fluoro-1-benzofuran-3-yl)-3,5-dihydro-2H-1,4-benzoxazepin-4-yl]methyl}pyrimidine-2-carbonitrile (60 mg, 69.41%) as a light yellow oil.

### 4-((2-(1H-tetrazol-5-yl)pyrimidin-5-yl)methyl)-9-chloro-7-(6-fluorobenzofuran-3-yl)-2,3,4,5-tetrahydrobenzo[f][1,4]oxazepane

To a stirred mixture of 5-{[9-chloro-7-(6-fluoro-1-benzofuran-3-yl)-3,5-dihydro-2H-1,4-benzoxazepin-4-yl]methyl}pyrimidine-2-carbonitrile (60 mg, 0.138 mmol, 1 equiv) in H₂O (1 mL) were added NaN₃ (27 mg, 0.414 mmol, 3 equiv) and ZnCl₂ (37 mg, 0.276 mmol, 2 equiv) and betaine (16 mg, 0.138 mmol, 1 equiv). The reaction was stirred at 80° C. for 36 h. Desired product could be detected by LCMS. The precipitated solids were collected by filtration and washed with water (3×2 mL). The filtration was dissolved in DMSO (3 mL). The crude product was purified by Prep-HPLC with the following conditions (Column: XBridge Prep Phenyl OBD Column, 19*100 mm, 5 μm; Mobile Phase A: Water (10 mmol/L NH₄HCO₃+0.1% NH₃·H₂O), Mobile Phase B: ACN; Flow rate: 60 mL/min; Gradient: 26% B to 56% B in 7 min, 56% B; Wave Length: 254 nm; RT1 (min): 6.55; Injection Volume: 1900 mL; Number Of Runs: 3) to afford 9-chloro-7-(6-fluoro-1-benzofuran-3-yl)-4-{[2-(1H-1,2,3,4-tetrazol-5-yl)pyrimidin-5-yl]methyl}-3,5-dihydro-2H-1,4-benzoxazepine (10.6 mg, 16.00%) as a white solid.

LC/MS: mass calcd. For C₂₃H₇ClFN₇O₂: 477.1, found: 478.0 [M+H−56]⁺ ¹H NMR (300 MHz, DMSO-d₆) δ 8.79 (s, 2H), 8.43 (s, 1H), 7.86-7.97 (m, 1H), 7.74 (d, J=2.1 Hz, 1H), 7.59-7.68 (m, 1H), 7.51 (d, J=2.2 Hz, 1H), 7.22-7.34 (m, 1H), 7.17-7.21 (m, 1H), 4.17-4.23 (m, 2H), 4.01 (s, 2H), 3.78 (s, 2H), 3.09-3.14 (s, 2H). ¹⁹F NMR (282 MHz, DMSO-d₆) δ −116.8.

### Preparation of N-(5-((9-chloro-7-(5-fluoro-1H-indol-1-yl)-2,3-dihydrobenzo[f][1,4]oxazepin-4 (5H)-yl)methyl)pyridazin-3-yl)methanesulfonamide

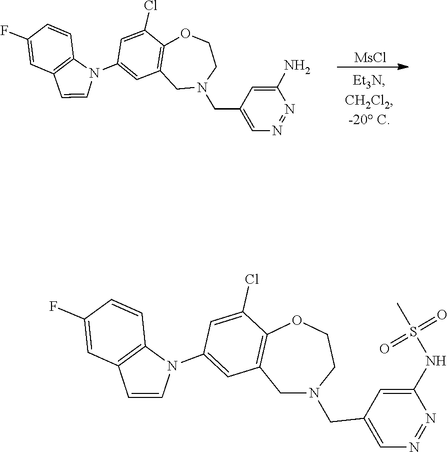 [SMILES: "MsCl" ; Nc1cc(CN2CCOc3c(Cl)cc(-n4ccc5cc(F)ccc54)cc3C2)cnn1 ; CS(=O)(=O)Nc1cc(CN2CCOc3c(Cl)cc(-n4ccc5cc(F)ccc54)cc3C2)cnn1]

To a stirred mixture of 5-{[9-chloro-7-(5-fluoroindol-1-yl)-3,5-dihydro-2H-1,4-benzoxazepin-4-yl]methyl}pyridazin-3-amine (15 mg, 0.035 mmol, 1 equiv) in CH₂Cl₂ (1 mL) were added Et₃N (7 mg, 0.070 mmol, 2 equiv) and MsCl (8 mg, 0.070 mmol, 2 equiv) dropwise at −20° C. The reaction was stirred at room temperature for 2 h. Desired product could be detected by LCMS. The resulting mixture was concentrated under vacuum. The residue was dissolved in DMF (1 mL). The crude product was purified by Prep-HPLC with the following conditions (Column: YMC-Actus Triart C18 ExRS, 30*150 mm, 5 μm; Mobile Phase A: Water (10 mmol/L NH₄HCO₃), Mobile Phase B: MeOH—HPLC; Flow rate: 60 mL/min; Gradient: 55% B to 85% B in 7 min, 85% B; Wave Length: 254 nm; RT1 (min): 5.25; Number of Runs: 0). The fractions were combined and lyophilized to afford N-(5-{[9-chloro-7-(5-fluoroindol-1-yl)-3,5-dihydro-2H-1,4-benzoxazepin-4-yl]methyl}pyridazin-3-yl)methanesulfonamide (1.5 mg, 8.14%) as an off-white solid.

LC/MS: mass calcd. For C₂₃H₂₁ClFN₅O₃S: 501.1, found: 502.1 [M+H]⁺. ¹H NMR (400 MHz, DMSO-d₆) δ 8.29 (s, 1H), 7.72 (d, J=3.3 Hz, 1H), 7.63 (d, J=2.6 Hz, 1H), 7.48-7.55 (m, 1H), 7.38-7.46 (m, 2H), 7.02-7.12 (m, 1H), 6.68 (d, J=3.3 Hz, 1H), 6.54 (s, 1H), 4.15-4.21 (m, 2H), 3.96 (s, 2H), 3.70 (s, 2H), 3.10-3.16 (m, 2H), 2.89 (s, 3H). ¹⁹F NMR (376 MHz, DMSO-d₆) δ −123.6.

### Preparation of 9-chloro-7-(4,5-difluoroindol-1-yl)-2,3,4,5-tetrahydro-1,4-benzoxazepine

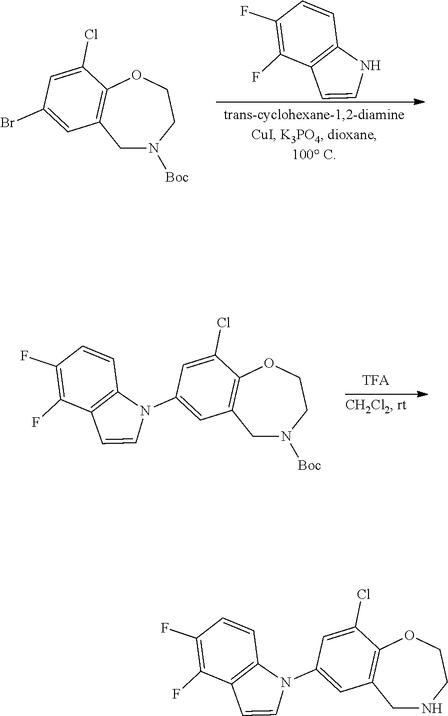 [SMILES: CC(C)(C)OC(=O)N1CCOc2c(Cl)cc(Br)cc2C1 ; Fc1ccc2[nH]ccc2c1F ; CC(C)(C)OC(=O)N1CCOc2c(Cl)cc(-n3ccc4c(F)c(F)ccc43)cc2C1 ; "CH2Cl2, rt" ; Fc1ccc2c(ccn2-c2cc(Cl)c3c(c2)CNCCO3)c1F]

### tert-butyl 9-chloro-7-(4,5-difluoroindol-1-yl)-3,5-dihydro-2H-1,4-benzoxazepine-4-carboxylate

To a stirred mixture of tert-butyl 7-bromo-9-chloro-3,5-dihydro-2H-1,4-benzoxazepine-4-carboxylate (600 mg, 1.654 mmol, 1 equiv) and 4,5-difluoro-1H-indole (0.38 g, 2.481 mmol, 1.5 equiv) in 1,4-dioxane (6 mL) were added CuI (0.09 g, 0.496 mmol, 0.3 equiv) and K3PO₄ (1.23 g, 5.789 mmol, 3.5 equiv) and (1R,2R)-cyclohexane-1,2-diamine (0.09 g, 0.827 mmol, 0.5 equiv). The reaction was stirred at 100° C. for 2 h. Desired product could be detected by LCMS. The resulting mixture was filtered; the filter cake was washed with 1,4-dioxane (3×5 mL). The residue was purified by reversed-phase flash chromatography with the following conditions: column, C18 silica gel; mobile phase, MeCN in Water (0.1% TFA), 10% to 100% gradient in 20 min; detector, UV 254 nm. The fractions were combined and concentrated to afford tert-butyl 9-chloro-7-(4,5-difluoroindol-1-yl)-3,5-dihydro-2H-1,4-benzoxazepine-4-carboxylate (400 mg, 52.82%) as a light yellow solid.

### 9-chloro-7-(4,5-difluoroindol-1-yl)-2,3,4,5-tetrahydro-1,4-benzoxazepine

To a stirred mixture of tert-butyl 9-chloro-7-(4,5-difluoroindol-1-yl)-3,5-dihydro-2H-1,4-benzoxazepine-4-carboxylate (400 mg, 0.920 mmol, 1 equiv) in CH₂Cl₂ (5.00 mL) were added TFA (1.00 ml). The reaction was stirred at room temperature for 2 h. Desired product could be detected by LCMS. The mixture was neutralized to pH 7 with saturated NaHCO₃(aq.). The resulting mixture was extracted with CH₂Cl₂ (3×15 mL). The combined organic layers were washed with water (2×7 mL), dried over anhydrous Na₂SO₄. After filtration, the filtrate was concentrated under reduced pressure to afford 9-chloro-7-(4,5-difluoroindol-1-yl)-2,3,4,5-tetrahydro-1,4-benzoxazepine (300 mg, 92.56%) as a brown yellow oil.

### Preparation of 1-(5-((9-chloro-7-(5-fluoro-1H-indol-1-yl)-2,3-dihydrobenzo[f][1,4]oxazepin-4 (5H)-yl)methyl)pyrimidin-2-yl)ethanone [319-3]

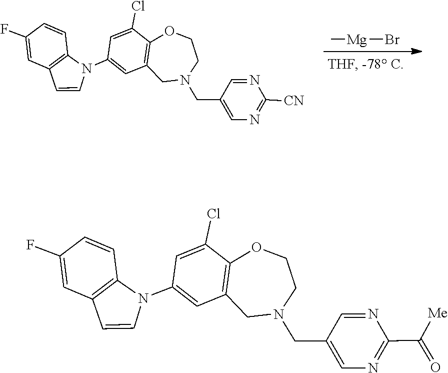

To a stirred mixture of 5-{[9-chloro-7-(5-fluoroindol-1-yl)-3,5-dihydro-2H-1,4-benzoxazepin-4-yl]methyl}pyrimidine-2-carbonitrile (20 mg, 0.046 mmol, 1 equiv) in THF (1 mL) were added bromo(methyl)magnesium (7 mg, 0.055 mmol, 1.2 equiv) dropwise at −78° C. under nitrogen atmosphere. The reaction was stirred at −78° C. for 3 h under nitrogen atmosphere. Desired product could be detected by LCMS. The reaction was quenched with sat. NH₄Cl (aq.) and extracted with EtOAc (3×15 mL). The combined organic layers were washed with brine (1×10 mL), dried over anhydrous Na₂SO₄. After filtration, the filtrate was concentrated under reduced pressure. The residue was dissolved in DMF (1 mL). The crude product was purified by Prep-HPLC with the following conditions (Column: XBridge Prep Phenyl OBD Column, 19*100 mm, 5 μm; Mobile Phase A: Water (10 mmol/L NH₄HCO₃+0.1% NH₃·H₂O), Mobile Phase B: ACN; Flow rate: 60 mL/min; Gradient: 42% B to 72% B in 7 min, 72% B; Wave Length: 254 nm; RT1 (min): 6; Number of Runs: 0). The fractions were combined and lyophilized to afford 1-(5-{[9-chloro-7-(5-fluoroindol-1-yl)-3,5-dihydro-2H-1,4-benzoxazepin-4-yl]methyl}pyrimidin-2-yl)ethanone (20 mg, 91.79%) as an off-white oil.

LC/MS: mass calcd. For C₂₄H₂₀ClFN₄O₂: 450.13, found: 451.05 [M+H]⁺. ¹H NMR (300 MHz, DMSO-d₆) δ 8.94 (s, 2H), 7.71 (d, J=3.3 Hz, 1H), 7.65 (d, J=2.6 Hz, 1H), 7.47-7.57 (m, 1H), 7.38-7.47 (m, 2H), 7.00-7.12 (m, 1H), 6.65-6.73 (m, 1H), 4.18-4.23 (m, 2H), 4.01 (s, 2H), 3.84 (s, 2H), 3.34 (s, 32H), 3.08-3.14 (m, 2H), 2.67 (s, 3H). ¹⁹F NMR (282 MHz, DMSO-d₆) δ −123.6.

### Preparation of 1-(5-{[9-chloro-7-(5-fluoroindol-1-yl)-3,5-dihydro-2H-1,4-benzoxazepin-4-yl]methyl}pyrimidin-2-yl)ethanone

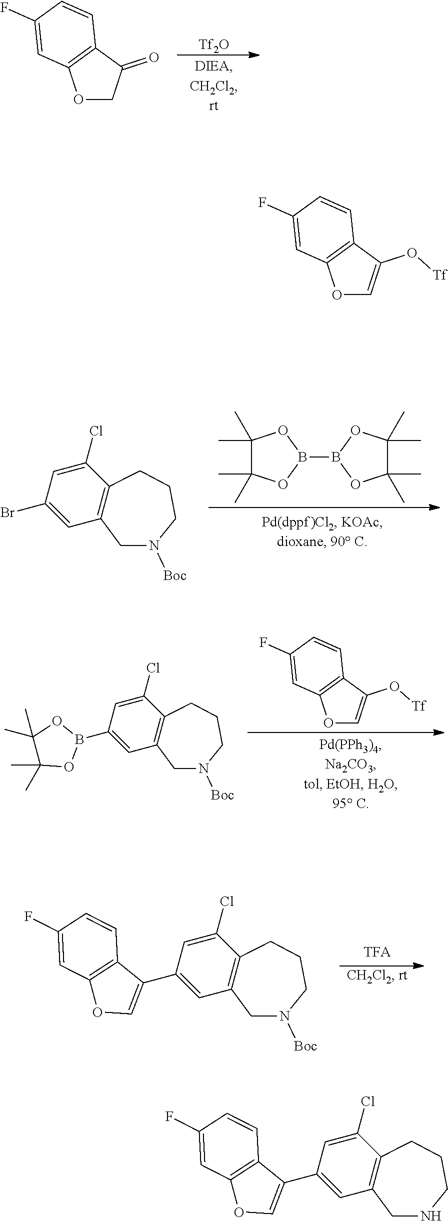

### 6-fluoro-2,3-dihydro-1-benzofuran-3-yl trifluoromethanesulfonate

To a stirred mixture of 6-fluoro-2H-1-benzofuran-3-one (500 mg, 3.287 mmol, 1 equiv) in CH₂Cl₂ (5 mL) was added DIEA (849 mg, 6.574 mmol, 2 equiv) dropwise at room temperature under air atmosphere. To the above mixture was added Tf₂O (1066 mg, 3.780 mmol, 1.15 equiv) dropwise at 0° C. The resulting mixture was stirred for additional 10 min at room temperature. The reaction was monitored by TLC (PE/EA=5:1, Rf=0.3). The resulting mixture was diluted with water (10 mL). The aqueous layer was extracted with CH₂Cl₂ (3×10 mL). The combined organic layers were washed with brine (1×30 mL), dried over anhydrous MgSO4. After filtration, the filtrate was concentrated under reduced pressure. This resulted in 6-fluoro-2,3-dihydro-1-benzofuran-3-yl trifluoromethanesulfonate (500 mg, 53.15%) as a colorless oil.

### tert-butyl 8-chloro-6-(4,4,5,5-tetramethyl-1,3,2-dioxaborolan-2-yl)-4,5-dihydro-2H-1,3-benzoxazepine-3-carboxylate)

To a stirred mixture of tert-butyl 6-bromo-8-chloro-4,5-dihydro-2H-1,3-benzoxazepine-3-carboxylate (500 mg, 1.379 mmol, 1.00 equiv) in 1,4-dioxane (5 mL) were added KOAc (406 mg, 4.137 mmol, 3 equiv) and 4,4,5,5-tetramethyl-2-(4,4,5,5-tetramethyl-1,3,2-dioxaborolan-2-yl)-1,3,2-dioxaborolane (385 mg, 1.517 mmol, 1.1 equiv) in portions at room temperature under air atmosphere. The resulting mixture was stirred overnight at 90° C. under nitrogen atmosphere. The reaction was monitored by LCMS. The resulting mixture was filtered; the filter cake was washed with CH₂Cl₂ (3×10 mL). The filtrate was concentrated under reduced pressure. The residue was purified by silica gel column chromatography, eluted with PE/EA (10:1) to afford tert-butyl 8-chloro-6-(4,4,5,5-tetramethyl-1,3,2-dioxaborolan-2-yl)-4,5-dihydro-2H-1,3-benzoxazepine-3-carboxylate) (460 mg, 81.43%) as a white solid.

### tert-butyl 9-chloro-7-(6-fluoro-1-benzofuran-3-yl)-3,5-dihydro-2H-1,4-benzoxazepine-4-carboxylate

To a stirred mixture of tert-butyl 9-chloro-7-(4,4,5,5-tetramethyl-1,3,2-dioxaborolan-2-yl)-3,5-dihydro-2H-1,4-benzoxazepine-4-carboxylate (350 mg, 0.854 mmol, 1 equiv) and 6-fluoro-1-benzofuran-3-yl trifluoromethanesulfonate (437 mg, 1.537 mmol, 1.8 equiv) in toluene (3 mL) and EtOH (1.5 mL) and H₂O (1.5 mL) were added Na₂CO₃ (163 mg, 1.537 mmol, 1.8 equiv) and Pd(pph3)₄ (49 mg, 0.043 mmol, 0.05 equiv). The reaction was stirred at 95° C. under nitrogen atmosphere for 12 h. Desired product could be detected by LCMS. The resulting mixture was extracted with toluene (3×30 mL). The combined organic layers were washed with water (lx 10 mL), dried over anhydrous Na₂SO₄. After filtration, the filtrate was concentrated under reduced pressure. The residue was purified by silica gel column chromatography, eluted with PE/EA (10:1) to afford tert-butyl 9-chloro-7-(6-fluoro-1-benzofuran-3-yl)-3,5-dihydro-2H-1,4-benzoxazepine-4-carboxylate (340 mg, 90.49%) as a light yellow oil.

### 9-chloro-7-(6-fluoro-1-benzofuran-3-yl)-2,3,4,5-tetrahydro-1,4-benzoxazepine

To a stirred mixture of tert-butyl 9-chloro-7-(6-fluoro-1-benzofuran-3-yl)-3,5-dihydro-2H-1,4-benzoxazepine-4-carboxylate (330 mg, 0.790 mmol, 1 equiv) in DCM (3 mL) were added TFA (0.6 mL). The reaction was stirred at room temperature for 2 h. Desired product could be detected by LCMS. The mixture neutralized to pH 7 with saturated NaHCO₃ (aq.). To the above mixture was added water (30 ml). The resulting mixture was extracted with CH₂Cl₂ (3×30 mL). The combined organic layers were washed with brine (1×30 mL), dried over anhydrous Na₂SO₄. After filtration, the filtrate was concentrated under reduced pressure to afford 9-chloro-7-(6-fluoro-1-benzofuran-3-yl)-2,3,4,5-tetrahydro-1,4-benzoxazepine (260 mg, 93.25%) as a light yellow oil.

### Preparation of 9-chloro-4-[(2-chloropyrimidin-5-yl)methyl]-7-(6-fluoro-1-benzofuran-3-yl)-3,5-dihydro-2H-1,4-benzoxazepine

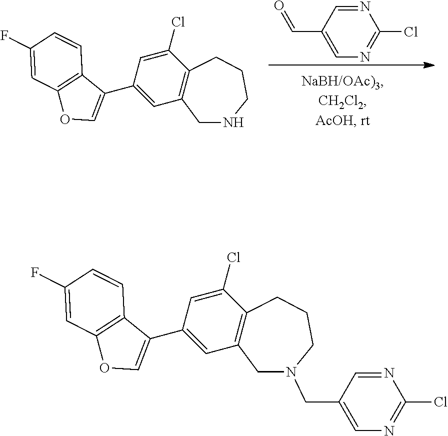 [SMILES: Fc1ccc2c(-c3cc(Cl)c4c(c3)CNCCC4)coc2c1 ; O=Cc1cnc(Cl)nc1 ; Fc1ccc2c(-c3cc(Cl)c4c(c3)CN(Cc3cnc(Cl)nc3)CCC4)coc2c1]

### 9-chloro-4-[(2-chloropyrimidin-5-yl)methyl]-7-(6-fluoro-1-benzofuran-3-yl)-3,5-dihydro-2H-1,4-benzoxazepine

To a stirred mixture of 9-chloro-7-(6-fluoro-1-benzofuran-3-yl)-2,3,4,5-tetrahydro-1,4-benzoxazepine (100 mg, 0.315 mmol, 1 equiv) and 2-chloropyrimidine-5-carbaldehyde (67 mg, 0.473 mmol, 1.5 equiv) in CH₂Cl₂ (1 ml) were added NaBH(OAc)₃ (133 mg, 0.630 mmol, 2 equiv) and AcOH (0.08 mg, 0.001 mmol). The reaction was stirred at room temperature for 2 h. Desired product could be detected by LCMS. The resulting mixture was filtered; the filter cake was washed with CH₂Cl₂ (3×3 mL). The filtrate was concentrated under reduced pressure. The residue was dissolved in DMF (2 mL). The residue was purified by reversed-phase flash chromatography with the following conditions: column, C18 silica gel; mobile phase, MeCN in Water (0.1% TFA), 10% to 100% gradient in 10 min; detector, UV 254 nm. The fractions were combined and concentrated to afford 9-chloro-4-[(2-chloropyrimidin-5-yl)methyl]-7-(6-fluoro-1-benzofuran-3-yl)-3,5-dihydro-2H-1,4-benzoxazepine (80 mg, 54.35%) as an off-white solid.

### Preparation of 9-chloro-7-(5-fluoroindazol-1-yl)-2,3,4,5-tetrahydro-1,4-benzoxazepine

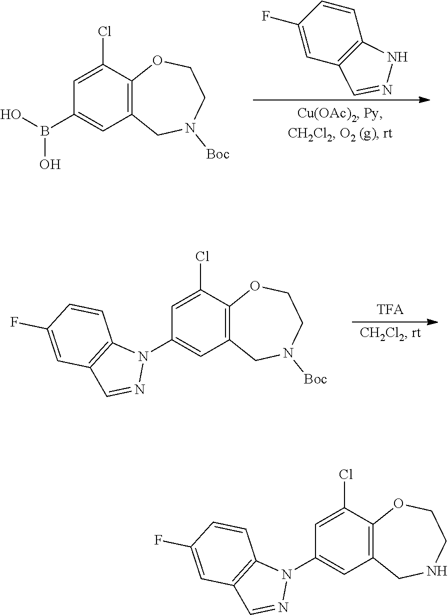 [SMILES: CC(C)(C)OC(=O)N1CCOc2c(Cl)cc(B(O)O)cc2C1 ; Fc1ccc2[nH]ncc2c1 ; CC(C)(C)OC(=O)N1CCOc2c(Cl)cc(-n3ncc4cc(F)ccc43)cc2C1 ; Fc1ccc2c(cnn2-c2cc(Cl)c3c(c2)CNCCO3)c1]

### 9-chloro-7-(5-fluoroindazol-1-yl)-3,5-dihydro-2H-1,4-benzoxazepine-4-carboxylate

To the mixture of 4-(tert-butoxycarbonyl)-9-chloro-3,5-dihydro-2H-1,4-benzoxazepin-7-ylboronic acid (300 mg, 0.916 mmol, 1 equiv) and 5-fluoro-1H-indazole (124 mg, 0.916 mmol, 1 equiv) in CH₂Cl₂ (5 mL) was added Copper(II) acetate (332 mg, 1.832 mmol, 2 equiv) and pyridine (289 mg, 3.664 mmol, 4 equiv). The mixture was stirred at room temperature for 16 h under O₂ atmosphere. Desired product could be detected by LCMS. The mixture was added with H₂O (5 mL) and extracted by EtOAc (5 mL×3). The organic layer was combined, washed by brine (15 mL), dried over Na₂SO₄, filtered and concentrated. The residue was purified by silica gel column chromatography, eluted with CH₂Cl₂/MeOH (10:1) to afford tert-butyl 9-chloro-7-(5-fluoroindazol-1-yl)-3,5-dihydro-2H-1,4-benzoxazepine-4-carboxylate (150 mg, 39.20%) as a yellow oil.

### 9-chloro-7-(5-fluoroindazol-1-yl)-2,3,4,5-tetrahydro-1,4-benzoxazepine

To the mixture of tert-butyl 9-chloro-7-(5-fluoroindazol-1-yl)-3,5-dihydro-2H-1,4-benzoxazepine-4-carboxylate (150 mg, 0.359 mmol, 1 equiv) in CH₂Cl₂ (5 mL) was added TFA (1 mL). The mixture was stirred at room temperature for 1 h. Desired product could be detected by LCMS. The mixture was concentrated under reduced pressure to afford 9-chloro-7-(5-fluoroindazol-1-yl)-2,3,4,5-tetrahydro-1,4-benzoxazepine (100 mg, 87.67%) as brown oil.

### Preparation of 9-chloro-7-(5-fluoro-1,3-benzodiazol-1-yl)-2,3,4,5-tetrahydro-1,4-benzoxazepine

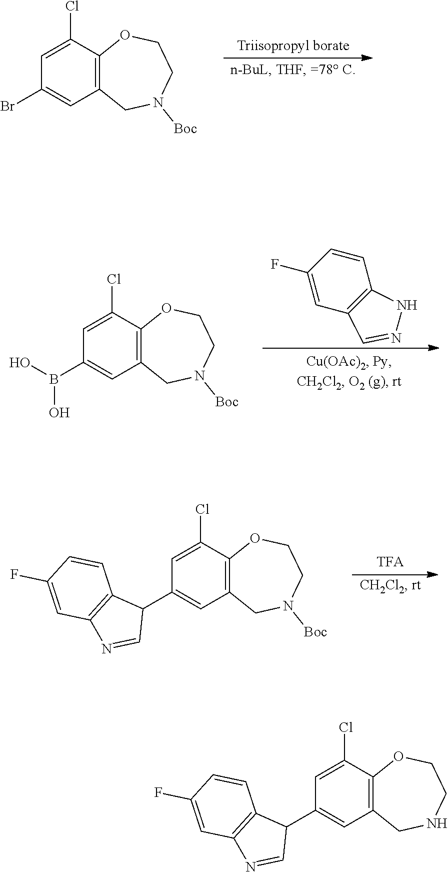 [SMILES: CC(C)(C)OC(=O)N1CCOc2c(Cl)cc(Br)cc2C1 ; CC(C)(C)OC(=O)N1CCOc2c(Cl)cc(B(O)O)cc2C1 ; Fc1ccc2[nH]ncc2c1 ; CC(C)(C)OC(=O)N1CCOc2c(Cl)cc(C3C=Nc4cc(F)ccc43)cc2C1 ; Fc1ccc2c(c1)N=CC2c1cc(Cl)c2c(c1)CNCCO2]

### 4-(tert-butoxycarbonyl)-9-chloro-3,5-dihydro-2H-1,4-benzoxazepin-7-ylboronic acid

To a stirred mixture of tert-butyl 7-bromo-9-chloro-3,5-dihydro-2H-1,4-benzoxazepine-4-carboxylate (1 g, 2.757 mmol, 1 equiv) and Triisopropyl borate (674 mg, 3.584 mmol, 1.3 equiv) in THF (20 mL) were added 2.5 M n-Butyllithium in hexane (1.4 mL) in portions at −78° C. under nitrogen atmosphere. The mixture was stirred at −78° C. for 1 h. Desired product could be detected by LCMS. H₂O (10 mL) was added to the mixture. The mixture was stirred at room temperature for 12 h. The resulting mixture was extracted with EtOAc (50 mL×3) and H₂O (50 mL). The combined organic layers were washed with brine (2×30 mL), dried over anhydrous Na₂SO₄. After filtration, the filtrate was concentrated under reduced pressure. The residue was purified by trituration with Et₂O (3 mL) and PE (5 mL). The precipitated solids were collected by filtration and washed with PE (3×10 mL) to afford 4-(tert-butoxycarbonyl)-9-chloro-3,5-dihydro-2H-1,4-benzoxazepin-7-ylboronic acid (270 mg, 20.92%) as a grey solid.

### tert-butyl 9-chloro-7-(5-fluoro-1,3-benzodiazol-1-yl)-3,5-dihydro-2H-1,4-benzoxazepine-4-carboxylate

To the mixture of 4-(tert-butoxycarbonyl)-9-chloro-3,5-dihydro-2H-1,4-benzoxazepin-7-ylboronic acid (150 mg, 0.458 mmol, 1 equiv) 5-fluoro-1H-1,3-benzodiazole (62 mg, 0.458 mmol, 1 equiv) and Cupric acetate (166 mg, 0.916 mmol, 2 equiv) in CH₂Cl₂ (5 mL) was added pyridine (10 mg, 0.124 mmol, 4 equiv). The mixture was stirred at room temperature for 16 h under 02 atmosphere. Desired product could be detected by LCMS. The mixture was added with H₂O (10 mL) and extracted by DCM (5 mL×3). The organic layer was combined, washed by brine (10 mL), dried over Na₂SO₄, filtered and concentrated. The residue was purified by silica gel column chromatography, eluted with CH₂Cl₂/MeOH (10:1) to afford tert-butyl 9-chloro-7-(5-fluoro-1,3-benzodiazol-1-yl)-3,5-dihydro-2H-1,4-benzoxazepine-4-carboxylate (50 mg, 26.13%) as a yellow oil.

### 9-chloro-7-(5-fluoro-1,3-benzodiazol-1-yl)-2,3,4,5-tetrahydro-1,4-benzoxazepine

To the mixture of tert-butyl 9-chloro-7-(5-fluoro-1,3-benzodiazol-1-yl)-3,5-dihydro-2H-1,4-benzoxazepine-4-carboxylate (100 mg, 0.239 mmol, 1 equiv) in CH₂Cl₂ (5.00 mL) was added TFA (1.00 mL). The mixture was stirred at room temperature for 1 h. Desired product could be detected by LCMS. The mixture was concentrated under reduced pressure to afford 9-chloro-7-(5-fluoro-1,3-benzodiazol-1-yl)-2,3,4,5-tetrahydro-1,4-benzoxazepine (60 mg, 78.91%) as yellow oil.

### Preparation of tert-butyl 6-bromoimidazo[4,5-b]pyridine-3-carboxylate

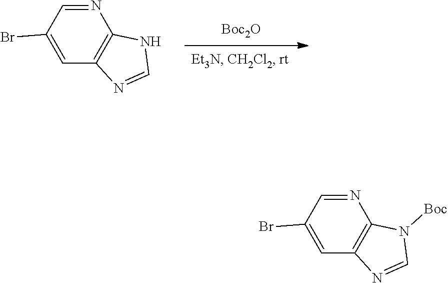 [SMILES: "Boc2O" ; Brc1cnc2[nH]cnc2c1 ; CC(C)(C)OC(=O)n1cnc2cc(Br)cnc21]

### tert-butyl 6-bromoimidazo[4,5-b]pyridine-3-carboxylate

To the mixture of 6-bromo-3H-imidazo[4,5-b]pyridine (200 mg, 1.010 mmol, 1 equiv) in THF (5 mL) was added Boc₂O (264 mg, 1.212 mmol, 1.2 equiv) and TEA (204 mg, 2.020 mmol, 2 equiv). The mixture was stirred at room temperature for 16 h. Desired product could be detected by LCMS. The mixture was concentrated under reduced pressure to afford tert-butyl 6-bromoimidazo[4,5-b]pyridine-3-carboxylate (100 mg, 33.21%) as white solid.

### Preparation of tert-butyl 9-chloro-7-(5-fluoroindol-1-yl)-4-[(tributylstannyl)methyl]-3,5-dihydro-2H-1,4-benzoxazepine

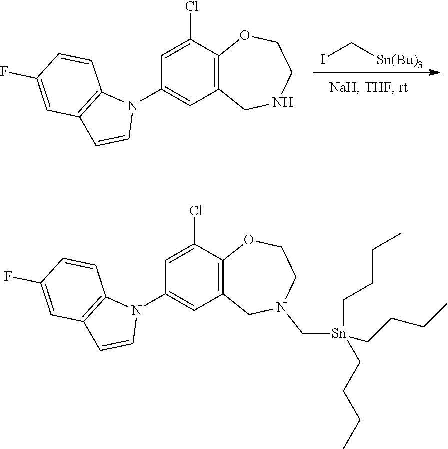

To a stirred mixture of 9-chloro-7-(5-fluoroindol-1-yl)-2,3,4,5-tetrahydro-1,4-benzoxazepine (100 mg, 0.316 mmol, 1 equiv) in THF (2 mL) was added NaH (38 mg, 1.579 mmol, 5.00 equiv) in portions at 0° C. for 30 min. To the above mixture was added tributyl(iodomethyl)stannane (680 mg, 1.578 mmol, 5.00 equiv) in portions at room temperature. The resulting mixture was stirred for additional 24 h at room temperature. The resulting mixture was extracted with EtOAc (3×50 mL). The combined organic layers were washed with water (3×10 mL), dried over anhydrous Na₂SO₄. After filtration, the filtrate was concentrated under reduced pressure. The residue was purified by Prep-TLC (PE/EA 1:1) to afford 9-chloro-7-(5-fluoroindol-1-yl)-4-[(tributylstannyl)methyl]-3,5-dihydro-2H-1,4-benzoxazepine (120 mg, 50.16%) as a white oil.

### Preparation of 9-chloro-7-(6,7-difluoro-1-benzofuran-3-yl)-2,3,4,5-tetrahydro-1,4-benzoxazepine

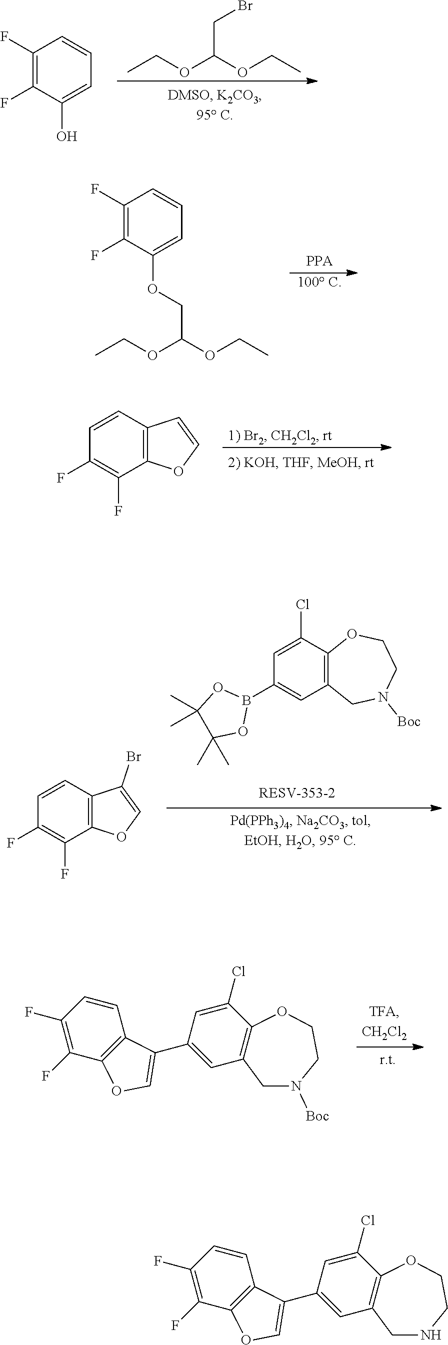

### 1-(2,2-diethoxyethoxy)-2,3-difluorobenzene

To the mixture of 2,3-difluorophenol (5 g, 38.434 mmol, 1 equiv) in DMSO (200 mL) was added 2-bromo-1,1-diethoxyethane (8.33 g, 42.277 mmol, 1.1 equiv) and K₂CO₃ (7.97 g, 57.651 mmol, 1.5 equiv). The mixture was stirred at 95° C. for 16 h. The mixture was diluted with EtOAc (300 mL) and filtered. The filtrate was washed by H₂O (500 mL), dried over Na₂SO₄, filtered and concentrated to afford 1-(2,2-diethoxyethoxy)-2,3-difluorobenzene (8 g, 84.53%) as yellow oil.

### 6,7-difluoro-1-benzofuran

To the mixture of 1-(2,2-diethoxyethoxy)-2,3-difluorobenzene (5 g, 20.304 mmol, 1 equiv) in toluene (100 mL) was added Polyphosphoric acid (9.63 g, 98.219 mmol, 4.84 equiv). The mixture was stirred at 100° C. for 6 h. The mixture was cooled to room temperature. The mixture was poured to H₂O (300 mL). The H₂O layer was extracted by EtOAc (200 mL×3). The organic layer was combined, washed by brine (500 mL), dried over Na₂SO₄, filtered and concentrated. The residue was purified by silica gel column chromatography, eluted with PE/EA (10:1) to afford 6,7-difluoro-1-benzofuran (500 mg, 15.98%) as a yellow oil.

### 3-bromo-6,7-difluoro-1-benzofuran

To the mixture of 6,7-difluoro-1-benzofuran (100 mg, 0.649 mmol, 1 equiv) in CH₂Cl₂ (3 mL) was added Br₂ (114 mg, 0.714 mmol, 1.1 equiv) at 0° C. The mixture was stirred at room temperature for 2 h. The reaction was monitored by TLC. The mixture was quenched by sat. aq. Na₂S₂O₃ (10 mL) and extracted by CH₂Cl₂ (5 mL×3). The organic layer was washed by brine (10 mL), dried over MgSO4, filtered and concentrated. The residue was dissolved in THF (2 mL). To the mixture was added KOH (36 mg, 0.649 mmol, 1 equiv) in MeOH (1 mL). The mixture was stirred at room temperature for 1 h. The mixture was added with H₂O (10 mL) and extracted by DCM (5 mL×3). The organic layer was washed by brine (10 mL), dried over MgSO₄, filtered and concentrated. The residue was purified by Prep-TLC (PE) to afford 3-bromo-6,7-difluoro-1-benzofuran (50 mg, 33.07%) as a white solid.

### tert-butyl 9-chloro-7-(6,7-difluoro-1-benzofuran-3-yl)-3,5-dihydro-2H-1,4-benzoxazepine-4-carboxylate

To the mixture of tert-butyl 9-chloro-7-(4,4,5,5-tetramethyl-1,3,2-dioxaborolan-2-yl)-3,5-dihydro-2H-1,4-benzoxazepine-4-carboxylate (40 mg, 0.098 mmol, 1 equiv) and 3-bromo-6,7-difluoro-1-benzofuran (34 mg, 0.147 mmol, 1.5 equiv) in toluene (1 mL) was added EtOH (0.5 mL), H₂O (0.5 mL), Na₂CO3 (20 mg, 0.196 mmol, 2 equiv), Pd(PPh₃)₄ (11 mg, 0.010 mmol, 0.1 equiv). The mixture was stirred at 95° C. for 16 h. Desired product could be detected by LCMS. The mixture was cooled to room temperature. The H₂O layer was extracted by toluene (3 mL×3). The organic layer was combined, washed by brine (5 mL), dried over Na₂SO₄, filtered and concentrated. The residue was purified by Prep-TLC (PE) to afford tert-butyl 9-chloro-7-(6,7-difluoro-1-benzofuran-3-yl)-3,5-dihydro-2H-1,4-benzoxazepine-4-carboxylate (20 mg, 47.00%) as a yellow oil.

### 9-chloro-7-(6,7-difluoro-1-benzofuran-3-yl)-2,3,4,5-tetrahydro-1,4-benzoxazepine

To the mixture of tert-butyl 9-chloro-7-(6,7-difluoro-1-benzofuran-3-yl)-3,5-dihydro-2H-1,4-benzoxazepine-4-carboxylate (30 mg, 0.069 mmol, 1 equiv) in CH₂Cl₂ (1 mL) was added TFA (0.2 mL). The mixture was stirred at room temperature for 1 h. Desired product could be detected by TLC. The mixture was concentrated under reduced pressure to afford 9-chloro-7-(6,7-difluoro-1-benzofuran-3-yl)-2,3,4,5-tetrahydro-1,4-benzoxazepine (20 mg, 86.55%) as a white solid.

### Biological Activity

### Example B1: EP2 Potency Assay

Compounds of the present disclosure are EP2 antagonists with half-max inhibitory concentration (IC₅₀) values below 25 μM. Compound potency was measured using a cAMP TR-FRET assay.

CHO-K1 cells (ATCC) were seeded at a density of 9.75×105 in 6 cm plates, and then on the following day cell media was changed to Opti-Mem I reduced serum media (Gibco), and transfected with a plasmid for expression of the EP2 receptor (the target receptor of interest), using the FuGENE 6 Transfection reagent (Promega). After 6 hours incubation, the cell media was replaced with F12 medium supplemented with 10% FBS and 100 U/ml Pen-Strep. 24 hours after transfection, cells were harvested and seeded at a density of 3000 cells/well in a 384 well plate to perform the cAMP assay, using the LANCE Ultra cAMP assay kit (PerkinElmer).

For each test compound of interest, 10 nl/well of serially diluted test compound was added to each well, resulting in a range of 10 serially diluted compound concentrations from 10000 nM to 0.038 nM, with duplicate wells for each concentration. Plates were then centrifuged at 1000 rpm for 1 min, agitated at 600 rpm at R.T. for 2 min, and incubated at 25° C. for 5 min. The reference agonist, prostaglandin E2 (MCE), was added to each well at the appropriate concentration to reach its EC80 value. Plates were then centrifuged at 1000 rpm for 1 min, agitated at 600 rpm at R.T. for 2 min, and incubated at 25° C. for 30 min.

To measure levels of cAMP, 5 μl/well of Eu-cAMP working solution and 5 μl/well of Ulight-anti-cAMP working solution were added to each well, and the plate was centrifuged at 1000 rpm for 1 min, agitated at 600 rpm at R.T. for 2 min, and incubated at 25° C. for 15 min. Levels of TR-FRET fluorescence were measured in each well using an EnVision microplate reader (excitation wavelength=337 nm and emission wavelength=615 and 665 nm). A dose-response curve was prepared by plotting percent inhibition for each compound concentration, and then IC₅₀ was calculated by fitting a curve to the plotted values and extrapolating the IC₅₀ concentration. Exemplary compounds disclosed herein exhibit potent EP2 antagonist activity. IC50 results are provided in Tables (IV) and (V) for selected compounds, wherein “--” indicates that the data are not available. Potency data is grouped into categories of AA (IC₅₀<100 nM); A (IC₅₀=100 to 500 nM); B (IC₅₀=500 nM to 1 M); C (IC₅₀=1 μM to 5 μM); and D (IC₅₀>5 μM).

Table (IV)

| ID | | |
|---|---|---|
| No. | Structure | IC₅₀ (nM) |
| I-1 | 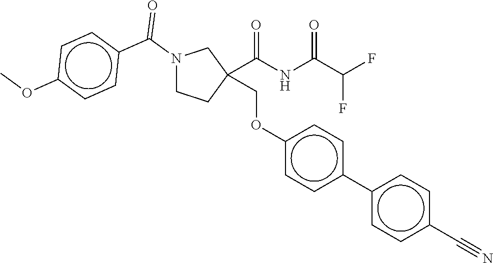 | — |
| I-2 | 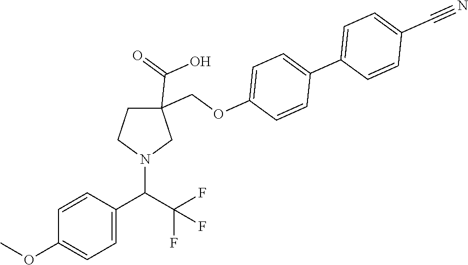 [SMILES: COc1ccc(C(N2CCC(COc3ccc(-c4ccc(C#N)cc4)cc3)(C(=O)O)C2)C(F)(F)F)cc1] | D |
| I-3 | 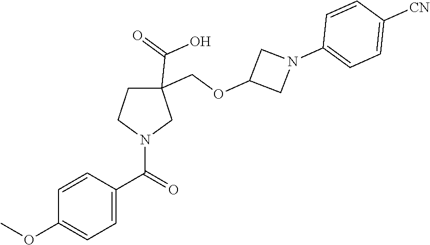 [SMILES: COc1ccc(C(=O)N2CCC(COC3CN(c4ccc(C#N)cc4)C3)(C(=O)O)C2)cc1] | — |
| I-4 | 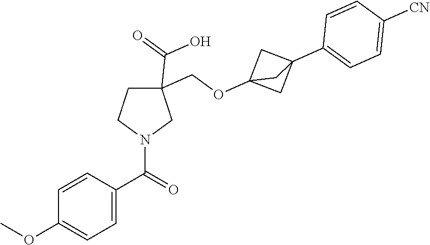 [SMILES: COc1ccc(C(=O)N2CCC(COC34CC(c5ccc(C#N)cc5)(C3)C4)(C(=O)O)C2)cc1] | — |
| I-5 | 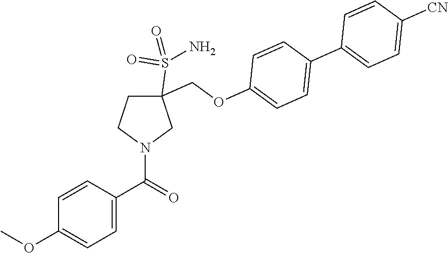 [SMILES: COc1ccc(C(=O)N2CCC(COc3ccc(-c4ccc(C#N)cc4)cc3)(S(N)(=O)=O)C2)cc1] | — |
| I-6 | 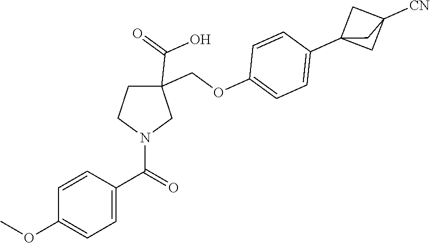 [SMILES: COc1ccc(C(=O)N2CCC(COc3ccc(C45CC(C#N)(C4)C5)cc3)(C(=O)O)C2)cc1] | AA |
| I-7 | 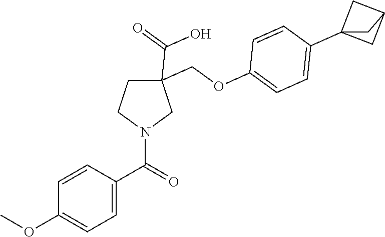 [SMILES: COc1ccc(C(=O)N2CCC(COc3ccc(C45CC(C4)C5)cc3)(C(=O)O)C2)cc1] | A |
| I-8 | 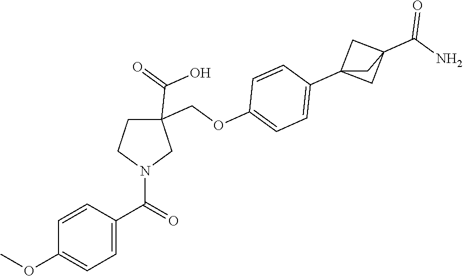 [SMILES: COc1ccc(C(=O)N2CCC(COc3ccc(C45CC(C(N)=O)(C4)C5)cc3)(C(=O)O)C2)cc1] | A |
| I-9 | 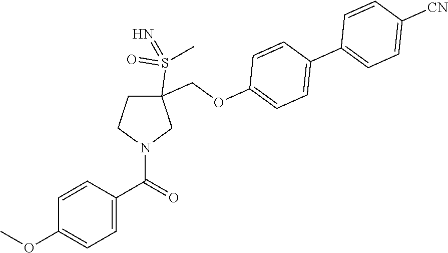 [SMILES: COc1ccc(C(=O)N2CCC(COc3ccc(-c4ccc(C#N)cc4)cc3)(S(C)(=N)=O)C2)cc1] | D |
| I-10 | 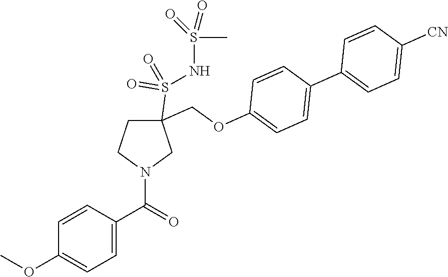 [SMILES: COc1ccc(C(=O)N2CCC(COc3ccc(-c4ccc(C#N)cc4)cc3)(S(=O)(=O)NS(C)(=O)=O)C2)cc1] | D |
| I-11 | 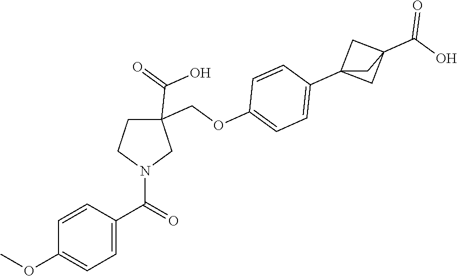 [SMILES: COc1ccc(C(=O)N2CCC(COc3ccc(C45CC(C(=O)O)(C4)C5)cc3)(C(=O)O)C2)cc1] | D |
| I-12 | 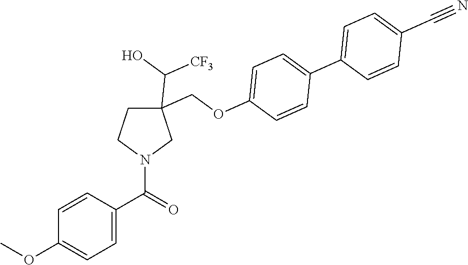 [SMILES: COc1ccc(C(=O)N2CCC(COc3ccc(-c4ccc(C#N)cc4)cc3)(C(O)C(F)(F)F)C2)cc1] | D |
| I-13 | 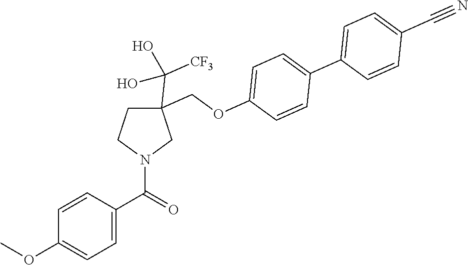 [SMILES: COc1ccc(C(=O)N2CCC(COc3ccc(-c4ccc(C#N)cc4)cc3)(C(O)(O)C(F)(F)F)C2)cc1] | D |
| I-14 | 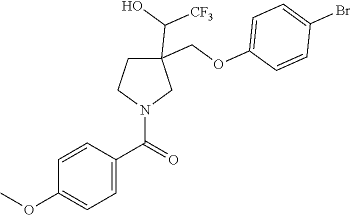 [SMILES: COc1ccc(C(=O)N2CCC(COc3ccc(Br)cc3)(C(O)C(F)(F)F)C2)cc1] | D |

Table (V)

| Cpd | | |
|---|---|---|
| No. | Structure | IC₅₀ (nM) |
| II-1 | 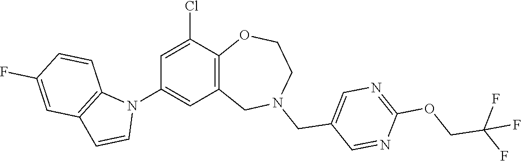 [SMILES: Fc1ccc2c(ccn2-c2cc(Cl)c3c(c2)CN(Cc2cnc(OCC(F)(F)F)nc2)CCO3)c1] | D |
| II-2 | 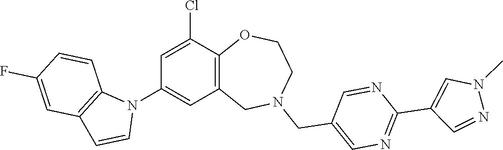 [SMILES: Cn1cc(-c2ncc(CN3CCOc4c(Cl)cc(-n5ccc6cc(F)ccc65)cc4C3)cn2)cn1] | C |
| II-3 | 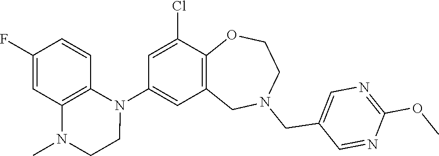 [SMILES: COc1ncc(CN2CCOc3c(Cl)cc(N4CCN(C)c5cc(F)ccc54)cc3C2)cn1] | D |
| II-4 | 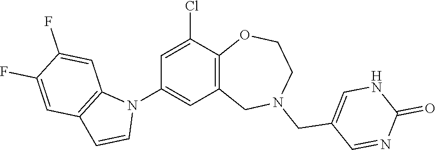 [SMILES: O=c1ncc(CN2CCOc3c(Cl)cc(-n4ccc5cc(F)c(F)cc54)cc3C2)c[nH]1] | C |
| II-5 | 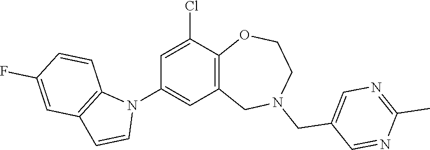 [SMILES: Cc1ncc(CN2CCOc3c(Cl)cc(-n4ccc5cc(F)ccc54)cc3C2)cn1] | A |
| II-6 | 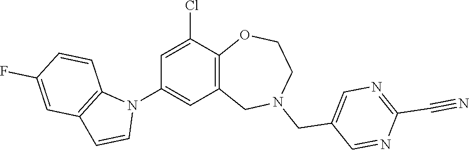 [SMILES: N#Cc1ncc(CN2CCOc3c(Cl)cc(-n4ccc5cc(F)ccc54)cc3C2)cn1] | B |
| II-7 | 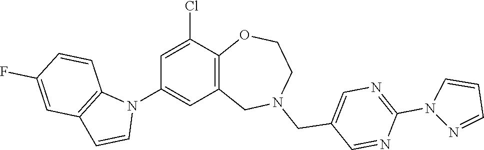 [SMILES: Fc1ccc2c(ccn2-c2cc(Cl)c3c(c2)CN(Cc2cnc(-n4cccn4)nc2)CCO3)c1] | C |
| II-8 | 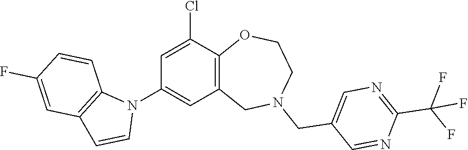 [SMILES: Fc1ccc2c(ccn2-c2cc(Cl)c3c(c2)CN(Cc2cnc(C(F)(F)F)nc2)CCO3)c1] | C |
| II-9 | 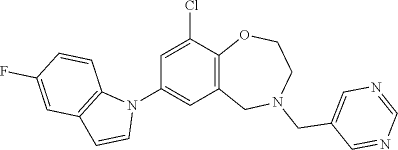 [SMILES: Fc1ccc2c(ccn2-c2cc(Cl)c3c(c2)CN(Cc2cncnc2)CCO3)c1] | A |
| II-10 | 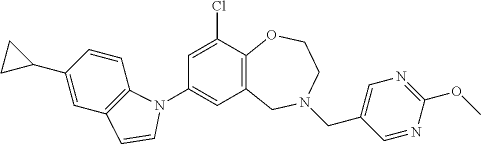 [SMILES: COc1ncc(CN2CCOc3c(Cl)cc(-n4ccc5cc(C6CC6)ccc54)cc3C2)cn1] | D |
| II-11 | 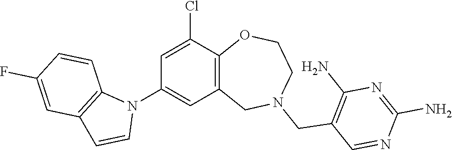 [SMILES: Nc1ncc(CN2CCOc3c(Cl)cc(-n4ccc5cc(F)ccc54)cc3C2)c(N)n1] | A |
| II-12 | 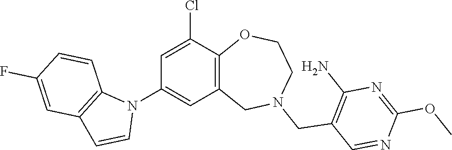 [SMILES: COc1ncc(CN2CCOc3c(Cl)cc(-n4ccc5cc(F)ccc54)cc3C2)c(N)n1] | A |
| II-13 | 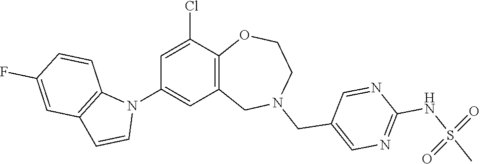 [SMILES: CS(=O)(=O)Nc1ncc(CN2CCOc3c(Cl)cc(-n4ccc5cc(F)ccc54)cc3C2)cn1] | C |
| II-14 | 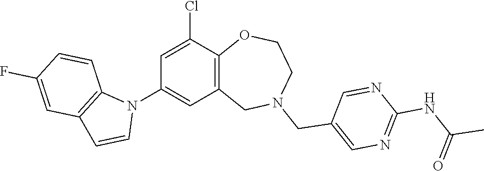 [SMILES: CC(=O)Nc1ncc(CN2CCOc3c(Cl)cc(-n4ccc5cc(F)ccc54)cc3C2)cn1] | C |
| II-15 | 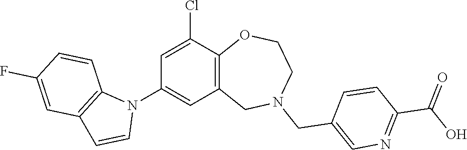 [SMILES: O=C(O)c1ccc(CN2CCOc3c(Cl)cc(-n4ccc5cc(F)ccc54)cc3C2)cn1] | AA |
| II-16 | 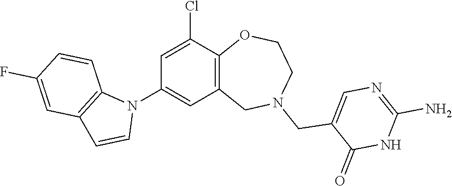 [SMILES: Nc1ncc(CN2CCOc3c(Cl)cc(-n4ccc5cc(F)ccc54)cc3C2)c(=O)[nH]1] | D |
| II-17 | 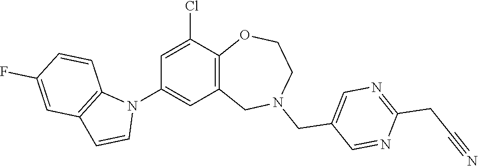 [SMILES: N#CCc1ncc(CN2CCOc3c(Cl)cc(-n4ccc5cc(F)ccc54)cc3C2)cn1] | A |
| II-18 | 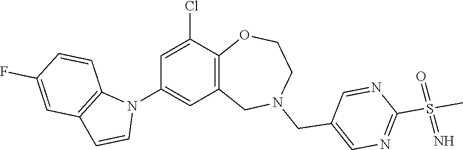 [SMILES: CS(=N)(=O)c1ncc(CN2CCOc3c(Cl)cc(-n4ccc5cc(F)ccc54)cc3C2)cn1] | 4 |
| II-19 | 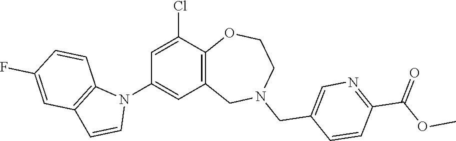 [SMILES: COC(=O)c1ccc(CN2CCOc3c(Cl)cc(-n4ccc5cc(F)ccc54)cc3C2)cn1] | A |
| II-20 | 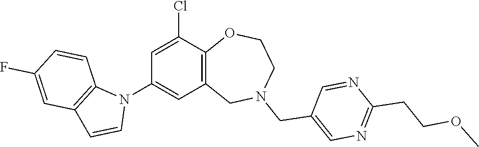 [SMILES: COCCc1ncc(CN2CCOc3c(Cl)cc(-n4ccc5cc(F)ccc54)cc3C2)cn1] | B |
| II-21 | 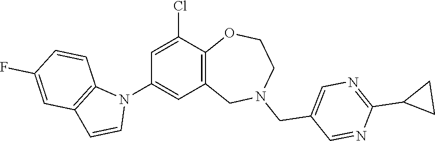 [SMILES: Fc1ccc2c(ccn2-c2cc(Cl)c3c(c2)CN(Cc2cnc(C4CC4)nc2)CCO3)c1] | B |
| II-22 | 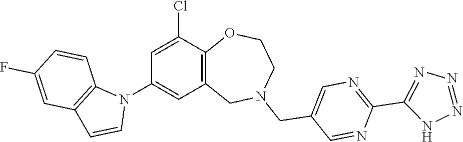 [SMILES: Fc1ccc2c(ccn2-c2cc(Cl)c3c(c2)CN(Cc2cnc(-c4nnn[nH]4)nc2)CCO3)c1] | AA |
| II-23 | 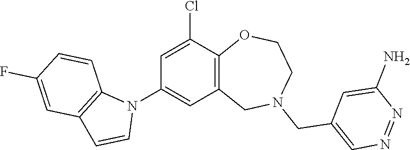 [SMILES: Nc1cc(CN2CCOc3c(Cl)cc(-n4ccc5cc(F)ccc54)cc3C2)cnn1] | B |
| II-24 | 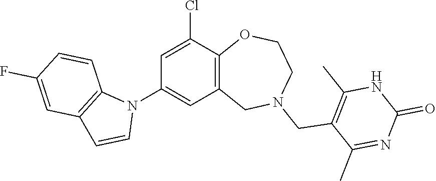 [SMILES: Cc1nc(=O)[nH]c(C)c1CN1CCOc2c(Cl)cc(-n3ccc4cc(F)ccc43)cc2C1] | B |
| II-25 | 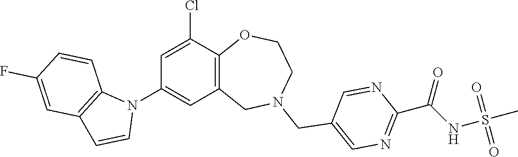 [SMILES: CS(=O)(=O)NC(=O)c1ncc(CN2CCOc3c(Cl)cc(-n4ccc5cc(F)ccc54)cc3C2)cn1] | AA |
| II-26 | 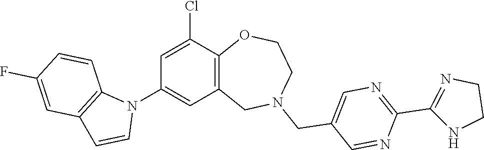 [SMILES: Fc1ccc2c(ccn2-c2cc(Cl)c3c(c2)CN(Cc2cnc(C4=NCCN4)nc2)CCO3)c1] | D |
| II-27 | 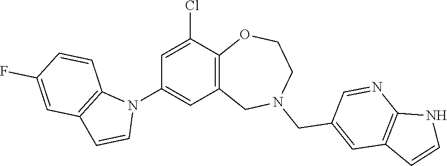 [SMILES: Fc1ccc2c(ccn2-c2cc(Cl)c3c(c2)CN(Cc2cnc4[nH]ccc4c2)CCO3)c1] | A |
| II-28 | 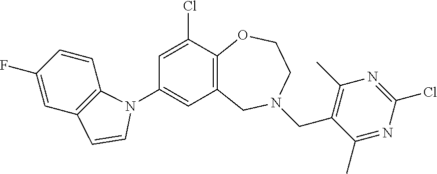 [SMILES: Cc1nc(Cl)nc(C)c1CN1CCOc2c(Cl)cc(-n3ccc4cc(F)ccc43)cc2C1] | C |
| II-29 | 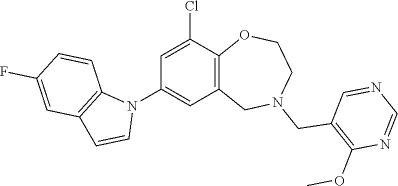 [SMILES: COc1ncncc1CN1CCOc2c(Cl)cc(-n3ccc4cc(F)ccc43)cc2C1] | B |
| II-30 | 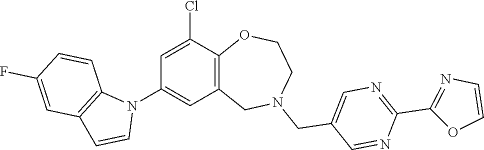 [SMILES: Fc1ccc2c(ccn2-c2cc(Cl)c3c(c2)CN(Cc2cnc(-c4ncco4)nc2)CCO3)c1] | 0 |
| II-31 | 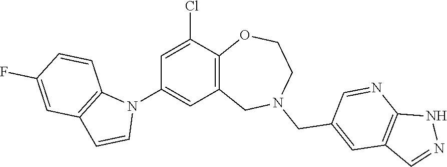 [SMILES: Fc1ccc2c(ccn2-c2cc(Cl)c3c(c2)CN(Cc2cnc4[nH]ncc4c2)CCO3)c1] | C |
| II-32 | 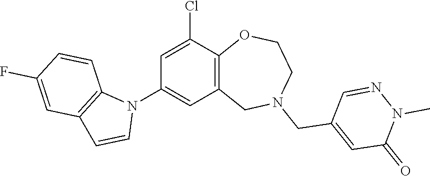 [SMILES: Cn1ncc(CN2CCOc3c(Cl)cc(-n4ccc5cc(F)ccc54)cc3C2)cc1=O] | A |
| II-33 | 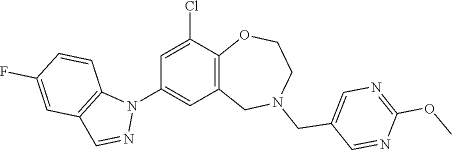 [SMILES: COc1ncc(CN2CCOc3c(Cl)cc(-n4ncc5cc(F)ccc54)cc3C2)cn1] | AA |
| II-34 | 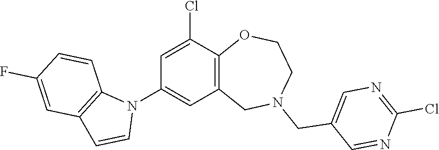 [SMILES: Fc1ccc2c(ccn2-c2cc(Cl)c3c(c2)CN(Cc2cnc(Cl)nc2)CCO3)c1] | — |
| II-35 | 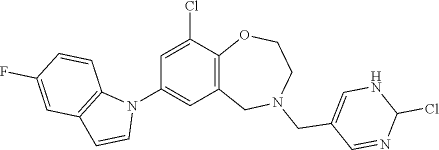 [SMILES: Fc1ccc2c(ccn2-c2cc(Cl)c3c(c2)CN(CC2=CNC(Cl)N=C2)CCO3)c1] | AA |
| II-36 | 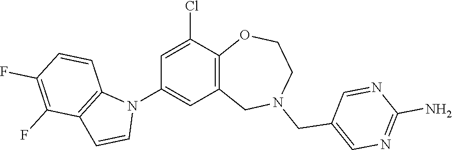 [SMILES: Nc1ncc(CN2CCOc3c(Cl)cc(-n4ccc5c(F)c(F)ccc54)cc3C2)cn1] | AA |
| II-37 | 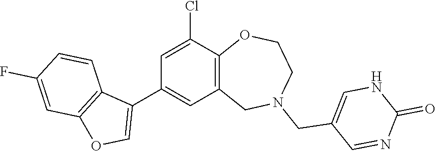 [SMILES: O=c1ncc(CN2CCOc3c(Cl)cc(-c4coc5cc(F)ccc45)cc3C2)c[nH]1] | B |
| II-38 | 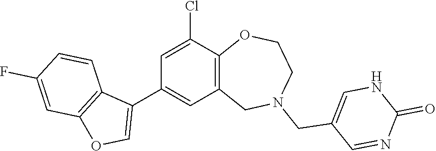 [SMILES: O=c1ncc(CN2CCOc3c(Cl)cc(-c4coc5cc(F)ccc45)cc3C2)c[nH]1] | AA |
| II-39 | 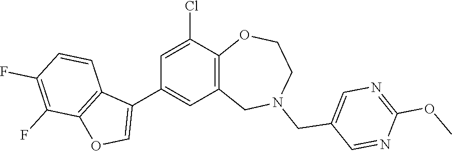 [SMILES: COc1ncc(CN2CCOc3c(Cl)cc(-c4coc5c(F)c(F)ccc45)cc3C2)cn1] | A |
| II-40 | 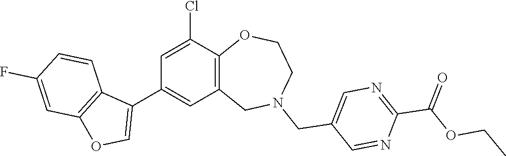 [SMILES: CCOC(=O)c1ncc(CN2CCOc3c(Cl)cc(-c4coc5cc(F)ccc45)cc3C2)cn1] | AA |
| II-41 | 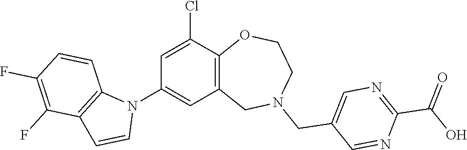 [SMILES: O=C(O)c1ncc(CN2CCOc3c(Cl)cc(-n4ccc5c(F)c(F)ccc54)cc3C2)cn1] | AA |
| II-42 | 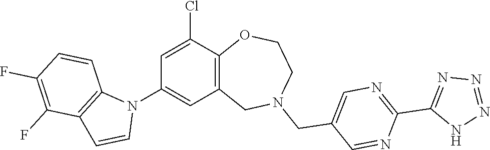 [SMILES: Fc1ccc2c(ccn2-c2cc(Cl)c3c(c2)CN(Cc2cnc(-c4nnn[nH]4)nc2)CCO3)c1F] | AA |
| II-43 | 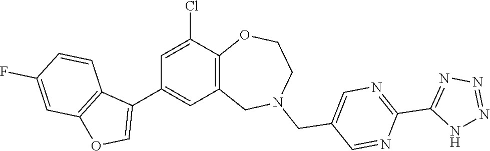 [SMILES: Fc1ccc2c(-c3cc(Cl)c4c(c3)CN(Cc3cnc(-c5nnn[nH]5)nc3)CCO4)coc2c1] | AA |
| II-44 | 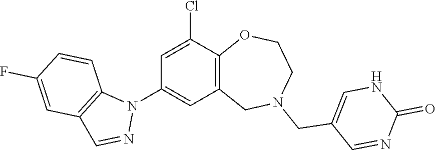 [SMILES: O=c1ncc(CN2CCOc3c(Cl)cc(-n4ncc5cc(F)ccc54)cc3C2)c[nH]1] | AA |
| II-45 | 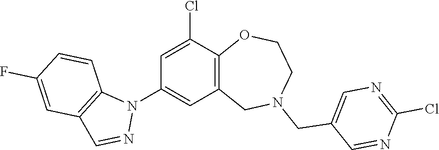 [SMILES: Fc1ccc2c(cnn2-c2cc(Cl)c3c(c2)CN(Cc2cnc(Cl)nc2)CCO3)c1] | -- |
| II-46 | 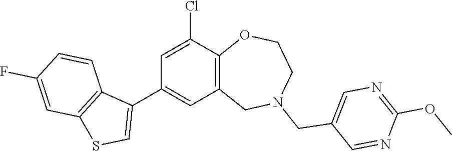 [SMILES: COc1ncc(CN2CCOc3c(Cl)cc(-c4csc5cc(F)ccc45)cc3C2)cn1] | A |
| II-47 | 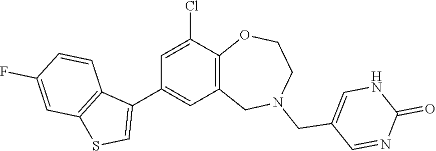 [SMILES: O=c1ncc(CN2CCOc3c(Cl)cc(-c4csc5cc(F)ccc45)cc3C2)c[nH]1] | AA |
| II-48 | 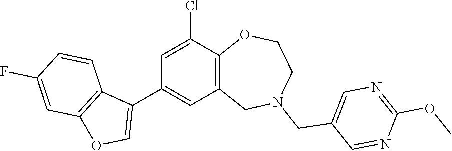 [SMILES: COc1ncc(CN2CCOc3c(Cl)cc(-c4coc5cc(F)ccc45)cc3C2)cn1] | AA |
| II-49 | 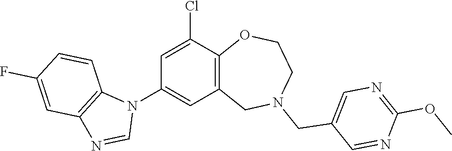 [SMILES: COc1ncc(CN2CCOc3c(Cl)cc(-n4cnc5cc(F)ccc54)cc3C2)cn1] | B |
| II-50 | 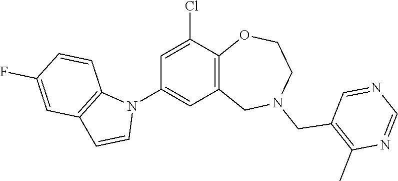 [SMILES: Cc1ncncc1CN1CCOc2c(Cl)cc(-n3ccc4cc(F)ccc43)cc2C1] | — |
| II-51 | 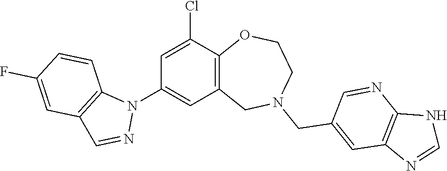 [SMILES: Fc1ccc2c(cnn2-c2cc(Cl)c3c(c2)CN(Cc2cnc4[nH]cnc4c2)CCO3)c1] | — |
| II-52 | 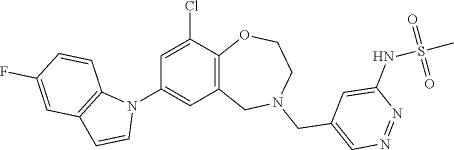 [SMILES: CS(=O)(=O)Nc1cc(CN2CCOc3c(Cl)cc(-n4ccc5cc(F)ccc54)cc3C2)cnn1] | AA |
| II-53 | 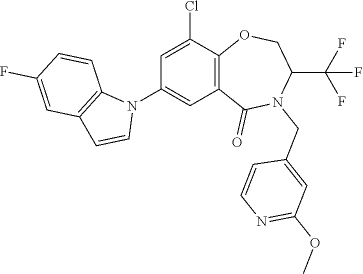 [SMILES: COc1cc(CN2C(=O)c3cc(-n4ccc5cc(F)ccc54)cc(Cl)c3OCC2C(F)(F)F)ccn1] | — |
| II-54 | 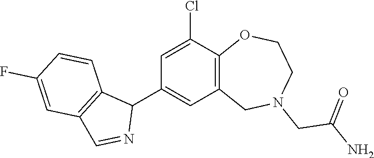 [SMILES: NC(=O)CN1CCOc2c(Cl)cc(C3N=Cc4cc(F)ccc43)cc2C1] | — |
| II-55 | 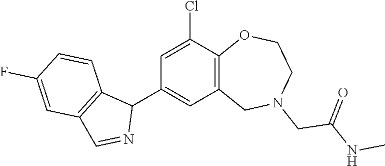 [SMILES: CNC(=O)CN1CCOc2c(Cl)cc(C3N=Cc4cc(F)ccc43)cc2C1] | — |
| II-56 | 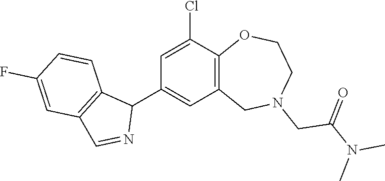 [SMILES: CN(C)C(=O)CN1CCOc2c(Cl)cc(C3N=Cc4cc(F)ccc43)cc2C1] | — |
| II-57 | 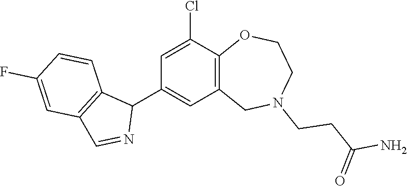 [SMILES: NC(=O)CCN1CCOc2c(Cl)cc(C3N=Cc4cc(F)ccc43)cc2C1] | — |
| II-58 | 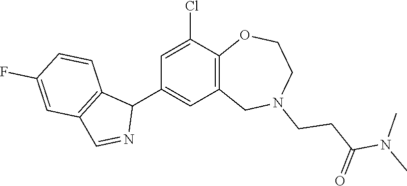 [SMILES: CN(C)C(=O)CCN1CCOc2c(Cl)cc(C3N=Cc4cc(F)ccc43)cc2C1] | — |
| | 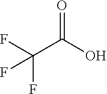 | |
| II-59 | 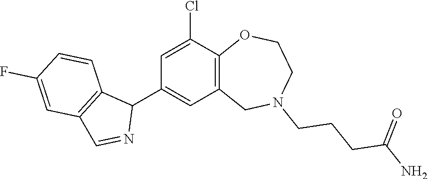 [SMILES: NC(=O)CCCN1CCOc2c(Cl)cc(C3N=Cc4cc(F)ccc43)cc2C1] | — |
| II-60 | 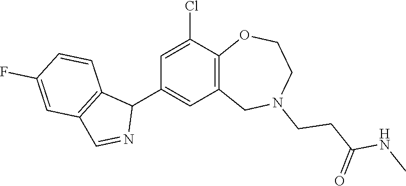 [SMILES: CNC(=O)CCN1CCOc2c(Cl)cc(C3N=Cc4cc(F)ccc43)cc2C1] | — |
| II-61 | 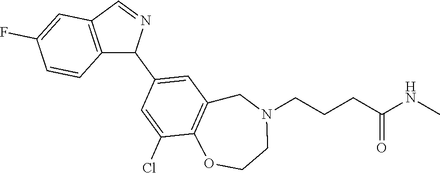 [SMILES: CNC(=O)CCCN1CCOc2c(Cl)cc(C3N=Cc4cc(F)ccc43)cc2C1] | — |
| II-62 | 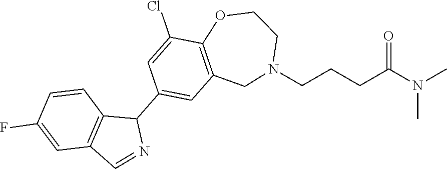 [SMILES: CN(C)C(=O)CCCN1CCOc2c(Cl)cc(C3N=Cc4cc(F)ccc43)cc2C1] | — |
| II-63 | 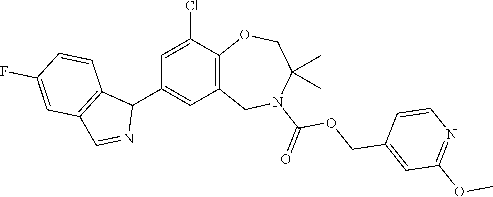 [SMILES: COc1cc(COC(=O)N2Cc3cc(C4N=Cc5cc(F)ccc54)cc(Cl)c3OCC2(C)C)ccn1] | — |
| II-64 | 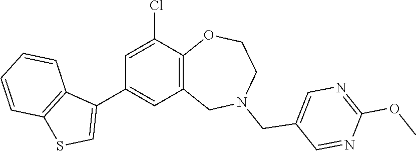 [SMILES: COc1ncc(CN2CCOc3c(Cl)cc(-c4csc5ccccc45)cc3C2)cn1] | — |
| II-65 | 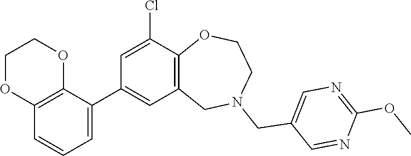 [SMILES: COc1ncc(CN2CCOc3c(Cl)cc(-c4cccc5c4OCCO5)cc3C2)cn1] | — |
| II-66 | 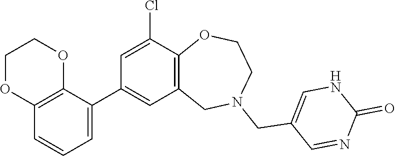 [SMILES: O=c1ncc(CN2CCOc3c(Cl)cc(-c4cccc5c4OCCO5)cc3C2)c[nH]1] | — |
| II-67 | 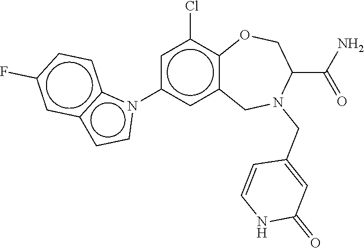 | — |
| II-68 | 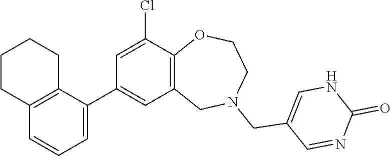 [SMILES: O=c1ncc(CN2CCOc3c(Cl)cc(-c4cccc5c4CCCC5)cc3C2)c[nH]1] | — |
| II-69 | 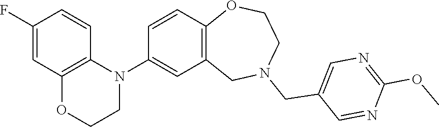 [SMILES: COc1ncc(CN2CCOc3ccc(N4CCOc5cc(F)ccc54)cc3C2)cn1] | — |
| II-70 | 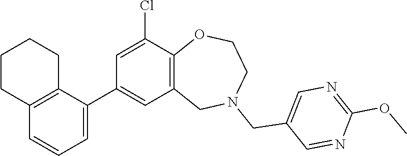 [SMILES: COc1ncc(CN2CCOc3c(Cl)cc(-c4cccc5c4CCCC5)cc3C2)cn1] | — |
| II-71 | 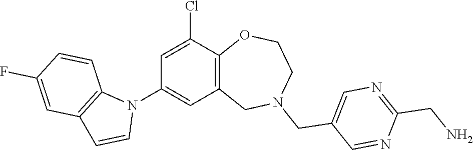 [SMILES: NCc1ncc(CN2CCOc3c(Cl)cc(-n4ccc5cc(F)ccc54)cc3C2)cn1] | — |
| II-72 | 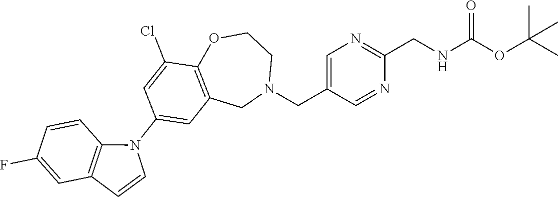 [SMILES: CC(C)(C)OC(=O)NCc1ncc(CN2CCOc3c(Cl)cc(-n4ccc5cc(F)ccc54)cc3C2)cn1] | — |
| II-73 | 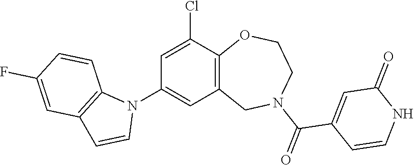 [SMILES: O=C(c1cc[nH]c(=O)c1)N1CCOc2c(Cl)cc(-n3ccc4cc(F)ccc43)cc2C1] | — |
| II-74 | 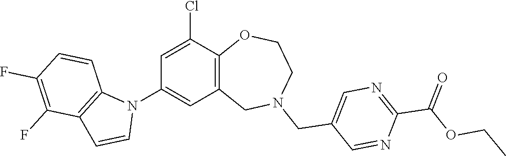 [SMILES: CCOC(=O)c1ncc(CN2CCOc3c(Cl)cc(-n4ccc5c(F)c(F)ccc54)cc3C2)cn1] | AA |
| II-75 | 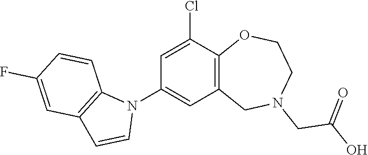 [SMILES: O=C(O)CN1CCOc2c(Cl)cc(-n3ccc4cc(F)ccc43)cc2C1] | — |
| II-76 | 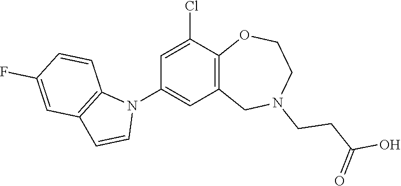 [SMILES: O=C(O)CCN1CCOc2c(Cl)cc(-n3ccc4cc(F)ccc43)cc2C1] | |
| II-77 | 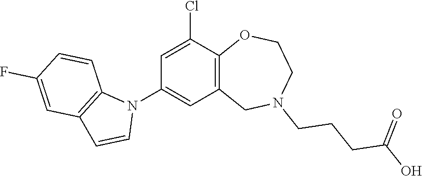 [SMILES: O=C(O)CCCN1CCOc2c(Cl)cc(-n3ccc4cc(F)ccc43)cc2C1] | — |
| II-78 | 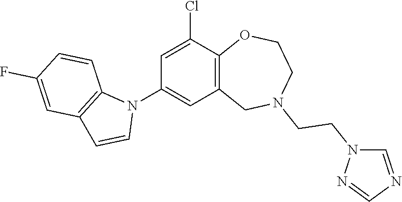 [SMILES: Fc1ccc2c(ccn2-c2cc(Cl)c3c(c2)CN(CCn2cncn2)CCO3)c1] | — |

### CERTAIN EMBODIMENTS

The present disclosure contemplates, among other things, the following numbered embodiments:
1. A compound having the formula

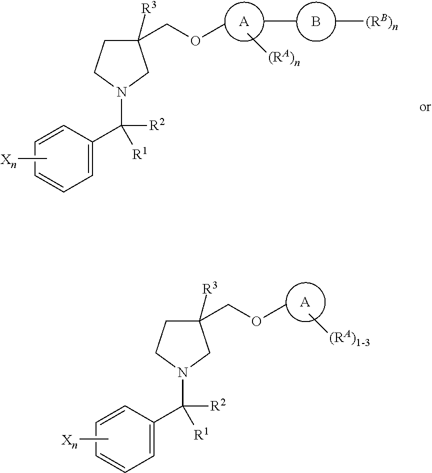

wherein
R¹ and R² are independently selected from R^{a}, —OR^{d}, —C₁₋₄ haloalkyl, or together R¹ and R² form an oxo, C₃₋₆ membered cycloalkyl or 3-6 membered cycloheteroalkyl;
R³ is —CH(R^{b})₂, —C(R^{b})₃, —S(O)₂R^{d}, —S(O)₂OR^{d}, —S(O)₂NR^{c}R^{c}, —S(O)(NR^{a})R^{d}, —S(O)₂N(R^{a})S(O)₂R^{a}, —S(O)₂N(R^{a})S(O)₂NR^{c}R^{c}, —C(O)R^{d}, —C(O)N(R^{a})C(O)R^{d}, —C(O)OR^{d}, —C(O)NR^{c}R^{c};
Ring A and Ring B independently are selected from phenyl and cycloalkyl;
X is selected from hydrogen and R^{b};
each R^{A} is independently selected from halogen; —CN, —C₁₋₄ alkyl, and —C₁₋₄ haloalkyl;
each R^{B} is independently selected from the group consisting of halogen, —CN, —C₁₋₄ alkyl, —C₁₋₄ haloalkyl, —C₁₋₄ aminoalkyl, —C₁₋₄ hydroxyalkyl, —C₁₋₄ methoxyalkyl, —(C₁₋₄ alkyl)O(C₁₋₄ alkyl), —C(O)(C₁₋₄ alkyl), —C(O)OH, —C(O)O(C₁₋₄ alkyl), —C(O)NR^{c}R^{c}, —NR^{c}R^{c}, —NH(C₃₋₆ cycloalkyl), —NH(C₃₋₆ heterocycloalkyl) and substituted or unsubstituted C₃₋₆ cycloalkyl;
each R^{a} is independently selected from the group consisting of hydrogen, (C₁₋₆) alkyl and (C₃₋₈) cycloalkyl;
each R^{b} is independently selected from the group consisting of —OR^{d}, —OCF₂H, —OCF₃, —NR^{c}R^{c}, halogen, —CF₃, —CN, —S(O)₂R^{d}, —S(O)₂OR^{d}, —S(O)₂NR^{c}R^{c}, —C(O)R^{d}, —C(O)OR^{d}, —C(O)NR^{c}R^{c},
each R^{c} is independently R^{a}, or, alternatively, two R^{c} are taken together with the nitrogen atom to which they are bonded to form a 5 to 8-membered cycloheteroalkyl or 5-membered heteroaryl, which may optionally include one or more of the same or different additional heteroatoms and which may optionally be substituted with one or more of the same or different —C(O)R^{a} and R^{a} groups;
each R^{d} is independently selected from hydrogen and (C₁₋₆) alkyl optionally substituted with 1, 2 or 3 groups selected from halogen and —O(C₁₋₄ alkyl); and
n is, for each occurrence, independently, 0 to 3;
provided that the compound is not a compound disclosed in PCT/US22/34903.
2. The compound of embodiment 1, wherein Ring A or Ring B is C₃₋₆ cycloalkyl.
3. The compound of embodiment 1, having the structure

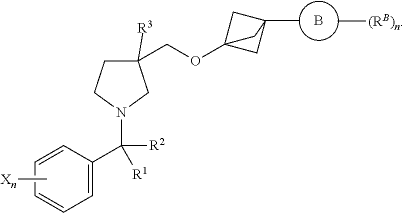

4. The compound of embodiment 1, having the structure

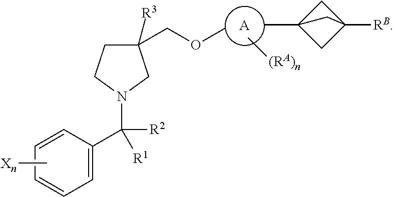

5. The compound of embodiment 1, having the structure

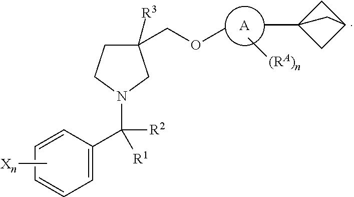

6. The compound of any one of embodiments 1-4, wherein R^{B} is —CN, —C(O)(C₁₋₄ alkyl), —C(O)OH, —C(O)O(C₁₋₄ alkyl) or —C(O)NR^{c}R^{c}.
7. The compound of any one of embodiments 1-5, wherein R³ is selected from —CH(R^{b})₂ and —C(R^{b})₃.
8. The compound of embodiment 7, wherein each R^{b} in R³ is independently selected from —OR^{d}, —OCF₂H, —OCF₃, and —CF₃.
9. The compound of embodiment 7, wherein R³ is —CH(OH)CF₃.
10. The compound of embodiment 7, wherein R³ is —C(OH)₂CF₃.
11. The compound of any one of embodiments 1-5, wherein R³ is selected from S(O)₂R^{d}, —S(O)(NR^{a})R^{d}, —S(O)₂OR^{d}, —S(O)₂NR^{c}R^{c}, —S(O)₂N(R^{a})S(O)₂R^{a} and —S(O)₂N(R^{a})S(O)₂NR^{c}R^{c}.
12. The compound of any one of embodiments 1-5, wherein R³ is selected from —C(O)R^{d}, —C(O)N(R^{a})C(O)R^{d}, —C(O)OR^{d} and —C(O)NR^{c}R^{c}.
13. The compound of any one of embodiments 1-5, wherein R³ is —COOH.
14. The compound of any one of embodiments 1-5, wherein R³ is S(O)₂NH₂.
15. The compound of any one of embodiments 1-5, wherein R³ is S(O)₂CH₃.
16. The compound of any one of embodiments 1-5, wherein R³ is —S(O)(NR^{a})R^{d}.
17. The compound of any one of embodiments 1-5, wherein R³ is —S(O)(NH)CH₃.
18. The compound of any one of embodiments 1-17, wherein R¹ and R² together form an oxo.
19. The compound of any one of embodiments 1-17, wherein one of R¹ and R² is hydrogen and the other is —CF₃.
20. The compound of any one of embodiments 1-19, wherein at least one R^{B} is —CN.
21. The compound of any one of embodiments 1-20, having the structure

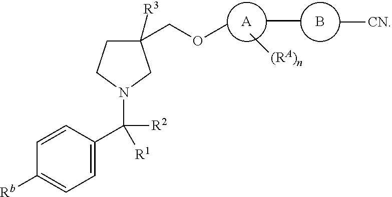

22. The compound of any one of embodiments 1-21, having the structure

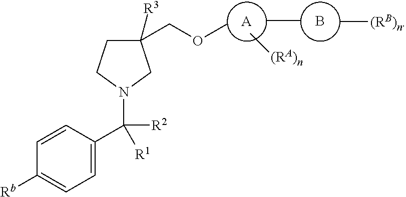

23. The compound of any one of embodiments 1-22, having the structure

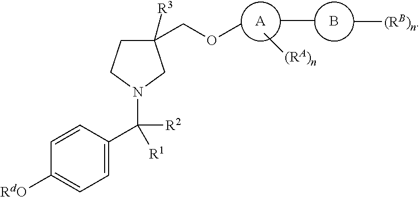

24. The compound of any one of embodiments 1-23, having the structure

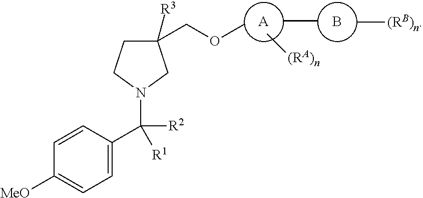

25. The compound of embodiment 1, having the structure

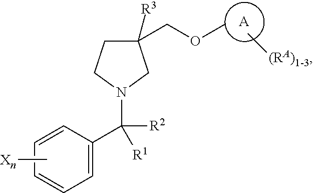

wherein each R^{A} independently is halo.
26. A compound that is:

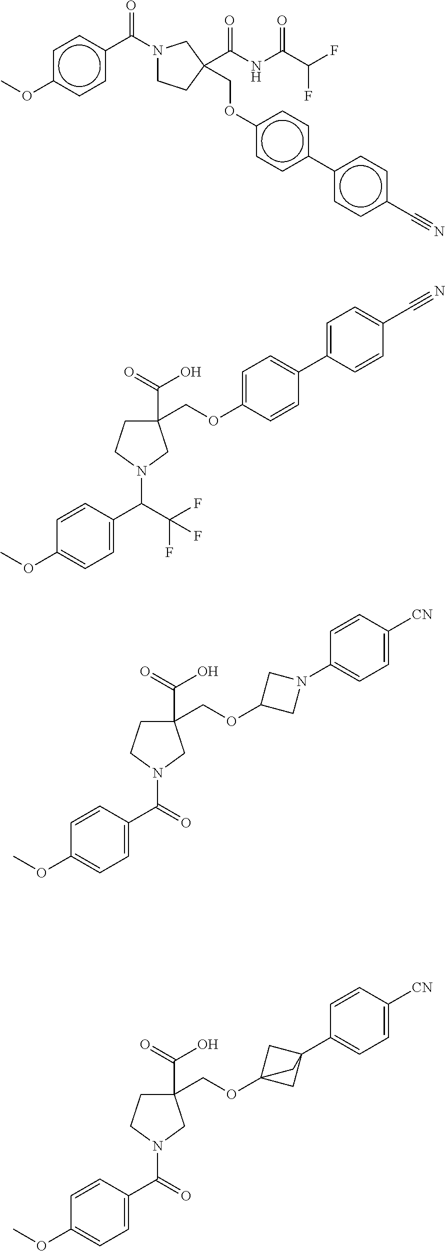

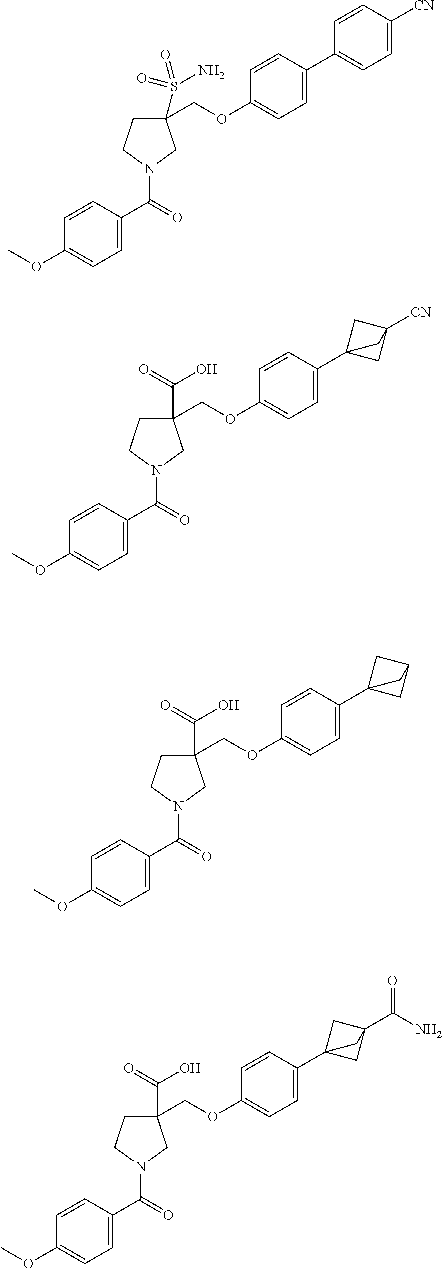 [SMILES: COc1ccc(C(=O)N2CCC(COc3ccc(-c4ccc(C#N)cc4)cc3)(S(N)(=O)=O)C2)cc1 ; COc1ccc(C(=O)N2CCC(COc3ccc(C45CC(C#N)(C4)C5)cc3)(C(=O)O)C2)cc1 ; COc1ccc(C(=O)N2CCC(COc3ccc(C45CC(C4)C5)cc3)(C(=O)O)C2)cc1 ; COc1ccc(C(=O)N2CCC(COc3ccc(C45CC(C(N)=O)(C4)C5)cc3)(C(=O)O)C2)cc1]

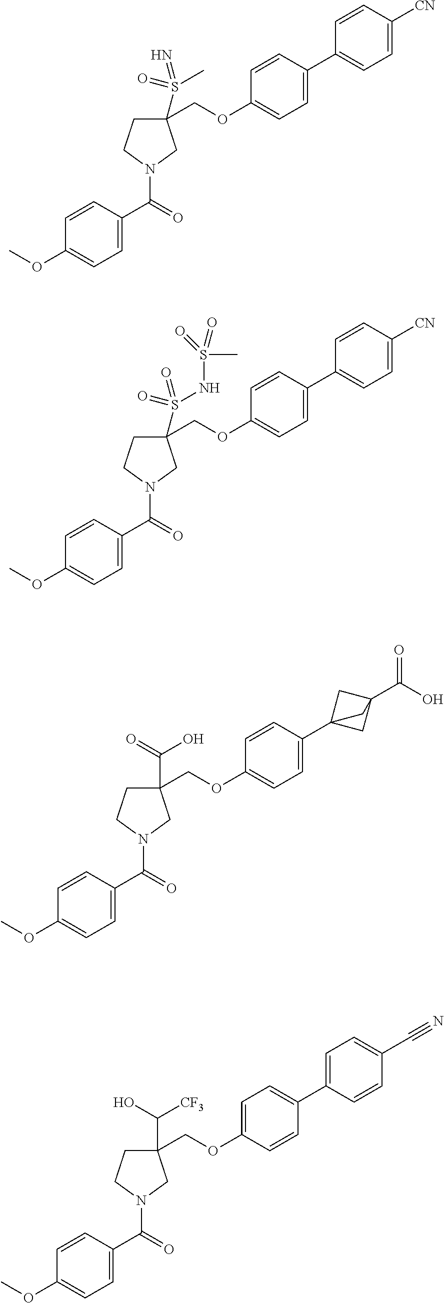 [SMILES: COc1ccc(C(=O)N2CCC(COc3ccc(-c4ccc(C#N)cc4)cc3)(S(C)(=N)=O)C2)cc1 ; COc1ccc(C(=O)N2CCC(COc3ccc(-c4ccc(C#N)cc4)cc3)(S(=O)(=O)NS(C)(=O)=O)C2)cc1 ; COc1ccc(C(=O)N2CCC(COc3ccc(C45CC(C(=O)O)(C4)C5)cc3)(C(=O)O)C2)cc1 ; COc1ccc(C(=O)N2CCC(COc3ccc(-c4ccc(C#N)cc4)cc3)(C(O)C(F)(F)F)C2)cc1]

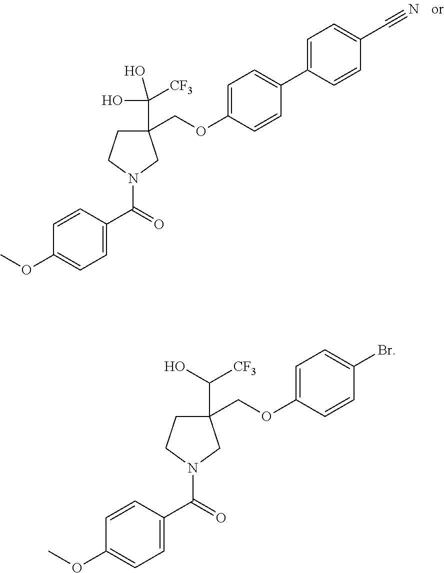 [SMILES: COc1ccc(C(=O)N2CCC(COc3ccc(-c4ccc(C#N)cc4)cc3)(C(O)(O)C(F)(F)F)C2)cc1 ; COc1ccc(C(=O)N2CCC(COc3ccc(Br)cc3)(C(O)C(F)(F)F)C2)cc1]

27. The compound of any one of embodiments 1-26 in the form of a pharmaceutically acceptable salt.
28. A pharmaceutical composition comprising a compound of any one of embodiments 1-27, and a pharmaceutically acceptable excipient.
29. A method of inhibiting the activity of the prostaglandin E2 receptor 2 (EP2) in a mammal comprising administering to the mammal an effective amount of a compound of any one of embodiments 1-27, or a pharmaceutical composition of embodiment 28.
30. A method of treating a disease or condition that would benefit from the modulation of prostaglandin E2 receptor 2 (EP2) activity comprising administering to the mammal an effective amount of a compound of any one of embodiments 1-27, or a pharmaceutical composition of embodiment 28.
31. A method for modulating prostaglandin E2 receptor 2 (EP2), comprising contacting the EP2 with a compound of any one of embodiments 1-27, or a pharmaceutical composition of embodiment 28.
32. The method of embodiment 31, wherein the contacting is in vitro.
33. The method of embodiment 31, wherein the contacting is in vivo.
34. A method for treating a disease or condition selected from neurological disorders, inflammatory disorders, autoimmune disorders, fibrotic disorders, allergic conditions, and combinations thereof, comprising administering to a subject in need thereof an effective amount of a compound of any one of embodiments 1-27, or a pharmaceutical composition of embodiment 28.
35. A method for treating a neurological disease selected from amyotrophic lateral sclerosis (ALS), epilepsy, dementia, Alzheimer's disease, concussion, delirium, chemotherapy-associated cognitive decline, radiation-associated cognitive decline, post-operative cognitive dysfunction including post-operative neurocognitive disorder, post-operative delirium, delayed neurocognitive disorder, and delayed neurocognitive recovery, vascular dementia, frontotemporal dementia, Lewy body dementia, pre-senile dementia (mild cognitive impairment or MCI), Binswanger's dementia (subcortical arteriosclerotic encephalopathy), HIV-associated dementia (including asymptomatic neurocognitive impairment (ANI), minor neurocognitive disorder (MND), and HIV-associated dementia (HAD) (also called AIDS dementia complex [ADC] or HIV encephalopathy), multiple system atrophy (MSA), spinocerebellar ataxias, Steel-Richardson-Olszewski disease (progressive supranuclear palsy), head injury, concussion, chronic traumatic encephalopathy, intracerebral hemorrhage, hematoma, diabetic retinopathy, macular degeneration, macular edema, glaucoma, multiple sclerosis, vasogenic edema including edema caused by treatment with antibody therapies (e.g. ARIA-E), migraine, Huntington's disease, ALS, and Parkinson's disease comprising administering to a subject in need thereof an effective amount of a compound of any one of embodiments 1-27, or a pharmaceutical composition of embodiment 28.
36. A method for treating an inflammatory or allergic disorder selected from systemic lupus erythematosus, rheumatoid arthritis, type I diabetes mellitus, inflammatory bowel disease, biliary cirrhosis, uveitis, multiple sclerosis, Crohn's disease, hyperimmunoglobulinemia D and periodic fever syndrome, cryopyrin-associated periodic syndromes, Schnitzler's syndrome, systemic juvenile idiopathic arthritis, adult's onset Still's disease, ulcerative colitis, necrotizing enterocolitis (NEC), peritonitis, gout, gout flares, gouty arthritis, bullous pemphigoid, sarcoidosis, psoriasis, autoimmune myositis, Wegener's granulomatosis, ichthyosis, Graves ophthalmopathy, asthma, atopic asthma, allergic asthma, atopic bronchial IgE-mediated asthma, non-atopic asthma, bronchial asthma, non-allergic asthma, essential asthma, true asthma, intrinsic asthma caused by pathophysiologic disturbances, essential asthma of unknown or unapparent cause, emphysematous asthma, exercise-induced asthma, emotion-induced asthma, extrinsic asthma caused by environmental factors, cold air induced asthma, occupational asthma, infective asthma caused by or associated with bacterial, fungal, protozoal, or viral infection, incipient asthma, wheezy infant syndrome, bronchiolitis, cough variant asthma or drug-induced asthma), allergic bronchopulmonary aspergillosis (ABPA), allergic rhinitis, perennial allergic rhinitis, perennial rhinitis, vasomotor rhinitis, post-nasal drip, purulent or non-purulent sinusitis, acute or chronic sinusitis, and ethmoid, frontal, maxillary, and sphenoid sinusitis, comprising administering to a subject in need thereof an effective amount of a compound of any one of embodiments 1-27, or a pharmaceutical composition of embodiment 28.
37. A method for treating an immune regulatory disorder selected from tissue or organ transplant, graft-versus-host diseases brought about by transplantation, rheumatoid arthritis, systemic lupus erythematosus, Hashimoto's thyroiditis, multiple sclerosis, systemic sclerosis, myasthenia gravis, type I diabetes, uveitis, posterior uveitis, allergic encephalomyelitis, glomerulonephritis, postinfectious autoimmune diseases, rheumatic fever and post-infectious glomerulonephritis, inflammatory and hyperproliferative skin diseases, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, celiac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, chronic lymphocytic leukemia, arteriosclerosis, atherosclerosis, aortitis syndrome, polyarteritis nodosa, myocardosis, scleroderma, Wegener's granuloma, Sjögren's syndrome, adiposis, eosinophilic fascitis, lesions of gingiva, periodontium, alveolar bone, substantia ossea dentis, glomerulonephritis, alopecia areata, male pattern alopecia or alopecia senilis by preventing epilation or providing hair germination and/or promoting hair generation and hair growth, vitiligo, muscular dystrophy, pyoderma and Sezary's syndrome, Addison's disease, ischemia-reperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA ballous dermatitis and cement dermatitis, gingivitis, periodontitis and sepsis, comprising administering to a subject in need thereof an effective amount of a compound of any one of embodiments 1-27, or a pharmaceutical composition of embodiment 28.
38. A method for treating a proliferative disorder selected from, solid tumors, carcinoma of the brain, kidney, liver, adrenal gland, bladder, breast, stomach, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins lymphoma, a mammary carcinoma, follicular carcinoma, undifferentiated carcinoma, papillary carcinoma, seminoma, melanoma, IL-1 driven disorders, ABC diffuse large B-cell lymphoma (DLBCL), Waldenström's macroglobulinemia, Hodgkin's lymphoma, primary cutaneous T-cell lymphoma, smoldering or indolent multiple myeloma, hematological malignancies, including leukemia, acute myeloid leukemia (AML), DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, myelodysplastic syndromes (MDS), myelofibrosis, polycythemia vera, Kaposi's sarcoma, splenic marginal zone lymphoma, multiple myeloma, plasmacytoma, intravascular large B-cell lymphoma, comprising administering to a subject in need thereof an effective amount of a compound of any one of embodiments 1-27, or a pharmaceutical composition of embodiment 28.
39. A method for treating a fibrotic condition selected from idiopathic pulmonary fibrosis, systemic sclerosis, uterine fibroids (leiomyomata), sclerodoma, autoimmune hepatitis, primary biliary cirrhosis, sclerosing cholangitis, partial liver resection, acute liver necrosis, necrosis caused by toxin, viral hepatitis, shock, or anoxia, B-virus hepatitis, non-A/non-B hepatitis, chronic hepatitis C virus (HCV) infection, cirrhosis, alcoholic liver disease, including alcoholic cirrhosis, non-alcoholic steatohepatitis (NASH), hepatic failure, fulminant hepatic failure, late-onset hepatic failure, “acute-on-chronic” liver failure, kidney disease, and chronic kidney disease, comprising administering to a subject in need thereof an effective amount of a compound of any one of embodiments 1-27, or a pharmaceutical composition of embodiment 28.
40. A method for treating cytokine release syndrome, comprising administering to a subject in need thereof an effective amount of a compound of any one of embodiments 1-27, or a pharmaceutical composition of embodiment 28.
41. A method for treating a condition selected from endometriosis, uterine fibroids (leiomyomata), menorrhagia, adenomyosis, primary and secondary dysmenorrhoea (including symptoms of dyspareunia, dyschexia and chronic pelvic pain), chronic pelvic pain syndrome, precocious puberty, cervical ripening, breast carcinoma, colon carcinoma, familial adenomatous polyposis, colorectal adenomas, endometrial carcinoma, prostate carcinoma, pulmonary carcinoma, testicular carcinoma, gastric carcinoma, macular degeneration, inflammatory and neuropathic pain conditions, migraine, headache, cluster headache, inflammation induced pain, cancer pain, polycystic kidney disease and polycystic ovarian syndrome, comprising administering to a subject in need thereof an effective amount of a compound of any one of embodiments 1-27, or a pharmaceutical composition of embodiment 28.
42. A method for treating a vascular disorder, comprising administering to a subject in need thereof an effective amount of a compound of any one of embodiments 1-27, or a pharmaceutical composition of embodiment 28.
43. The method of embodiment 42, wherein the vascular disorder is selected from head injury, stroke, aneurysm, vascular dementia, vascular cognitive impairment and dementia (VCID), cerebral small vessel disease, subcortical ischemic vascular disease, mixed dementia, and ischemic vascular damage.
44. A method for treating a condition selected from Alzheimer's disease, ALS, multiple sclerosis, Parkinson's disease and Huntington's disease, comprising administering to a subject in need thereof an effective amount of a compound of any one of embodiments 1-27, or a pharmaceutical composition of embodiment 28.

The present disclosure contemplates, among other things, the following numbered embodiments:
1. A compound having the formula

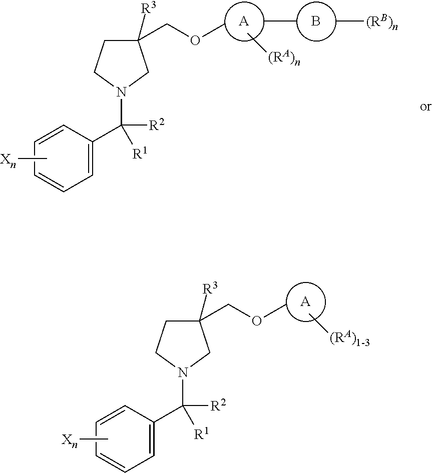

wherein
R¹ and R² are independently selected from R^{a}, —OR^{d}, —C₁₋₄ haloalkyl, or together R¹ and R² form an oxo, C₃₋₆ membered cycloalkyl or 3-6 membered cycloheteroalkyl;
R³ is —CH(R^{b})₂, —C(R^{b})₃, —S(O)₂R^{d}, —S(O)₂OR^{d}, —S(O)₂NR^{c}R^{c}, —S(O)(NR^{a})R^{d}, —S(O)₂N(R^{a})S(O)₂R^{a}, —S(O)₂N(R^{a})S(O)₂NR^{c}R^{c}, —C(O)R^{d}, —C(O)N(R^{a})C(O)R^{d}, —C(O)OR^{d}, —C(O)NR^{c}R^{c};
Ring A and Ring B independently are selected from phenyl and cycloalkyl;
X is selected from hydrogen and R^{b};
each R^{A} is independently selected from halogen; —CN, —C₁₋₄ alkyl, and —C₁₋₄ haloalkyl;
each R^{B} is independently selected from the group consisting of halogen, —CN, —C₁₋₄ alkyl, —C₁₋₄ haloalkyl, —C₁₋₄ aminoalkyl, —C₁₋₄ hydroxyalkyl, —C₁₋₄ methoxyalkyl, —(C₁₋₄ alkyl)O(C₁₋₄ alkyl), —C(O)(C₁₋₄ alkyl), —C(O)OH, —C(O)O(C₁₋₄ alkyl), —C(O)NR^{c}R^{c}, —NR^{c}R^{c}, —NH(C₃₋₆ cycloalkyl), —NH(C₃₋₆ heterocycloalkyl) and substituted or unsubstituted C₃₋₆ cycloalkyl;
each R^{a} is independently selected from the group consisting of hydrogen, (C₁₋₆) alkyl and (C₃₋₈) cycloalkyl;
each R^{b} is independently selected from the group consisting of —OR^{d}, —OCF₂H, —OCF₃, —NR^{c}R^{c}, halogen, —CF₃, —CN, —S(O)₂R^{d}, —S(O)₂OR^{d}, —S(O)₂NR^{c}R^{c}, —C(O)R^{d}, —C(O)OR^{d}, —C(O)NR^{c}R^{c},
each R^{c} is independently R^{a}, or, alternatively, two R^{c} are taken together with the nitrogen atom to which they are bonded to form a 5 to 8-membered cycloheteroalkyl or 5-membered heteroaryl, which may optionally include one or more of the same or different additional heteroatoms and which may optionally be substituted with one or more of the same or different —C(O)R^{a} and R^{a} groups;
each R^{d} is independently selected from hydrogen and (C₁₋₆) alkyl optionally substituted with 1, 2 or 3 groups selected from halogen and —O(C₁₋₄ alkyl); and
n is, for each occurrence, independently, 0 to 3;
provided that the compound is not a compound disclosed in PCT/US22/34903.
2. The compound of embodiment 1, wherein Ring A or Ring B, or both is bicyclic.
3. The compound of embodiment 1 or embodiment 2, wherein Ring A or Ring B, or both is bridged bicyclic.
4. The compound of embodiment 1 or embodiment 2, wherein Ring A or Ring B, or both is bridged bicyclic cycloalkyl.
5. The compound of embodiment 1 or embodiment 2, wherein Ring A is bridged cycloalkyl.
6. The compound of embodiment 1 or embodiment 2, wherein Ring B is bridged cycloalkyl.
7. The compound of embodiment 1, wherein Ring A or Ring B is C₃₋₆ cycloalkyl.
8. The compound of embodiment 1, having the structure

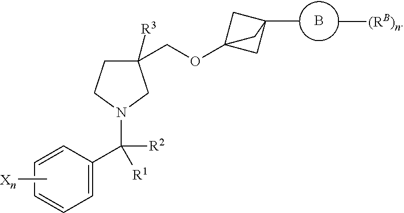

9. The compound of embodiment 1, having the structure

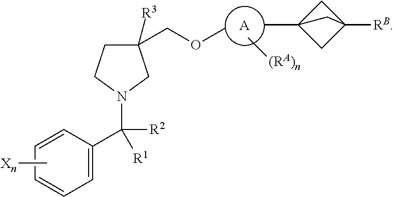

10. The compound of embodiment 1, having the structure

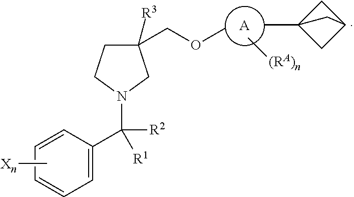

11. The compound of any one of embodiments 1-10, wherein R^{B} is —CN, —C(O)(C₁₋₄ alkyl), —C(O)OH, —C(O)O(C₁₋₄ alkyl) or —C(O)NR^{c}R^{c}.
12. The compound of any one of embodiments 1-10, wherein R³ is selected from —CH(R^{b})₂ and —C(R^{b})₃.
13. The compound of embodiment 12, wherein each R^{b} in R³ is independently selected from —OR^{d}, —OCF₂H, —OCF₃, and —CF₃.
14. The compound of embodiment 12, wherein R³ is —CH(OH)CF₃.
15. The compound of embodiment 12, wherein R³ is —C(OH)₂CF₃.
16. The compound of any one of embodiments 1-10, wherein R³ is selected from S(O)₂R^{d}, —S(O)(NR^{a})R^{d}, —S(O)₂OR^{d}, —S(O)₂NR^{c}R^{c}, —S(O)₂N(R^{a})S(O)₂R^{a} and —S(O)₂N(R^{a})S(O)₂NR^{c}R^{c}.
17. The compound of any one of embodiments 1-10, wherein R³ is selected from —C(O)R^{d}, —C(O)N(R^{a})C(O)R^{d}, —C(O)OR^{d} and —C(O)NR^{c}R^{c}.
18. The compound of any one of embodiments 1-10, wherein R³ is —COOH.
19. The compound of any one of embodiments 1-10, wherein R³ is S(O)₂NH₂.
20. The compound of any one of embodiments 1-10, wherein R³ is S(O)₂CH₃.
21. The compound of any one of embodiments 1-10, wherein R³ is —S(O)(NR^{a})R^{d}.
22. The compound of any one of embodiments 1-10, wherein R³ is —S(O)(NH)CH₃.
23. The compound of any one of embodiments 1-22, wherein R¹ and R² together form an oxo.
24. The compound of any one of embodiments 1-22, wherein one of R¹ and R² is hydrogen and the other is —CF₃.
25. The compound of any one of embodiments 1-24, wherein at least one R^{B} is —CN.
26. The compound of any one of embodiments 1-25, having the structure

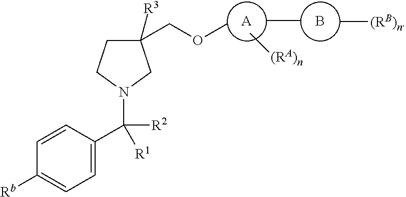

27. The compound of any one of embodiments 1-25, having the structure

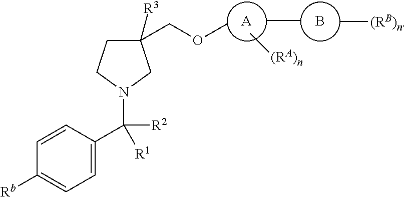

28. The compound of any one of embodiments 1-25, having the structure

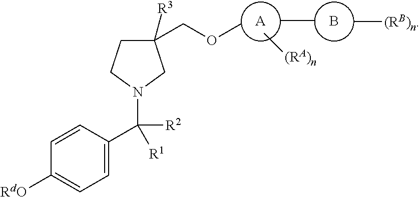

29. The compound of any one of embodiments 1-25, having the structure

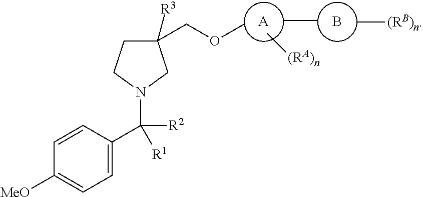

30. The compound of embodiment 1, having the structure

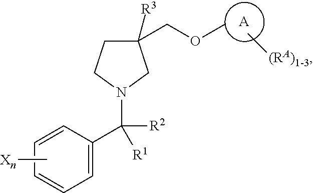

, wherein each R^{A} independently is halo.
31. The compound of any one of embodiments 1-30 in the form of a pharmaceutically acceptable salt.
32. A pharmaceutical composition comprising a compound of any one of embodiments 1-31, and a pharmaceutically acceptable excipient.
33. A method of inhibiting the activity of the prostaglandin E2 receptor 2 (EP2) in a mammal comprising administering to the mammal an effective amount of a compound of any one of embodiments 1-31, or a pharmaceutical composition of embodiment 32.
34. A method of treating a disease or condition that would benefit from the modulation of prostaglandin E2 receptor 2 (EP2) activity comprising administering to the mammal an effective amount of a compound of any one of embodiments 1-31, or a pharmaceutical composition of embodiment 32.
35. A method for modulating prostaglandin E2 receptor 2 (EP2), comprising contacting the EP2 with a compound of any one of embodiments 1-31, or a pharmaceutical composition of embodiment 32.
36. The method of embodiment 35, wherein the contacting is in vitro.
37. The method of embodiment 35, wherein the contacting is in vivo.
38. A method for treating a disease or condition selected from neurological disorders, inflammatory disorders, autoimmune disorders, fibrotic disorders, allergic conditions, and combinations thereof, comprising administering to a subject in need thereof an effective amount of a compound of any one of embodiments 1-31, or a pharmaceutical composition of embodiment 32.
39. A method for treating a neurological disease selected from amyotrophic lateral sclerosis (ALS), epilepsy, dementia, Alzheimer's disease, concussion, delirium, chemotherapy-associated cognitive decline, radiation-associated cognitive decline, post-operative cognitive dysfunction including post-operative neurocognitive disorder, post-operative delirium, delayed neurocognitive disorder, and delayed neurocognitive recovery, vascular dementia, frontotemporal dementia, Lewy body dementia, pre-senile dementia (mild cognitive impairment or MCI), Binswanger's dementia (subcortical arteriosclerotic encephalopathy), HIV-associated dementia (including asymptomatic neurocognitive impairment (ANI), minor neurocognitive disorder (MND), and HIV-associated dementia (HAD) (also called AIDS dementia complex [ADC] or HIV encephalopathy), multiple system atrophy (MSA), spinocerebellar ataxias, Steel-Richardson-Olszewski disease (progressive supranuclear palsy), head injury, concussion, chronic traumatic encephalopathy, intracerebral hemorrhage, hematoma, diabetic retinopathy, macular degeneration, macular edema, glaucoma, multiple sclerosis, vasogenic edema including edema caused by treatment with antibody therapies (e.g. ARIA-E), migraine, Huntington's disease, ALS, and Parkinson's disease comprising administering to a subject in need thereof an effective amount of a compound of any one of embodiments 1-31, or a pharmaceutical composition of embodiment 32.
40. A method for treating an inflammatory or allergic disorder selected from systemic lupus erythematosus, rheumatoid arthritis, type I diabetes mellitus, inflammatory bowel disease, biliary cirrhosis, uveitis, multiple sclerosis, Crohn's disease, hyperimmunoglobulinemia D and periodic fever syndrome, cryopyrin-associated periodic syndromes, Schnitzler's syndrome, systemic juvenile idiopathic arthritis, adult's onset Still's disease, ulcerative colitis, necrotizing enterocolitis (NEC), peritonitis, gout, gout flares, gouty arthritis, bullous pemphigoid, sarcoidosis, psoriasis, autoimmune myositis, Wegener's granulomatosis, ichthyosis, Graves ophthalmopathy, asthma, atopic asthma, allergic asthma, atopic bronchial IgE-mediated asthma, non-atopic asthma, bronchial asthma, non-allergic asthma, essential asthma, true asthma, intrinsic asthma caused by pathophysiologic disturbances, essential asthma of unknown or unapparent cause, emphysematous asthma, exercise-induced asthma, emotion-induced asthma, extrinsic asthma caused by environmental factors, cold air induced asthma, occupational asthma, infective asthma caused by or associated with bacterial, fungal, protozoal, or viral infection, incipient asthma, wheezy infant syndrome, bronchiolitis, cough variant asthma or drug-induced asthma), allergic bronchopulmonary aspergillosis (ABPA), allergic rhinitis, perennial allergic rhinitis, perennial rhinitis, vasomotor rhinitis, post-nasal drip, purulent or non-purulent sinusitis, acute or chronic sinusitis, and ethmoid, frontal, maxillary, and sphenoid sinusitis, comprising administering to a subject in need thereof an effective amount of a compound of any one of embodiments 1-31, or a pharmaceutical composition of embodiment 32.
41. A method for treating an immune regulatory disorder selected from tissue or organ transplant, graft-versus-host diseases brought about by transplantation, rheumatoid arthritis, systemic lupus erythematosus, Hashimoto's thyroiditis, multiple sclerosis, systemic sclerosis, myasthenia gravis, type I diabetes, uveitis, posterior uveitis, allergic encephalomyelitis, glomerulonephritis, postinfectious autoimmune diseases, rheumatic fever and post-infectious glomerulonephritis, inflammatory and hyperproliferative skin diseases, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, celiac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, chronic lymphocytic leukemia, arteriosclerosis, atherosclerosis, aortitis syndrome, polyarteritis nodosa, myocardosis, scleroderma, Wegener's granuloma, Sjögren's syndrome, adiposis, eosinophilic fascitis, lesions of gingiva, periodontium, alveolar bone, substantia ossea dentis, glomerulonephritis, alopecia areata, male pattern alopecia or alopecia senilis by preventing epilation or providing hair germination and/or promoting hair generation and hair growth, vitiligo, muscular dystrophy, pyoderma and Sezary's syndrome, Addison's disease, ischemia-reperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA ballous dermatitis and cement dermatitis, gingivitis, periodontitis and sepsis, comprising administering to a subject in need thereof an effective amount of a compound of any one of embodiments 1-31, or a pharmaceutical composition of embodiment 32.
42. A method for treating a proliferative disorder selected from, solid tumors, carcinoma of the brain, kidney, liver, adrenal gland, bladder, breast, stomach, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins lymphoma, a mammary carcinoma, follicular carcinoma, undifferentiated carcinoma, papillary carcinoma, seminoma, melanoma, IL-1 driven disorders, ABC diffuse large B-cell lymphoma (DLBCL), Waldenstrom's macroglobulinemia, Hodgkin's lymphoma, primary cutaneous T-cell lymphoma, smoldering or indolent multiple myeloma, hematological malignancies, including leukemia, acute myeloid leukemia (AML), DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, myelodysplastic syndromes (MDS), myelofibrosis, polycythemia vera, Kaposi's sarcoma, splenic marginal zone lymphoma, multiple myeloma, plasmacytoma, intravascular large B-cell lymphoma, comprising administering to a subject in need thereof an effective amount of a compound of any one of embodiments 1-31, or a pharmaceutical composition of embodiment 32.
43. A method for treating a fibrotic condition selected from idiopathic pulmonary fibrosis, systemic sclerosis, uterine fibroids (leiomyomata), sclerodoma, autoimmune hepatitis, primary biliary cirrhosis, sclerosing cholangitis, partial liver resection, acute liver necrosis, necrosis caused by toxin, viral hepatitis, shock, or anoxia, B-virus hepatitis, non-A/non-B hepatitis, chronic hepatitis C virus (HCV) infection, cirrhosis, alcoholic liver disease, including alcoholic cirrhosis, non-alcoholic steatohepatitis (NASH), hepatic failure, fulminant hepatic failure, late-onset hepatic failure, “acute-on-chronic” liver failure, kidney disease, and chronic kidney disease, comprising administering to a subject in need thereof an effective amount of a compound of any one of embodiments 1-31, or a pharmaceutical composition of embodiment 32.
44. A method for treating cytokine release syndrome, comprising administering to a subject in need thereof an effective amount of a compound of any one of embodiments 1-31, or a pharmaceutical composition of embodiment 32.
45. A method for treating a condition selected from endometriosis, uterine fibroids (leiomyomata), menorrhagia, adenomyosis, primary and secondary dysmenorrhoea (including symptoms of dyspareunia, dyschexia and chronic pelvic pain), chronic pelvic pain syndrome, precocious puberty, cervical ripening, breast carcinoma, colon carcinoma, familial adenomatous polyposis, colorectal adenomas, endometrial carcinoma, prostate carcinoma, pulmonary carcinoma, testicular carcinoma, gastric carcinoma, macular degeneration, inflammatory and neuropathic pain conditions, migraine, headache, cluster headache, inflammation induced pain, cancer pain, polycystic kidney disease and polycystic ovarian syndrome, comprising administering to a subject in need thereof an effective amount of a compound of any one of embodiments 1-31, or a pharmaceutical composition of embodiment 32.
46. A method for treating a vascular disorder, comprising administering to a subject in need thereof an effective amount of a compound of any one of embodiments 1-31, or a pharmaceutical composition of embodiment 32.
47. The method of embodiment 46, wherein the vascular disorder is selected from head injury, stroke, aneurysm, vascular dementia, vascular cognitive impairment and dementia (VCID), cerebral small vessel disease, subcortical ischemic vascular disease, mixed dementia, and ischemic vascular damage.
48. A method for treating a condition selected from Alzheimer's disease, ALS, multiple sclerosis, Parkinson's disease and Huntington's disease, comprising administering to a subject in need thereof an effective amount of a compound of any one of embodiments 1-31, or a pharmaceutical composition of embodiment 32.

The present disclosure contemplates, among other things, the following numbered embodiments:
1. A compound having the structure of Formula (III)

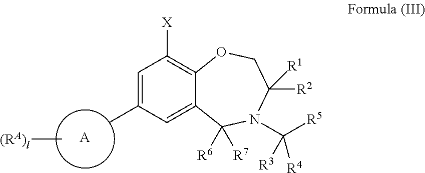

wherein
   Ring A is a bicyclic heterocycle having one or more nitrogen atoms; or Ring A is Ring A′;
   each R^{A} is selected from halogen; —CN, —C₁₋₄ alkyl, —C₁₋₄ haloalkyl, —(C₁₋₄ alkyl)O(C₁₋₄ alkyl), —C(O)OH, —C(O)O(C₁₋₄ alkyl), —C(O)NH₂, —C(O)NH(C₁₋₄ alkyl), —C(O)N(C₁₋₄ alkyl)₂, —NH₂, —NH(C₁₋₄ alkyl), —N(C₁₋₄ alkyl)₂, —OH, —O(C₁₋₄ alkyl), —O(C₁₋₄ haloalkyl), —S(C₁₋₄ alkyl), —SO₂C₁₋₄ alkyl, —SO₂NHC₁₋₄ alkyl, substituted or unsubstituted C₃₋₆ cycloalkyl, or substituted or unsubstituted 3- to 6-membered heterocyclyl; or two R^{A} taken together form a carbonyl;
   Ring A′ is selected from the group consisting of:

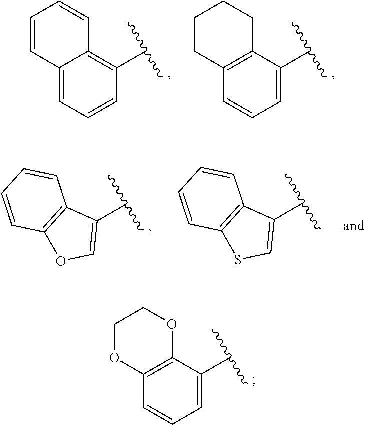 [SMILES: *c1cccc2ccccc12 ; *c1cccc2c1CCCC2 ; *c1coc2ccccc12 ; *c1csc2ccccc12 ; *c1cccc2c1OCCO2]

R¹ and R² are independently selected from hydrogen, —C₁₋₄ alkyl, —CN, —C(O)NR^{c}R^{c}, or together R¹ and R² form an oxo, C₃₋₆ membered cycloalkyl or 3-6 membered cycloheteroalkyl;
X is selected from hydrogen and halogen;
R³ and R⁴ are independently selected from hydrogen, —C₁₋₄ alkyl, together R³ and R⁴ form an oxo, or one of R³ and R⁴ form a double bond with R⁶ and the other of R³ and R⁴ is hydrogen or —C₁₋₄ alkyl;
R⁵ is selected from —(CH₂)ₘ—R^{b}, —(CHR^{a})ₘ—R^{b}, —O—(CH₂)ₘ—R^{b}, —O—(CHR^{a})ₘ—R^{b}, —C(O)NH—(CH₂)ₘ—R^{b}, —C(O)NH—(CHR^{a})ₘ—R^{b}, —NHC(O)OR^{d}, —NHC(O)NR^{c}R^{c};
R⁶ and R⁷ are independently selected from hydrogen, —C₁₋₄ alkyl, or together R⁶ and R⁷ form an oxo;
each R^{a} is independently selected from the group consisting of hydrogen, (C₁₋₆) alkyl and (C₃₋₈) cycloalkyl;
each R^{b} is independently selected from the group consisting of ═O, —OR^{d}, —OCF₂H, —OCF₃, —NR^{c}R^{c}, halogen, —CF₃, —CN, —S(O)₂R^{d}, —S(O)₂OR^{d}, —S(O)₂NR^{c}R^{c}, —C(O)R^{d}, —C(O)OR^{d}, —C(O)NR^{c}R^{c}, and Ring, B

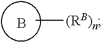

Ring B is C₃₋₆ cycloalkyl, phenyl, 5-membered heteroaryl, 6-membered heteroaryl containing two or more nitrogen atoms, 9-10 membered heteroaryl; or Ring B is Ring B′;
each R^{B} is independently selected from the group consisting of halogen, —CN, —C₁₋₄ alkyl, —C₁₋₄ haloalkyl, —C₁₋₄ aminoalkyl, —C₁₋₄ hydroxyalkyl, —C₁₋₄ methoxyalkyl, —(C₁₋₄ alkyl)O(C₁₋₄ alkyl), —C(O)(C₁₋₄ alkyl), —C(O)OH, —C(O)O(C₁₋₄ alkyl), —C(O)NH₂, —C(O)NH(C₁₋₄ alkyl), —C(O)N(C₁₋₄ alkyl)₂, —NH₂, —NH(C₁₋₄ alkyl), —NH(C₃₋₆ cycloalkyl), —NH(C₃₋₆ heterocycloalkyl), —(CH₂)ₘ—N(R^{c})₂, —(CH₂)ₘ—NHC(O)C₁₋₄ alkyl, —(CH₂)ₘ—NHC(O)O(C₁₋₄ alkyl), —NHS(O)₂C₁₋₄ alkyl, —OH, —O(C₁₋₄ alkyl), —O(C₁₋₄ haloalkyl), —SH, —S(C₁₋₄ alkyl), —S(O)(C₁₋₄ alkyl), —S(O)(NH)(C₁₋₄ alkyl), —S(O)₂(C₁₋₄ alkyl), —S(O)₂NH₂, —S(O₂)NHCH₃, substituted or unsubstituted C₃₋₆ cycloalkyl, or substituted or unsubstituted 3- to 6-membered heterocyclyl; or two R^{B} taken together form a carbonyl;
each R^{c} is independently R^{a}, or, alternatively, two R^{c} are taken together with the nitrogen atom to which they are bonded to form a 5 to 8-membered cycloheteroalkyl or 5-membered heteroaryl, which may optionally include one or more of the same or different additional heteroatoms and which may optionally be substituted with one or more of the same or different —C(O)R^{a} and R^{a} groups;
each R^{d} is independently selected from hydrogen and (C₁₋₆) alkyl optionally substituted with 5-membered heteroaryl, 6-membered heteroaryl and 6-membered aryl, each optionally substituted with 1, 2 or 3 groups selected from halogen, C₁₋₄ alkyl, and —O(C₁₋₄ alkyl);
l is 0 to 3;
m is 0 to 3;
n is 0 to 3; and
Ring B′ is selected from the group consisting of:

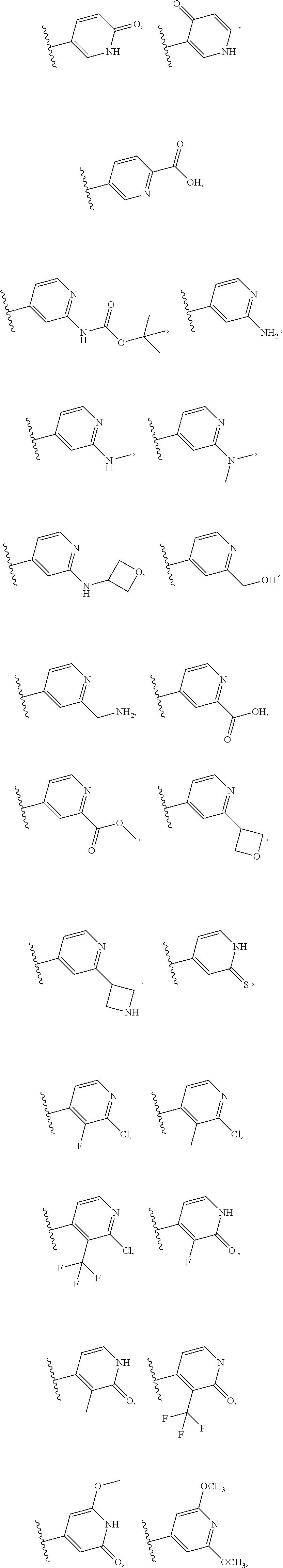

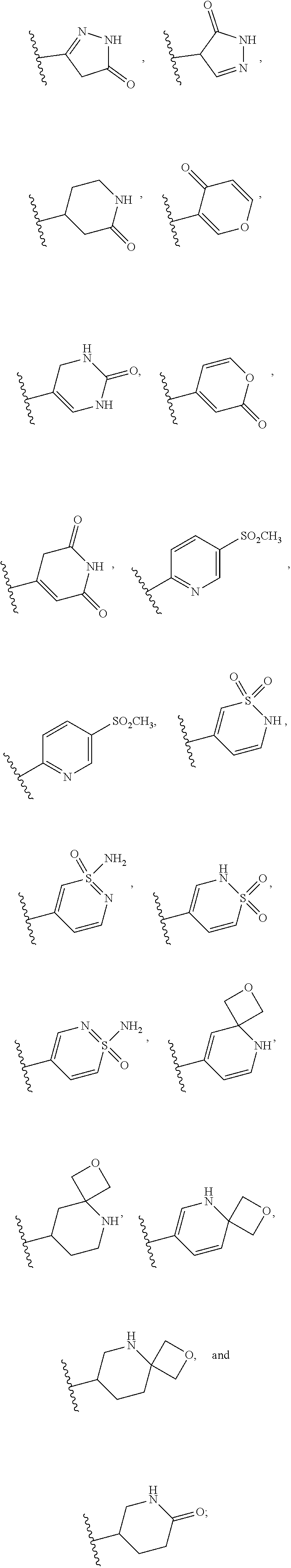

provided that the compound is not a compound disclosed in PCT/US2022/034901.
2. The compound of embodiment 1, having Formula (IIIa)

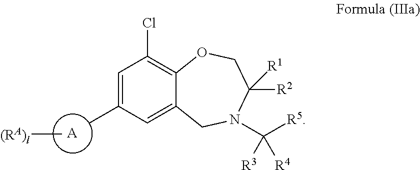

3. The compound of embodiment 1 or embodiment 2, wherein Ring A is

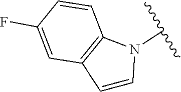 [SMILES: *n1ccc2cc(F)ccc21]

4. The compound of embodiment 1 or embodiment 2, wherein Ring A is

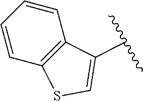 [SMILES: *c1csc2ccccc12]

optionally substituted with 1-3 R^{A}.
5. The compound of embodiment 4, wherein Ring A is

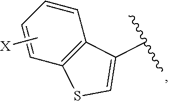

and X is halogen.
6. The compound of embodiment 4 wherein Ring A is

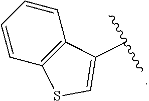 [SMILES: *c1csc2ccccc12]

7. The compound of embodiment 5 wherein Ring A is

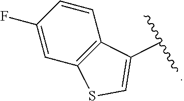 [SMILES: *c1csc2cc(F)ccc12]

8. The compound of embodiment 1 or embodiment 2, wherein Ring A is

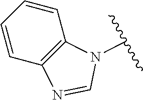 [SMILES: *n1cnc2ccccc21]

optionally substituted with 1-3 R^{A}.
9. The compound of embodiment 8, wherein Ring A is

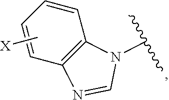

and X is halogen.
10. The compound of embodiment 9, wherein Ring A is

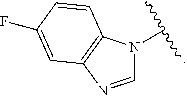 [SMILES: *n1cnc2cc(F)ccc21]

11. The compound of embodiment 1 or embodiment 2, wherein Ring A is

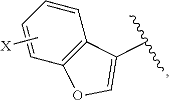

and X is halogen.
12. The compound of embodiment 11, wherein Ring A is

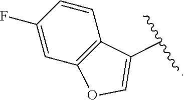 [SMILES: *c1coc2cc(F)ccc12]

13. The compound of embodiment 11, wherein Ring A is

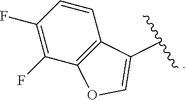 [SMILES: *c1coc2c(F)c(F)ccc12]

14. The compound of any one of embodiments 1-13, wherein R³ and R⁴ together form an oxo group.
15. The compound of embodiment 1 or embodiment 2, wherein R⁵ is selected from —(CH₂)ₘ—R^{b}, —(CHR^{a})ₘ—R^{b}, —O—(CH₂)ₘ—R^{b}, —O—(CHR^{a})ₘ—R^{b}, —C(O)NH—(CH₂)ₘ—R^{b}, —C(O)NH—(CHR^{a})ₘ—R^{b}, —NHC(O)OR^{d}, —NHC(O)NR^{c}R^{c};
   each R^{a} is independently selected from the group consisting of hydrogen, (C₁₋₆) alkyl and (C₃₋₈) cycloalkyl; and
   each R^{b} is independently selected from the group consisting of ═O, —OR^{d}, —OCF₂H, —OCF₃, —NR^{c}R^{c}, halogen, —CF₃, —CN, —S(O)₂R^{d}, —S(O)₂OR^{d}, —S(O)₂NR^{c}R^{c}, —C(O)R^{d}, —C(O)OR^{d}, and —C(O)NR^{c}R^{c}.
16. The compound of embodiment 15, wherein Ring A is a bicyclic heterocycle selected from the group consisting of:

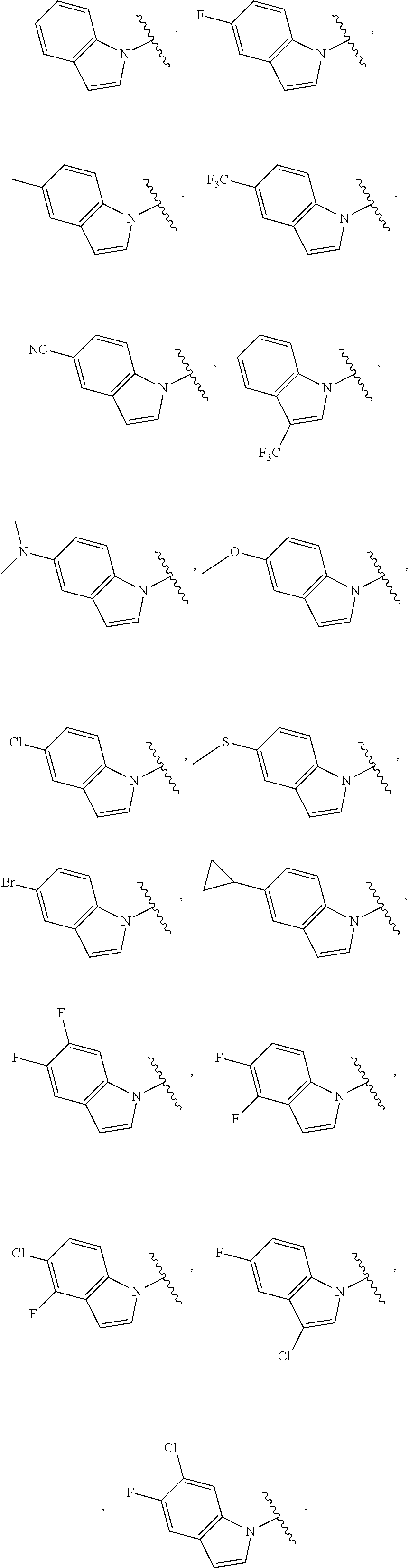

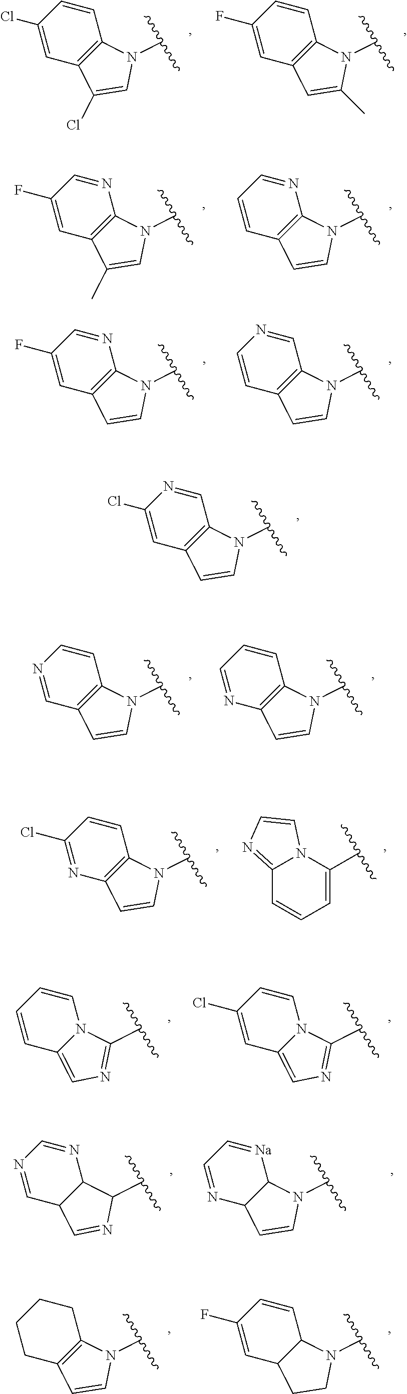

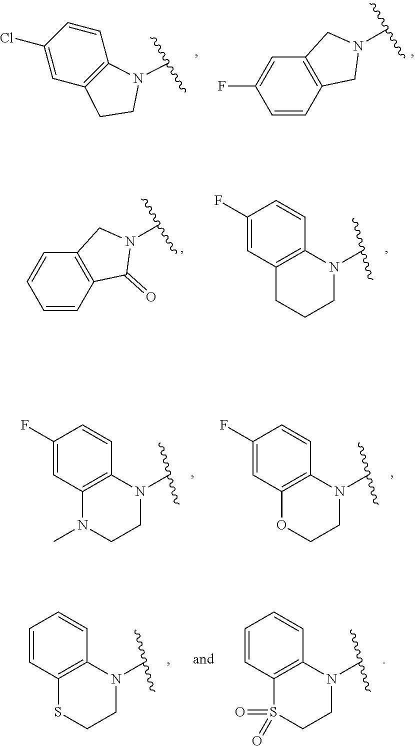

17. The compound of embodiment 15, wherein Ring A is a bicyclic heterocycle selected from the group consisting of:

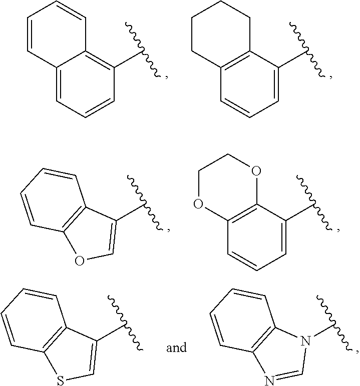

optionally substituted with 1-3 R^{A}.
18. A compound of the formula

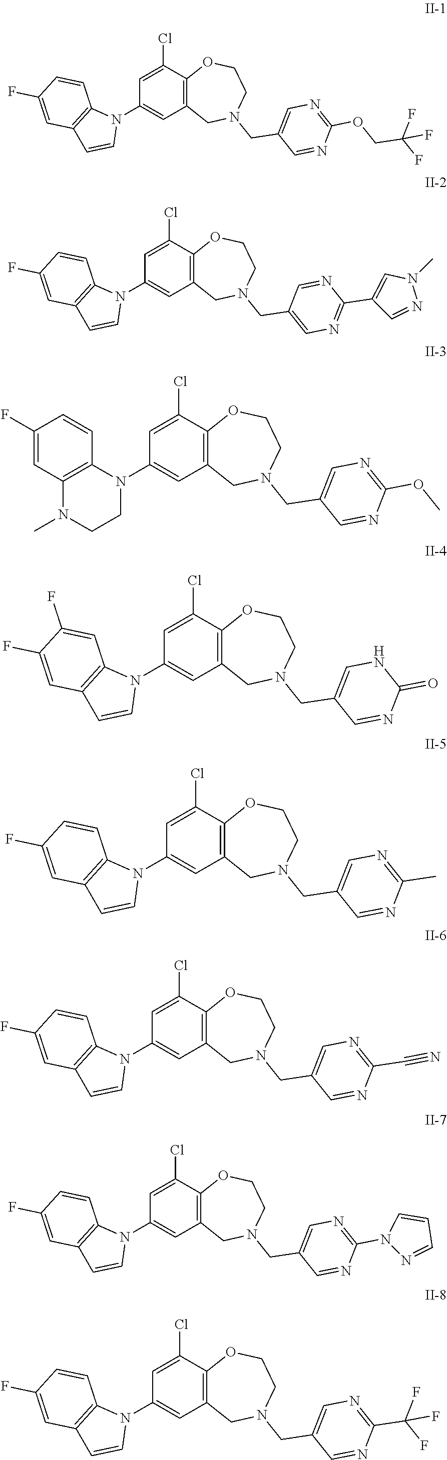

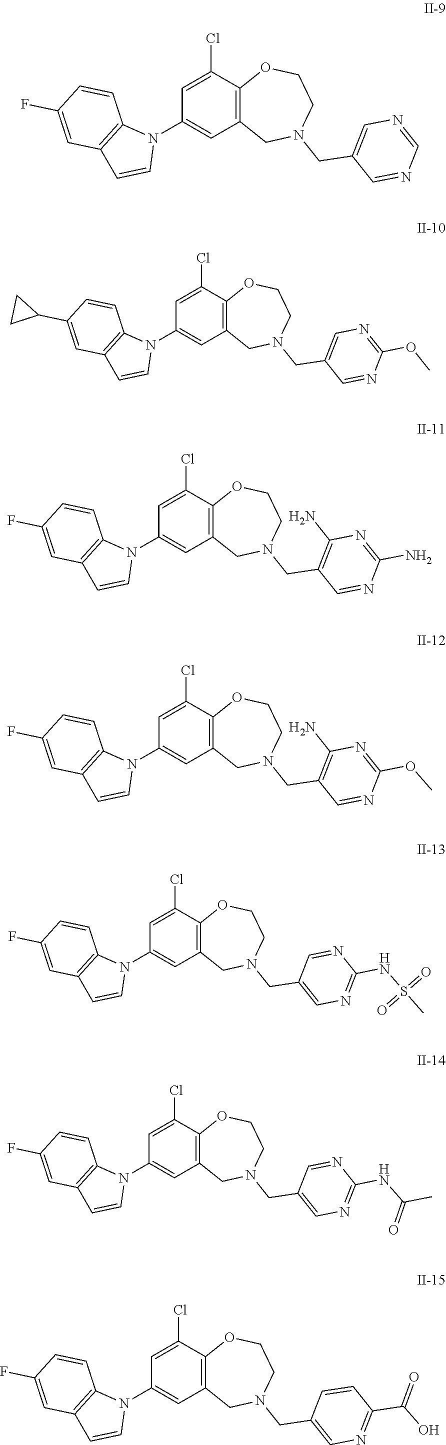

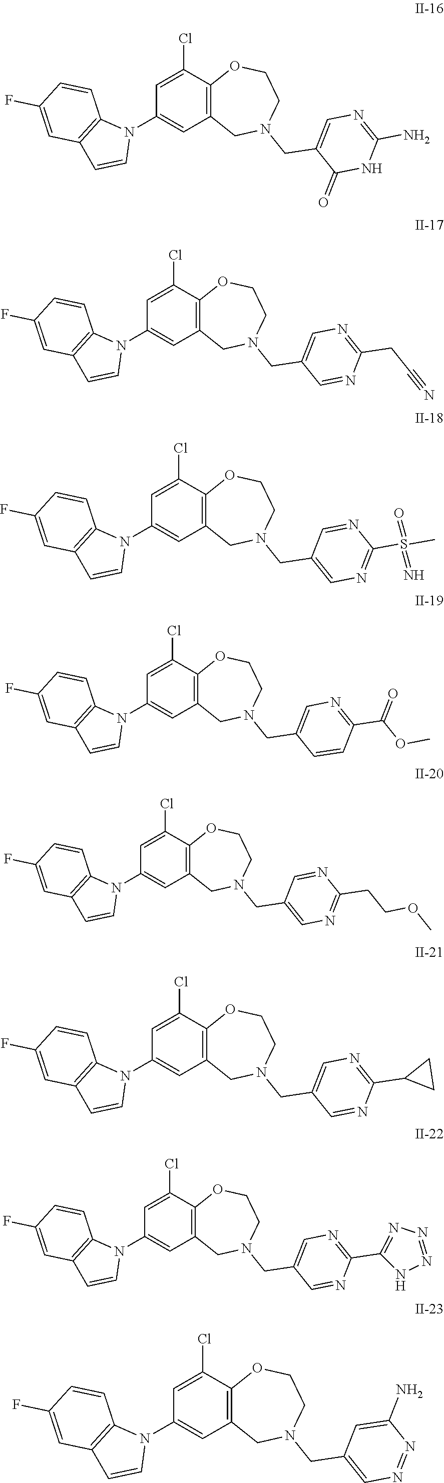

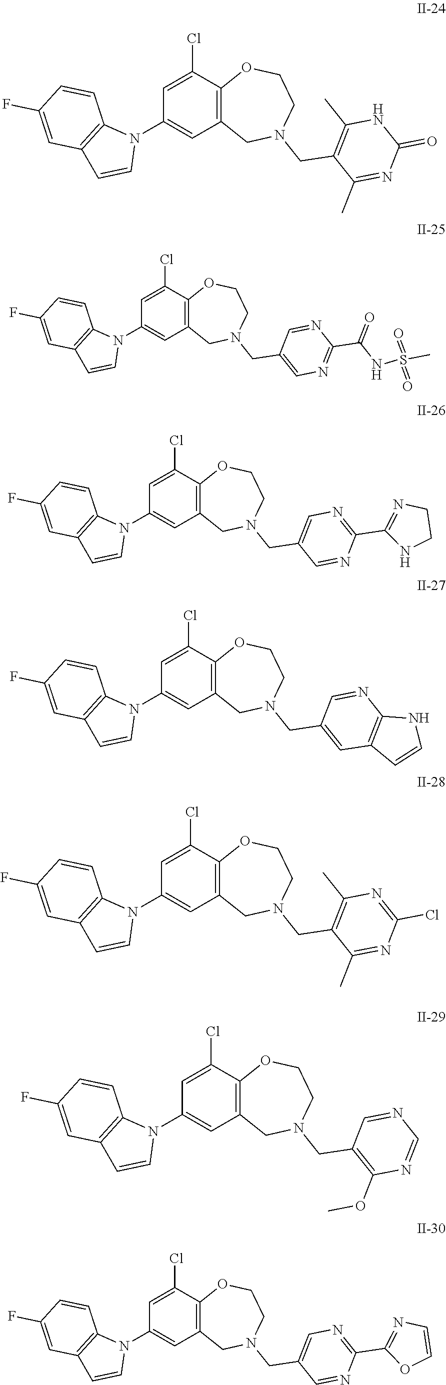

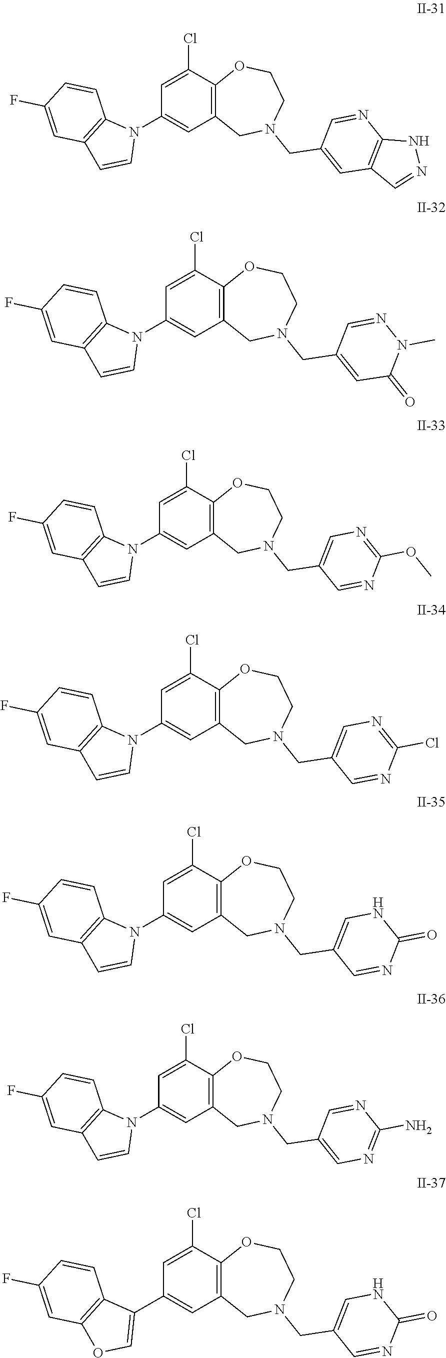

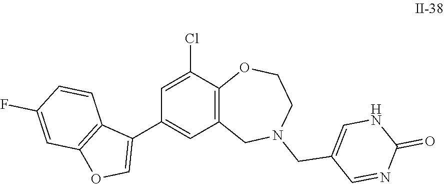 [SMILES: O=c1ncc(CN2CCOc3c(Cl)cc(-c4coc5cc(F)ccc45)cc3C2)c[nH]1]

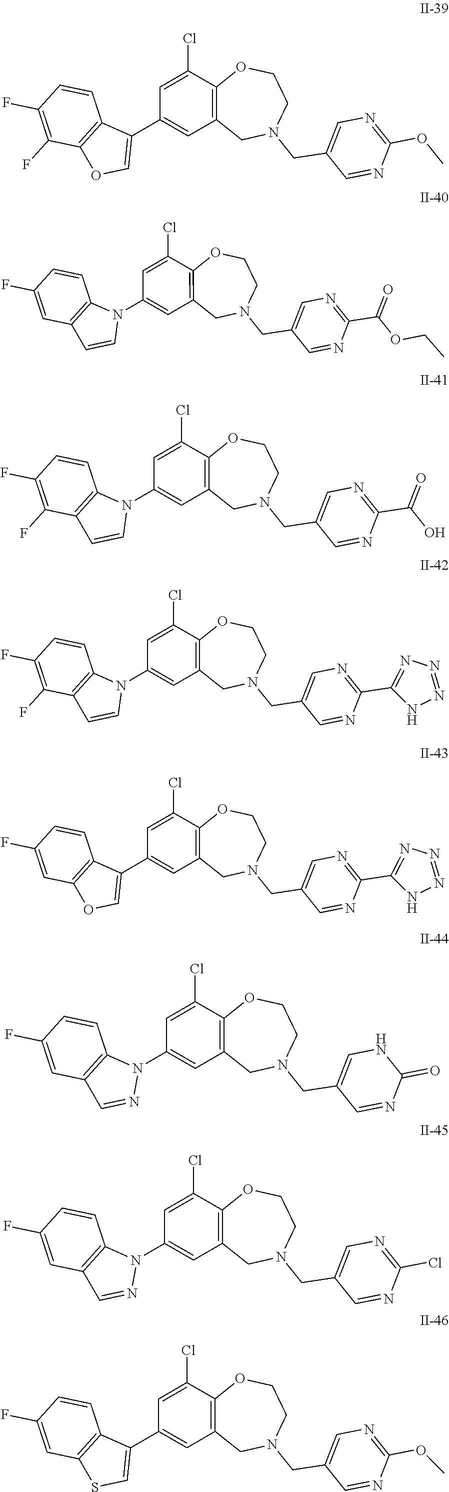

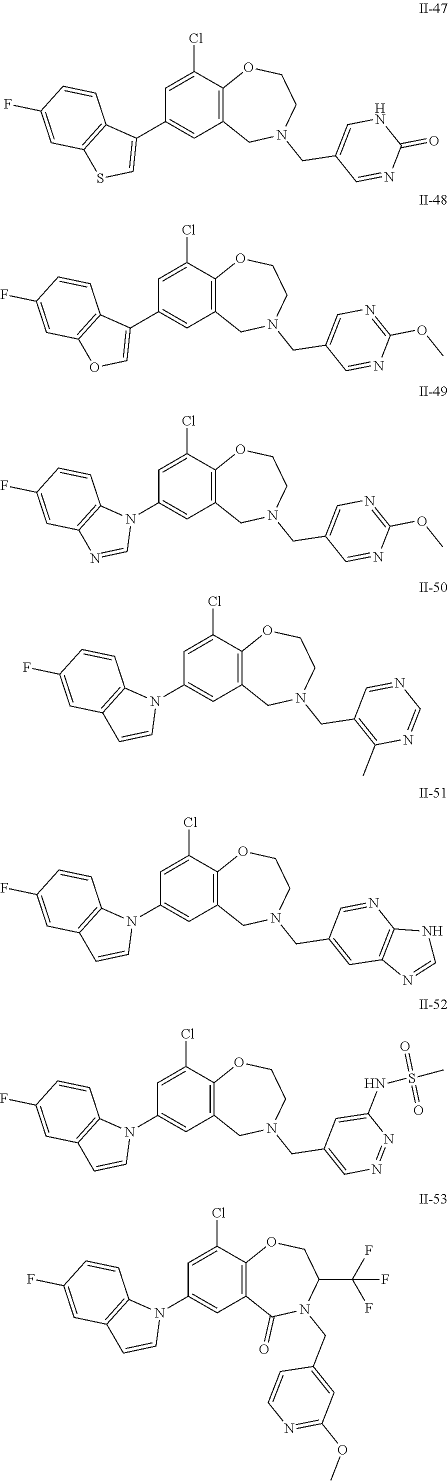

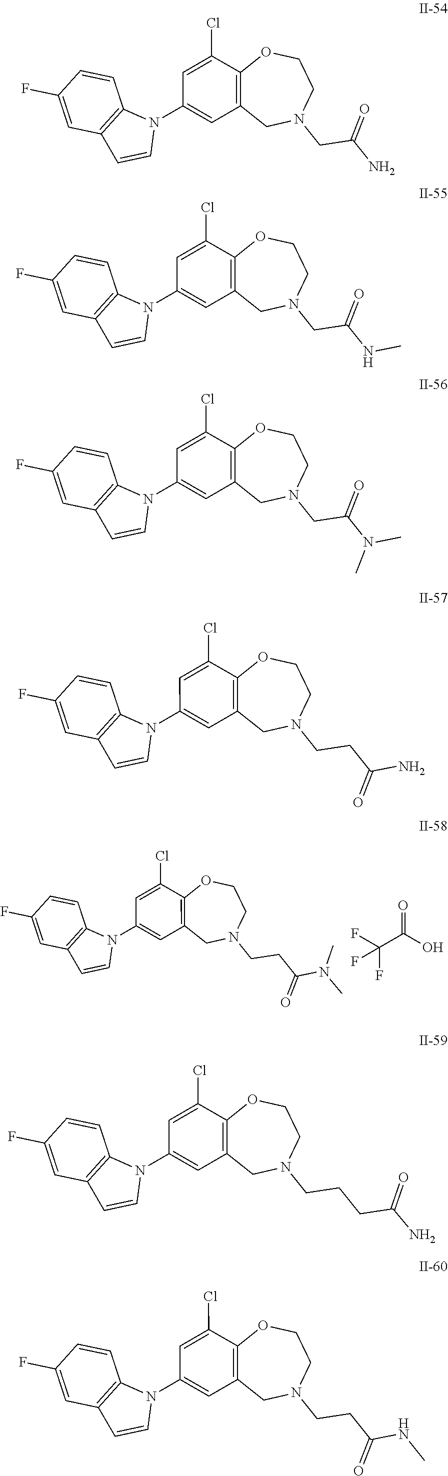

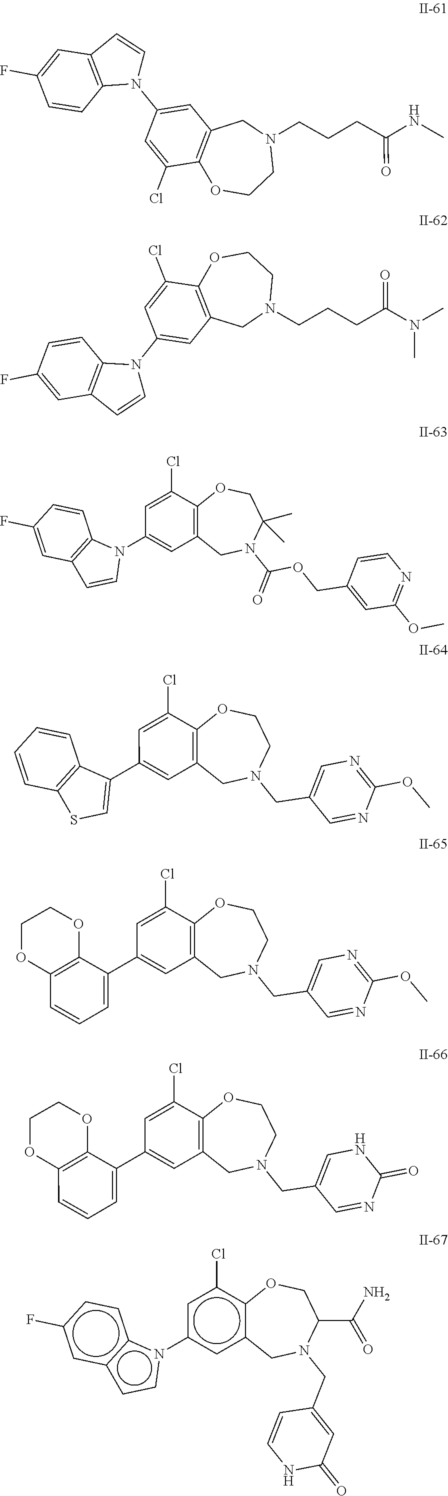

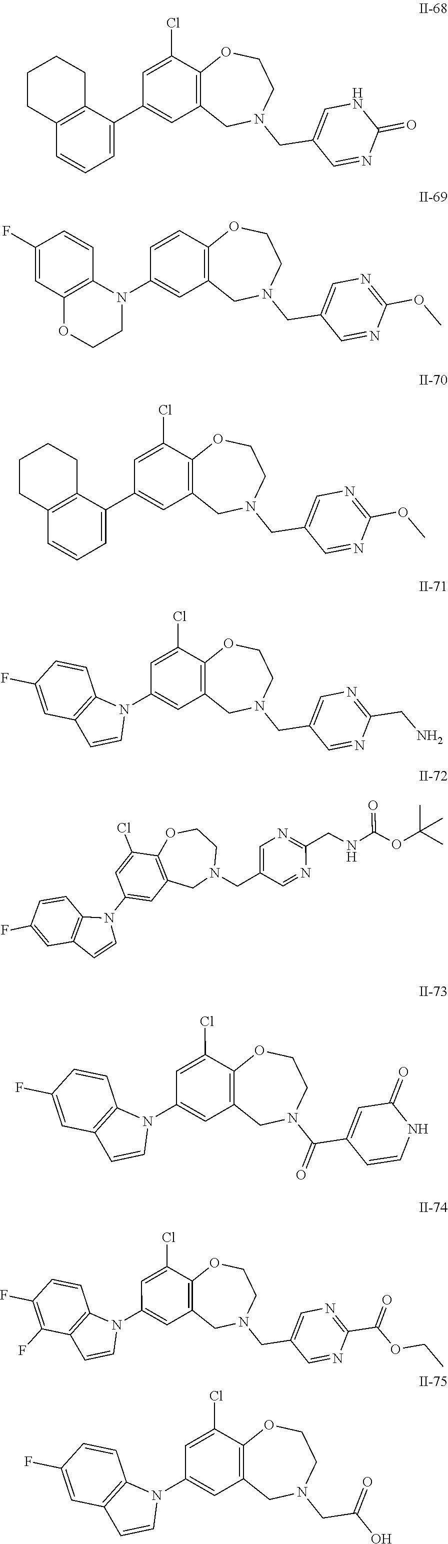

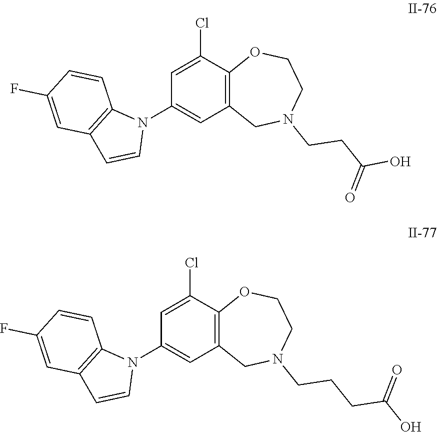 [SMILES: O=C(O)CCN1CCOc2c(Cl)cc(-n3ccc4cc(F)ccc43)cc2C1 ; O=C(O)CCCN1CCOc2c(Cl)cc(-n3ccc4cc(F)ccc43)cc2C1]

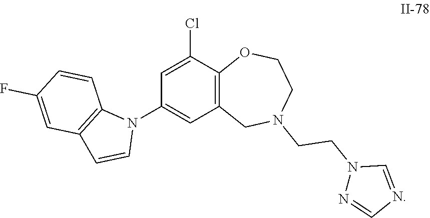 [SMILES: Fc1ccc2c(ccn2-c2cc(Cl)c3c(c2)CN(CCn2cncn2)CCO3)c1]

19. The compound of any one of embodiments 1-18 in the form of a pharmaceutically acceptable salt.
20. A pharmaceutical composition comprising a compound of any one of embodiments 1-19, and a pharmaceutically acceptable excipient.
21. A method of inhibiting the activity of the prostaglandin E2 receptor 2 (EP2) in a mammal comprising administering to the mammal an effective amount of a compound of any one of embodiments 1-19, or a pharmaceutical composition of embodiment 20.
22. A method of treating a disease or condition that would benefit from the modulation of prostaglandin E2 receptor 2 (EP2) activity comprising administering to the mammal an effective amount of a compound of any one of embodiments 1-19, or a pharmaceutical composition of embodiment 20.
23. A method for treating a disease or condition selected from neurological disorders, inflammatory disorders, autoimmune disorders, fibrotic disorders, allergic conditions, and combinations thereof, comprising administering to a subject in need thereof an effective amount of a compound of any one of embodiments 1-19, or a pharmaceutical composition of embodiment 20.
24. A method for treating a neurological disease selected from amyotrophic lateral sclerosis (ALS), epilepsy, dementia, Alzheimer's disease, concussion, delirium, chemotherapy-associated cognitive decline, radiation-associated cognitive decline, post-operative cognitive dysfunction including post-operative neurocognitive disorder, post-operative delirium, delayed neurocognitive disorder, and delayed neurocognitive recovery, vascular dementia, frontotemporal dementia, Lewy body dementia, pre-senile dementia (mild cognitive impairment or MCI), Binswanger's dementia (subcortical arteriosclerotic encephalopathy), HIV-associated dementia (including asymptomatic neurocognitive impairment (ANI), minor neurocognitive disorder (MND), and HIV-associated dementia (HAD) (also called AIDS dementia complex [ADC] or HIV encephalopathy), multiple system atrophy (MSA), spinocerebellar ataxias, Steel-Richardson-Olszewski disease (progressive supranuclear palsy), head injury, concussion, chronic traumatic encephalopathy, intracerebral hemorrhage, hematoma, diabetic retinopathy, macular degeneration, macular edema, glaucoma, multiple sclerosis, vasogenic edema including edema caused by treatment with antibody therapies (e.g. ARIA-E), migraine, Huntington's disease, ALS, and Parkinson's disease, comprising administering to a subject in need thereof an effective amount of any one of embodiments 1-19, or a pharmaceutical composition of embodiment 20.
25. A method for treating an inflammatory or allergic disorder selected from systemic lupus erythematosus, rheumatoid arthritis, type I diabetes mellitus, inflammatory bowel disease, biliary cirrhosis, uveitis, multiple sclerosis, Crohn's disease, hyperimmunoglobulinemia D and periodic fever syndrome, cryopyrin-associated periodic syndromes, Schnitzler's syndrome, systemic juvenile idiopathic arthritis, adult's onset Still's disease, ulcerative colitis, necrotizing enterocolitis (NEC), peritonitis, gout, gout flares, gouty arthritis, bullous pemphigoid, sarcoidosis, psoriasis, autoimmune myositis, Wegener's granulomatosis, ichthyosis, Graves ophthalmopathy, asthma, atopic asthma, allergic asthma, atopic bronchial IgE-mediated asthma, non-atopic asthma, bronchial asthma, non-allergic asthma, essential asthma, true asthma, intrinsic asthma caused by pathophysiologic disturbances, essential asthma of unknown or unapparent cause, emphysematous asthma, exercise-induced asthma, emotion-induced asthma, extrinsic asthma caused by environmental factors, cold air induced asthma, occupational asthma, infective asthma caused by or associated with bacterial, fungal, protozoal, or viral infection, incipient asthma, wheezy infant syndrome, bronchiolitis, cough variant asthma or drug-induced asthma), allergic bronchopulmonary aspergillosis (ABPA), allergic rhinitis, perennial allergic rhinitis, perennial rhinitis, vasomotor rhinitis, post-nasal drip, purulent or non-purulent sinusitis, acute or chronic sinusitis, and ethmoid, frontal, maxillary, and sphenoid sinusitis, comprising administering to a subject in need thereof an effective amount of a compound of any one of embodiments 1-19, or a pharmaceutical composition of embodiment 20.
26. A method for treating an immune regulatory disorder selected from tissue or organ transplant, graft-versus-host diseases brought about by transplantation, rheumatoid arthritis, systemic lupus erythematosus, Hashimoto's thyroiditis, multiple sclerosis, systemic sclerosis, myasthenia gravis, type I diabetes, uveitis, posterior uveitis, allergic encephalomyelitis, glomerulonephritis, postinfectious autoimmune diseases, rheumatic fever and post-infectious glomerulonephritis, inflammatory and hyperproliferative skin diseases, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, celiac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, chronic lymphocytic leukemia, arteriosclerosis, atherosclerosis, aortitis syndrome, polyarteritis nodosa, myocardosis, scleroderma, Wegener's granuloma, Sjögren's syndrome, adiposis, eosinophilic fascitis, lesions of gingiva, periodontium, alveolar bone, substantia ossea dentis, glomerulonephritis, alopecia areata, male pattern alopecia or alopecia senilis by preventing epilation or providing hair germination and/or promoting hair generation and hair growth, vitiligo, muscular dystrophy, pyoderma and Sezary's syndrome, Addison's disease, ischemia-reperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA ballous dermatitis and cement dermatitis, gingivitis, periodontitis and sepsis, comprising administering to a subject in need thereof an effective amount of a compound of any one of embodiments 1-19, or a pharmaceutical composition of embodiment 20.
27. A method for treating a proliferative disorder selected from, solid tumors, carcinoma of the brain, kidney, liver, adrenal gland, bladder, breast, stomach, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins lymphoma, a mammary carcinoma, follicular carcinoma, undifferentiated carcinoma, papillary carcinoma, seminoma, melanoma, IL-1 driven disorders, ABC diffuse large B-cell lymphoma (DLBCL), Waldenström's macroglobulinemia, Hodgkin's lymphoma, primary cutaneous T-cell lymphoma, smoldering or indolent multiple myeloma, hematological malignancies, including leukemia, acute myeloid leukemia (AML), DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, myelodysplastic syndromes (MDS), myelofibrosis, polycythemia vera, Kaposi's sarcoma, splenic marginal zone lymphoma, multiple myeloma, plasmacytoma, intravascular large B-cell lymphoma, comprising administering to a subject in need thereof an effective amount of a compound of any one of embodiments 1-19, or a pharmaceutical composition of embodiment 20.
28. A method for treating a fibrotic condition selected from idiopathic pulmonary fibrosis, systemic sclerosis, uterine fibroids (leiomyomata), sclerodoma, autoimmune hepatitis, primary biliary cirrhosis, sclerosing cholangitis, partial liver resection, acute liver necrosis, necrosis caused by toxin, viral hepatitis, shock, or anoxia, B-virus hepatitis, non-A/non-B hepatitis, chronic hepatitis C virus (HCV) infection, cirrhosis, alcoholic liver disease, including alcoholic cirrhosis, non-alcoholic steatohepatitis (NASH), hepatic failure, fulminant hepatic failure, late-onset hepatic failure, “acute-on-chronic” liver failure, kidney disease, and chronic kidney disease, comprising administering to a subject in need thereof an effective amount of any one of embodiments 1-19, or a pharmaceutical composition of embodiment 20.
29. A method for treating cytokine release syndrome, comprising administering to a subject in need thereof an effective amount of a compound of any one of embodiments 1-19, or a pharmaceutical composition of embodiment 20.
30. A method for treating a condition selected from endometriosis, uterine fibroids (leiomyomata), menorrhagia, adenomyosis, primary and secondary dysmenorrhoea (including symptoms of dyspareunia, dyschexia and chronic pelvic pain), chronic pelvic pain syndrome, precocious puberty, cervical ripening, breast carcinoma, colon carcinoma, familial adenomatous polyposis, colorectal adenomas, endometrial carcinoma, prostate carcinoma, pulmonary carcinoma, testicular carcinoma, gastric carcinoma, macular degeneration, inflammatory and neuropathic pain conditions, migraine, headache, cluster headache, inflammation induced pain, cancer pain, polycystic kidney disease and polycystic ovarian syndrome, comprising administering to a subject in need thereof an effective amount of a compound of any one of embodiments 1-19, or a pharmaceutical composition of embodiment 20.
31. A method for treating a vascular disorder, comprising administering to a subject in need thereof an effective amount of a compound of any one of embodiments 1-19, or a pharmaceutical composition of embodiment 20.
32. The method of embodiment 31, wherein the vascular disorder is selected from head injury, stroke, aneurysm, vascular dementia, vascular cognitive impairment and dementia (VCID), cerebral small vessel disease, subcortical ischemic vascular disease, mixed dementia, and ischemic vascular damage.
33. A method for treating a condition selected from Alzheimer's disease, ALS, multiple sclerosis, Parkinson's disease and Huntington's disease, comprising administering to a subject in need thereof an effective amount of a compound of any one of embodiments 1-19, or a pharmaceutical composition of embodiment 20.

The present disclosure contemplates, among other things, the following numbered embodiments:
1. A compound having the structure:

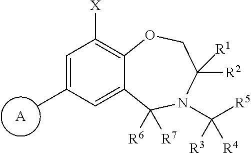

wherein Ring A is selected from the group consisting of

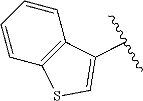 [SMILES: *c1csc2ccccc12]

optionally substituted with 1-3 R^{A},

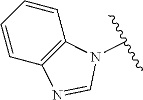 [SMILES: *n1cnc2ccccc21]

optionally substituted with 1-3 R^{A},

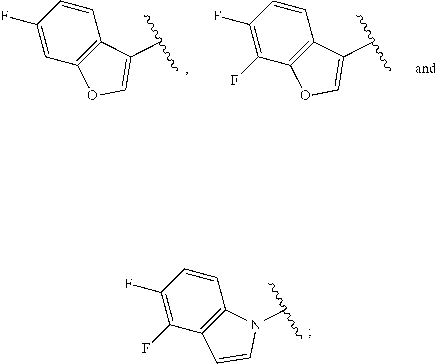 [SMILES: *c1coc2cc(F)ccc12 ; *c1coc2c(F)c(F)ccc12 ; *n1ccc2c(F)c(F)ccc21]

each R^{A} is selected from halogen; —CN, —C₁₋₄ alkyl, —C₁₋₄ haloalkyl, —(C₁₋₄ alkyl)O(C₁₋₄ alkyl), —C(O)OH, —C(O)O(C₁₋₄ alkyl), —C(O)NH₂, —C(O)NH(C₁₋₄ alkyl), —C(O)N(C₁₋₄ alkyl)₂, —NH₂, —NH(C₁₋₄ alkyl), —N(C₁₋₄ alkyl)₂, —OH, —O(C₁₋₄ alkyl), —O(C₁₋₄ haloalkyl), —S(C₁₋₄ alkyl), —SO₂C₁₋₄ alkyl, —SO₂NHC₁₋₄ alkyl, substituted or unsubstituted C₃₋₆ cycloalkyl, or substituted or unsubstituted 3- to 6-membered heterocyclyl; or two R^{A} taken together form a carbonyl;
R¹ and R² are independently selected from hydrogen, —C₁₋₄ alkyl, —CN, —C(O)NR^{c}R^{c}, or together R¹ and R² form an oxo, C₃₋₆ membered cycloalkyl or 3-6 membered cycloheteroalkyl;
X is selected from hydrogen and halogen;
R³ and R⁴ are independently selected from hydrogen, —C₁₋₄ alkyl, together R³ and R⁴ form an oxo, or one of R³ and R⁴ form a double bond with R⁵ and the other of R³ and R⁴ is hydrogen or —C₁₋₄ alkyl;
R⁵ is selected from —(CH₂)ₘ—R^{b}, —(CHR^{a})ₘ—R^{b}, —O—(CH₂)ₘ—R^{b}, —O—(CHR^{a})ₘ—R^{b}, —C(O)NH—(CH₂)ₘ—R^{b}, —C(O)NH—(CHR^{a})ₘ—R^{b}.
R⁶ and R⁷ are independently selected from hydrogen, —C₁₋₄ alkyl, or together R⁶ and R⁷ form an oxo;
each R^{a} is independently selected from the group consisting of hydrogen, (C₁₋₆) alkyl and (C₃₋₈) cycloalkyl;
each R^{b} is independently selected from the group consisting of ═O, —OR^{d}, —OCF₂H, —OCF₃, —NR^{c}R^{c}, halogen, —CF₃, —CN, —S(O)₂R^{d}, —S(O)₂OR^{d}, —S(O)₂NR^{c}R^{c}, —C(O)R^{d}, —C(O)OR^{d}, —C(O)NR^{c}R^{c}, and Ring, B

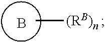

Ring B is C₃₋₆ cycloalkyl, phenyl, 5-membered heteroaryl, 6-membered heteroaryl containing two or more nitrogen atoms, 9-10 membered heteroaryl; or Ring B is Ring B′, provided that Ring B is not

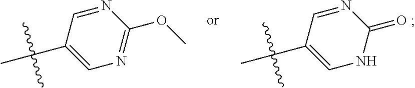

each R^{B} is independently selected from the group consisting of halogen, —CN, —C₁₋₄ alkyl, —C₁₋₄ haloalkyl, —C₁₋₄ aminoalkyl, —C₁₋₄ hydroxyalkyl, —C₁₋₄ methoxyalkyl, —(C₁₋₄ alkyl)O(C₁₋₄ alkyl), —C(O)(C₁₋₄ alkyl), —C(O)OH, —C(O)O(C₁₋₄ alkyl), —C(O)NH₂, —C(O)NH(C₁₋₄ alkyl), —C(O)N(C₁₋₄ alkyl)₂, —NH₂, —NH(C₁₋₄ alkyl), —NH(C₃₋₆ cycloalkyl), —NH(C₃₋₆ heterocycloalkyl), —(CH₂)ₘ—N(R^{c})₂, —(CH₂)ₘ—NHC(O)C₁₋₄ alkyl, —(CH₂)ₘ—NHC(O)O(C₁₋₄ alkyl), —NHS(O)₂C₁₋₄ alkyl, —OH, —O(C₁₋₄ alkyl), —O(C₁₋₄ haloalkyl), —SH, —S(C₁₋₄ alkyl), —S(O)(C₁₋₄ alkyl), —S(O)(NH)(C₁₋₄ alkyl), —S(O)₂(C₁₋₄ alkyl), —S(O)₂NH₂, —S(O₂)NHCH₃, substituted or unsubstituted C₃₋₆ cycloalkyl, or substituted or unsubstituted 3- to 6-membered heterocyclyl; or two R^{B} taken together form a carbonyl;
each R^{c} is independently R^{a}, or, alternatively, two R^{c} are taken together with the nitrogen atom to which they are bonded to form a 5 to 8-membered cycloheteroalkyl or 5-membered heteroaryl, which may optionally include one or more of the same or different additional heteroatoms and which may optionally be substituted with one or more of the same or different —C(O)R^{a} and R^{a} groups;
each R^{d} is independently selected from hydrogen and (C₁₋₆) alkyl optionally substituted with 5-membered heteroaryl, 6-membered heteroaryl and 6-membered aryl, each optionally substituted with 1, 2 or 3 groups selected from halogen, C₁₋₄ alkyl, and —O(C₁₋₄ alkyl);
m is 0 to 3; and
n is 0 to 3.
2. The compound of embodiment 1, wherein R^{b} is selected from

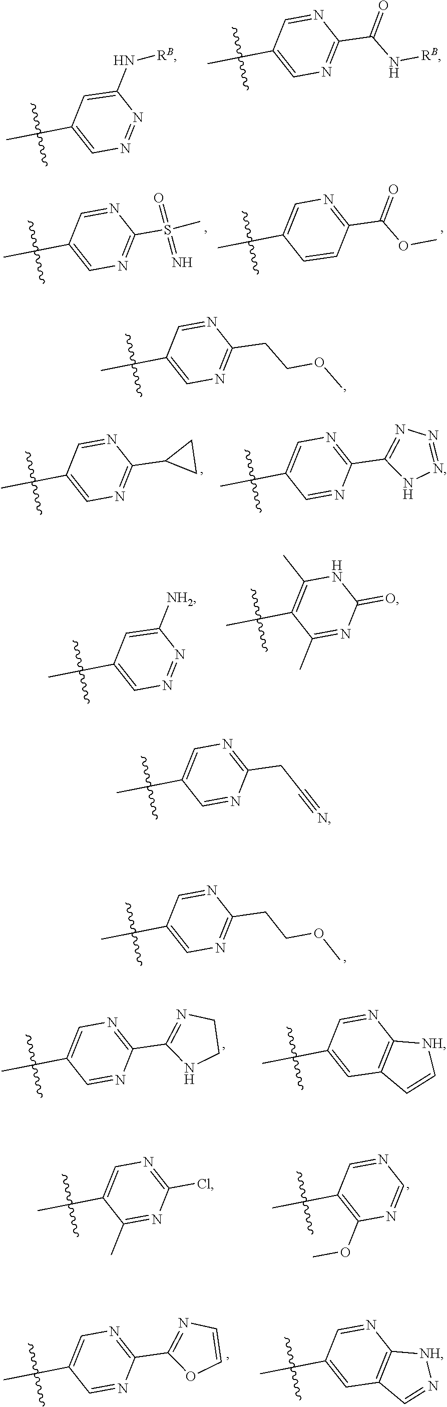

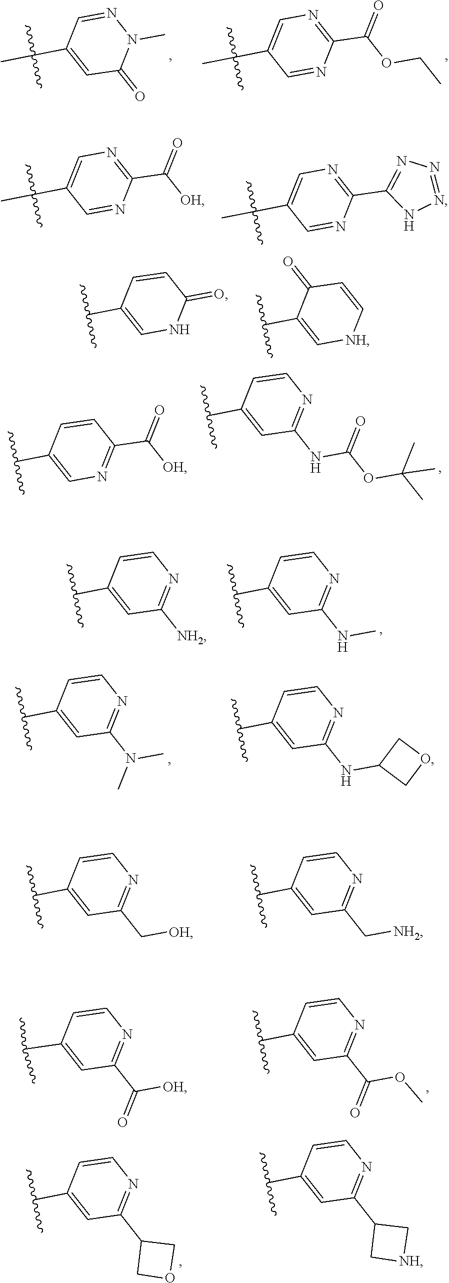

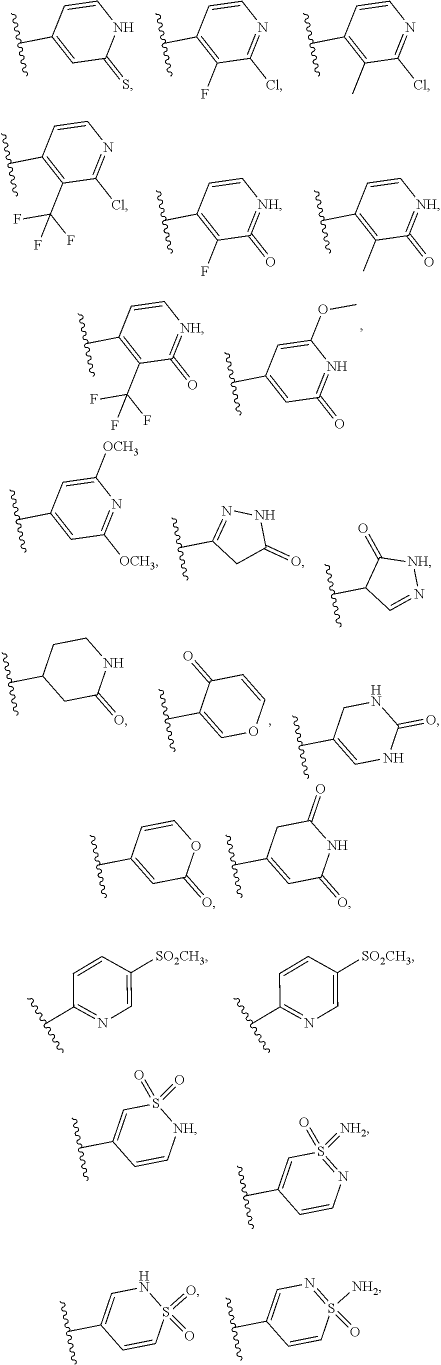

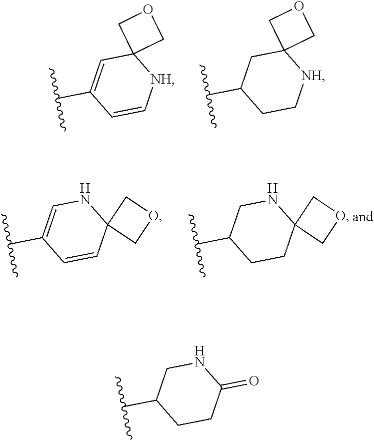 [SMILES: *C1=CC2(COC2)NC=C1 ; *C1CCNC2(COC2)C1 ; *C1=CNC2(C=C1)COC2 ; *C1CCC2(COC2)NC1 ; *C1CCC(=O)NC1]

each substituted by n R^{b}.
3. The compound of embodiment 1, wherein each R^{B} is selected from —C(O)(C₁₋₄ alkyl), —C(O)OH, —C(O)O(C₁₋₄ alkyl), —C(O)NH₂, —C(O)NH(C₁₋₄ alkyl), —C(O)N(C₁₋₄ alkyl)₂, —S(O)(C₁₋₄ alkyl), —S(O)(NH)(C₁₋₄ alkyl), —S(O)₂(C₁₋₄ alkyl), —S(O)₂NH₂, —S(O₂)NHCH₃, —S(O)₂(C₆₋₁₃aryl); and —S(O)₂NHS(O)₂ (C₁₋₄ alkyl).
4. The compound of embodiment 1, wherein R⁵ is —(CH₂)ₘ—R^{b},
and R^{b} is selected from the group consisting of

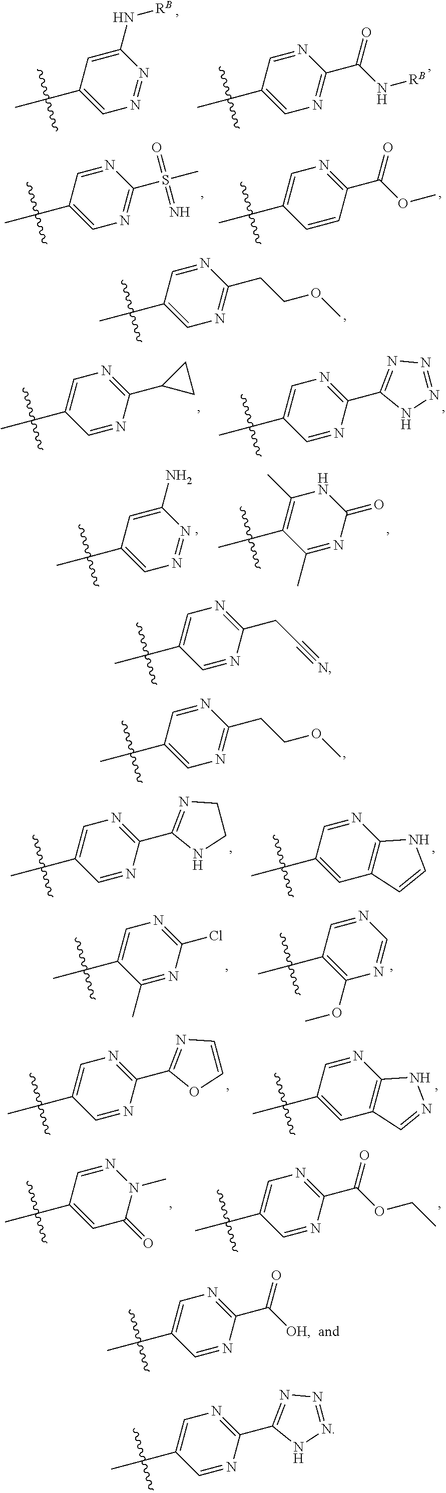

5. The compound of any one of embodiments 1-4, wherein R^{B} is selected from —S(O)₂(C₁₋₄ alkyl), —S(O)₂NH₂, —S(O₂)NHCH₃, and —S(O)₂(C₆₋₁₃aryl).
6. The compound of embodiment 1, wherein R^{b} is N

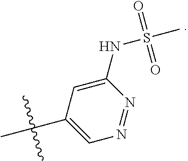 [SMILES: *c1cnnc(NS(C)(=O)=O)c1]

7. The compound of embodiment 1, wherein R^{b} is

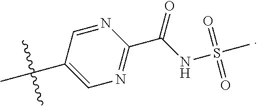 [SMILES: *c1cnc(C(=O)NS(C)(=O)=O)nc1]

8. The compound of any one of embodiments 1-7, wherein Ring A is F

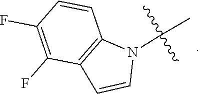 [SMILES: *n1ccc2c(F)c(F)ccc21]

9. The compound of any one of embodiments 1-8 in the form of a pharmaceutically acceptable salt.
10. A pharmaceutical composition comprising a compound of any one of embodiments 1-9, and a pharmaceutically acceptable excipient.
11. A method for modulating prostaglandin E2 receptor 2 (EP2), comprising contacting the EP2 with a compound of any one of embodiments 1-9 or a pharmaceutical composition according to embodiment 10.
12. The method of embodiment 11, wherein the contacting is in vitro.
13. The method of embodiment 11, wherein the contacting is in vivo.
14. A method for treating a disease or condition that would benefit from the modulation of prostaglandin E2 receptor 2 (EP2), comprising administering to a subject in need thereof an effective amount of a compound of any one of embodiments 1-9 or a pharmaceutical composition according to embodiment 10.
15. A method for treating a disease or condition selected from neurological disorders, inflammatory disorders, autoimmune disorders, fibrotic disorders, allergic conditions, and combinations thereof, comprising administering to a subject in need thereof an effective amount of a compound of any one of embodiments 1-9 or a pharmaceutical composition according to embodiment 10.
16. A method for treating a neurological disease selected from amyotrophic lateral sclerosis (ALS), epilepsy, dementia, Alzheimer's disease, concussion, delirium, chemotherapy-associated cognitive decline, radiation-associated cognitive decline, post-operative cognitive dysfunction including post-operative neurocognitive disorder, post-operative delirium, delayed neurocognitive disorder, and delayed neurocognitive recovery, vascular dementia, frontotemporal dementia, Lewy body dementia, pre-senile dementia (mild cognitive impairment or MCI), Binswanger's dementia (subcortical arteriosclerotic encephalopathy), HIV-associated dementia (including asymptomatic neurocognitive impairment (ANI), minor neurocognitive disorder (MND), and HIV-associated dementia (HAD) (also called AIDS dementia complex [ADC] or HIV encephalopathy), multiple system atrophy (MSA), spinocerebellar ataxias, Steel-Richardson-Olszewski disease (progressive supranuclear palsy), head injury, concussion, chronic traumatic encephalopathy, intracerebral hemorrhage, hematoma, diabetic retinopathy, macular degeneration, macular edema, glaucoma, multiple sclerosis, vasogenic edema including edema caused by treatment with antibody therapies (e.g. ARIA-E), migraine, Huntington's disease, ALS, and Parkinson's disease, comprising administering to a subject in need thereof an effective amount of a compound of any one of embodiments 1-9 or a pharmaceutical composition according to embodiment 10.
17. A method for treating an inflammatory or allergic disorder selected from systemic lupus erythematosus, rheumatoid arthritis, type I diabetes mellitus, inflammatory bowel disease, biliary cirrhosis, uveitis, multiple sclerosis, Crohn's disease, hyperimmunoglobulinemia D and periodic fever syndrome, cryopyrin-associated periodic syndromes, Schnitzler's syndrome, systemic juvenile idiopathic arthritis, adult's onset Still's disease, ulcerative colitis, necrotizing enterocolitis (NEC), peritonitis, gout, gout flares, gouty arthritis, bullous pemphigoid, sarcoidosis, psoriasis, autoimmune myositis, Wegener's granulomatosis, ichthyosis, Graves ophthalmopathy, asthma, atopic asthma, allergic asthma, atopic bronchial IgE-mediated asthma, non-atopic asthma, bronchial asthma, non-allergic asthma, essential asthma, true asthma, intrinsic asthma caused by pathophysiologic disturbances, essential asthma of unknown or unapparent cause, emphysematous asthma, exercise-induced asthma, emotion-induced asthma, extrinsic asthma caused by environmental factors, cold air induced asthma, occupational asthma, infective asthma caused by or associated with bacterial, fungal, protozoal, or viral infection, incipient asthma, wheezy infant syndrome, bronchiolitis, cough variant asthma or drug-induced asthma), allergic bronchopulmonary aspergillosis (ABPA), allergic rhinitis, perennial allergic rhinitis, perennial rhinitis, vasomotor rhinitis, post-nasal drip, purulent or non-purulent sinusitis, acute or chronic sinusitis, and ethmoid, frontal, maxillary, and sphenoid sinusitis, comprising administering to a subject in need thereof an effective amount of a compound of any one of embodiments 1-9 or a pharmaceutical composition according to embodiment 10.
18. A method for treating an immune regulatory disorder selected from tissue or organ transplant, graft-versus-host diseases brought about by transplantation, rheumatoid arthritis, systemic lupus erythematosus, Hashimoto's thyroiditis, multiple sclerosis, systemic sclerosis, myasthenia gravis, type I diabetes, uveitis, posterior uveitis, allergic encephalomyelitis, glomerulonephritis, postinfectious autoimmune diseases, rheumatic fever and post-infectious glomerulonephritis, inflammatory and hyperproliferative skin diseases, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, celiac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, chronic lymphocytic leukemia, arteriosclerosis, atherosclerosis, aortitis syndrome, polyarteritis nodosa, myocardosis, scleroderma, Wegener's granuloma, Sjögren's syndrome, adiposis, eosinophilic fascitis, lesions of gingiva, periodontium, alveolar bone, substantia ossea dentis, glomerulonephritis, alopecia areata, male pattern alopecia or alopecia senilis by preventing epilation or providing hair germination and/or promoting hair generation and hair growth, vitiligo, muscular dystrophy, pyoderma and Sezary's syndrome, Addison's disease, ischemia-reperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA ballous dermatitis and cement dermatitis, gingivitis, periodontitis and sepsis, comprising administering to a subject in need thereof an effective amount of a compound of any one of embodiments 1-9 or a pharmaceutical composition according to embodiment 10.
19. A method for treating a proliferative disorder selected from, solid tumors, carcinoma of the brain, kidney, liver, adrenal gland, bladder, breast, stomach, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins lymphoma, a mammary carcinoma, follicular carcinoma, undifferentiated carcinoma, papillary carcinoma, seminoma, melanoma, IL-1 driven disorders, ABC diffuse large B-cell lymphoma (DLBCL), Waldenström's macroglobulinemia, Hodgkin's lymphoma, primary cutaneous T-cell lymphoma, smoldering or indolent multiple myeloma, hematological malignancies, including leukemia, acute myeloid leukemia (AML), DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, myelodysplastic syndromes (MDS), myelofibrosis, polycythemia vera, Kaposi's sarcoma, splenic marginal zone lymphoma, multiple myeloma, plasmacytoma, intravascular large B-cell lymphoma, comprising administering to a subject in need thereof an effective amount of a compound of any one of embodiments 1-9 or a pharmaceutical composition according to embodiment 10.
20. A method for treating a fibrotic condition selected from idiopathic pulmonary fibrosis, systemic sclerosis, uterine fibroids (leiomyomata), sclerodoma, autoimmune hepatitis, primary biliary cirrhosis, sclerosing cholangitis, partial liver resection, acute liver necrosis, necrosis caused by toxin, viral hepatitis, shock, or anoxia, B-virus hepatitis, non-A/non-B hepatitis, chronic hepatitis C virus (HCV) infection, cirrhosis, alcoholic liver disease, including alcoholic cirrhosis, non-alcoholic steatohepatitis (NASH), hepatic failure, fulminant hepatic failure, late-onset hepatic failure, “acute-on-chronic” liver failure, kidney disease, and chronic kidney disease, comprising administering to a subject in need thereof an effective amount of a compound of any one of embodiments 1-9 or a pharmaceutical composition according to embodiment 10.
21. A method for treating cytokine release syndrome, comprising administering to a subject in need thereof an effective amount of a compound of any one of embodiments 1-9 or a pharmaceutical composition according to embodiment 10.
22. A method for treating a condition selected from endometriosis, uterine fibroids (leiomyomata), menorrhagia, adenomyosis, primary and secondary dysmenorrhoea (including symptoms of dyspareunia, dyschexia and chronic pelvic pain), chronic pelvic pain syndrome, precocious puberty, cervical ripening, breast carcinoma, colon carcinoma, familial adenomatous polyposis, colorectal adenomas, endometrial carcinoma, prostate carcinoma, pulmonary carcinoma, testicular carcinoma, gastric carcinoma, macular degeneration, inflammatory and neuropathic pain conditions, migraine, headache, cluster headache, inflammation induced pain, cancer pain, polycystic kidney disease and polycystic ovarian syndrome, comprising administering to a subject in need thereof an effective amount of a compound of any one of embodiments 1-9 or a pharmaceutical composition according to embodiment 10.
23. A method for treating a vascular disorder, comprising administering to a subject in need thereof an effective amount of a compound of any one of embodiments 1-9 or a pharmaceutical composition according to embodiment 10.
24. The method of embodiment 23, wherein the vascular disorder is selected from head injury, stroke, aneurysm, vascular dementia, vascular cognitive impairment and dementia (VCID), cerebral small vessel disease, subcortical ischemic vascular disease, mixed dementia, and ischemic vascular damage.
25. A method for treating a condition selected from Alzheimer's disease, ALS, multiple sclerosis, Parkinson's disease and Huntington's disease, comprising administering to a subject in need thereof an effective amount of a compound of any one of embodiments 1-9 or a pharmaceutical composition according to embodiment 10.

The present disclosure contemplates, among other things, the following numbered embodiments:
1. A compound having the structure of Formula (III)

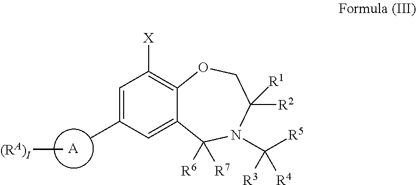

wherein
   Ring A is a bicyclic heterocycle having one or more nitrogen atoms; or Ring A is Ring A′;
   each R^{A} is selected from halogen; —CN, —C₁₋₄ alkyl, —C₁₋₄ haloalkyl, —(C₁₋₄ alkyl)O(C₁₋₄ alkyl), —C(O)OH, —C(O)O(C₁₋₄ alkyl), —C(O)NH₂, —C(O)NH(C₁₋₄ alkyl), —C(O)N(C₁₋₄ alkyl)₂, —NH₂, —NH(C₁₋₄ alkyl), —N(C₁₋₄ alkyl)₂, —OH, —O(C₁₋₄ alkyl), —O(C₁₋₄ haloalkyl), —S(C₁₋₄ alkyl), —SO₂C₁₋₄ alkyl, —SO₂NHC₁₋₄ alkyl, substituted or unsubstituted C₃₋₆ cycloalkyl, or substituted or unsubstituted 3- to 6-membered heterocyclyl; or two R^{A} taken together form a carbonyl;
   Ring A′ is selected from the group consisting of:

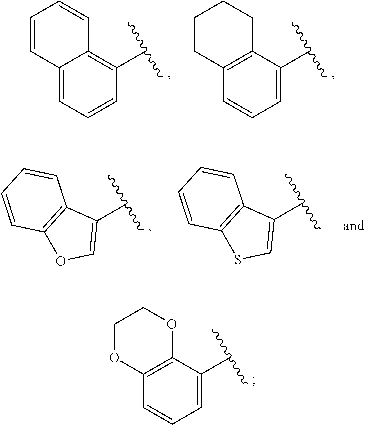 [SMILES: *c1cccc2ccccc12 ; *c1cccc2c1CCCC2 ; *c1coc2ccccc12 ; *c1csc2ccccc12 ; *c1cccc2c1OCCO2]

R¹ and R² are independently selected from hydrogen, —C₁₋₄ alkyl, —CN, —C(O)NR^{c}R^{c}, or together R¹ and R² form an oxo, C₃₋₆ membered cycloalkyl or 3-6 membered cycloheteroalkyl;
X is selected from hydrogen and halogen;
R³ and R⁴ are independently selected from hydrogen, —C₁₋₄ alkyl, together R³ and R⁴ form an oxo, or one of R³ and R⁴ form a double bond with R⁵ and the other of R³ and R⁴ is hydrogen or —C₁₋₄ alkyl;
R⁵ is selected from —(CH₂)ₘ—R^{b}, —(CHR^{a})ₘ—R^{b}, —O—(CH₂)ₘ—R^{b}, —O—(CHR^{a})ₘ—R^{b}, —C(O)NH—(CH₂)ₘ—R^{b}, —C(O)NH—(CHR^{a})ₘ—R^{b};
R⁶ and R⁷ are independently selected from hydrogen, —C₁₋₄ alkyl, or together R⁶ and R⁷ form an oxo;
each R^{a} is independently selected from the group consisting of hydrogen, (C₁₋₆) alkyl and (C₃₋₈) cycloalkyl;
R⁶ is selected from

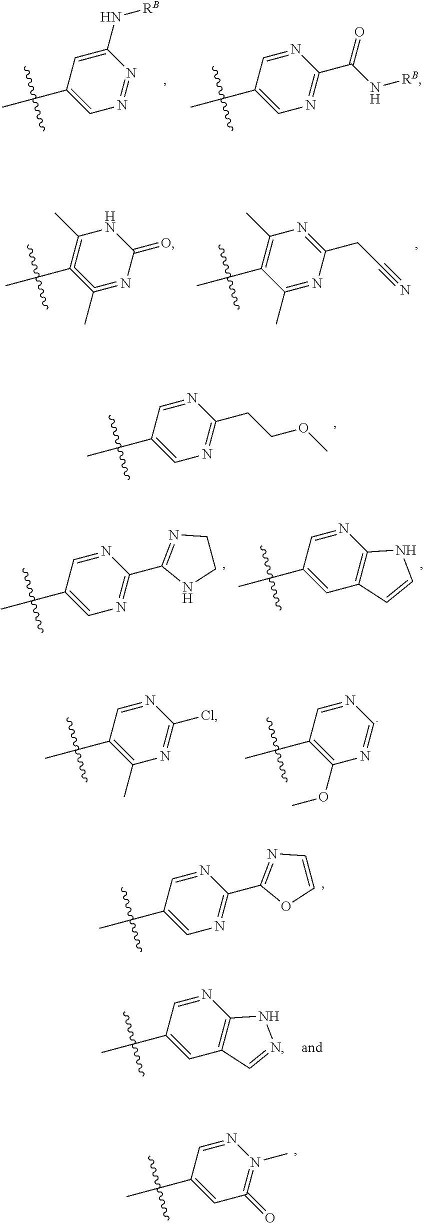

each R^{B} is selected from —C(O)(C₁₋₄ alkyl), —C(O)OH, —C(O)O(C₁₋₄ alkyl), —C(O)NH₂, —C(O)NH(C₁₋₄ alkyl), —C(O)N(C₁₋₄ alkyl)₂, —S(O)(C₁₋₄ alkyl), —S(O)(NH)(C₁₋₄ alkyl), —S(O)₂(C₁₋₄ alkyl), —S(O)₂NH₂, —S(O₂)NHCH₃, —S(O)₂(C₆₋₁₃aryl); and —S(O)₂NHS(O)₂ (C₁₋₄ alkyl);
each R^{c} is independently R^{a}, or, alternatively, two R^{c} are taken together with the nitrogen atom to which they are bonded to form a 5 to 8-membered cycloheteroalkyl or 5-membered heteroaryl, which may optionally include one or more of the same or different additional heteroatoms and which may optionally be substituted with one or more of the same or different —C(O)R^{a} and R^{a} groups;
each R^{d} is independently selected from hydrogen and (C₁₋₆) alkyl optionally substituted with 5-membered heteroaryl, 6-membered heteroaryl and 6-membered aryl, each optionally substituted with 1, 2 or 3 groups selected from halogen, C₁₋₄ alkyl, and —O(C₁₋₄ alkyl);
l is 0 to 3;
m is 0 to 3; and
n is 0 to 3.
2. The compound of embodiment 1, wherein R^{B} is selected from —S(O)₂(C₁₋₄ alkyl), —S(O)₂NH₂, —S(O₂)NHCH₃, and —S(O)₂(C₆₋₁₃aryl).
3. The compound of embodiment 1, wherein R^{b} is

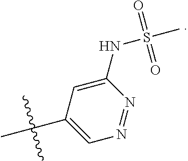 [SMILES: *c1cnnc(NS(C)(=O)=O)c1]

4. The compound of embodiment 1, wherein R^{b} is H

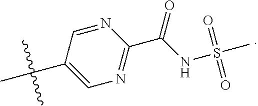 [SMILES: *c1cnc(C(=O)NS(C)(=O)=O)nc1]

5. The compound of embodiment 1, wherein Ring A is F

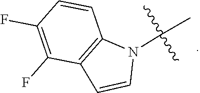 [SMILES: *n1ccc2c(F)c(F)ccc21]

6. A compound having the structure:

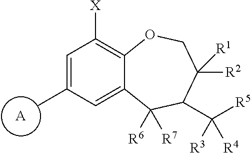

wherein Ring A is selected from the group consisting of

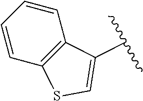 [SMILES: *c1csc2ccccc12]

optionally substituted with 1-3 R^{A},

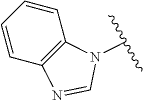 [SMILES: *n1cnc2ccccc21]

optionally substituted with 1-3 R^{A},

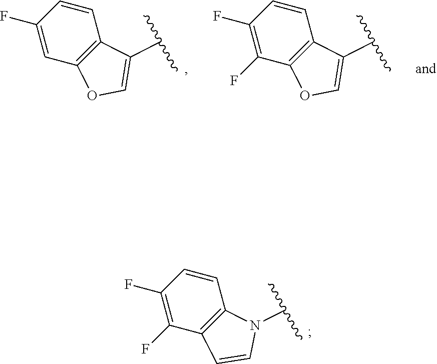 [SMILES: *c1coc2cc(F)ccc12 ; *c1coc2c(F)c(F)ccc12 ; *n1ccc2c(F)c(F)ccc21]

each R^{A} is selected from halogen; —CN, —C₁₋₄ alkyl, —C₁₋₄ haloalkyl, —(C₁₋₄ alkyl)O(C₁₋₄ alkyl), —C(O)OH, —C(O)O(C₁₋₄ alkyl), —C(O)NH₂, —C(O)NH(C₁₋₄ alkyl), —C(O)N(C₁₋₄ alkyl)₂, —NH₂, —NH(C₁₋₄ alkyl), —N(C₁₋₄ alkyl)₂, —OH, —O(C₁₋₄ alkyl), —O(C₁₋₄ haloalkyl), —S(C₁₋₄ alkyl), —SO₂C₁₋₄ alkyl, —SO₂NHC₁₋₄ alkyl, substituted or unsubstituted C₃₋₆ cycloalkyl, or substituted or unsubstituted 3- to 6-membered heterocyclyl; or two R^{A} taken together form a carbonyl;
R¹ and R² are independently selected from hydrogen, —C₁₋₄ alkyl, —CN, —C(O)NR^{c}R^{c}, or together R¹ and R² form an oxo, C₃₋₆ membered cycloalkyl or 3-6 membered cycloheteroalkyl;
X is selected from hydrogen and halogen;
R³ and R⁴ are independently selected from hydrogen, —C₁₋₄ alkyl, together R³ and R⁴ form an oxo, or one of R³ and R⁴ form a double bond with R⁵ and the other of R³ and R⁴ is hydrogen or —C₁₋₄ alkyl;
R⁵ is selected from —(CH₂)ₘ—R^{b}, —(CHR^{a})ₘ—R^{b}, —O—(CH₂)ₘ—R^{b}, —O—(CHR^{a})ₘ—R^{b}, —C(O)NH—(CH₂)ₘ—R^{b}, —C(O)NH—(CHR^{a})ₘ—R^{b};
R⁶ and R⁷ are independently selected from hydrogen, —C₁₋₄ alkyl, or together R⁶ and R⁷ form an oxo;
each R^{a} is independently selected from the group consisting of hydrogen, (C₁₋₆) alkyl and (C₃₋₈) cycloalkyl;
R^{b} is selected from

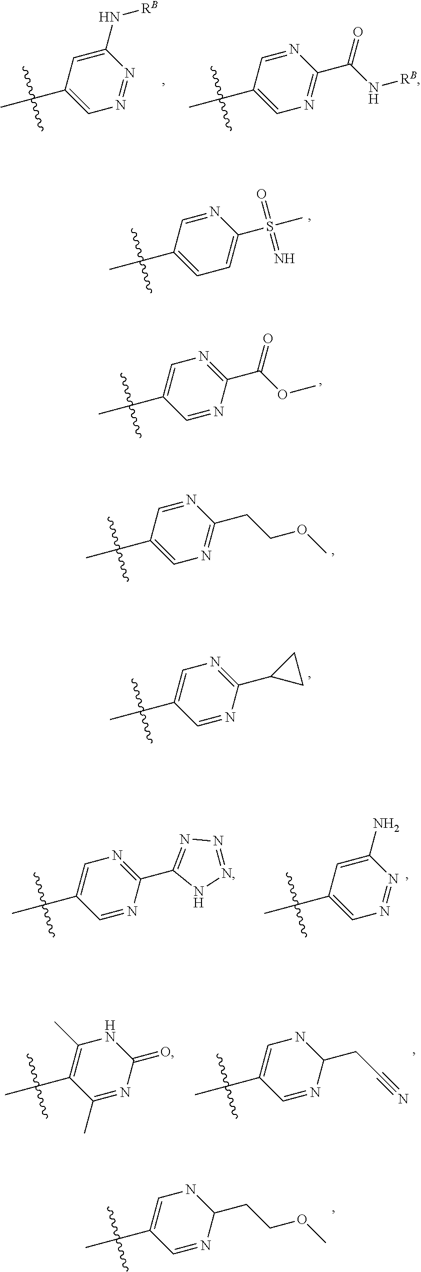

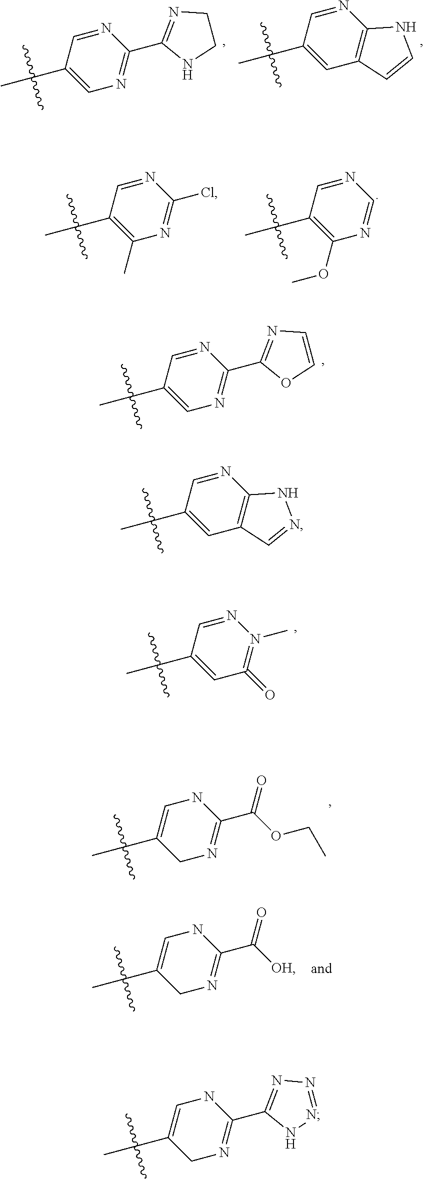

each R^{B} is selected from —C(O)(C₁₋₄ alkyl), —C(O)OH, —C(O)O(C₁₋₄ alkyl), —C(O)NH₂—C(O)NH(C₁₋₄ alkyl), —C(O)N(C₁₋₄ alkyl)₂, —S(O)(C₁₋₄ alkyl), —S(O)(NH)(C₁₋₄ alkyl), —S(O)₂(C₁₋₄ alkyl), —S(O)₂NH₂, —S(O₂)NHCH₃, —S(O)₂(C₆₋₁₃aryl); and —S(O)₂NHS(O)₂ (C₁₋₄ alkyl);
each R^{c} is independently R^{a}, or, alternatively, two R^{c} are taken together with the nitrogen atom to which they are bonded to form a 5 to 8-membered cycloheteroalkyl or 5-membered heteroaryl, which may optionally include one or more of the same or different additional heteroatoms and which may optionally be substituted with one or more of the same or different —C(O)R^{a} and R^{a} groups;
each R^{d} is independently selected from hydrogen and (C₁₋₆) alkyl optionally substituted with 5-membered heteroaryl, 6-membered heteroaryl and 6-membered aryl, each optionally substituted with 1, 2 or 3 groups selected from halogen, C₁₋₄ alkyl, and —O(C₁₋₄ alkyl);
m is 0 to 3; and
n is 0 to 3.
7. A compound having the structure:

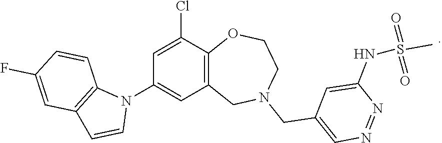 [SMILES: CS(=O)(=O)Nc1cc(CN2CCOc3c(Cl)cc(-n4ccc5cc(F)ccc54)cc3C2)cnn1]

8. A compound having the structure:

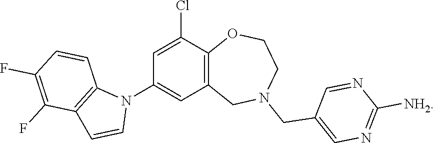 [SMILES: Nc1ncc(CN2CCOc3c(Cl)cc(-n4ccc5c(F)c(F)ccc54)cc3C2)cn1]

9. A compound having the structure:

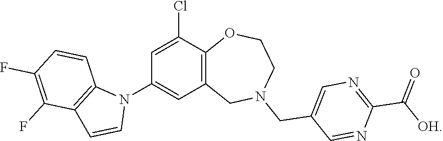 [SMILES: O=C(O)c1ncc(CN2CCOc3c(Cl)cc(-n4ccc5c(F)c(F)ccc54)cc3C2)cn1]

10. A compound having the structure:

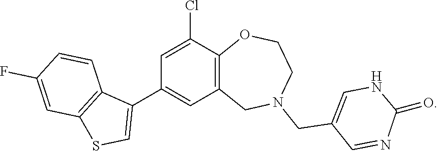 [SMILES: O=c1ncc(CN2CCOc3c(Cl)cc(-c4csc5cc(F)ccc45)cc3C2)c[nH]1]

11. A compound having the structure:

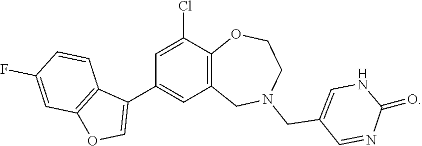 [SMILES: O=c1ncc(CN2CCOc3c(Cl)cc(-c4coc5cc(F)ccc45)cc3C2)c[nH]1]

12. A compound having the structure:

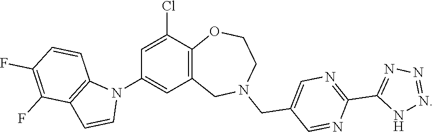 [SMILES: Fc1ccc2c(ccn2-c2cc(Cl)c3c(c2)CN(Cc2cnc(-c4nnn[nH]4)nc2)CCO3)c1F]

13. The compound of any one of embodiments 1-12 in the form of a pharmaceutically acceptable salt.
14. A pharmaceutical composition comprising a compound of any one of embodiments 1-13, and a pharmaceutically acceptable excipient.
15. A method for modulating prostaglandin E2 receptor 2 (EP2), comprising contacting the EP2 with a compound of any one of embodiments 1-13 or a composition according to embodiment 14.
16. The method of embodiment 15, wherein the contacting is in vitro.
17. The method of embodiment 15, wherein the contacting is in vivo.
18. A method for treating a disease or condition that would benefit from the modulation of prostaglandin E2 receptor 2 (EP2), comprising administering to a subject in need thereof an effective amount of a compound of any one of embodiments 1-13 or a composition according to embodiment 14.
19. A method for treating a disease or condition selected from neurological disorders, inflammatory disorders, autoimmune disorders, fibrotic disorders, allergic conditions, and combinations thereof, comprising administering to a subject in need thereof an effective amount of a compound of any one of embodiments 1-13 or a composition according to embodiment 14.
20. A method for treating a neurological disease selected from amyotrophic lateral sclerosis (ALS), epilepsy, dementia, Alzheimer's disease, delirium, post-operative cognitive dysfunction, including post-operative neurocognitive disorder, postoperative cognitive decline, postoperative delirium, vascular dementia, frontotemporal dementia, Lewy body dementia, head injury, concussion, chronic traumatic encephalopathy, intracerebral hemorrhage, hematoma, post-operative cognitive decline and dementia, diabetic retinopathy, macular degeneration, macular edema, multiple sclerosis, vasogenic edema including edema caused by treatment with antibody therapies (e.g. ARIA-E), migraine, Huntington's disease, ALS, and Parkinson's disease, comprising administering to a subject in need thereof an effective amount of a compound of any one of embodiments 1-13 or a composition according to embodiment 14.
21. A method for treating an inflammatory or allergic disorder selected from systemic lupus erythematosus, rheumatoid arthritis, type I diabetes mellitus, inflammatory bowel disease, biliary cirrhosis, uveitis, multiple sclerosis, Crohn's disease, hyperimmunoglobulinemia D and periodic fever syndrome, cryopyrin-associated periodic syndromes, Schnitzler's syndrome, systemic juvenile idiopathic arthritis, adult's onset Still's disease, ulcerative colitis, necrotizing enterocolitis (NEC), peritonitis, gout, gout flares, gouty arthritis, bullous pemphigoid, sarcoidosis, psoriasis, autoimmune myositis, Wegener's granulomatosis, ichthyosis, Graves ophthalmopathy, asthma, atopic asthma, allergic asthma, atopic bronchial IgE-mediated asthma, non-atopic asthma, bronchial asthma, non-allergic asthma, essential asthma, true asthma, intrinsic asthma caused by pathophysiologic disturbances, essential asthma of unknown or unapparent cause, emphysematous asthma, exercise-induced asthma, emotion-induced asthma, extrinsic asthma caused by environmental factors, cold air induced asthma, occupational asthma, infective asthma caused by or associated with bacterial, fungal, protozoal, or viral infection, incipient asthma, wheezy infant syndrome, bronchiolitis, cough variant asthma or drug-induced asthma), allergic bronchopulmonary aspergillosis (ABPA), allergic rhinitis, perennial allergic rhinitis, perennial rhinitis, vasomotor rhinitis, post-nasal drip, purulent or non-purulent sinusitis, acute or chronic sinusitis, and ethmoid, frontal, maxillary, and sphenoid sinusitis, comprising administering to a subject in need thereof an effective amount of a compound of any one of embodiments 1-13 or a composition according to embodiment 14.
22. A method for treating an immune regulatory disorder selected from tissue or organ transplant, graft-versus-host diseases brought about by transplantation, rheumatoid arthritis, systemic lupus erythematosus, Hashimoto's thyroiditis, multiple sclerosis, systemic sclerosis, myasthenia gravis, type I diabetes, uveitis, posterior uveitis, allergic encephalomyelitis, glomerulonephritis, postinfectious autoimmune diseases, rheumatic fever and post-infectious glomerulonephritis, inflammatory and hyperproliferative skin diseases, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, celiac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, chronic lymphocytic leukemia, arteriosclerosis, atherosclerosis, aortitis syndrome, polyarteritis nodosa, myocardosis, scleroderma, Wegener's granuloma, Sjögren's syndrome, adiposis, eosinophilic fascitis, lesions of gingiva, periodontium, alveolar bone, substantia ossea dentis, glomerulonephritis, alopecia areata, male pattern alopecia or alopecia senilis by preventing epilation or providing hair germination and/or promoting hair generation and hair growth, vitiligo, muscular dystrophy, pyoderma and Sezary's syndrome, Addison's disease, ischemia-reperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA ballous dermatitis and cement dermatitis, gingivitis, periodontitis and sepsis, comprising administering to a subject in need thereof an effective amount of a compound of any one of embodiments 1-13 or a composition according to embodiment 14.
23. A method for treating a proliferative disorder selected from, solid tumors, carcinoma of the brain, kidney, liver, adrenal gland, bladder, breast, stomach, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins lymphoma, a mammary carcinoma, follicular carcinoma, undifferentiated carcinoma, papillary carcinoma, seminoma, melanoma, IL-1 driven disorders, ABC diffuse large B-cell lymphoma (DLBCL), Waldenström's macroglobulinemia, Hodgkin's lymphoma, primary cutaneous T-cell lymphoma, smoldering or indolent multiple myeloma, hematological malignancies, including leukemia, acute myeloid leukemia (AML), DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, myelodysplastic syndromes (MDS), myelofibrosis, polycythemia vera, Kaposi's sarcoma, splenic marginal zone lymphoma, multiple myeloma, plasmacytoma, intravascular large B-cell lymphoma, comprising administering to a subject in need thereof an effective amount of a compound of any one of embodiments 1-13 or a composition according to embodiment 14.
24. A method for treating a fibrotic condition selected from idiopathic pulmonary fibrosis, systemic sclerosis, uterine fibroids (leiomyomata), sclerodoma, autoimmune hepatitis, primary biliary cirrhosis, sclerosing cholangitis, partial liver resection, acute liver necrosis, necrosis caused by toxin, viral hepatitis, shock, or anoxia, B-virus hepatitis, non-A/non-B hepatitis, chronic hepatitis C virus (HCV) infection, cirrhosis, alcoholic liver disease, including alcoholic cirrhosis, non-alcoholic steatohepatitis (NASH), hepatic failure, fulminant hepatic failure, late-onset hepatic failure, “acute-on-chronic” liver failure, kidney disease, and chronic kidney disease, comprising administering to a subject in need thereof an effective amount of a compound of any one of embodiments 1-13 or a composition according to embodiment 14.
25. A method for treating cytokine release syndrome, comprising administering to a subject in need thereof an effective amount of a compound of any one of embodiments 1-13 or a composition according to embodiment 14.
26. A method for treating a condition selected from endometriosis, uterine fibroids (leiomyomata), menorrhagia, adenomyosis, primary and secondary dysmenorrhoea (including symptoms of dyspareunia, dyschexia and chronic pelvic pain), chronic pelvic pain syndrome, precocious puberty, cervical ripening, breast carcinoma, colon carcinoma, familial adenomatous polyposis, colorectal adenomas, endometrial carcinoma, prostate carcinoma, pulmonary carcinoma, testicular carcinoma, gastric carcinoma, macular degeneration, inflammatory and neuropathic pain conditions, migraine, headache, cluster headache, inflammation induced pain, cancer pain, polycystic kidney disease and polycystic ovarian syndrome, comprising administering to a subject in need thereof an effective amount of a compound of any one of embodiments 1-13 or a composition according to embodiment 14.
27. A method for treating a vascular disorder, comprising administering to a subject in need thereof an effective amount of a compound of any one of embodiments 1-13 or a composition according to embodiment 14.
28. The method of embodiment 24, wherein the vascular disorder is selected from head injury, stroke, aneurysm, vascular dementia, vascular cognitive impairment and dementia (VCID), cerebral small vessel disease, subcortical ischemic vascular disease, mixed dementia, and ischemic vascular damage.
29. A method for treating a condition selected from Alzheimer's disease, ALS, multiple sclerosis, Parkinson's disease and Huntington's disease, comprising administering to a subject in need thereof an effective amount of a compound of any one of embodiments 1-13 or a composition according to embodiment 14.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1-13. (canceled)

14. A compound having the formula 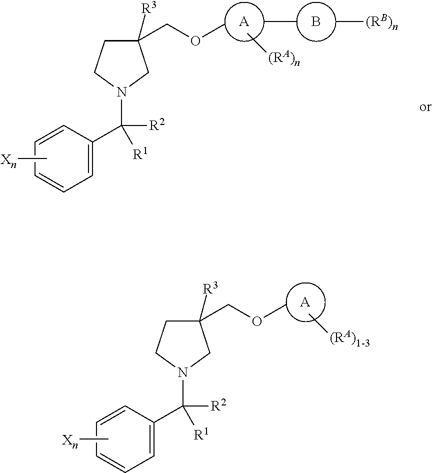
wherein
R¹ and R² are independently selected from R^{a}, —OR^{d}, —C₁₋₄ haloalkyl, or together R¹ and R² form an oxo, C₃₋₆ membered cycloalkyl or 3-6 membered cycloheteroalkyl;
R³ is —CH(R^{b})₂, —C(R^{b})₃, —S(O)₂R^{d}, —S(O)₂OR^{d}, —S(O)₂NR^{c}R^{c}, —S(O)(NR^{a})R^{d}, —S(O)₂N(R^{a})S(O)₂R^{a}, —S(O)₂N(R^{a})S(O)₂NR^{c}R^{c}, —C(O)R^{d}, —C(O)N(R^{a})C(O)R^{d}, —C(O)OR^{d}, —C(O)NR^{c}R^{c};
Ring A and Ring B independently are selected from phenyl and cycloalkyl;
X is selected from hydrogen and R^{b};
each R^{A} is independently selected from halogen; —CN, —C₁₋₄ alkyl, and —C₁₋₄ haloalkyl;
each R^{B} is independently selected from the group consisting of halogen, —CN, —C₁₋₄ alkyl, —C₁₋₄ haloalkyl, —C₁₋₄ aminoalkyl, —C₁₋₄ hydroxyalkyl, —C₁₋₄ methoxyalkyl, —(C₁₋₄ alkyl)O(C₁₋₄ alkyl), —C(O)(C₁₋₄ alkyl), —C(O)OH, —C(O)O(C₁₋₄ alkyl), —C(O)NR^{c}R^{c}, —NR^{c}R^{c}, —NH(C₃₋₆ cycloalkyl), —NH(C₃₋₆ heterocycloalkyl) and substituted or unsubstituted C₃₋₆ cycloalkyl;
each R^{a} is independently selected from the group consisting of hydrogen, (C₁₋₆) alkyl and (C₃₋₈) cycloalkyl;
each R^{b} is independently selected from the group consisting of —OR^{d}, —OCF₂H, —OCF₃, —NR^{c}R^{c}, halogen, —CF₃, —CN, —S(O)₂R^{d}, —S(O)₂OR^{d}, —S(O)₂NR^{c}R^{c}, —C(O)R^{d}, —C(O)OR^{d}, —C(O)NR^{c}R^{c},
each R^{c} is independently R^{a}, or, alternatively, two R^{c} are taken together with the nitrogen atom to which they are bonded to form a 5 to 8-membered cycloheteroalkyl or 5-membered heteroaryl, which may optionally include one or more of the same or different additional heteroatoms and which may optionally be substituted with one or more of the same or different —C(O)R^{a} and R^{a} groups;
each R^{d} is independently selected from hydrogen and (C₁₋₆) alkyl optionally substituted with 1, 2 or 3 groups selected from halogen and —O(C₁₋₄ alkyl); and
n is, for each occurrence, independently, 0 to 3;
provided that the compound is not a compound disclosed in PCT/US22/34903.

15. The compound of claim 14, wherein Ring A or Ring B, or both is bicyclic.

16. The compound of claim 14, wherein Ring A or Ring B, or both is bridged bicyclic.

17. The compound of claim 14, wherein Ring A or Ring B, or both is bridged bicyclic cycloalkyl.

18. The compound of claim 14, wherein Ring A is bridged cycloalkyl.

19. The compound of claim 14, wherein Ring B is bridged cycloalkyl.

20. The compound of claim 14, wherein Ring A or Ring B is C₃₋₆ cycloalkyl.

21. The compound of claim 14, having the structure

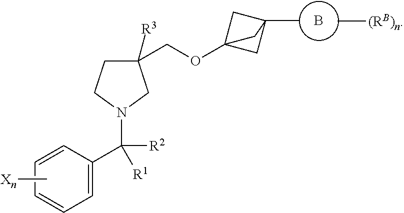

22. The compound of claim 14, having the structure

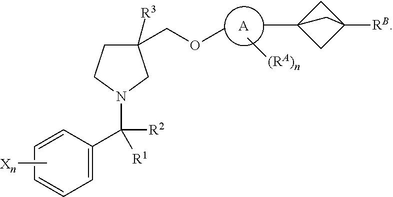

23. The compound of claim 14, having the structure

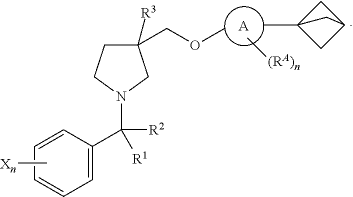

24. The compound of claim 14, wherein R^{B} is —CN, —C(O)(C₁₋₄ alkyl), —C(O)OH, —C(O)O(C₁₋₄ alkyl) or —C(O)NR^{c}R^{c}.

25-28. (canceled)

29. The compound of claim 14, wherein R³ is selected from S(O)₂R^{d}, —S(O)(NR^{a})R^{d}, —S(O)₂OR^{d}, —S(O)₂NR^{c}R^{c}, —S(O)₂N(R^{a})S(O)₂R^{a} and —S(O)₂N(R^{a})S(O)₂NR^{c}R^{c}.

30. The compound of claim 22, wherein R³ is selected from —C(O)R^{d}, —C(O)N(R^{a})C(O)R^{d}, —C(O)OR^{d} and —C(O)NR^{c}R^{c}.

31-43. (canceled)

44. A compound that is:

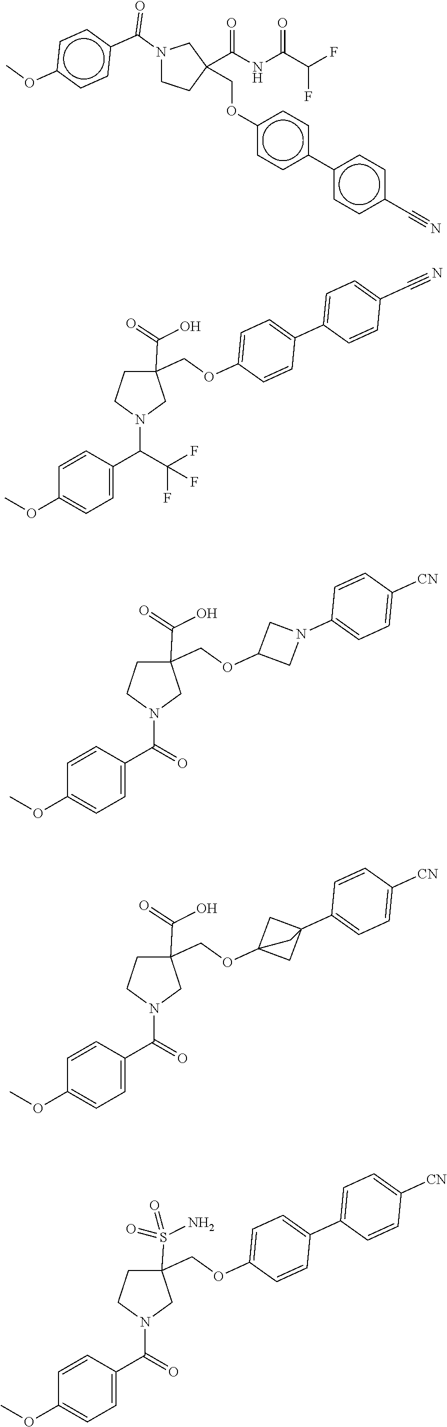

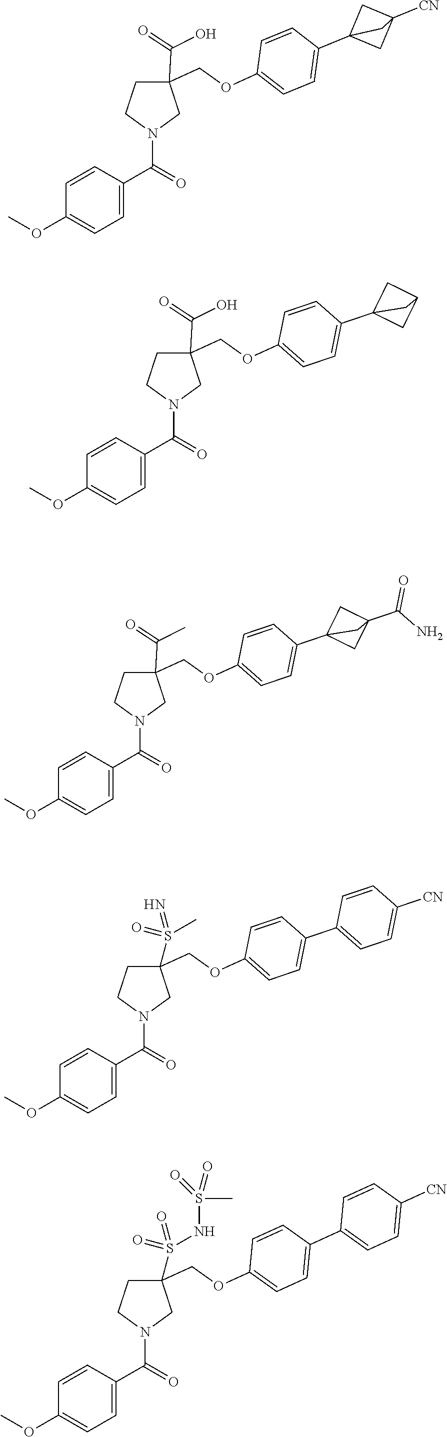 [SMILES: COc1ccc(C(=O)N2CCC(COc3ccc(C45CC(C#N)(C4)C5)cc3)(C(=O)O)C2)cc1 ; COc1ccc(C(=O)N2CCC(COc3ccc(C45CC(C4)C5)cc3)(C(=O)O)C2)cc1 ; COc1ccc(C(=O)N2CCC(COc3ccc(C45CC(C(N)=O)(C4)C5)cc3)(C(C)=O)C2)cc1 ; COc1ccc(C(=O)N2CCC(COc3ccc(-c4ccc(C#N)cc4)cc3)(S(C)(=N)=O)C2)cc1 ; COc1ccc(C(=O)N2CCC(COc3ccc(-c4ccc(C#N)cc4)cc3)(S(=O)(=O)NS(C)(=O)=O)C2)cc1]

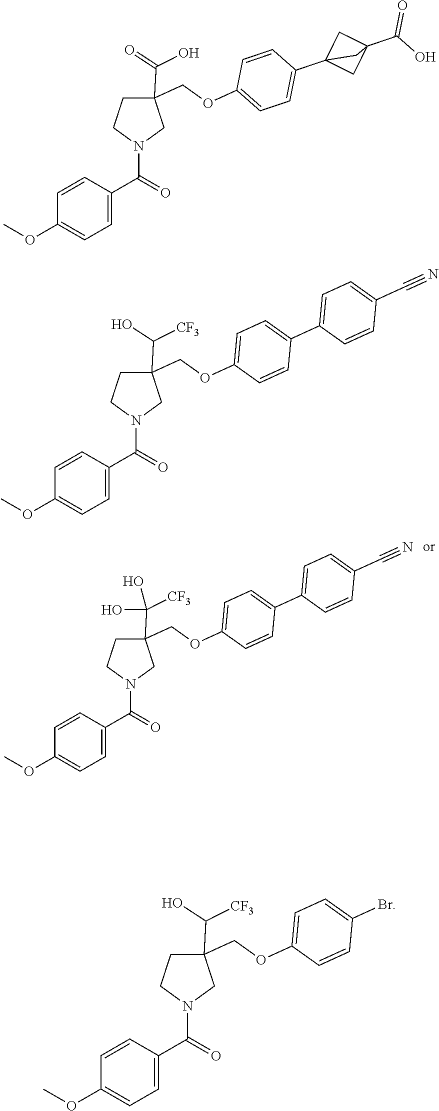 [SMILES: COc1ccc(C(=O)N2CCC(COc3ccc(C45CC(C(=O)O)(C4)C5)cc3)(C(=O)O)C2)cc1 ; COc1ccc(C(=O)N2CCC(COc3ccc(-c4ccc(C#N)cc4)cc3)(C(O)C(F)(F)F)C2)cc1 ; COc1ccc(C(=O)N2CCC(COc3ccc(-c4ccc(C#N)cc4)cc3)(C(O)(O)C(F)(F)F)C2)cc1 ; COc1ccc(C(=O)N2CCC(COc3ccc(Br)cc3)(C(O)C(F)(F)F)C2)cc1]

45. The compound of claim 14 in the form of a pharmaceutically acceptable salt.

46. A pharmaceutical composition comprising a compound of claim 14, and a pharmaceutically acceptable excipient.

47. A method for modulating prostaglandin E2 receptor 2 (EP2), comprising contacting the EP2 with a compound of claim 14.

48. The method of claim 47, wherein the contacting is in vitro.

49. The method of claim 47, wherein the contacting is in vivo.

50. A method for treating a disease or condition that would benefit from the modulation of prostaglandin E2 receptor 2 (EP2), comprising administering to a subject in need thereof an effective amount of a compound of claim 14.

51-67. (canceled)
